(19) United States
(12) Patent Application Publication
(10) Pub. No.: US 2003/0220373 A1
(43) Pub. Date: Nov. 27, 2003

(54) Therapeutic uses of PPAR mediators

(76) Inventors: Michael Jaye, Glenside, PA (US); Nicolas Duverger, Paris (FR); George Searfoss, New Palestine, IN (US); Anne Minnich, Flemington, NJ (US)

Correspondence Address: ROSS J. OEHLER, AVENTIS PHARMACEUTICALS INC., ROUTE 202-206, MAIL CODE: D303A, BRIDGEWATER, NJ, 08807, US

(21) Appl. No.: 10/237,578

(22) Filed: Sep. 9, 2002

Related U.S. Application Data
(63) Continuation of application No. PCT/EP01/02482, filed on Mar. 6, 2001.
(60) Provisional application No. 60/188,323, filed on Mar. 9, 2000.

(30) Foreign Application Priority Data
Jun. 6, 2000 (GB) 0013589.7

(51) Int. Cl. A61K 31/4439; A61K 31/426; A61K 31/202; A61K 31/557

(52) U.S. Cl. 514/342; 514/367; 514/560; 514/573

(57) ABSTRACT

Use of PPAR mediators, and their pharmaceutical compositions, as ATP binding cassette transporter 1 (ABC-1) expression modulators, wherein the PPAR ligand receptor agonists of this invention are useful as inducers of ABC-1 expression.

### BACKGROUND OF THE INVENTION

This invention is directed to the use of PPAR mediators, and their pharmaceutical compositions, as ATP binding cassette transporter 1 (ABC-1) expression modulators, wherein the PPAR ligand receptor agonists of this invention are useful as inducers of ABC-1 expression.

### FIELD OF THE INVENTION

Peroxisome proliferator-activated receptors (PPAR) are three receptors: PPARα, PPARδ and PPARγ. These are encoded by different genes (Motojima, Cell Structure and Function, 18:267-277, 1993). Moreover, 2 isoforms of PPARγ also exist, PPARγ₁ and γ₂. These 2 proteins differ in their NH₂-terminal-30 amino acids and are the result of alternative promoter usage and differential mRNA splicing (Vidal-Puig, Jimenez, Linan, Lowell, Hamann, Hu, Spiegelman, Flier, Moller, J. Clin. Invest., 97:2553-2561, 1996).

Biological processes modulated by PPAR are those modulated by receptors, or receptor combinations, which are responsive to the PPAR ligand receptor binders described herein. Biological processes known to be modulated by PPAR include, for example, cell differentiation to produce lipid accumulating cells, regulation of insulin sensitivity and blood glucose levels, which are involved in hypoglycemia/hyperinsulinism (resulting from, for example, abnormal pancreatic beta cell function, insulin secreting tumors and/or autoimmune hypoglycemia due to autoantibodies to insulin, the insulin receptor, or autoantibodies that are stimulatory to pancreatic beta cells), macrophage differentiation which lead to the formation of atherosclerotic plaques, inflammatory response, carcinogenesis, hyperplasia, and adipocyte differentiation.

Peroxisomes are cellular organelles which play a role in controlling the redox potential and oxidative stress of cells by metabolizing a variety of substrates such as hydrogen peroxide. There are a number of disorders associated with oxidative stress. For example, inflammatory response to tissue injury, pathogenesis of emphysema, ischemia-associated organ injury (shock), doxorubicin-induced cardiac injury, drug-induced hepatotoxicity, atherosclerosis, and hyperoxic lung injuries, are each associated with the production of reactive oxygen species and a change in the reductive capacity of the cell. Therefore, it is envisaged that PPAR activators which control the redox potential and oxidative stress in cells, would be effective in the treatment of these disorders.

Peroxisome proliferators activate PPAR , which acts as a transcription factor, and causes differentiation, cell growth and proliferation of peroxisomes. PPAR activators are also thought to play a role in hyperplasia and carcinogenesis as well as altering the enzymatic capability of animal cells, such as rodent cells, but these PPAR activators appear to have minimal negative effects in human cells (Green, Biochem. Pharm. 43(3):393, 1992). Activation of PPAR results in the rapid increase of gamma glutamyl transpeptidase and catalase.

It is also known that PPAR agonists inhibit the inducible nitric oxide synthase (NOS) enzyme pathway and thus can be used in the therapeutic intervention of a wide variety of inflammatory diseases and other pathologies (Colville-Nash, et al., Journal of Immunology, 161, 978-84, 1998; Staels et al, Nature, 393, 790-3, 1998).

PPARα is activated by a number of medium and long-chain fatty acids and is involved in stimulating β-oxidation of fatty acids in tissues such as liver, heart, and brown adipose tissue (Isseman and Green, supra; Beck et al., Proc. R. Soc. Lond. 247:83-87, 1992; Gottlicher et al., Proc. Natl. Acad. Sci. USA 89:4653-4657, 1992). PPARα activators are also involved in substantial reduction in plasma triglycerides along with moderate reduction in LDL cholesterol, and they are used particularly for the treatment of hypertriglyceridemia, hyperlipidemia and obesity. PPARα is also known to be involved in inflammatory disorders. (Schoonjans, K., Current Opionion in Lipidology, 8, 159-66, 1997).

The human nuclear receptor PPARδ has been cloned from a human osteosarcoma cell cDNA library and is fully described in A. Schmidt et al., Molecular Endocrinology, 6:1634-1641 (1992), the contents of which are hereby incorporated herein by reference. It should be noted that PPARδ is also referred to in the literature as PPARδ and as NUC1, and each of these names refers to the same receptor. For example, in A. Schmidt et al., Molecular Endocrinology, 6: pp. 1634-1641, 1992, the receptor is referred to as NUC1. PPARδ is observed in both embryo and adult tissues. This receptor has been reported to be involved in regulating the expression of some fat-specific genes, and plays a role in the adipogenic process (Amri, E. et al., J. Biol. Chem. 270, 2367-71, 1995).

Atherosclerotic disease is known to be caused by a number of factors, for example, hypertension, diabetes, low levels of high density lipoprotein (HDL), and high levels of low density lipoprotein (LDL). It has recently been discovered that PPARδ agonists are useful in raising HDL levels and therefore useful in treating atherosclerotic diseases (Leibowitz et al.; WO/9728149) such as vascular disease, coronary heart disease, cerebrovascular disease and peripheral vessel disease. Coronary heart disease includes CHD death, myocardial infarction, and coronary revascularization. Cerebrovascular disease includes ischemic or hemorrhagic stroke and transient ischemic attacks.

The DNA sequences for the PPARγ receptors are described in Elbrecht et al., BBRC 224;43 1-437 (1996). PPARγ receptor subtypes are involved in activating adipocyte differentiation, and are not involved in stimulating peroxisome proliferation in the liver. Activation of PPARγ is implicated in adipocyte differentiation through the activation of adipocyte-specific gene expression (Lehmann, Moore, Smith-Oliver, Wilkison, Willson, Kliewer, J. Biol. Chem., 270:12953-12956, 1995).

Obesity is an excessive accumulation of adipose tissue. Recent work in this area indicates that PPARγ plays a central role in the adipocyte gene expression and differentiation. Excess adipose tissue is associated with the development of serious medical conditions, for example, non-insulin-dependent diabetes mellitus (NIDDM), hypertension, coronary artery disease, hyperlipidemia and certain malignancies. The adipocyte may also influence glucose homeostasis through the production of tumor necrosis factor α (TNFα) and other molecules.

Non-insulin-dependent diabetes mellitus (NIDDM), or Type II diabetes, is the more common form of diabetes, with 90-95% of hyperglycemic patients experiencing this form of the disease. In NIDDM there appears to be a reduction in the pancreatic β-cell mass, several distinct defects in insulin secretion or a decrease in tissue sensitivity to insulin. The symptoms of this form of diabetes include fatigue, frequent urination, thirst, blurred vision, frequent infections and slow healing of sores, diabetic nerve damage and renal disease.

Resistance to the metabolic actions of insulin is one of the key features of non-insulin dependent diabetes (NIDDM). Insulin resistance is characterised by impaired uptake and utilization of glucose in insulin-sensitive target organs, for example, adipocytes and skeletal muscle, and by impaired inhibition of hepatic glucose output. The functional insulin deficiency and the failure of insulin to supress hepatic glucose output results in fasting hyperglycemia. Pancreatic β-cells compensate for the insulin resistance by secreting increased levels of insulin. However, the β-cells are unable to maintain this high output of insulin, and, eventually, the glucose-induced insulin secretion falls, leading to the deterioration of glucose homeostasis and to the subsequent development of overt diabetes.

Hyperinsulinemia is also linked to insulin resistance, hypertriglyceridaemia and increased plasma concentration of low density lipoproteins. The association of insulin resistance and hyperinsulinemia with these metabolic disorders has been termed “Syndrome X” and has been strongly linked to an increased risk of hypertension and coronary artery disease.

Metformin is known in the art to be used in the treatment of diabetes in humans (U.S. Pat. No. 3,174,901). Metformin acts primarily to decrease liver glucose production. Troglitazone® is known to work primarily on enhancing the ability of skeletal muscle to respond to insulin and take up glucose. It is known that combination therapy comprising metformin and troglitazone can be used in the treatment of abnormalities associated with diabetes (DDT 3:79-88, 1998).

PPARγ activators, in particular Troglitazone®, have been found to convert cancerous tissue to normal cells in liposarcoma, a tumor of fat (PNAS 96:3951-3956, 1999). Furthermore, it has been suggested that PPARγ activators may be useful in the treatment of breast and colon cancer (PNAS 95:8806-8811, 1998, Nature Medicine 4:1046-1052, 1998).

Moreover, PPARγ activators, for example Troglitazone®, have been implicated in the treatment of polycystic ovary syndrome (PCO). This is a syndrome in women that is characterized by chronic anovulation and hyperandrogenism. Women with this syndrome often have insulin resistance and an increased risk for the development of noninsulin-dependent diabetes mellitus. (Dunaif, Scott, Finegood, Quintana, Whitcomb, J. Clin. Endocrinol. Metab., 81:3299, 1996.

Furthermore, PPARγ activators have recently been discovered to increase the production of progesterone and inhibit steroidogenesis in granulosa cell cultures and therefore may be useful in the treatment of climacteric. (U.S. Pat. No. 5,814,647 Urban et al. Sep. 29, 1998; B. Lohrke et al. Journal of Edocrinology, 159, 429-39, 1998). Climacteric is defined as the syndrome of endocrine, somatic and psychological changes occurring at the termination of the reproductive period in the female. The menstrual irregularities are episodes of prolonged menstrual bleeding caused by a loss of ovulation. The loss of ovulation is caused by a failure of development of ovarian follicles.

Although peroxisome proliferators, including fibrates and fatty acids, activate the transcriptional activity of PPAR's, only prostaglandin J₂ derivatives such as the arachidonic acid metabolite 15-deoxy-delta¹²,14-prostaglandin J₂ (15d-PGJ₂) have been identified as natural ligands specific for the PPARγ subtype, which also binds thiazolidinediones. This prostaglandin activates PPARγ-dependent adipogenesis, but activates PPARα only at high concentrations (Forman, Tontonoz, Chen, Brun, Spiegelman, Evans, Cell, 83:803-812, 1995; Kliewer, Lenhard, Wilson, Patel, Morris, Lehman, Cell, 83:813-819, 1995). This is further evidence that the PPAR family subtypes are distinct from one another in their pharmacological response to ligands.

It has been suggested that compounds activating both PPARα and PPARγ should be potent hypotriglyceridemic drugs, which could be used in the treatment of dyslipidemia associated with atherosclerosis, non-insulin dependent diabetes mellitus and Syndrome X. (Staels, B. et al., Curr. Pharm. Des., 3 (1), 1-14 (1997)). Syndrome X is the syndrome characterized by an initial insulin resistant state, generating hyperinsulinaemia, dyslipidaemia and impaired glucose tolerance, which can progress to non-insulin dependent diabetes mellitus (Type II diabetes), characterized by hyperglycemia.

ABC-1 gene, is a causal gene for pathologies linked to a cholesterol metabolism dysfunction inducing diseases such as atherosclerosis, more particularly disruption in the reverse transport of cholesterol, and more particularly familial HDL deficiencies (FHD), such as Tangier disease.

ABC (ATP-binding cassette) is a member of the ATP-dependent transporter proteins involved in membrane transport of various substrates, for example ions, amino acids, peptides, sugars, vitamins or steroid hormones. In particular, ABC-1 is involved in the control of cholesterol efflux from macrophages and in maintaining the level of circulating HDL (Lawn, R. M. et al. J. Clin. Invest. 104, R25-R31 (1999); and Brooks-Wilson, A. et al., Nature Genet. 22, 336-345 (1999)).

The ABC1 gene has been shown to be a causal gene for pathologies linked to a cholesterol metabolism dysfunction inducing diseases such as atherosclerosis, more particularly disruption in the reverse transport of cholesterol, and more particularly familial HDL deficiencies (FHD), such as Tangier disease. Nucleic acids corresponding to various exons and introns of the ABC1 gene have been described in U.S. application No. 60/147,128, filed on Aug. 4, 1999, the contents of which are hereby incorporated herein by reference. ABC1 cDNAs encoding the novel full length ABC1 protein and other exons and introns of the ABC1 gene has been described in European patent application EP 99.402 668.0., filed on Oct. 26, 1999, the contents of which are hereby incorporated herein by reference.

PPARα and PPARγ are transcription factors expressed in human macrophages (Chinetti, G. et al., J. Biol. Chem. 273, 25573-25580 (1998)) and are known to modulate lipoprotein metabolism. For example, activation of the PPAR pathway increases the level of HDL-cholesterol (Pineda Torra, I., Gervois, P. & Staels, B., Curr. Opin. Lipidol. 10, 151-159 (1999)). Patients who have Tangiers disease lack the functional ABC-1 and are defective in cholesterol efflux (Remaley, A. T. et al., Proc. Natl. Acad. Sci. USA 96, 12685-12690 (1999)).

Cholesterol is the metabolic precursor of steroid hormones and bile acids as well as an essential constituent of cell membranes. In humans and other animals, cholesterol is ingested in the diet and also synthesized by the liver and other tissues. Cholesterol is transported between tissues in the form of cholesteryl esters in LDLs and other lipoproteins.

High-density lipoproteins (HDL) are one of the four major classes of lipoproteins circulating in blood plasma. These lipoproteins are involved in various metabolic pathways such as lipid transport, the formation of bile acids, steroidogenesis, cell proliferation and, in addition, interfere with the plasma proteinase systems.

HDLs are perfect free cholesterol acceptors and, in combination with the cholesterol ester transfer proteins (CETP), lipoprotein lipase (LPL), hepatic lipase (HL) and lecithin:cholesterol acyltransferase (LCAT), play a major role in the reverse transport of cholesterol, that is to say the transport of excess cholesterol in the peripheral cells to the liver for its elimination from the body in the form of bile acid. It has been demonstrated that the HDLs play a central role in the transport of cholesterol from the peripheral tissues to the liver.

Various diseases linked to an HDL deficiency have been described, including Tangier and/or FHD disease, HDL deficiency, LCAT deficiency, and Fish-Eye Disease (FED). In addition, HDL-cholesterol deficiencies have been observed in patients suffering from malaria and diabetes (Kittl et al., 1992; Nilsson et al., 1990; Djoumessi, 1989; Mohanty et al., 1992; Maurois et al., 1985; Grellier et al., 1997; Agbedana et al., 1990; Erel et al., 1998; Cuisinier et al., 1990; Chander et al., 1998; Efthimiou et al., 1992; Baptista et al., 1996; Davis et al., 1993; Davis et al., 1995; Pirich et al., 1993; Tomlinson and Raper, 1996; Hager and Hajduk, 1997, Kwiterovich, 1995, Syvanne et al., 1995a, Syvanne et al., 1995b, and French et al., 1993). The deficiency involved in Tangier and/or FHD disease is linked to a cellular defect in the translocation of cellular cholesterol which causes a degradation of the HDLs and leads to a disruption in the lipoprotein metabolism. Nevertheless, for Tangier and/or FHD disease, the exact nature of the defect has not yet been precisely defined.

Tangier disease is an autosomal co-dominant condition characterized in the homozygous state by the absence of HDL-cholesterol (HDL-C) from plasma, hepatosplenomegaly, peripheral neuropathy, and frequently premature coronary artery disease (CAD). In heterozygotes, HDL-C levels are about one-half those of normal individuals. Impaired cholesterol efflux from macrophages leads to the presence of foam cells throughout the body, which may explain the increased risk of CAD in some Tangier disease families.

In Tangier disease patients, the HDL particles do not incorporate cholesterol from the peripheral cells, are not metabolized correctly, and are rapidly eliminated from the body. The plasma HDL concentration in these patients is therefore, extremely reduced and the HDLs no longer ensure the return of cholesterol to the liver. Cholesterol accumulates in these peripheral cells and causes characteristic clinical manifestations such as the formation of orange-colored tonsils. Furthermore, other lipoprotein disruptions, such as overproduction of triglycerides as well as increased synthesis and intracellular catabolism of phospholipids are also observed in Tangier disease patients.

Tangier disease, whose symptoms have been described above, is classified among the familial conditions linked to the metabolism of HDLs, which are the ones most commonly detected in patients affected by coronary diseases. Numerous studies have shown that a reduced level of HDL cholesterol is an excellent indicator of an individual's risk of developing or already having a cardiovascular condition. In this context, syndromes linked to HDL deficiencies have been of increasing interest for the past decade because they make it possible to increase understanding of the role of HDLs in atherogenesis.

Atherosclerosis is defined in histological terms by deposits (lipid or fibrolipid plaques) of lipids and of other blood derivatives in blood vessel walls, especially the large arteries (aorta, coronary arteries, carotid). These plaques, which are more or less calcified according to the degree of progression of the atherosclerotic process, may be coupled with lesions and are associated with the accumulation in the vessels of fatty deposits consisting essentially of cholesteryl esters. These plaques are accompanied by a thickening of the vessel wall, hypertrophy of the smooth muscle, appearance of foam cells (lipid-laden cells resulting from uncontrolled uptake of cholesterol by recruited macrophages) and accumulation of fibrous tissue. The atheromatous plaque protrudes markedly from the wall, endowing it with a stenosing character responsible for vascular occlusions by atheroma, thrombosis or embolism, which occur in those patients who are most affected. These lesions can lead to serious cardiovascular pathologies such as infarction, sudden death, cardiac insufficiency, and stroke.

Applicants have discovered that PPAR activators induce ABC-1 expression in humans cells. In addition, Applicants have discovered that PPAR activators decrease lipid accumulation, by increasing apoAl-induced cholesterol efflux from normal macrophages. . This discovery identifies a central role for PPARs in the control of the reverse cholesterol transport pathway by inducing ABC-1 mediated cholesterol removal from human macrophages.

Therefore, the present invention discloses the use of PPAR mediators, and their pharmaceutical compositions, in regulating ATP binding cassette transporter 1 (ABC-1) expression, as well as a number of therapeutic uses associated with it.

PPAR mediators useful for practicing the present invention, and the methods of making these compounds are described herein or are disclosed in the literature, for example Nafenopin (U.S. Pat. No. 5,726,041), UF-5 (WO 97/36579), ETYA: 5,8,11,14-eicosatetraynoic acid (Tontonez et al., Cell 79:1147-1156 (1994), it also purchasable from Sigma), GW2331: 2-(4-[2-(3-[2,4-difluorophenyl]1-1heptylureidoethyl]phenoxy)-2-methylbutyric acid (Sundseth et al., Proc. Natl. Acad. Sci. USA, 94, 4318, 1997), 15-deoxy-Δ^{12,14}-prostaglandin J₂ (Lohrke et al., Journal of Endocrinology 159, 429, 1998) AD 5075, clofibric, linoleic acid (Tontonoz et al. Cell, 79, 1147, 1994), BRL-49653: 5-[4-{2-[N-Methyl-N-(pyridin-2-yl)amino]ethoxy}benzyl]-thiazolidine-2,4-di one, (Japanese Patent Kokai Application No. Hei 1-131169 and in U.S. Pat. Nos. 5,002,953, 5,194,443, 5,232,925 and 5,260,445), fenofibrate, WR-1339: Tyloxapol®, (Lefebvre et al. Arteriosclerosis, Thrombosis, and Vasclular Biology, 17, 9, 1977), Pioglitazone: 5-{4-[2-(5-Ethylpyridin-2-yl)ethoxy]benzyl}thiazolidine-2,4-dione, (Japanese Patent Publication No. Sho 62-42903 and No. Hei 5-66956, U.S. Pat. Nos. 4,287,200, 4,340,605, 4,438,141, 4,444,779 and 4,725,610), Ciglitazone, (Lehmann et al. The Journal of Biological Chemistry, 270, 22, 12953, 1995), Englitazone: 5-(2-Benzyl-3,4-dihydro-2H-benzopyran-6-ylmethyl)-thiazolidine-2,4-dione (Japanese Patent Publication No. Hei 5-86953 and U.S. Pat. No. 4,703,052); Troglitazone: 5-[[4-[3,4-dihydro-6-hydro-6-hydroxy-2,5,-7,8-tetramethyl-2H-1-bnzopyran-2-yl)ethoxy]phenyl]methyl]-2,4-thiazolidinedione (U.S. Pat. No. 4,572,912), Wyl4,643 : pyrinixic acid (Biomol Research Laboratories, Plymouth Rock, Pa.), LY-171883 (Biomol Research Laboratories), AD 5075: 5-[[4-[2-hydroxy-2-(5-methyl-2-phenyl-4-oxazolyl)ethoxy]phenyl]methyl-2,4-thiazolidinedione (WO 97/10819, WO 97/12853, WO 97/10813, and WO 97/37656), 5-[[4-[2-(methyl-2-pyridinylamino)ethoxy]phenyl]methyl]-2,4-thiazolidinedi one, WAY-120,744, darglitazone (U.S. Pat. No. 5,972,881), and their pharmaceutically acceptable salts. Compounds useful for practicing the present invention, and methods of making these compounds are known. Some of these compounds are disclosed in WO 91/07107; WO 92/02520; WO 94/01433; WO 89/08651; JP Kokai 69383/92; U.S. Pat. Nos. 4,287,200; 4,340,605; 4,438,141; 4,444,779; 4,461,902; 4,572,912; 4,687,777; 4,703,052; 4,725,610; 4,873,255; 4,897,393; 4,897,405; 4,918,091; 4,948,900; 5,002,953; 5,061,717; 5,120,754; 5,132,317; 5,194,443; 5,223,522; 5,232,925; and 5,260,445, and Tontonez et al., Genes & Develop. 8:1224-1234 (1994), Tontonez et al., Cell 79:1147-1156 (1994), Lehmann et al., J. Biol. Chem. 270(22):1-4, 1995, Amri et al., J. Lipid Res. 32:1449-1456 (1991), Amri et al., J. Lipid Res. 32:1457-1463, (1991) and Grimaldi et al., Proc. Natl. Acad. Sci, USA 89:10930-10934 (1992). Further PPAR activators are disclosed in WO 99/20275. The disclosure of these publications are incorporated herein by reference in particular with respect to the active compounds disclosed therein, and methods of preparation thereof.

### SUMMARY OF THE INVENTION

The present invention is directed to PPAR mediators that are useful in regulating ABC-1 expression, as well as to a number of other pharmaceutical uses associated therewith. More particularly, the present invention is directed to PPAR agonists that are useful in inducing ABC-1 expression, as well as to a number of other pharmaceutical uses associated therewith.

The compounds for use according to the invention, including the new compounds of the present invention, are of Formula I

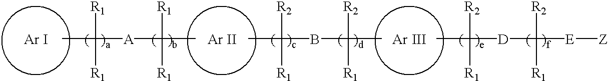

wherein:

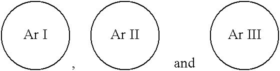

are independently aryl, fused arylcycloalkenyl, fused arylcycloalkyl, fused arylheterocyclenyl, fused arylheterocyclyl, heteroaryl, fused heteroarylcycloalkenyl, fused heteroarylcycloalkyl, fused heteroarylheterocyclenyl, or fused heteroarylheterocyclyl;

A is O, S, SO, SO₂, NR₅, a chemical bond,

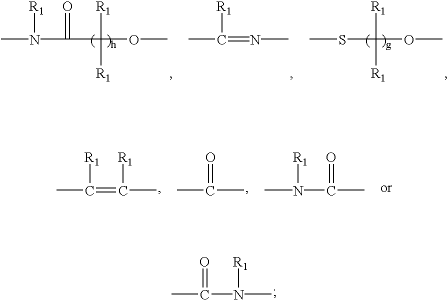

B is O, S, SO, SO₂, NR₄, a chemical bond,

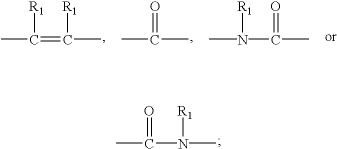

D is O, S, NR₄,

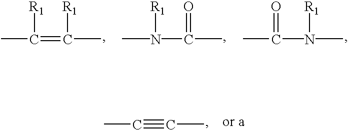

chemical bond;

E is a chemical bond or

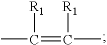

a is 0-4;

b is 0-4;

c is 0-4;

d is 0-5;

e is 0-4;

f is 0-6;

g is 2-4;

h is 0-4;

R₁ is independently hydrogen, halogen, alkyl, carboxyl, alkoxycarbonyl or aralkyl, or geminal R₁ radicals, taken together with the carbon atom to which the geminal R₁ radicals are attached, form ═CHR, or carbonyl, or two R₁ radicals taken together with the carbon atoms to which the R₁ are linked, form cycloalkylene, or two vicinal R₁ radicals, taken together with the carbon atoms to which the vicinal R₁ radicals are linked form

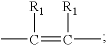

R₂ is independently —(CH2)_{q}—X, or two R₂ radicals taken together with the carbon atoms through which the two R₂ radicals are linked form cycloalkylene, or geminal R₁ and R₂ radicals, taken together with the carbon atom to which the geminal R₁ and R₂ radicals are attached, form cycloalkylene, ═CHR₁, or carbonyl, or two vicinal R₂ radicals, taken together with the carbon atoms to which the vicinal R₂ radicals are linked, form

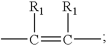

q is 0-3;

X is hydrogen, halogen, alkyl, alkenyl, cycloalkyl, heterocyclyl, aryl, heteroaryl, aralkyl, heteroaralkyl, hydroxy, alkoxy, aralkoxy, heteroaralkoxy, carboxy, alkoxycarbonyl, tetrazolyl, acyl, acylHNSO₂—, —SR₃,

Y¹Y²N— or Y³Y⁴NCO—;

Y¹ and Y² are independently hydrogen, alkyl, aryl, aralkyl or heteroaralkyl, or one of Y¹ and Y² is hydrogen or alkyl and the other of Y¹ and Y² is acyl or aroyl;

Y³ and Y⁴ are independently hydrogen, alkyl, aryl, aralkyl or heteroaralkyl;

Z is R₃O₂C—, R₃OC—, cyclo-imide, —CN, R₃O₂SHNCO—, R₃O₂SHN—, (R₃)₂NCO—,R₃O— or tetrazolyl; and

R₃ and R₄ are independently hydrogen, alkyl, aryl, cycloalkyl, or aralkyl;

R₅ is R₆OC—, R₆NHOC—, hydrogen, alkyl, aryl, heteroaryl, cycloalkyl, heterocyclyl, heteroaralkyl, or aralkyl; and

R₆ is hydrogen, alkyl, aryl, heteroaryl, cycloalkyl, heterocyclyl, heteroaralkyl, or aralkyl; or

a pharmaceutically acceptable salt thereof.

### BRIEF DESCRIPTION OF THE FIGURES

FIG. 1 represents a Northern blotting analysis of up-regulation of ABC1 expression of THP-1 cells using RPR64 and RPR52 at different concentrations.

FIG. 2 represents the corresponding bar graph of FIG. 1 of up-regulation of ABC1 expression of THP-1 cells with RPR64 and RPR52 at different concentrations.

FIG. 3 represents a standard curve ABC1 standard curve with TaqMan 5P primer/probe set.

FIG. 4 represents a Northern blotting analysis of up-regulation of ABC1 in primary hepatocytes using Fenofibric acid and Wy 14,643.

FIG. 5 represents a Northern blotting analysis of up-regulation of ABC1 in human monocytes derived macrophages using Fenofibric acid, PG-J2 and Wy 14,643.

FIG. 6 represents a bar graph of apolipoprotein A-I-mediated cholesterol efflux in human macrophages using AcLDL, Wy 14,643 and AcLDL+Wy 14,643.

As employed above and throughout the disclosure, the following terms, unless otherwise indicated, shall be understood to have the following meanings:

### DEFINITIONS

In the present specification, the term “compounds for use according to the invention”, and equivalent expressions, are meant to embrace compounds of general Formula (I) as hereinbefore described, which expression includes the prodrugs, the pharmaceutically acceptable salts, and the solvates, e.g. hydrates, where the context so permits. Similarly, reference to intermediates, whether or not they themselves are claimed, is meant to embrace their salts, and solvates, where the context so permits. For the sake of clarity, particular instances when the context so permits are sometimes indicated in the text, but these instances are purely illustrative and it is not intended to exclude other instances when the context so permits.

“Prodrug” means a compound which is convertible in vivo by metabolic means (e.g. by hydrolysis) to a compound of Formula (I), including N-oxides thereof. For example an ester of a compound of Formula (I) containing a hydroxy group may be convertible by hydrolysis in vivo to the parent molecule. Alternatively an ester of a compound of Formula (T) containing a carboxy group may be convertible by hydrolysis in vivo to the parent molecule.

“Patient” includes both human and other mammals.

In the present invention, the moiety

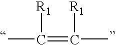

encompasses both the syn and anti configurations.

“Chemical bond” means a direct single bond between atoms.

“Acyl” means an H—CO— or alkyl-CO— group wherein the alkyl group is as herein described. Preferred acyls contain a lower alkyl. Exemplary acyl groups include formyl, acetyl, propanoyl, 2-methylpropanoyl, butanoyl and palmitoyl.

“Alkenyl” means an aliphatic hydrocarbon group containing a carbon-carbon double bond and which may be a straight or branched chain having about 2 to about 15 carbon atoms in the chain. Preferred alkenyl groups have 2 to about 12 carbon atoms in the chain and more preferably about 2 to about 4 carbon atoms in the chain. Branched means that one or more lower alkyl groups such as methyl, ethyl or propyl are attached to a linear alkenyl chain. “Lower alkenyl” means about 2 to about 4 carbon atoms in the chain, which may be straight or branched. The alkenyl group is optionally substituted by one or more halo groups. Exemplary alkenyl groups include ethenyl, propenyl, n-butenyl, i-butenyl, 3-methylbut-2-enyl, n-pentenyl, heptenyl, octenyl and decenyl.

“Alkoxy” means an alkyl-O- group wherein the alkyl group is as herein described. Exemplary alkoxy groups include methoxy, ethoxy, n-propoxy, i-propoxy, n-butoxy and heptoxy.

“Alkoxycarbonyl” means an alkyl-O-CO— group, wherein the alkyl group is as herein defined. Exemplary alkoxycarbonyl groups include methoxycarbonyl, ethoxycarbonyl, or t-butyloxycarbonyl.

“Alkyl” means an aliphatic hydrocarbon group which may be a straight or branched chain having about 1 to about 20 carbon atoms in the chain. Preferred alkyl groups have 1 to about 13 carbon atoms in the chain. Branched means that one or more lower alkyl groups such as methyl, ethyl or propyl are attached to a linear alkyl chain. “Lower alkyl” means that there are about 1 to about 4 carbon atoms in the chain, which may be straight or branched. The alkyl is optionally substituted with one or more “alkyl group substituents” which may be the same or different, and include halo, carboxy, cycloalkyl, cycloalkenyl, heterocyclyl, heterocyclenyl, aryl, alkoxy, alkoxycarbonyl, aralkoxycarbonyl, heteroaralkoxycarbonyl, Y¹Y²NCO—, wherein Y¹ and Y² are independently hydrogen, alkyl, aryl, aralkyl or heteroaralkyl, or Y¹ and Y² taken together with the nitrogen atom to which Y¹ and Y² are attached form heterocyclyl. Exemplary alkyl groups include methyl, trifluoromethyl, ethyl, n-propyl, i-propyl, n-butyl, t-butyl, n-pentyl, and 3-pentyl. Preferably, the alkyl group substituent is selected from acyl, carboxy, carboxymethyl, methoxycarbonylethyl, benzyloxycarbonylmethyl, and pyridylmethyloxycarbonylmethyl and alkoxycarbonyl.

“Alkylsulfinyl” means an alkyl-SO— group wherein the alkyl group is as defined above. Preferred groups are those wherein the alkyl group is lower alkyl.

“Alkylsulfonyl” means an alkyl-SO₂— group wherein the alkyl group is as defined above. Preferred groups are those wherein the alkyl group is lower alkyl.

“Alkylthio” means an alkyl-S— group wherein the alkyl group is as defined above. Exemplary alkylthio groups include methylthio, ethylthio, i-propylthio and heptylthio.

“Aralkoxy” means an aralkyl-O— group wherein the aralkyl group is as defined herein. Exemplary aralkoxy groups include benzyloxy and 1- and 2-naphthalenemethoxy.

“Aralkoxycarbonyl” means an aralkyl-O—CO— group wherein the aralkyl group is as defined herein. An exemplary aralkoxycarbonyl group is benzyloxycarbonyl.

“Aralkyl” means an aryl-alkyl- group wherein the aryl and alkyl groups are as defined herein. Preferred aralkyls contain a lower alkyl moiety. Exemplary aralkyl groups include benzyl, 2-phenethyl and naphthalenemethyl.

“Aralkylsulfonyl” means an aralkyl-SO₂— group wherein the aralkyl group is as defined herein.

“Aralkylsulfinyl” means an aralkyl-SO— group wherein the aralkyl group is as defined herein.

“Aralkylthio” means an aralkyl-S— group wherein the aralkyl group is as defined herein. An exemplary aralkylthio group is benzylthio.

“Aroyl” means an aryl-CO— group wherein the aryl group is as defined herein. Exemplary aroyl groups include benzoyl and 1- and 2-naphthoyl.

“Aryl” means an aromatic monocyclic or multicyclic ring system of about 6 to about 14 carbon atoms, preferably of about 6 to about 10 carbon atoms. The aryl is optionally substituted with one or more “ring group substituents” which may be the same or different, and are as defined herein. Exemplary aryl groups include phenyl, naphthyl, substituted phenyl, and substituted naphthyl.

“Aryldiazo” means an aryl-diazo- group wherein the aryl and diazo groups are as defined herein.

“Fused arylcycloalkenyl” means a fused aryl and cycloalkenyl as defined herein. Preferred fused arylcycloalkenyls are those wherein the aryl thereof is phenyl and the cycloalkenyl consists of about 5 to about 6 ring atoms. A fused arylcycloalkenyl group may be bonded to the rest of the compound through any atom of the fused system capable of such bondage. The fused arylcycloalkenyl may be optionally substituted by one or more ring group substituents, wherein the “ring group substituent” is as defined herein. Exemplary fused arylcycloalkenyl groups include 1,2-dihydronaphthylenyl; indenyl; 1,4-naphthoquinonyl, and the like.

“Fused arylcycloalkyl” means a fused aryl and cycloalkyl as defined herein. Preferred fused arylcycloalkyls are those wherein the aryl thereof is phenyl and the cycloalkyl consists of about 5 to about 6 ring atoms. A fused arylcycloalkyl group may be bonded to the rest of the compound through any atom of the fused system capable of such bonding. The fused arylcycloalkyl may be optionally substituted by one or more ring group substituents, wherein the “ring group substituent” is as defined herein. Exemplary fused arylcycloalkyl groups include 1,2,3,4-tetrahydronaphthylenyl; 1,4-dimethyl-2,3-dihydronaphthalenyl; 2,3-dihydro-1,4-naphthoquinonyl, α-tetralonyl, and the like.

“Fused arylheterocyclenyl” means a fused aryl and heterocyclenyl wherein the aryl and heterocyclenyl groups are as defined herein. Preferred fused arylheterocyclenyl groups are those wherein the aryl thereof is phenyl and the heterocyclenyl consists of about 5 to about 6 ring atoms. A fused arylheterocyclenyl group may be bonded to the rest of the compound through any atom of the fused system capable of such bonding. The designation of aza, oxa or thia as a prefix before the heterocyclenyl portion of the fused arylheterocyclenyl means that a nitrogen, oxygen or sulfur atom respectively, is present as a ring atom. The fused arylheterocyclenyl may be optionally substituted by one or more ring group substituents, wherein the “ring group substituent” is as defined herein. The nitrogen atom of a fused arylheterocyclenyl may be a basic nitrogen atom. The nitrogen or sulphur atom of the heterocyclenyl portion of the fused arylheterocyclenyl is also optionally oxidized to the corresponding N-oxide, S-oxide or S,S-dioxide. Exemplary fused arylheterocyclenyl include 3H-indolinyl, 2(1H)quinolinonyl, 2H-1-oxoisoquinolyl, 1,2-dihydroquinolinyl, (2H)quinolinyl N-oxide, 3,4-dihydroquinolinyl, 1,2-dihydroisoquinolinyl, 3,4-dihydroisoquinolinyl, chromonyl, 3,4-dihydroisoquinoxalinyl, 4-(3H)quinazolinonyl, 4H-chromen-2yl, and the like. Preferably, 2(1H)quinolinonyl, 1,2-dihydroquinolinyl, (2H)quinolinyl N-oxide, or 4-(3H)quinazolinonyl.

“Fused arylheterocyclyl” means a fused aryl and heterocyclyl wherein the aryl and heterocyclyl groups are as defined herein. Preferred fused arylheterocyclyls are those wherein the aryl thereof is phenyl and the heterocyclyl consists of about 5 to about 6 ring atoms. A fused arylheterocyclyl may be bonded to the rest of the compound through any atom of the fused system capable of such bonding. The designation of aza, oxa or thia as a prefix before the heterocyclyl portion of the fused arylheterocyclyl means that a nitrogen, oxygen or sulphur atom respectively is present as a ring atom. The fused arylheterocyclyl group may be optionally substituted by one or more ring group substituents, wherein the

“ring group substituent” is as defined herein. The nitrogen atom of a fused arylheterocyclyl may be a basic nitrogen atom. The nitrogen or sulphur atom of the heterocyclyl portion of the fused arylheterocyclyl is also optionally oxidized to the corresponding N-oxide, S-oxide or S,S-dioxide. Exemplary fused arylheterocyclyl ring systems include indolinyl, 1,2,3,4-tetrahydroisoquinolinyl, 1,2,3,4-tetrahydroquinolinyl, 1H-2,3-dihydroisoindol-2-yl, 2,3-dihydrobenz[f]isoindol-2-yl, 1,2,3,4-tetrahydrobenz[g]isoquinolin-2-yl, chromanyl, isochromanonyl, 2,3-dihydrochromonyl, 1,4-benzodioxan, 1,2,3,4-tetrahydroquinoxalinyl, and the like. Preferably, 1,2,3,4-tetrahydroisoquinolinyl, 1,2,3,4-tetrahydroquinoxalinyl, and 1,2,3,4-tetrahydroquinolinyl.

“Aryloxy” means an aryl-O— group wherein the aryl group is as defined herein. Exemplary groups include phenoxy and 2-naphthyloxy.

“Aryloxycarbonyl” means an aryl-O—CO— group wherein the aryl group is as defined herein. Exemplary aryloxycarbonyl groups include phenoxycarbonyl and naphthoxycarbonyl.

“Arylsulfonyl” means an aryl-SO₂— group wherein the aryl group is as defined herein.

“Arylsulfinyl” means an aryl-SO— group wherein the aryl group is as defined herein.

“Arylthio” means an aryl-S— group wherein the aryl group is as defined herein. Exemplary arylthio groups include phenylthio and naphthylthio.

“Carbamoyl” is an NH₂—CO— group.

“Carboxy” means a HO(O)C— (carboxylic acid) group.

“Compounds of the invention,” and equivalent expressions, are meant to embrace compounds of general Formula (1) as hereinbefore described, which expression includes the prodrugs, the pharmaceutically acceptable salts, and the solvates, e.g. hydrates, where the context so permits. Similarly, reference to intermediates, whether or not they themselves are claimed, is meant to embrace their salts, and solvates, where the context so permits. For the sake of clarity, particular instances when the context so permits are sometimes indicated in the text, but these instances are purely illustrative and it is not intended to exclude other instances when the context so permits.

“Cycloalkoxy” means an cycloalkyl-O— group wherein the cycloalkyl group is as defined herein. Exemplary cycloalkoxy groups include cyclopentyloxy and cyclohexyloxy.

“Cycloalkenyl” means a non-aromatic mono- or multicyclic ring system of about 3 to about 10 carbon atoms, preferably of about 5 to about 10 carbon atoms, and which contains at least one carbon-carbon double bond. Preferred ring sizes of rings of the ring system include about 5 to about 6 ring atoms. The cycloalkenyl is optionally substituted with one or more “ring group substituents” which may be the same or different, and are as defined herein. Exemplary monocyclic cycloalkenyl include cyclopentenyl, cyclohexenyl, cycloheptenyl, and the like. An exemplary multicyclic cycloalkenyl is norbornylenyl.

“Cycloalkyl” means a non-aromatic mono- or multicyclic ring system of about 3 to about 10 carbon atoms, preferably of about 5 to about 10 carbon atoms. Preferred ring sizes of rings of the ring system include about 5 to about 6 ring atoms. The cycloalkyl is optionally substituted with one or more “ring group substituents” which may be the same or different, and are as defined herein. Exemplary monocyclic cycloalkyl include cyclopentyl, cyclohexyl, cycloheptyl, and the like. Exemplary multicyclic cycloalkyl include 1-decalin, norbornyl, adamant-(1- or 2-)yl, and the like.

“Cycloalkylene” means a bivalent, saturated carbocyclic group having about 3 to about 6 carbon atoms. Preferred cycloalkylene groups include 1,1-, 1,2-, 1,3-, and 1,4-cis or trans-cyclohexylene; and 1,1-, 1,2-, and 1,3-cyclopentylene.

“Cyclo-imide” means a compound of formulae

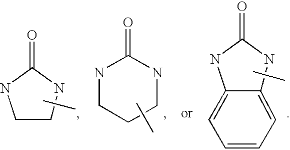

The cyclo-imide moiety may be attached to the parent molecule through either a carbon atom or nitrogen atom of the carbamoyl moiety. An exemplary imide group is N-phthalimide.

“Diazo” means a bivalent —N═N— radical.

“Halo” means fluoro, chloro, bromo, or iodo. Preferred are fluoro, chloro and bromo, more preferably fluoro and chloro.

“Heteroaralkyl” means a heteroaryl-alkyl- group wherein the heteroaryl and alkyl groups are as defined herein. Preferred heteroaralkyls contain a lower alkyl moiety. Exemplary heteroaralkyl groups include thienylmethyl, pyridylmethyl, imidazolylmethyl and pyrazinylmethyl.

“Heteroaralkylthio” means a heteroaralkyl-S— group wherein the heteroaralkyl group is as defined herein. An exemplary heteroaralkylthio group is 3-pyridinepropanthiol.

“Heteroaralkoxy” means an heteroaralkyl-O— group wherein the heteroaralkyl group is as defined herein. An exemplary heteroaralkoxy group is 4-pyridylmethyloxy.

“Heteroaroyl” means an means an heteroaryl-CO— group wherein the heteroaryl group is as defined herein. Exemplary heteroaryl groups include thiophenoyl, nicotinoyl, pyrrol-2-ylcarbonyl and 1- and 2-naphthoyl and pyridinoyl.

“Heteroaryldiazo” means an heteroaryl-diazo- group wherein the heteroaryl and diazo groups are as defined herein.

“Heteroaryl” means an aromatic monocyclic or multicyclic ring system of about 5 to about 14 carbon atoms, preferably about 5 to about 10 carbon atoms, in which at least one of the carbon atoms in the ring system is replaced by a hetero atom, i.e., other than carbon, for example nitrogen, oxygen or sulfur. Preferred ring sizes of rings of the ring system include about 5 to about 6 ring atoms. The heteroaryl ring is optionally substituted by one or more “ring group substituents” which may be the same or different, and are as defined herein. The designation of aza, oxa or thia as a prefix before the heteroaryl means that a nitrogen, oxygen or sulfur atom is present, respectively, as a ring atom. A nitrogen atom of an heteroaryl may be a basic nitrogen atom and also may be optionally oxidized to the corresponding N-oxide. Exemplary heteroaryl and substituted heteroaryl groups include pyrazinyl, thienyl, isothiazolyl, oxazolyl, pyrazolyl, cinnolinyl, pteridinyl, benzofuryl, furazanyl, pyrrolyl, 1,2,4-thiadiazolyl, pyridazinyl, indazolyl, quinoxalinyl, phthalazinyl, imidazo[1,2-a]pyridine, imidazo[2,1-b]thiazolyl, benzofurazanyl, azaindolyl, benzimidazolyl, benzothienyl, thienopyridyl, thienopyrimidyl, pyrrolopyridyl, imidazopyridyl, naphthyridinyl, benzoazaindole, 1,2,4-triazinyl, benzothiazolyl, furyl, imidazolyl, indolyl, isoindolyl, indolizinyl, isoxazolyl, isoquinolinyl, isothiazolyl, oxadiazolyl, pyrazinyl, pyridazinyl, pyrazolyl, pyridyl, pyrimidinyl, pyrrolyl, quinazolinyl, quinolinyl, 1,3,4-thiadiazolyl, thiazolyl, thienyl and triazolyl. Preferred heteroaryl and substituted heteroaryl groups include quinolinyl, indazolyl, indolyl, quinazolinyl, pyridyl, pyrimidinyl, furyl, benzothiazolyl, quinoxalinyl, benzimidazolyl, benzothienyl, and isoquinolinyl.

“Fused heteroarylcycloalkenyl” means a fused heteroaryl and cycloalkenyl wherein the heteroaryl and cycloalkenyl groups are as defined herein. Preferred fused heteroarylcycloalkenyls are those wherein the heteroaryl thereof is phenyl and the cycloalkenyl consists of about 5 to about 6 ring atoms. A fused heteroarylcycloalkenyl may be bonded to the rest of the compound through any atom of the fused system capable of such bonding. The designation of aza, oxa or thia as a prefix before the heteroaryl portion of the fused heteroarylcycloalkenyl means that a nitrogen, oxygen or sulfur atom is present, respectively, as a ring atom. The fused heteroarylcycloalkenyl may be optionally substituted by one or more ring group substituents, wherein the “ring group substituent” is as defined herein. The nitrogen atom of a fused heteroarylcycloalkenyl may be a basic nitrogen atom. The nitrogen atom of the heteroaryl portion of the fused heteroarylcycloalkenyl may also be optionally oxidized to the corresponding N-oxide. Exemplary fused heteroarylcycloalkenyl groups include 5,6-dihydroquinolyl; 5,6-dihydroisoquinolyl; 5,6-dihydroquinoxalinyl; 5,6-dihydroquinazolinyl; 4,5-dihydro-1H-benzimidazolyl; 4,5-dihydrobenzoxazolyl; 1,4-naphthoquinolyl, and the like.

“Fused heteroarylcycloalkyl” means a fused heteroaryl and cycloalkyl wherein the heteraryl and cycloalkyl groups are as defined herein. Preferred fused heteroarylcycloalkyls are those wherein the heteroaryl thereof consists of about 5 to about 6 ring atoms and the cycloalkyl consists of about 5 to about 6 ring atoms. A fused heteroarylcycloalkyl may be bonded to the rest of the compoun through any atom of the fused system capable of such bonding. The designation of aza, oxa or thia as a prefix before the heteroaryl portion of the fused heteroarylcycloalkyl means that a nitrogen, oxygen or sulfur atom is present respectively as a ring atom. The fused heteroarylcycloalkyl may be optionally substituted by one or more ring group substituents, wherein the “ring group substituent” is as defined herein. The nitrogen atom of a fused heteroarylcycloalkyl may be a basic nitrogen atom. The nitrogen atom of the heteroaryl portion of the fused heteroarylcycloalkyl may also be optionally oxidized to the corresponding N-oxide. Exemplary fused heteroarylcycloalkyl include 5,6,7,8-tetrahydroquinolinyl; 5,6,7,8-tetrahydroisoquinolyl; 5,6,7,8-tetrahydroquinoxalinyl; 5,6,7,8-tetrahydroquinazolyl; 4,5,6,7-tetrahydro-1H-benzimidazolyl; 4,5,6,7-tetrahydrobenzoxazolyl; 1H-4-oxa-1,5-diazanaphthalen-2-only; 1,3-dihydroimidizole-[4,5]-pyridin-2-only; 2,3-dihydro-1,4-dinaphthoquinonyl and the like, preferably, 5,6,7,8-tetrahydroquinolinyl or 5,6,7,8-tetrahydroisoquinolyl.

“Fused heteroarylheterocyclenyl” means a fused heteroaryl and heterocyclenyl wherein the heteraryl and heterocyclenyl groups are as defined herein. Preferred fused heteroarylheterocyclenyls are those wherein the heteroaryl thereof consists of about 5 to about 6 ring atoms and the heterocyclenyl consists of about 5 to about 6 ring atoms. A fused heteroarylheterocyclenyl may be bonded to the rest of the compound through any atom of the fused system capable of such bonding. The designation of aza, oxa or thia as a prefix before the heteroaryl or heterocyclenyl portion of the fused heteroarylheterocyclenyl means that a nitrogen, oxygen or sulfur atom is present respectively as a ring atom. The fused heteroarylheterocyclenyl may be optionally substituted by one or more ring group substituent, wherein the “ring group substituent” is as defined herein. The nitrogen atom of a fused heteroarylazaheterocyclenyl may be a basic nitrogen atom. The nitrogen or sulphur atom of the heteroaryl or heterocyclenyl portion of the fused heteroarylheterocyclenyl may also be optionally oxidized to the corresponding N-oxide, S-oxide or S,S-dioxide. Exemplary fused heteroarylheterocyclenyl groups include 7,8-dihydro[1,7]naphthyridinyl; 1,2-dihydro[2,7]naphthyridinyl; 6,7-dihydro-3H-imidazo[4,5-c]pyridyl; 1,2-dihydro-1,5-naphthyridinyl; 1,2-dihydro-1,6-naph 1,2-dihydro-1,7-naphthyridinyl; 1,2-dihydro-1,8-naphthyridinyl; 1,2-dihydro-2,6-naphthyridinyl, and the like.

“Fused heteroarylheterocyclyl” means a fused heteroaryl and heterocyclyl wherein the heteroaryl and heterocyclyl groups are as defined herein. Preferred fused heteroarylheterocyclyls are those wherein the heteroaryl thereof consists of about 5 to about 6 ring atoms and the heterocyclyl consists of about 5 to about 6 ring atoms. A fused heteroarylheterocyclyl may be bonded to the rest of the compound through any atom of the fused system capable of such bonding. The designation of aza, oxa or thia as a prefix before the heteroaryl or heterocyclyl portion of the fused heteroarylheterocyclyl means that a nitrogen, oxygen or sulfur atom is present respectively as a ring atom. The fused heteroarylheterocyclyl may be optionally substituted by one or more ring group substituent, wherein the “ring group substituent” is as defined herein. The nitrogen atom of a fused heteroarylheterocyclyl may be a basic nitrogen atom. The nitrogen or sulphur atom of the heteroaryl or heterocyclyl portion of the fused heteroarylheterocyclyl may also be optionally oxidized to the corresponding N-oxide, S-oxide or S,S-dioxide. Exemplary fused heteroarylheterocyclyl groups include 2,3-dihydro-1H pyrrol[3,4-b]quinolin-2-yl; 1,2,3,4-tetrahydrobenz[b][1,7]naphthyridin-2-yl; 1,2,3,4-tetrahydrobenz[b][1,6]naphthyridin-2-yl; 1,2,3,4-tetrahydro-9H-pyrido[3,4-b]indol-2yl; 1,2,3,4-tetrahydro-9H-pyrido[4,3-b]indol-2yl, 2,3,-dihydro-1H-pyrrolo[3,4-b]indol-2-yl; 1H-2,3,4,5-tetrahydroazepino[3,4-b]indol-2-yl; 1H-2,3,4,5-tetrahydroazepino[4,3-b]indol-3-yl; 1H-2,3,4,5-tetrahydroazepino[4,5-b]indol-2yl, 5,6,7,8-tetrahydro[1,7]napthyridinyl; 1,2,3,4-tetrhydro[2,7]naphthyridyl; 2,3-dihydro[1,4]dioxino[2,3-b]pyridyl; 2,3-dihydro[1,4]dioxino[2,3-b]pryidyl; 3,4-dihydro-2H-1-oxa[4,6]diazanaphthalenyl; 4,5,6,7-tetrahydro-3H-imidazo[4,5-c]pyridyl; 6,7-dihydro[5 ,8]diazanaphthalenyl; 1,2,3,4-tetrahydro[1,5]napthyridinyl; 1,2,3,4-tetrahydro[1,6]napthyridinyl; 1,2,3,4-tetrahydro[1,7]napthyridinyl; 1,2,3,4-tetrahydro[1,8]napthyridinyl; 1,2,3,4-tetrahydro[2,6]napthyridinyl, and the like.

“Heteroarylsulfonyl” means an heteroaryl-SO₂— group wherein the heteroaryl group is as defined herein. An examplary heterarylsulfonyl groups is 3-pyridinepropansulfonyl.

“Heteroarylsulfinyl” means an heteroaryl —SO— group wherein the heteroaryl group is as defined herein.

“Heteroarylthio” means an heteroaryl —S— group wherein the heteroaryl group is as defined herein. Exemplary heteroaryl thio groups include pyridylthio and quinolinylthio.

“Heterocyclenyl” means a non-aromatic monocyclic or multicyclic hydrocarbon ring system of about 3 to about 10 carbon atoms, preferably about 5 to about 10 carbon atoms, in which at least one or more of the carbon atoms in the ring system is replaced by a hetero atom, for example a nitrogen, oxygen or sulfur atom, and which contains at least one carbon-carbon double bond or carbon-nitrogen double bond. Preferred ring sizes of rings of the ring system include about 5 to about 6 ring atoms. The designation of aza, oxa or thia as a prefix before the heterocyclenyl means that a nitrogen, oxygen or sulfur atom is present respectively as a ring atom. The heterocyclenyl may be optionally substituted by one or more ring group substituents, wherein the “ring group substituent” is as defined herein. The nitrogen atom of an heterocyclenyl may be a basic nitrogen atom. The nitrogen or sulphur atom of the heterocyclenyl is also optionally oxidized to the corresponding N-oxide, S-oxide or S,S-dioxide. Exemplary monocyclic azaheterocyclenyl groups include 1,2,3,4-tetrahydrohydropyridine, 1,2-dihydropyridyl, 1,4-dihydropyridyl, 1,2,3,6-tetrahydropyridine, 1,4,5,6-tetrahydropyrimidine, 2-pyrrolinyl, 3-pyrrolinyl, 2-imidazolinyl, 2-pyrazolinyl, and the like. Exemplary oxaheterocyclenyl groups include 3,4-dihydro-2H-pyran, dihydrofuryl, and fluorodihydrofuryl An exemplary multicyclic oxaheterocyclenyl group is 7-oxabicyclo[2.2.1]heptenyl. Exemplary monocyclic thiaheterocycleny rings include dihydrothiophenyl and dihydrothiopyranyl.

“Heterocyclyl” means a non-aromatic saturated monocyclic or multicyclic ring system of about 3 to about 10 carbon atoms, preferably about 5 to about 10 carbon atoms, in which at least one of the carbon atoms in the ring system is replaced by a hetero atom, for example nitrogen, oxygen or sulfur. Preferred ring sizes of rings of the ring system include about 5 to about 6 ring atoms. The designation of aza, oxa or thia as a prefix before the heterocyclyl means that a nitrogen, oxygen or sulfur atom is present respectively as a ring atom. The heterocyclyl may be optionally substituted by one or more “ring group substituents” which may be the same or different, and are as defined herein. The nitrogen atom of an heterocyclyl may be a basic nitrogen atom. The nitrogen or sulphur atom of the heterocyclyl is also optionally oxidized to the corresponding N-oxide, S-oxide or S,S-dioxide. Exemplary monocyclic heterocyclyl rings include piperidyl, pyrrolidinyl, piperazinyl, morpholinyl, thiomorpholinyl, thiazolidinyl, 1,3-dioxolanyl, 1,4-dioxanyl, tetrahydrofuryl, tetrahydrothiophenyl, tetrahydrothiopyranyl, and the like. Exemplary multicyclic heterocyclyl rings include 1,4 diazabicyclo-[2.2.2]octane and 1,2-cyclohexanedicarboxylic acid anhydride.

“Ring group substituent” includes hydrogen, alkyl, cycloalkyl, heterocyclyl, aryl, heteroaryl, aralkyl, heteroaralkyl, hydroxy, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, fused cycloalkyl, fused cycloalkenyl, fused heterocyclyl, fused heterocyclenyl, arylazo, heteroarylazo, R^{a}R^{b}N—, R^{c}R^{d}NCO—, R^{c}O₂CN—, and R^{c}R^{d}NSO₂— wherein R^{a} and R^{b} are independently hydrogen, alkyl, aryl, aralkyl or heteroaralkyl, or one of R^{a} and R^{b} is hydrogen or alkyl and the other of R^{a} and R^{b} is aroyl or heteroaroyl. R^{c} and R^{d} are independently hydrogen, alkyl, aryl, heteroaryl, cycloalkyl, cycloalkenyl, heterocyclyl, heterocyclenyl, aralkyl or heteroaralkyl. Where the ring is cycloalkyl, cycloalkenyl, heterocyclyl or heterocyclenyl, the ring group substituent may also include methylene (H₂C═), oxo (0═), thioxo (S═), on carbon atom(s) thereof. Preferably, the ring substituents are selected from oxo (O═), alkyl, aryl, alkoxy, aralkoxy, halo, carboxy, alkoxycarbonyl, and R^{e}O₂CN—, wherein R^{e} is cycloalkyl.

“Tetrazolyl” means a group of formula

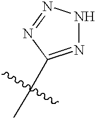

wherein the hydrogen atom thereof is optionally replaced by alkyl, carboxyalkyl or alkoxycarbonylalkyl.

“PPAR ligand receptor binder” means a ligand which binds to the PPAR receptor. PPAR ligand receptor binders of this invention are useful as agonists or antagonists of the PPAR-α, PPAR-δ, or PPAR-γ receptor.

The term “pharmaceutically acceptable salt” refers to a relatively non-toxic, inorganic or organic acid addition salt of a compound of the present invention. A salt can be prepared in situ during the final isolation and purification of a compound or by separately reacting the purified compound in its free base form with a suitable organic or inorganic acid and isolating the salt thus formed. Representative salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, laurylsulphonate salts, and the like. (See, for example S. M. Berge, et al., “Pharmaceutical Salts,” J. Pharm. Sci., 66: 1-19, 1977, the contents of which are hereby incorporated herein by reference.)

“Treating” means the partial or complete relieving or preventing of one or more physiological or biochemical parameters associated with ABC-1 activity.

The term “modulate” refers to the ability of a compound to either directly (by binding to the receptor as a ligand) or indirectly (as a precursor for a ligand or an inducer which promotes production of a ligand from a precursor) induce expression of gene(s) maintained under hormone control, or to repress expression of gene (s) maintained under such control.

The term “obesity” refers generally to individuals who are at least about 20-30% over the average weight for the person's age, sex and height. Technically, “obese” is defined, for males, as individuals whose body mass index is greater than 27.3 kg/m². Those skilled in the art readily recognize that the invention method is not limited to those who fall within the above criteria. Indeed, the invention method can also be advantageously practiced by individuals who fall outside of these traditional criteria, for example by those who are prone to obesity.

The phrase “amount effective to lower blood glucose levels” refers to levels of a compound sufficient to provide circulating concentrations high enough to accomplish the desired effect. Such a concentration typically falls in the range of about 10 nM up to 2 μM, with concentrations in the range of about 100 nm up to about 500 nM being preferred.

The phrase “amount effective to lower triclyceride levels” refers to levels of a compound sufficient to provide circulating concentrations high enough to accomplish the desired effect. Such a concentration typically falls in the range of about 10 nM up to 20 μM; with concentrations in the range of about 100 nm up to about 500 nM being preferred.

Preferred Embodiments

Preferred embodiments according to the invention include the method for modulating ABC-1 gene expression comprising contacting a PPAR receptor with a PPAR mediator.

Another preferred embodiment according to the invention includes the method for modulating ABC-1 gene expression comprising contacting a PPAR receptor with a PPAR-α mediator.

Another preferred embodiment according to the invention includes the method for modulating ABC-1 gene expression comprising contacting a PPAR receptor with a PPAR-δ mediator.

Another preferred embodiment according to the invention includes the method for modulating ABC-1 gene expression comprising contacting a PPAR receptor with a PPAR-γ mediator.

Another preferred embodiments according to the invention includes the method for modulating ABC-1 gene expression comprising contacting a PPAR receptor with a PPAR agonists.

Another preferred embodiments according to the invention includes the method for repressing ABC-1 gene expression comprising contacting a PPAR receptor with a PPAR antagonist.

Another preferred embodiment according to the invention includes the method of treating a physiological condition in a patient associated with ABC-1 gene expression comprising administering to a patient in need of such treatment, a pharmaceutically effective amount of a PPAR mediator.

Another preferred embodiment according to the invention includes the method of treating a physiological condition in a patient associated with deficient levels of ABC-1 gene expression comprising administering to a patient in need of such treatment, a pharmaceutically effective amount of a PPAR agonist.

Another preferred embodiment according to the invention includes the method of treating a physiological condition in a patient associated with deficient levels of ABC-1 gene expression comprising administering to a patient in need of such treatment, a pharmaceutically effective amount of a PPAR-α agonist, PPAR-δ agonist or PPAR-γ agonist.

Another preferred embodiment according to the invention includes the method of treating a physiological condition in a patient associated with elevated levels ABC-1 gene expression comprising administering to a patient in need of such treatment, a pharmaceutically effective amount of a PPAR antagonist.

Another preferred embodiment according to the invention includes the method of treating a physiological condition in a patient associated with elevated levels ABC-1 gene expression comprising administering to a patient in need of such treatment, a pharmaceutically effective amount of a PPAR-α antagonist, PPAR-δ antagonist or PPAR-γ antagonist.

Another preferred embodiment according to the invention includes the method of treating a physiological condition in a patient associated with ABC-1 gene expression comprising administering to a patient in need of such treatment, a pharmaceutically effective amount of a compound of Formula I.

Another preferred embodiment according to the invention includes the method of treating a physiological condition in a patient associated with ABC-1 gene expression comprising administering to a patient in need of such treatment, a pharmaceutically effective amount of compound selected from the group consisting of Nafenopn , UF-5, ETYA, GW2331, 15-deoxy-Δ^{12,14}-prostaglandin J₂, clofibric, linoleic acid, BRL-49653, fenofibrate, WR-1339, Pioglitazone, Ciglitazone, Englitazone, Troglitazone, LY-171883, AD 5075, 5-[[4-[2-(methyl-2-pyridinylamino)ethoxy]phenyl]methyl]-2,4-thiazolidinedione, WAY-120,744, and Darglitazone and their pharmaceutically acceptable salts.

Another preferred embodiment according to the invention includes the method of treating a disease associated with deficient levels of ABC1 gene expression, selected from the group consisting of atherosclerosis, fish-eye disease, familial HDL deficiencies (FHD), Tangier disease, LCAT deficiency, cholesterol efflux, malaria and diabetes, comprising administering to a patient in need of such treatment, a pharmaceutically effective amount of a PPAR agonist.

Another preferred embodiment according to the invention includes the method of treating a disease associated with deficient levels of ABC1 gene expression, selected from the group consisting of atherosclerosis, fish-eye disease, familial HDL deficiencies (FHD), Tangier disease, LCAT deficiency, cholesterol efflux, malaria and diabetes, comprising administering to a patient in need of such treatment, a pharmaceutically effective amount of a PPAR agonist of formula (I).

An embodiment according to the invention is the use of compounds of Formula I (and their pharmaceutical compositions) as binders for PPAR receptors.

More particularly, the use of compounds of Formula I that bind to the PPAR-α receptor,

compounds of Formula I that bind to the PPAR-δ receptor,

compounds of Formula I that bind to the PPAR-γ receptor,

compounds of Formula I that bind to the PPAR-α and the PPAR-γ receptor,

compounds of Formula I that bind to the PPAR-α and the PPAR-δ receptor,

compounds of Formula I that bind to the PPAR-γ and the PPAR-δ receptor,

compounds of Formula I that act as PPAR receptor agonists,

compounds of Formula I that act as PPAR-α receptor agonists,

compounds of Formula I that act as PPAR-δ receptor agonists,

compounds of Formula I that act as PPAR-γ receptor agonists,

compounds of Formula I that act as both PPAR-α and PPAR-γ receptor agonists,

compounds of Formula I that act as both PPAR-α and PPAR-δ receptor agonists,

compounds of Formula I that act as both PPAR-γ and PPAR-δ receptor agonists,

compounds of Formula I that act as both PPAR-α receptor antagonists and PPAR-γ receptor agonists,

compounds of Formula I that act as both PPAR-α receptor antagonists and PPAR-δ receptor agonists,

compounds of Formula I and act as both PPAR-γ receptor antagonists and PPAR-δ receptor agonists,

compounds of Formula I that act as both PPAR-α receptor agonists and PPAR-γ receptor antagonists,

compounds of Formula I that act as both PPAR-α receptor agonists and PPAR-δ receptor antagonists,

compounds of Formula I that act as both PPAR-γ receptor agonists and PPAR-δ receptor antagonists,

compounds of Formula I that act as PPAR receptor antagonists,

compounds of Formula I that act as PPAR-α receptor antagonists,

compounds of Formula I that act as PPAR-δ receptor antagonists,

compounds of Formula I that act as PPAR-γ receptor antagonists,

compounds of Formula I that act as both PPAR-α and PPAR-γ receptor antagonists,

compounds of Formula I that act as both PPAR-α and PPAR-δ receptor antagonists, and

compounds of Formula I that act as both PPAR-γ and PPAR-δ receptor antagonists.

An embodiment according to the invention is directed to treating a patient suffering from a physiological disorder capable of being modulated by a compound of Formula I having PPAR ligand binding activity, comprising administering to the patient a pharmaceutically effective amount of the compound, or a pharmaceutically acceptable salt thereof. Physiological disorders capable of being so modulated include, for example, cell differentiation to produce lipid accumulating cells, regulation of insulin sensitivity and blood glucose levels, which are involved in hypoglycemia/hyperinsulinism (resulting from, for example, abnormal pancreatic beta cell function, insulin secreting tumors and/or autoimmune hypoglycemia due to autoantibodies to insulin, autoantibodies to the insulin receptor, or autoantibodies that are stimulatory to pancreatic beta cells), macrophage differentiation which leads to the formation of atherosclerotic plaques, inflammatory response, carcinogenesis, hyperplasia, adipocyte gene expression, adipocyte differentiation, reduction in the pancreatic β-cell mass, insulin secretion, tissue sensitivity to insulin, liposarcoma cell growth, chronic anovulation, hyperandrogenism, progesterone production, steroidogenesis, redox potential and oxidative stress in cells, nitric oxide synthase (NOS) production, increased gamma glutamyl transpeptidase, catalase, plasma triglycerides, HDL and LDL cholesterol levels and the like.

Another embodiment according to the invention is directed to a method of treating a disease state in a patient with a pharmaceutically effective amount of a compound of Formula I, or a pharmaceutically acceptable salt thereof, wherein the disease is associated with a physiological detrimental blood level of insulin, glucose, free fatty acids (FFA), or triglycerides.

An embodiment according to the invention is directed to treating a patient suffering from a physiological disorder associated with physiologically detrimental levels of triglycerides in the blood, by administering to the patient a pharmaceutically effective amount of the compound, or of a pharmaceutically acceptable salt thereof.

An embodiment according to the invention is the use of compounds of Formula I and their pharmaceutical compositions as anti-diabetic, anti-lipidemic, anti-hypertensive or anti-arteriosclerotic agents, or in the treatment of obesity.

Another embodiment according to the invention is directed to a method of treating hyperglycemia in a patient, by administering to the patient a pharmaceutically effective amount to lower blood glucose levels of a compound of Formula I, or a pharmaceutically acceptable salt thereof. Preferably, the form of hyperglycemia treated in accordance with this invention is Type II diabetes.

Another embodiment according to the invention is directed to a method of reducing triglyceride levels in a patient, comprising administering to the patient a therapeutically effective amount (to lower triglyceride levels) of a compound of Formula I, or a pharmaceutically acceptable salt thereof.

Another embodiment according to the invention is directed to a method of treating hyperinsulinism in a patient, comprising administering to the patient a therapeutically effective amount of a compound of Formula I, or a pharmaceutically acceptable salt thereof.

Another embodiment according to the invention is directed to a method of treating insulin resistance in a patient, comprising administering to the patient a therapeutically effective amount of a compound of Formula I, or a pharmaceutically acceptable salt thereof.

Another embodiment according to the invention is directed to a method of treating cardiovascular disease, such as atherosclerosis in a patient, comprising administering to the patient a therapeutically effective amount of a compound of Formula I, or a pharmaceutically acceptable salt thereof.

Another embodiment according to the invention is directed to treating of hyperlipidemia in a patient, comprising administering to the patient a therapeutically effective amount of a compound of Formula I, or a pharmaceutically acceptable salt thereof.

Another embodiment according to the invention is directed to treating of hypertension in a patient, comprising administering to the patient a therapeutically effective amount of a compound of Formula I, or a pharmaceutically acceptable salt thereof.

Another embodiment according to the invention is directed to treating eating disorders in a patient, comprising administering to the patient a therapeutically effective amount of a compound of Formula I, or a pharmaceutically acceptable salt thereof. Treatment of eating disorders includes the regulation of appetite or food intake in patients suffering from under-eating disorders such as anorexia nervosa as well as over-eating disorders such as obesity and anorexia bulimia.

Another embodiment according to the invention is directed to treating a disease state associated with low levels of HDL comprising administering to the patient a therapeutically effective amount of a compound of Formula I, or a pharmaceutically acceptable salt thereof. Diseases associated with low levels of HDL include atherosclerotic diseases.

Another embodiment according to the invention is directed to treating polycystic ovary syndrome comprising administering to the patient a therapeutically effective amount of a compound of Formula I, or a pharmaceutically acceptable salt thereof.

Another embodiment according to the invention is directed to treating climacteric comprising administering to the patient a therapeutically effective amount of a compound of Formula I, or a pharmaceutically acceptable salt thereof.

Another embodiment according to the invention is directed to treating inflammatory diseases comprising administering to the patient a therapeutically effective amount of a compound of Formula I, or a pharmaceutically acceptable salt thereof.

Another aspect of the invention is to provide a novel pharmaceutical composition which is effective, in and of itself, for utilization in a beneficial combination therapy because it includes a plurality of active ingredients which may be utilized in accordance with the invention.

In another aspect, the present invention provides a method for treating a disease state in a patient, wherein the disease is associated with a physiological detrimental level of insulin, glucose, free fatty acids (FFA), or triglycerides, in the blood, comprising administering to the patient a therapeutically effective amount of a compound of Formula I, and also administering a therapeutically effective amount of an additional hypoglycemic agent.

In another aspect, the present invention provides a method for treating a disease state in a patient, wherein the disease is associated with a physiological detrimental level of insulin, glucose, free fatty acids (FFA), or triglycerides, in the blood, comprising administering to the patient a therapeutically effective amount of a compound of Formula I, and also administering a therapeutically effective amount of a biguanidine compound.

In another aspect, the present invention provides a method for treating a disease state in a patient, wherein the disease is associated with a physiological detrimental level of insulin, glucose, free fatty acids (FFA), or triglycerides, in the blood, comprising administering to the patient a therapeutically effective amount of a compound of Formula I, and also administering a therapeutically effective amount of metformin.

The invention also provides kits or single packages combining two or more active ingredients useful in treating the disease. A kit may provide (alone or in combination with a pharmaceutically acceptable diluent or carrier), a compound of Formula (I) and an additional hypoglycaemic agent (alone or in combination with diluent or carrier).

There are many known hypoglycemic agents in the art, for example, insulin; biguanidines, such as metformin and buformin; sulfonylureas, such as acetohexamide, chloropropamide, tolazamide, tolbutamide, glyburide, glypizide and glyclazide; thiazolidinediones, such as troglitazone; α-glycosidase inhibitors, such as acarbose and miglatol; and B₃ adrenorecptor agonists such as CL-316, 243.

Since sulfonylureas are known to be capable of stimulating insulin release, but are not capable of acting on insulin resistance, and compounds of Formula I are able to act on insulin resistance, it is envisaged that a combination of these medicaments could be used as a remedy for conditions associated with both deficiency in insulin secretion and insulin-resistance.

Therefore, the invention also provides a method of treating diabetes mellitus of type II in a patient comprising administering a compound of Formula I and one or more additional hypoglycemic agents selected from the group consisting of sulfonylureas, biguanidines, thiazolidinediones, B₃-adrenoreceptor agonists, α-glycosidase inhibitors and insulin.

The invention also provides a method of treating diabetes mellitus of type II in a patient comprising administering a compound of Formula I and a sulfonylurea selected from the group consisting of acetohexamide, chlorpropamide, tolazamide, tolbutamide, glyburide, glypizide and glyclazide.

The invention also provides a method of treating diabetes mellitus of type II in a patient comprising administering a compound of Formula I and a biguanidine selected from the group consisting of metformin and buformin.

The invention also provides a method of treating diabetes mellitus of type II in a patient comprising administering a compound of Formula I and an α-glycosidase inhibitor selected from the group consisting acarbose and miglatol.

The invention also provides a method of treating diabetes mellitus of type II in a patient comprising administering a compound of Formula I and an thiazolidinedione, for example, troglitazone.

As indicated above, a compound of Formula I may be administered alone or in combination with one or more additional hypoglycemic agents. Combination therapy includes administration of a single pharmaceutical dosage formulation which contains a compound of Formula I and one or more additional hypoglycemic agent, as well as administration of the compound of Formula I and each additional hypoglycemic agents in its own separate pharmaceutical dosage formulation. For example, a compound of Formula I and hypoglycemic agent can be administered to the patient together in a single oral dosage composition such as a tablet or capsule, or each agent administered in separate oral dosage formulations. Where separate dosage formulations are used, the compound of Formula I and one or more additional hypoglycemic agents can be administered at essentially the same time, i.e., concurrently, or at separately staggered times, i.e., sequentially.

For example, the compound of Formula I may be administered in combination with one or more of the following additional hypoglycemic agents: insulin; biguanidines such as metformin or buformin; sulfonylureas such as acetohexamide, chloropropamide, tolazamide, tolbutamide, glyburide, glypizide or glyclazide; thiazolidinediones such as troglitazone; α-glycosidase inhibitors such as acarbose or miglatol; or B₃ adrenorecptor agonists such as CL-316, 243.

The compound of Formula I is preferably administered with a biguanidine, in particular, metformin.

The compounds of Formula I contain at least three aromatic or hetero-aromatic rings, which may be designated as shown in Formula II below, and for which their substitution pattern along the chain with respect to each other also is shown below.

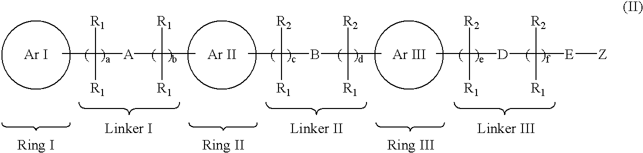

A preferred aspect of the compounds of Formula II, is a compound wherein

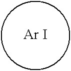

is selected from quinolinyl, benzothiophenyl, benzoimidazolyl, quinazolinyl, benzothiazolyl, quinoxalinyl, naphthyl, pyridyl, 1H-indazolyl, 1,2,3,4-tetrahydroquinolinyl, benzofuranyl, thienyl, or indolyl, and one end of the linker, Linker I, is attached to

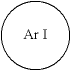

preferably at the 2-position of the ring moiety.

Another aspect of the compounds of Formula II is a compound wherein

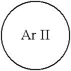

is a 6-membered aryl or heteroaryl group and Linker I and Linker II are attached to

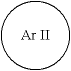

at positions 1,2-, 1,3-, or 1,4- to each other.

Another aspect of the compounds of Formula II is a compound wherein

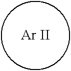

is a naphthyl group, Linker I and Linker II are attached to

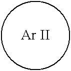

at positions 1,4-, or 2,4- to each other on the naphthyl moiety.

Another aspect of the compounds of Formula II, is a compound wherein

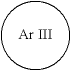

is 6-membered aryl or heteroaryl, and has a preferred position of attachment of Linker II and Linker III to Ring III at positions 1,2-, to each other.

Another aspect of the compounds of Formula II, is a compound wherein

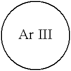

is 6-membered aryl or heteroaryl, and has a preferred position of attachment of Linker II and Linker III to Ring III at positions 1,2-, 1,3-, to each other.

Another aspect of the compounds of Formula II, is a compound wherein

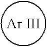

is 6-membered aryl or heteroaryl, and has a preferred position of attachment of Linker II and Linker III to Ring III at positions 1,4- to each other.

A further preferred aspect of the compound of Formula TI is described by Formula V below:

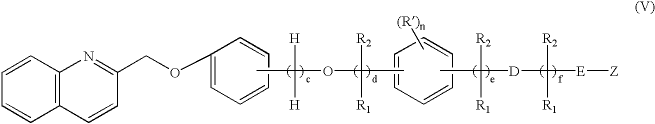

where R₁, R₂, c, d, e, f, n, D, E and Z are as defined above, c+d =1-3, and R′ is a ring group substituent.

A further preferred aspect of the compound of Formula I is a compound wherein

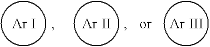

or is independently phenyl, naphthyl, phenyl, naphthyl, 1,2-dihydronaphthylenyl, indenyl, 1,4-naphthoquinonyl, 1,2,3,4-tetrahydronaphthylenyl, 1,4-tetramethyl-2,3-dihydronaphthalenyl, 2,3-dihydro-1,4-naphthoquinonyl, α-tetralonyl, 3H-indolinyl, 2(1H)quinolinonyl, 2H-1-oxoisoquinolyl, 1,2-dihydroquinolinyl, 3,4-dihydroquinolinyl, 1,2-dihydroisoquinolinyl, 3,4-dihydroisoquinolinyl, chromonyl, 3,4-dihydroisoquinoxalinyl, 4-quinazolinonyl, 4H-chromen-2yl, indolinyl, 1,2,3,4-tetrahydroisoquinolinyl, 1,2,3,4-tetrahydroquinolinyl, 1H-2,3-dihydroisoindol-2-yl, 2,3-dihydrobenz[f]isoindol-2-yl, 1,2,3,4-tetrahydrobenz[g]isoquinolin-2-yl, chromanyl, isochromanonyl, 2,3-dihydrochromonyl, 1,4-benzodioxan, 1,2,3,4-tetrahydroquinoxalinyl, quinolinyl, indazolyl, indolyl, quinazolinyl, pyridyl, pyrimidinyl, furyl, benzothiazol, quinoxalinyl, benzimidazolyl, benzothienyl, or isoquinolinyl, 5,6-dihydroquinolyl, 5,6-dihydroisoquinolyl, 5,6-dihydroquinoxalinyl, 5,6-dihydroquinazolinyl, 4,5-dihydro-1H-benzimidazolyl, 4,5-dihydrobenzoxazolyl, 1,4-naphthoquinolyl, 5,6,7,8-tetrahydroquinolinyl, 5,6,7,8-tetrahydroisoquinolyl, 5,6,7,8-tetrahydroquinoxalinyl, 5,6,7,8-tetrahydroquinazolyl, 4,5,6,7-tetrahydro-1H-benzimidazolyl, 4,5,6,7-tetrahydrobenzoxazolyl, 1H-4-oxa-1,5-diazanaphthalen-2-onyl, 1,3-dihydroimidizole-[4,5]-pyridin-2-onyl, 2,3-dihydro-1,4-dinaphthoquinonyl, 7,8-dihydro[1,7]naphthyridinyl, 1,2-dihydro[2,7]naphthyridinyl, 6,7-dihydro-3H-imidazo[4,5-c]pyridyl, 1,2-dihydro-1,5-naphthyridinyl, 1,2-dihydro-1,6-naphthyridinyl, 1,2-dihydro-1,7-naphthyridinyl, 1,2-dihydro-1,8-naphthyridinyl, 1,2-dihydro-2,6-naphthyridinyl, 2,3-dihydro-1H pyrrol[3,4-b]quinolin-2-yl, 1,2,3,4-tetrahydrobenz[b][1,7]naphthyridin-2-yl, 1,2,3,4-tetrahydrobenz[b][1,6]naphthyridin-2-yl, 1,2,3,4-tetrahydro-9H-pyrido[3,4-b]indol-2yl, 1,2,3,4-tetrrahydro-9H-pyrido [4,3-b)indol-2yl, 2,3,-dihydro-1H-pyrrolo[3,4-b]indol-2-yl, 1H-2,3,4,5-tetrahydroazepino[3,4-b]indol-2-yl, 1H-2,3,4,5-tetrahydroazepino[4,3-b]indol-3-yl, 1H-2,3,4,5-tetrahydroazepino[4,5-b]indol-2yl, 5,6,7,8-tetrahydro[1,7]napthyridinyl, 1,2,3,4-tetrhydro2,7]naphthyridyl, 2,3-dihydro[1,4]dioxino[2,3-b]pyridyl, 2,3-dihydro[1,4]dioxino[2,3-b]pryidyl, 3,4-dihydro-2H-1-oxa[4,6]diazanaphthalenyl, 4,5,6,7,-tetrahydro-3H-imidazo[4,5-c]pyridyl, 6,7-dihydro[5,8]diazanaphthalenyl, 1,2,3,4-tetrahydro[1,5]napthyridinyl, 1,2,3,4-tetrahydro[1,6]napthyridinyl, 1,2,3,4-tetrahydro[1,7]napthyridinyl, 1,2,3,4-tetrahydro[1,8]napthyridinyl, 1,2,3,4-tetrahydro[2,6]napthyridinyl.

More particularly, a further preferred aspect of the compound of Formula I is

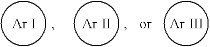

is independently phenyl, naphthyl, quinolyl, isoquinolyl, 1,2,3,4,-tetrahydronaphthyl, pyridyl, pyrazinyl, pyrimidinyl, pyridazinyl, quinazolinyl, phthalazinyl, naphthyridinyl, quinoxalinyl, quinazolinyl, cinnolinyl, pteridinyl, benzofuryl, benzimidazolyl, thienyl, oxazolyl, indolyl, furyl, α-tetralonyl, isochromanonyl, 1,4-naphthoquinolyl, 2,3-dihydro-1,4-dinaphthoquinonyl.

A further preferred aspect of compounds of Formula I is the compound wherein at least one of a, b, e, f, h is independently 0.

A further preferred aspect of compounds of Formula I is the compound wherein at least one of a, b, e, f or h is independently 1.

A further preferred aspect of the compound of Formula I is the compound wherein at least one of a, b, e, f, g, or h is independently 2.

A further preferred aspect of compounds of Formula I is the compound wherein at least one of a, b, e, f, g, or h is independently 3.

A further preferred aspect of compounds of Formula I is the compound wherein at least one of a, b, e, f, g, or h is independently 4.

A further preferred aspect of compounds of Formula I is the compound wherein f is 5.

A further preferred aspect of compounds of Formula I is the compound wherein f is 6.

A further preferred aspect of the compound of Formula I is the compound wherein a=1, A is O, and b=0.

A further preferred aspect of the compound of Formula I is a compound wherein a=0, A is

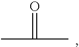

and b=0.

A further preferred aspect of compounds of Formula I is a compound wherein a=0, A is

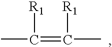

and b=0.

A further preferred aspect of compounds of Formula I is a compound wherein c=0, and d=1.

A further preferred aspect of compounds of Formula I is a compound wherein c=0, B is O, and d=1.

A further preferred aspect of compounds of Formula I is a compound wherein c=0, B is

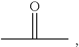

d=1, R₁ is hydrogen, R₂ is —(CH₂)_{q}—X, q is 1, is heteroaryl.

A further preferred aspect of compounds of Formula I is a compound wherein a+b=0-2.

A further preferred aspect of compounds of Formula I is a compound wherein a+b=1.

A further preferred aspect of compounds of Formula I is a compound wherein c=1, d=0.

A further preferred aspect of compounds of Formula I is a compound wherein B is a chemical bond.

A further preferred aspect of compounds of Formula I is a compound wherein c=1, d=0, and B is a chemical bond.

A further preferred aspect of compounds of Formula I is a compound wherein c=0, d=0, and B is a chemical bond.

A further preferred aspect of compounds of Formula I is a compound wherein e+f=0-4.

A further preferred aspect of compounds of Formula I is a compound wherein e+f=3.

A further preferred aspect of compounds of Formula I is a compound wherein e+f=1.

A further preferred aspect of compounds of Formula I is a compound wherein e+f=1, and D and E are chemical bonds.

A further preferred aspect of compounds of Formula I is a compound wherein f=1, 2, or 3.

A further preferred aspect of compounds of Formula I is a compound wherein A is NR₅

A further preferred aspect of compounds of Formula I is a compound wherein A is

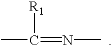

A further preferred aspect of compounds of Formula I is a compound wherein A is

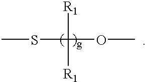

A further preferred aspect of compounds of Formula I is a compound wherein A is

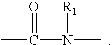

A further preferred aspect of compounds of Formula I is a compound wherein A is

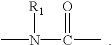

A further preferred aspect of compounds of Formula I is a compound wherein D is

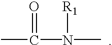

A further preferred aspect of compounds of Formula I is a compound wherein D is

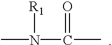

A further preferred aspect of compounds of Formula I is a compound wherein D is

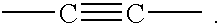

A further preferred aspect of compounds of Formula I is a compound wherein D is O.

A further preferred aspect of compounds of Formula I is a compound wherein D is S.

A further preferred aspect of compounds of Formula I is a compound wherein D is a chemical bond.

A further preferred aspect of compounds of Formula I is a compound wherein D is NR₄.

A further preferred aspect of compounds of Formula I is a compound wherein e=0, and D is O.

A further preferred aspect of compounds of Formula I is a compound wherein e=0 and D is a chemical bond.

A further preferred aspect of compounds of Formula I is a compound wherein e=0, D is a chemical bond, and E is a chemical bond.

A further preferred aspect of compounds of Formula I is a compound wherein e=1 and geminal R₁ and R₂ taken together with the carbon atom to which the geminal R₁ and R₂ are attached form carbonyl.

A further preferred aspect of compounds of Formula I is a compound wherein e=1 and geminal R₁ and R₂ taken together with the carbon atom to which the geminal R₁ and R₂ are attached form cycloalkylene.

A further preferred aspect of compounds of Formula I is a compound wherein two R₁ taken together with the carbons atom to which the R₁ are linked form cycloalkylene.

A further preferred aspect of compounds of Formula I is a compound wherein two vicinal R₁ taken together with the carbons atom to which the vicinal R₁ are linked form

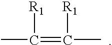

A further preferred aspect of compounds of Formula I is a compound wherein geminal R₁ and R₁ taken together with the carbon atom to which the geminal R₁ and R₁ are attached to form carbonyl.

A further preferred aspect of the compound of Formula I is a compound wherein R₁ is carboxyl.

A further preferred aspect of the compound of Formula I is a compound wherein R₁ is alkoxycarbonyl.

A further preferred aspect of compounds of Formula I is a compound wherein e=2, and geminal R₁ and R₂ taken together with the carbon atom to which the geminal R₁ and R₂ are attached independently form cycloalkylene or carbonyl.

A further preferred aspect of compounds of Formula I is a compound wherein e=2, R₁ and R₂ are independently alkyl, or geminal R₁ and R₂ taken together with the carbon atom to which the geminal R₁ and R₂ are attached form carbonyl.

A further preferred aspect of compounds of Formula I is a compound wherein D is O, e=2, R₁ and R₂ are independently alkyl, or geminal R₁ and R₂ taken together with the carbon atom to which the geminal R₁ and R₂ are attached form carbonyl.

A further preferred aspect of compounds of Formula I is a compound wherein f=2, R₁ and R₂ are independently alkyl, or geminal R₁ and R₂ taken together with the carbon atom to which the geminal R₁ and R₂ are attached form carbonyl.

A further preferred aspect of compounds of Formula I is a compound wherein f=2, R₁ is independently hydrogen or alkyl, and R₂ is independently alkyl or alkoxy.

A further preferred aspect of compounds of Formula I is a compound wherein f=1 and geminal R₁ and R₂ taken together with the carbon atom to which the geminal R₁ and R₂ are attached form carbonyl.

A further preferred aspect of compounds of Formula I is a compound wherein f=1, R₁ is hydrogen, and R₂ is hydrogen.

A further preferred aspect of compounds of Formula I is a compound wherein f=1, R₁ is hydrogen, and R₂ is phenyl.

A further preferred aspect of compounds of Formula I is a compound wherein f=1, R₁ is hydrogen, R₂ is —(CH₂)_{q}—X, q=1, and X is carboxy.

A further preferred aspect of compounds of Formula I is a compound wherein f=2, R₁ is hydrogen, R₂ is —(CH₂)_{q}—X, q=1, and X is independently hydrogen or carboxy.

A further preferred aspect of compounds of Formula I is a compound wherein f=3, R₁ is hydrogen, R₂ is —(CH₂)_{q}—X, q=1, and X is independently hydrogen or carboxy.

A further preferred aspect of compounds of Formula I is a compound wherein f=1, R₁ is hydrogen, and R₂ is carboxy.

A further preferred aspect of compounds of Formula I is a compound wherein f=1, R₁ is hydrogen, and R₂ is alkoxycarbonyl.

A further preferred aspect of compounds of Formula I is a compound wherein f=2, R₁ is hydrogen, and R₂ is independently hydrogen or alkoxycarbonyl.

A further preferred aspect of compounds of Formula I is a compound wherein f=3, R₁ is hydrogen, and R₂ is independently hydrogen or alkoxycarbonyl.

A further preferred aspect of compounds of Formula I is a compound wherein f=1, R₁ is hydrogen, and R₂ is alkoxy.

A further preferred aspect of compounds of Formula I is a compound wherein f=2, R₁ is hydrogen, and R₂ is independently hydrogen or alkoxy.

A further preferred aspect of compounds of Formula I is a compound wherein f=3, R₁ is hydrogen, and R₂ is independently hydrogen or alkoxy.

A further preferred aspect of compounds of Formula I is a compound wherein f=1, R₁ is halogen, and R₂ is halogen.

A further preferred aspect of compounds of Formula I is a compound wherein f=2, R₁ is halogen, and R₂ is independently hydrogen or halogen.

A further preferred aspect of compounds of Formula I is a compound wherein f=3, R₁ is halogen, and R₂ is independently hydrogen or halogen.

A further preferred aspect of compounds of Formula I is a compound wherein f=1, R₁ is fluoro, and R₂ is fluoro.

A further preferred aspect of compounds of Formula I is a compound wherein f=2, R₁ is fluoro, and R₂ is independently hydrogen or fluoro.

A further preferred aspect of compounds of Formula I is a compound wherein f=3, R₁ is fluoro, and R₂ is independently hydrogen or fluoro.

A further preferred aspect of compounds of Formula I is a compound wherein f=1, R₁ is alkyl, and R₂ is alkyl.

A further preferred aspect of compounds of Formula I is a compound wherein f=2, R₁ is alkyl, and R₂ is independently hydrogen or alkyl.

A further preferred aspect of compounds of Formula I is a compound wherein f=3, R₁ is alkyl, and R₂ is independently hydrogen or alkyl.

A further preferred aspect of compounds of Formula I is a compound wherein f=1, R₁ is aralkyl, and R₂ is alkyl.

A further preferred aspect of compounds of Formula I is a compound wherein f=1, R₁ is aralkyl, and R₂ is aralkyl.

A further preferred aspect of compounds of Formula I is a compound wherein f=1, R₁ is aralkyl, and R₂ is aryl.

A further preferred aspect of compounds of Formula I is a compound wherein f=1, R₁ is aralkyl, and R₂ is heteroaryl.

A further preferred aspect of compounds of Formula I is a compound wherein f=1, R₁ is aralkyl, and R₂ is heteroaralkyl.

A further preferred aspect of compounds of Formula I is a compound wherein R₅ is R₆OC—, R₆NHOC—, hydrogen, alkyl, aryl, heteroaryl, cycloalkyl, heterocyclyl, heteroaralkyl, or aralkyl.

A further preferred aspect of compounds of Formula I is a compound wherein R₅ is R₆OC—, or R₆NHOC—.

A further preferred aspect of compounds of Formula I is a compound wherein R₆ is alkyl, aryl, heteroaryl, cycloalkyl, heterocyclyl, heteroaralkyl, or aralkyl.

A further preferred aspect of compounds of Formula I is a compound wherein R₆ is alky, aryl, cycloalkyl, or aralkyl.

A further preferred aspect of compounds of Formula I is a compound wherein R₆ is, heteroaryl, heterocyclyl, heteroaralkyl, or aralkyl.

A further preferred aspect of compounds of Formula I is a compound wherein R₆ is hydrogen.

A further preferred aspect of compounds of Formula I is a compound wherein E is a chemical bond.

A more preferred aspect of the compound of Formula I are those compounds wherein Z is —COOR₁, —CN, R₃O₂SHNCO—, or tetrazolyl.

A further preferred aspect of compounds of Formula I is a compound wherein Z is tetrazolyl.

A further preferred aspect of compounds of Formula I is a compound wherein Z is R₃O₂C—, and R₃ is hydrogen or alky.

A further preferred aspect of compounds of Formula I is a compound wherein Z is R₃OC—, and each R₃ is independently hydrogen, alkyl, or aryl A further preferred aspect of compounds of Formula I is a compound wherein Z is CN.

A further preferred aspect of compounds of Formula I is a compound wherein Z is R₃O₂SHNCO—, and R₃ is hydrogen, alkyl, or aryl.

A further preferred aspect of compounds of Formula I is a compound wherein Z is R₃O₂SHNCO—, and R₃ is phenyl.

A further preferred aspect of compounds of Formula I is a compound wherein Z is R₃O₂SHN—.

A further preferred aspect of compounds of Formula I is a compound wherein Z is (R₃)₂NCO—, and R₃ is hydrogen or alkyl.

A further preferred aspect of compounds of Formula I is a compound wherein Z is R₃O— and R₃ is hydrogen, alkyl, or aryl.

A further preferred aspect of compounds of Formula I is a compound wherein f=1, R₁ is hydrogen, R₂ is —(CH2)_{q}—X, q=1, and X is alkyl.

A further preferred aspect of compounds of Formula I is a compound wherein R₁ is H, alkyl, or aryl.

A further preferred aspect of compounds of Formula I is a compound wherein A is

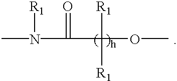

A further preferred aspect of compounds of Formula I is a compound wherein A is

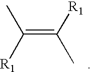

A further preferred aspect of compounds of Formula I is a compound wherein B is

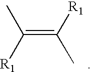

A further preferred aspect of compounds of Formula I is a compound wherein B is

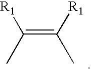

A further preferred aspect of compounds of Formula I is a compound wherein D is

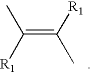

A further preferred aspect of compounds of Formula I is a compound wherein E is

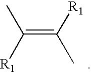

A more preferred aspect of the compound of Formula I are those where X is hydrogen, alkyl, alkenyl, cycloalkyl, aryl, aralkyl, hydroxy, alkoxy, aralkoxy, carboxy, alkoxycarbonyl, tetrazolyl, acylHNSO₂—, Y¹Y²N— or Y³Y⁴NCO—.

A more preferred aspect of the compound of Formula I are those compounds wherein Y¹ and Y² are independently hydrogen, alkyl, or aralkyl or one of Y¹ and Y² is hydrogen and the other of Y¹ and Y² is acyl.

A more preferred aspect of the compound of Formula I are those where Y³ and Y⁴ are hydrogen.

A more preferred aspect of the compounds of Formula V are those compounds wherein Z is —COOR₁, —CN, R₃O₂SHNCO—, or tetrazolyl.

A preferred compound according to the invention is selected from the group consisting of

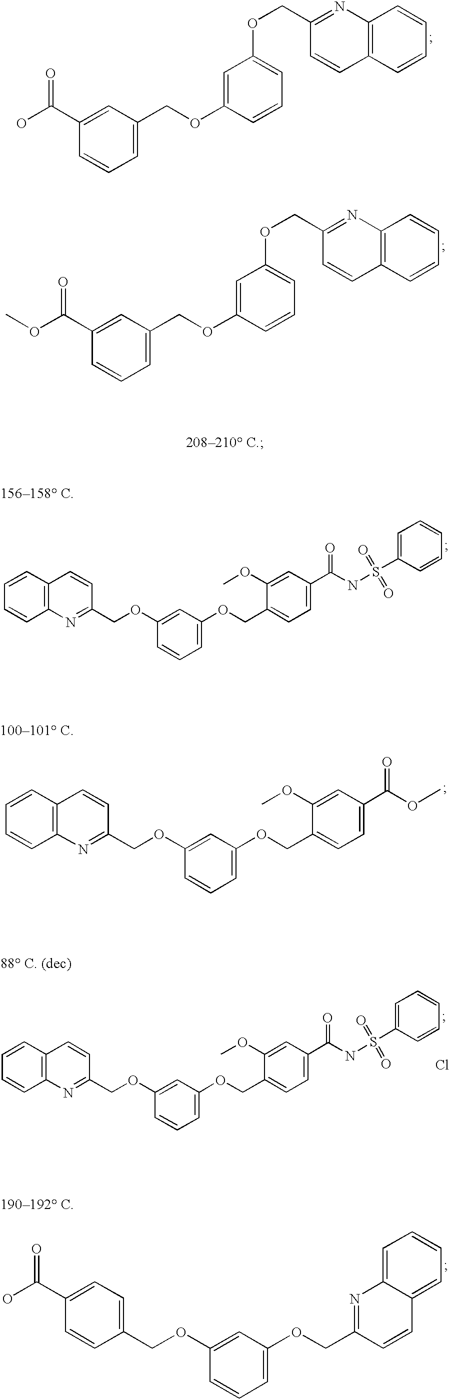

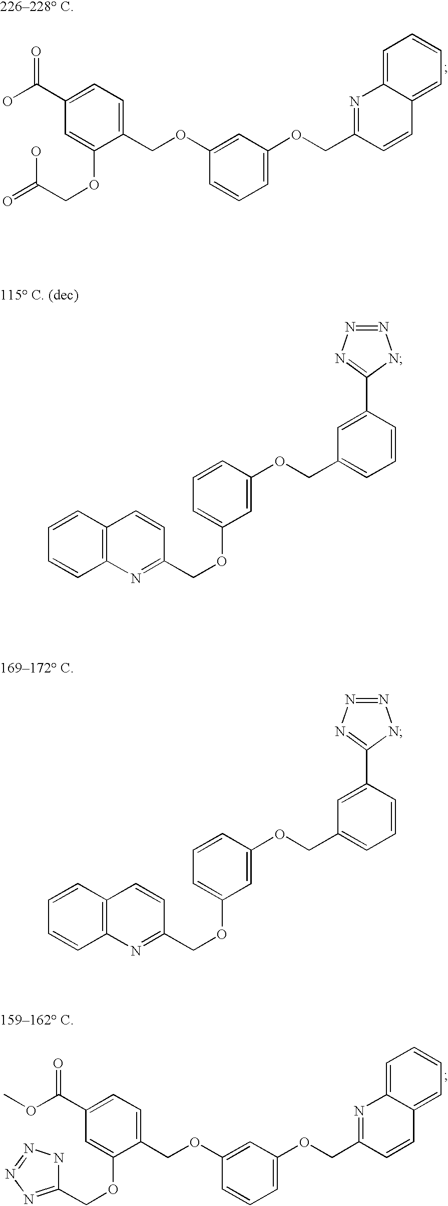

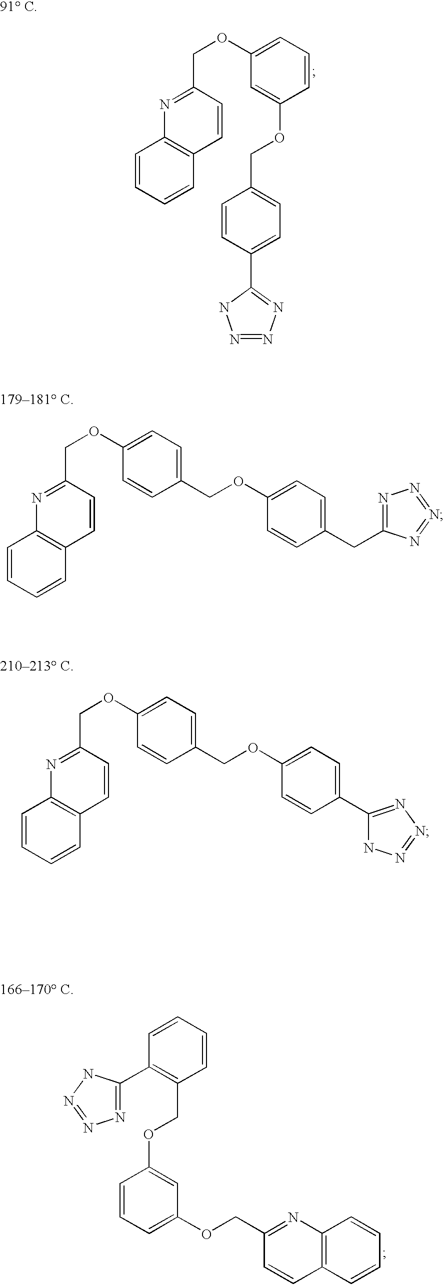

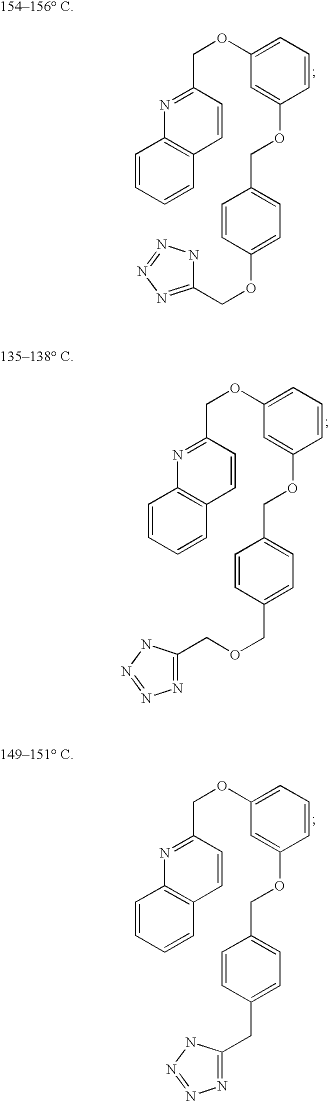

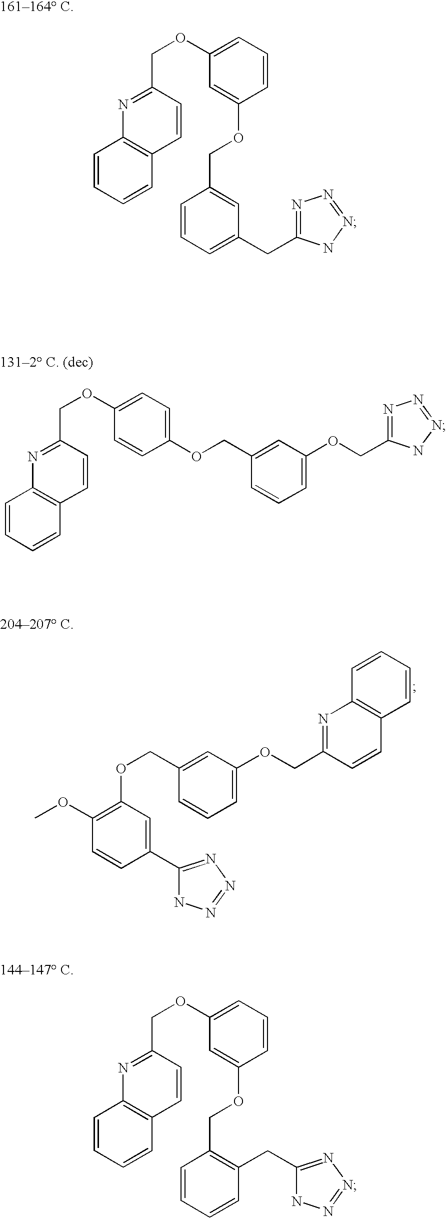

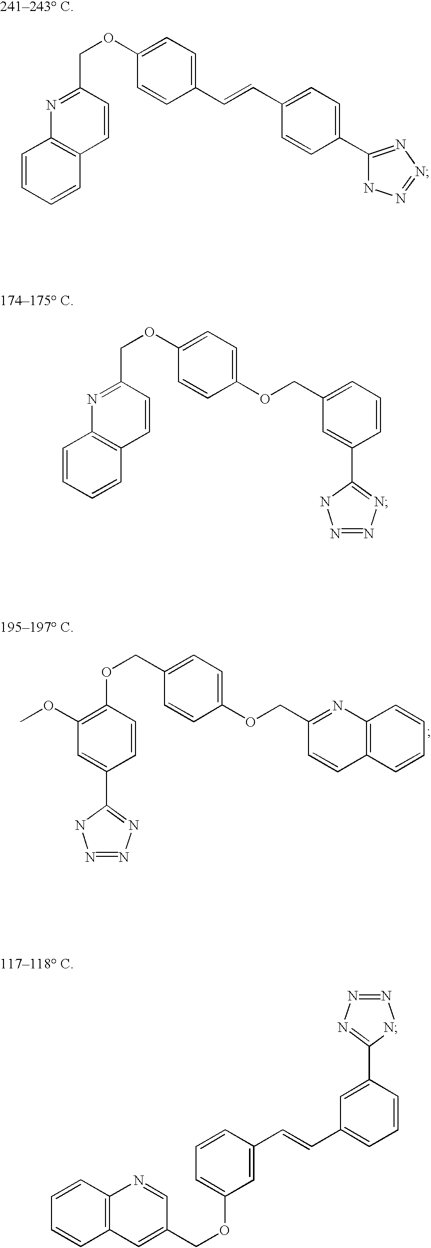

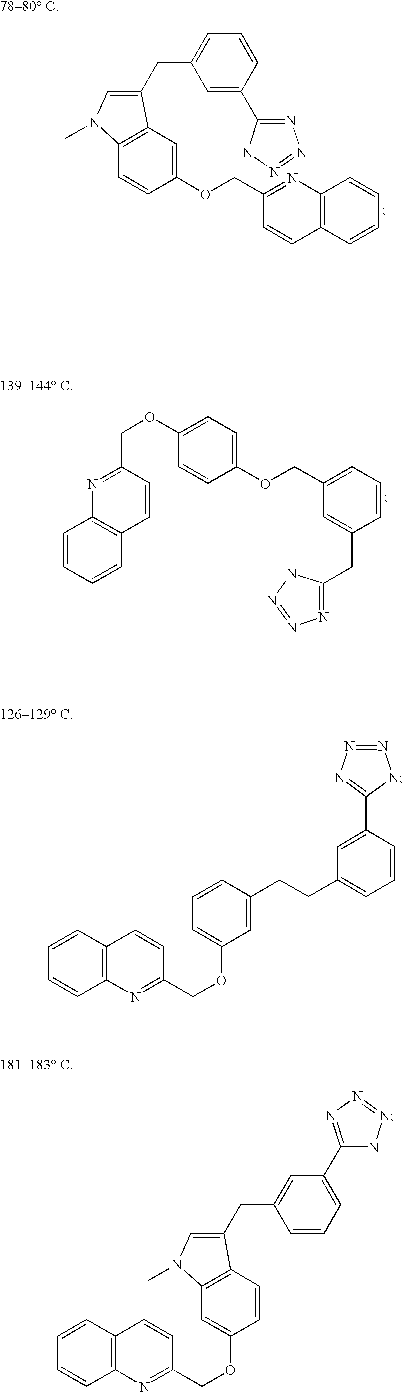

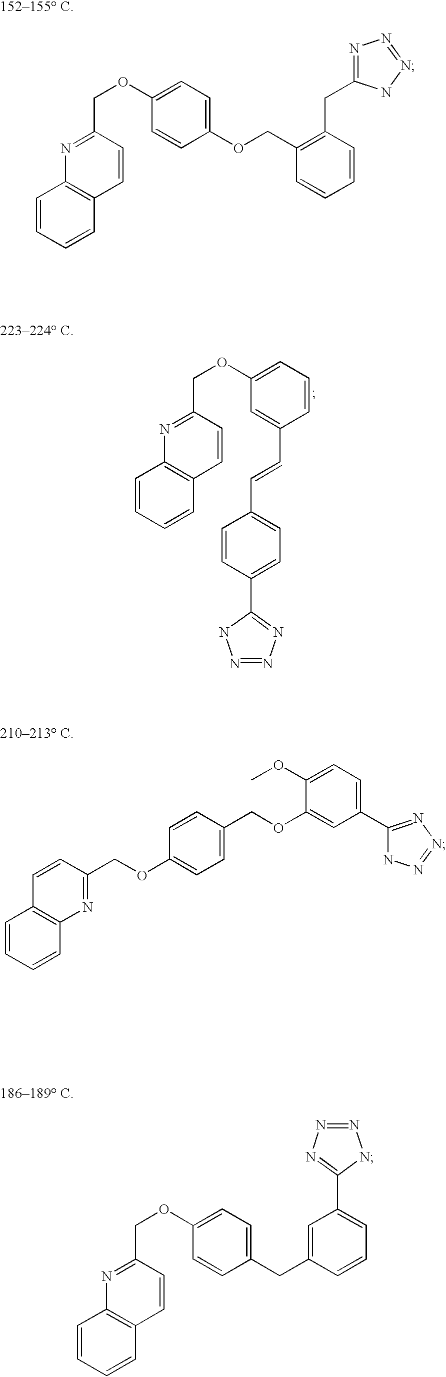

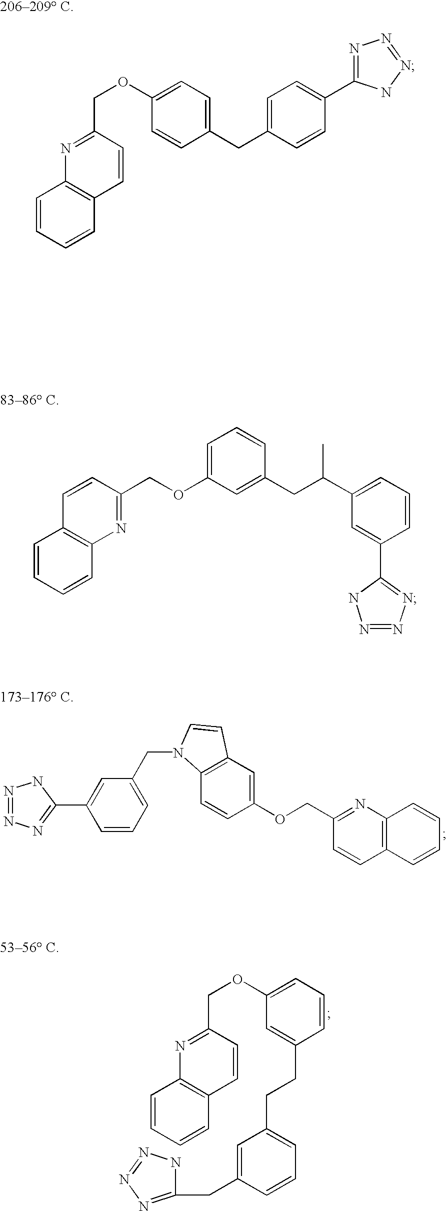

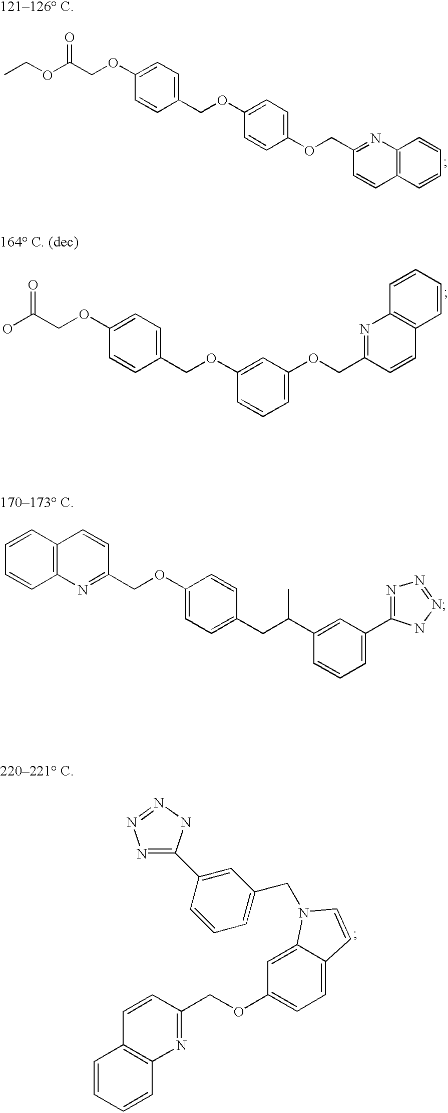

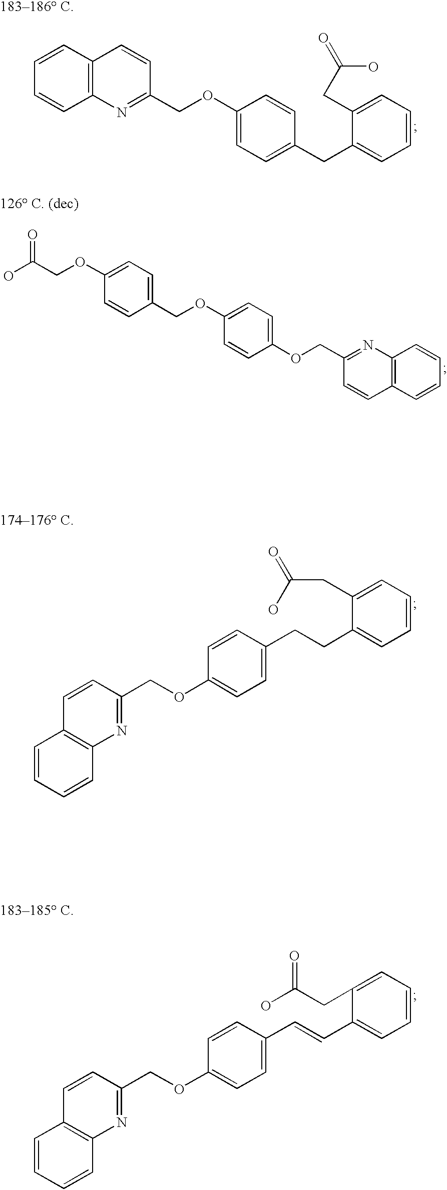

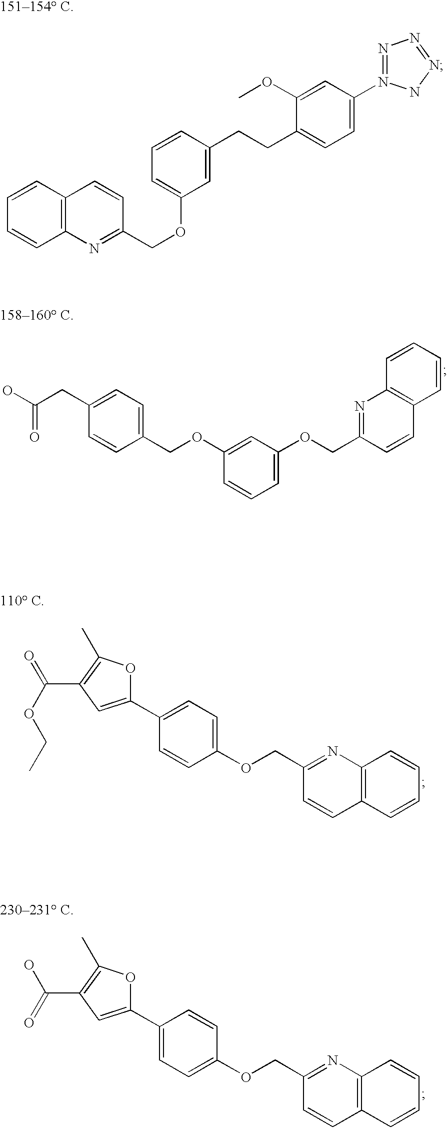

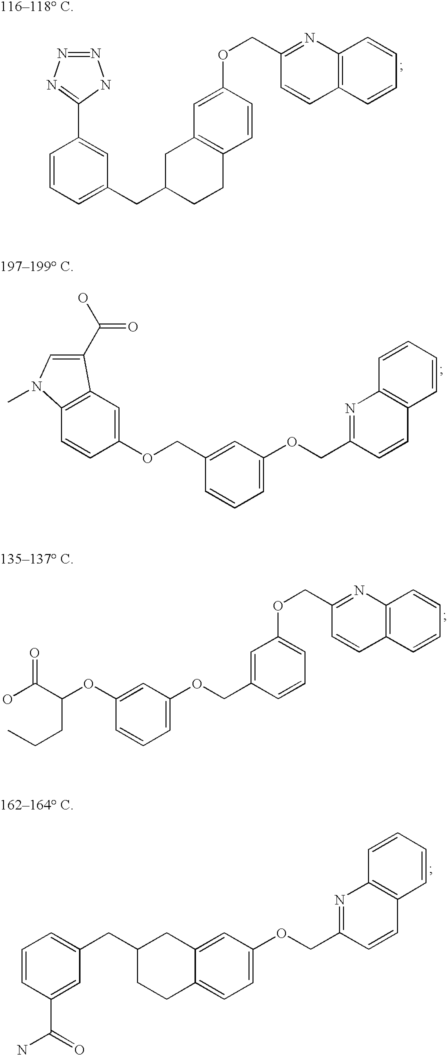

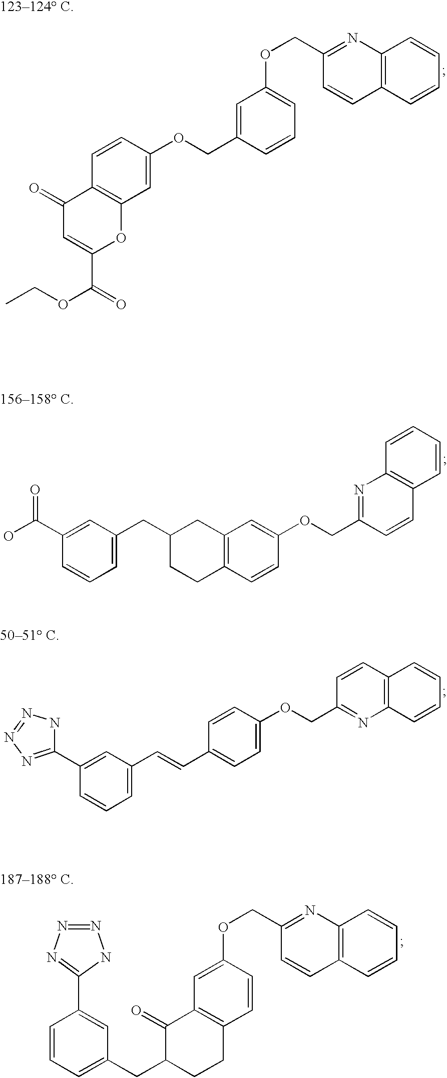

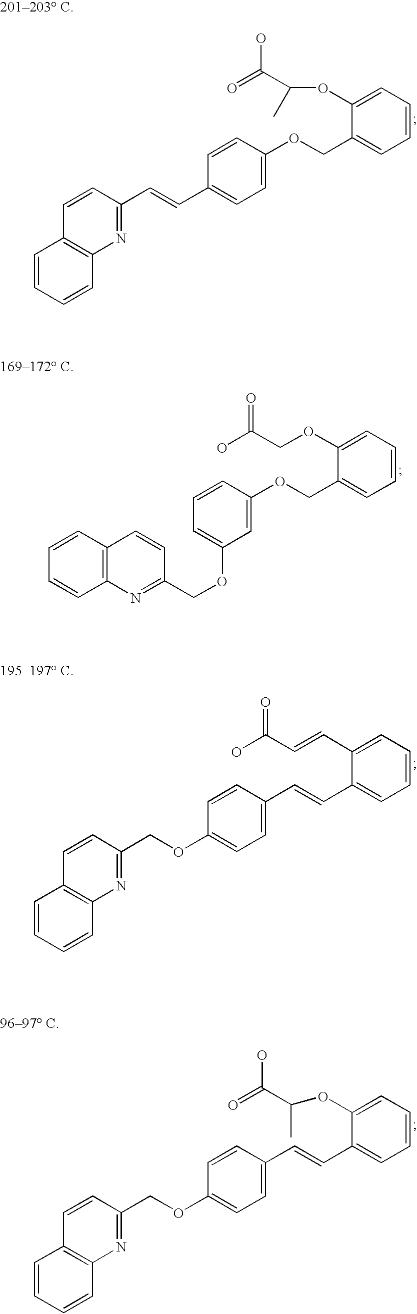

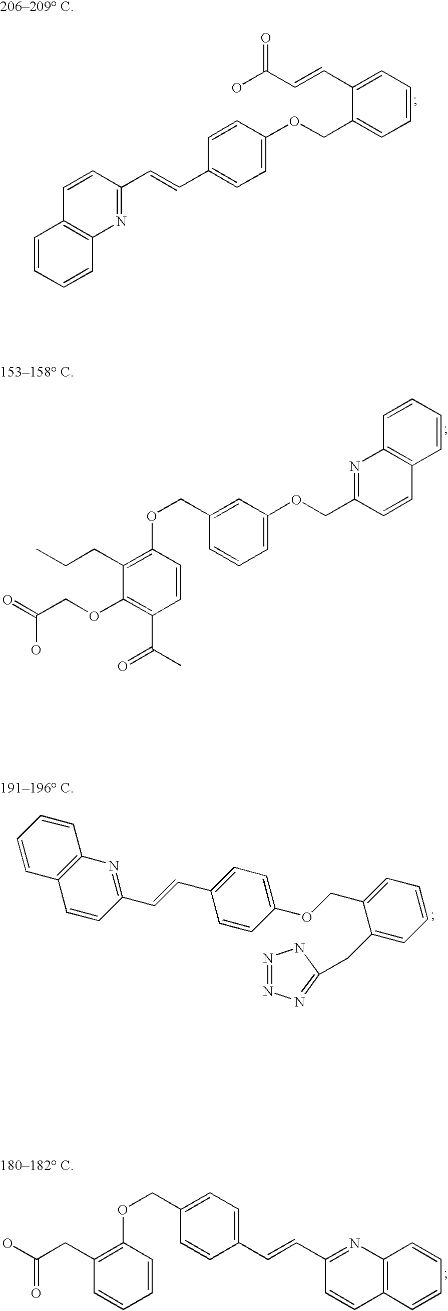

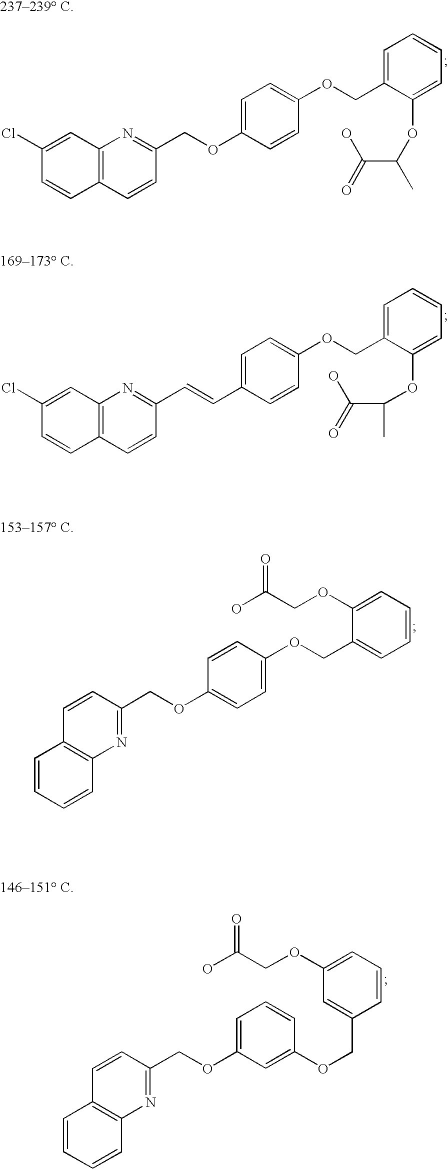

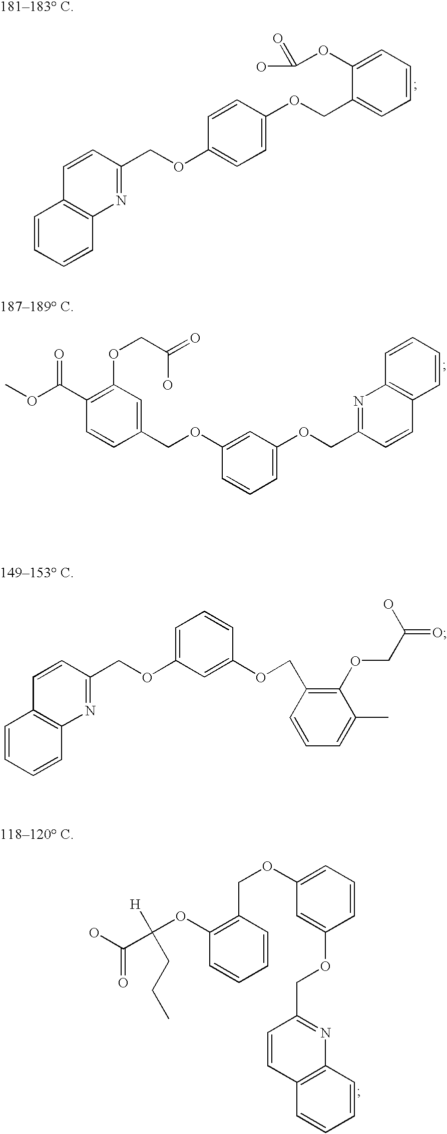

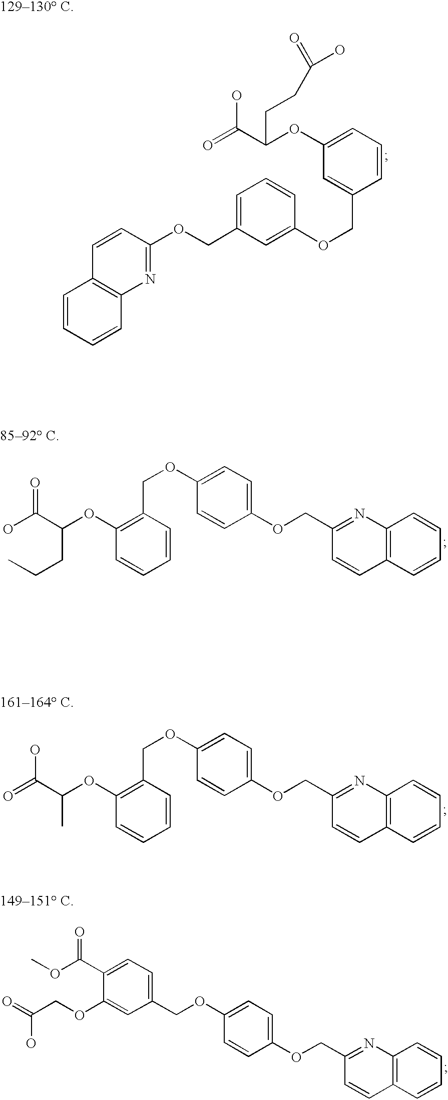

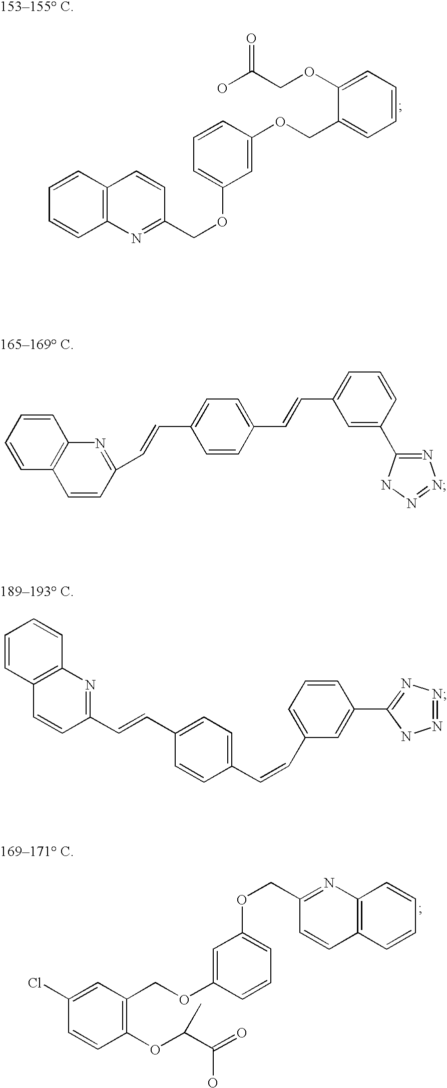

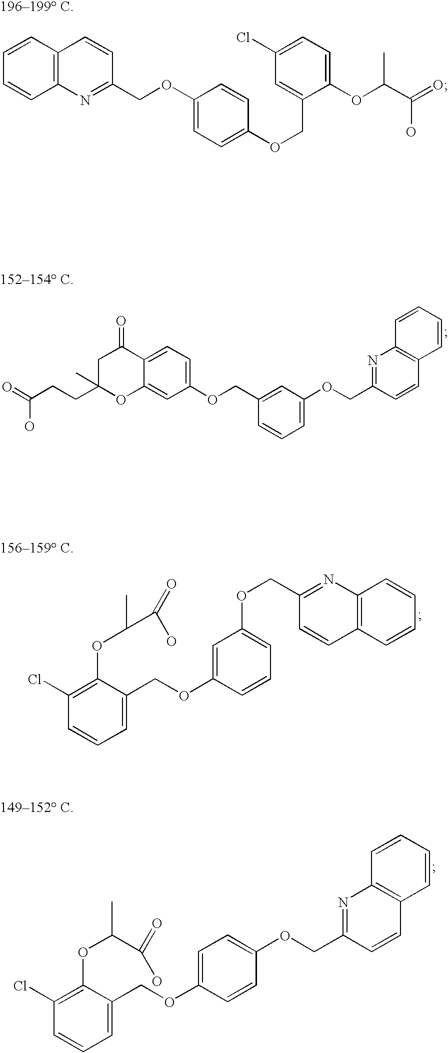

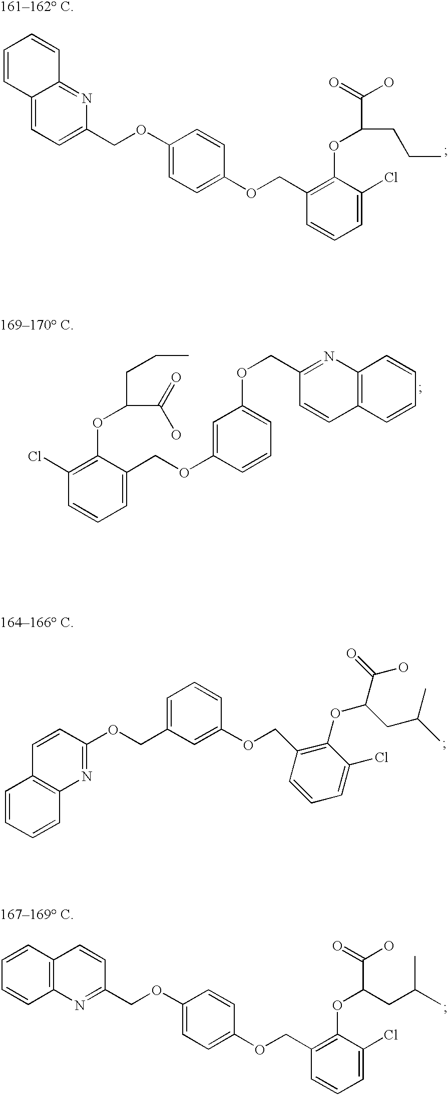

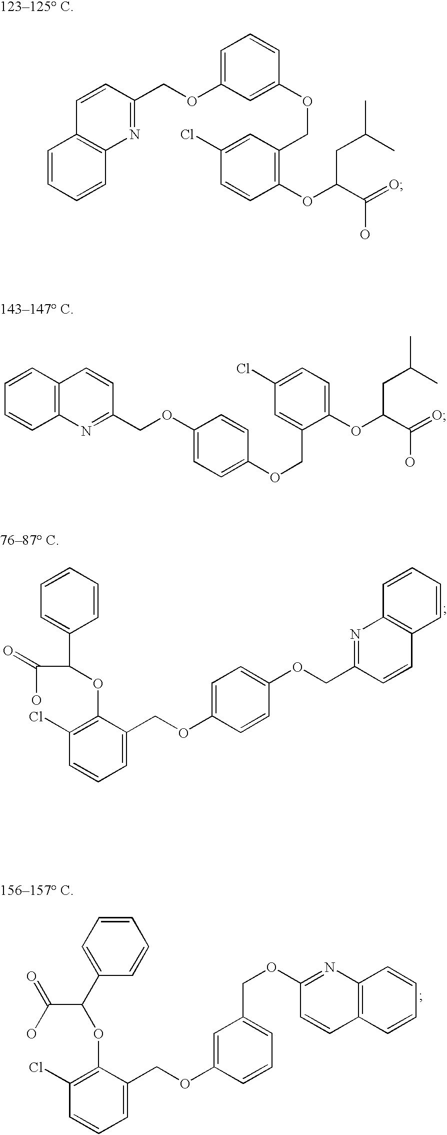

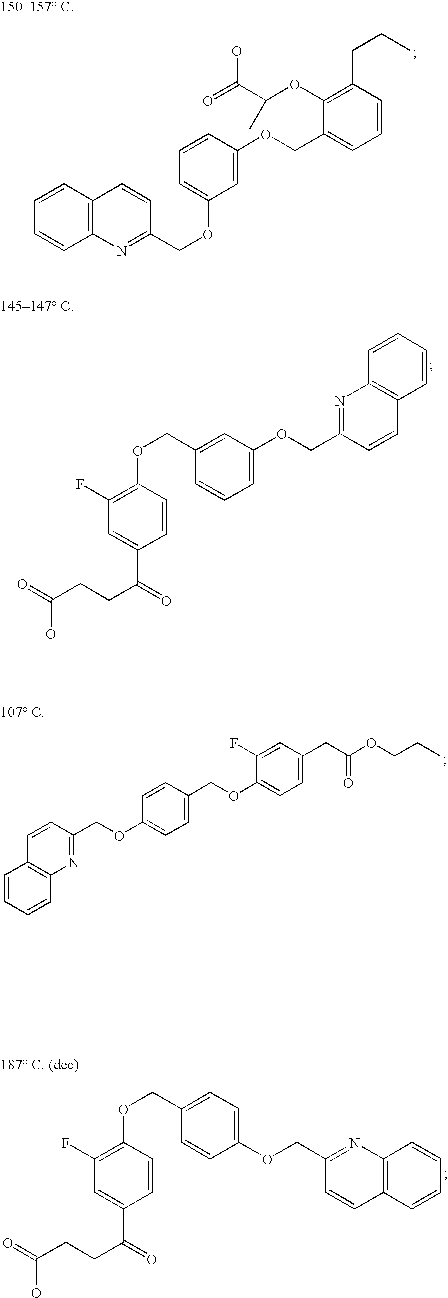

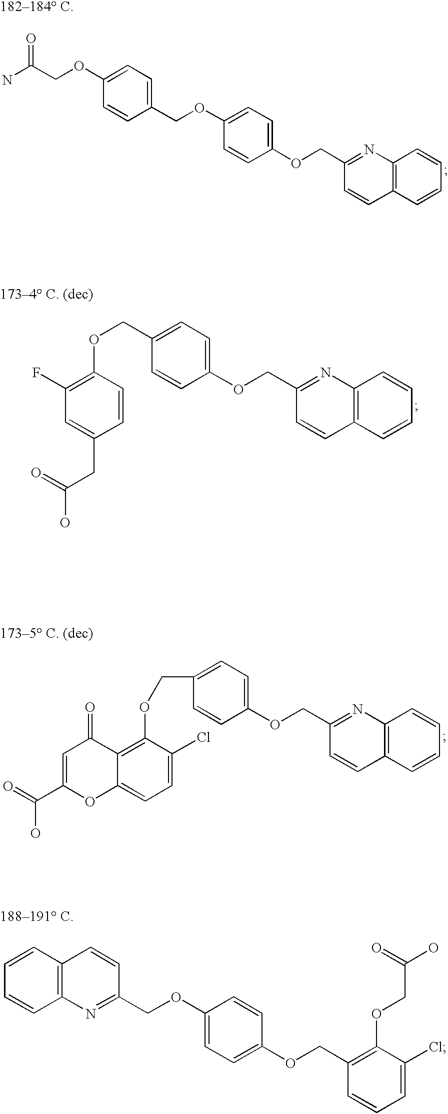

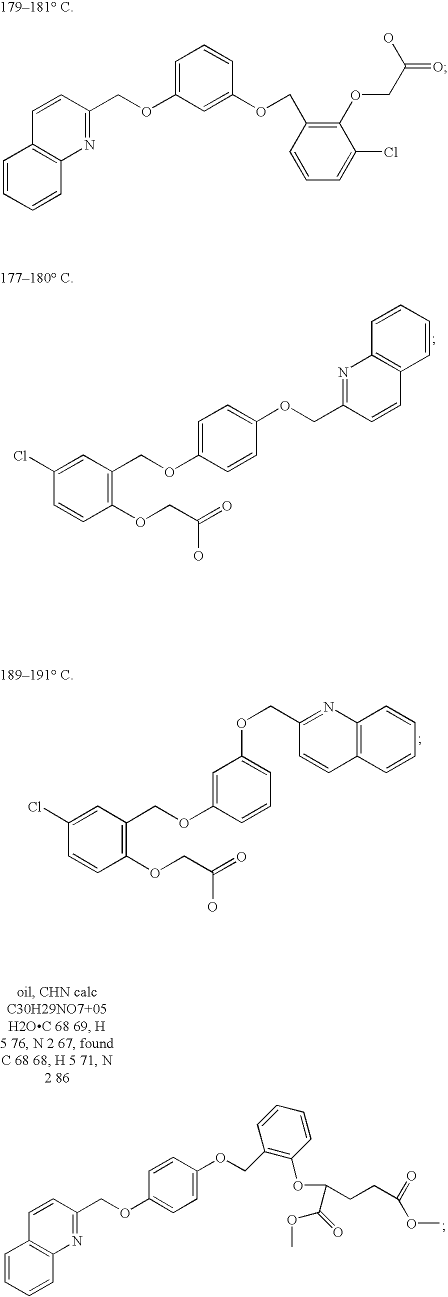

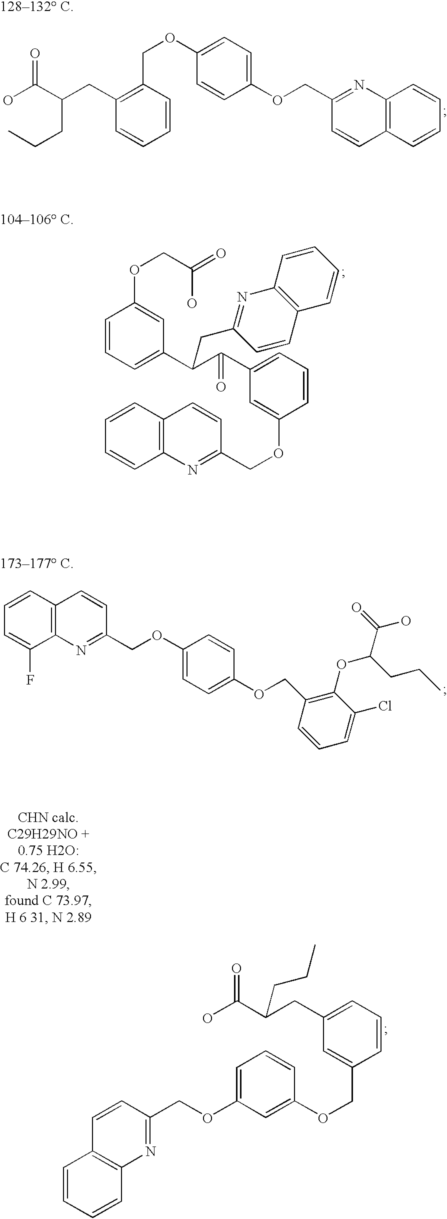

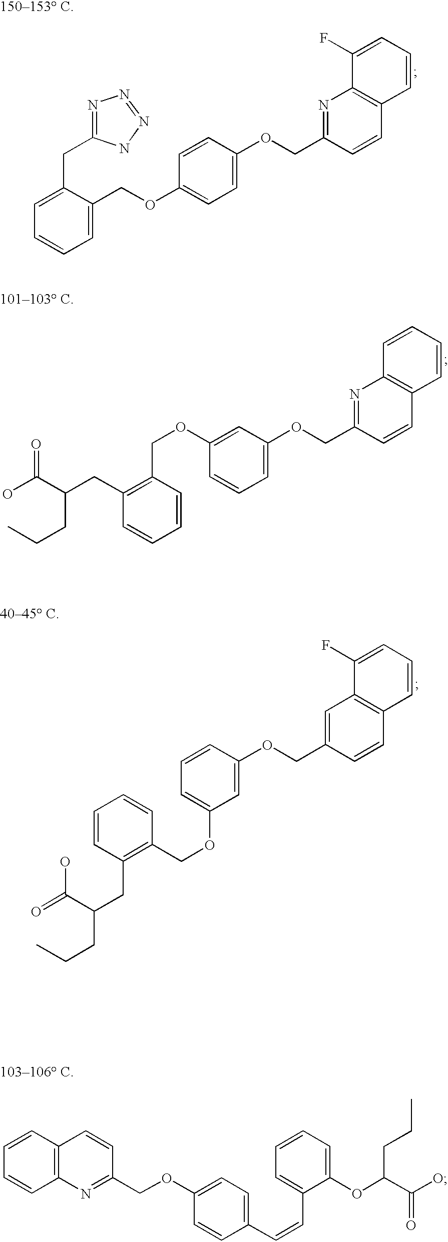

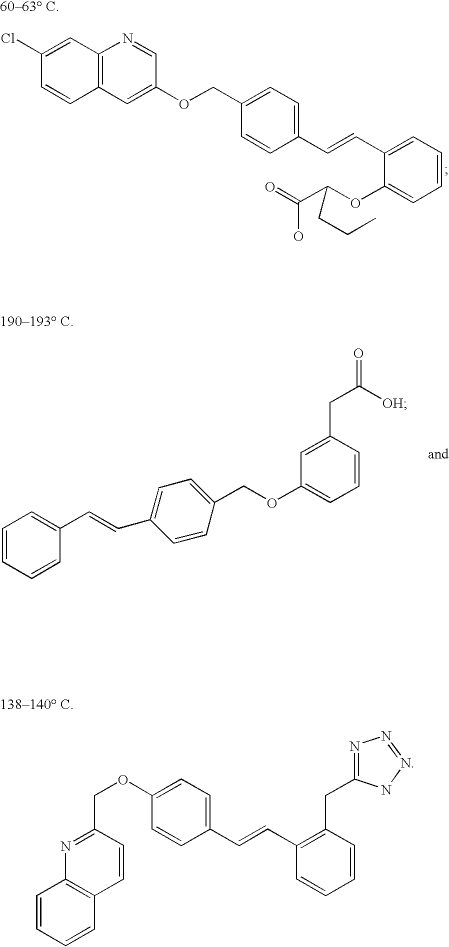

A preferred compound according to the invention is selected from the group consisting of

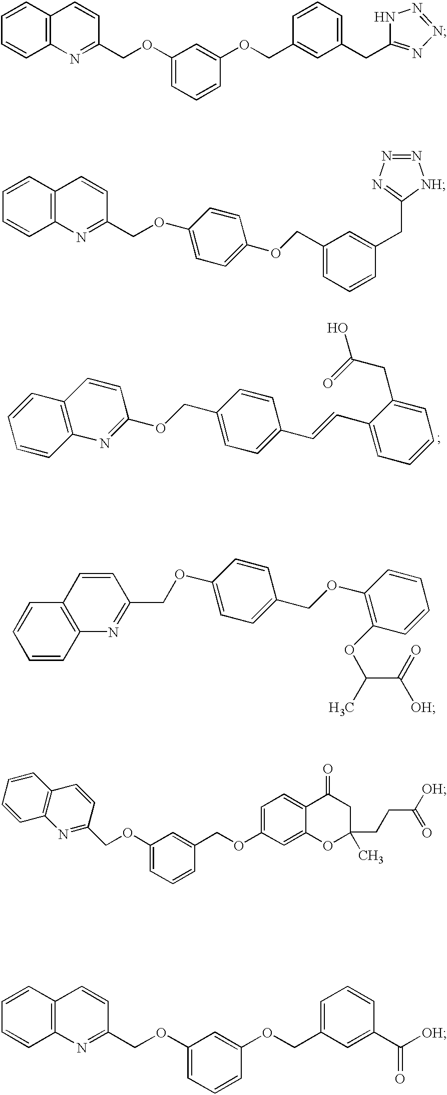

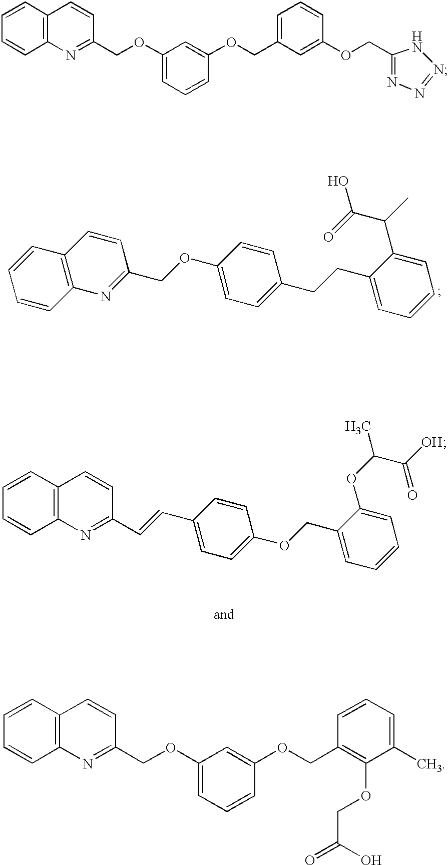

A more preferred compound according to the invention is selected from the group consisting of

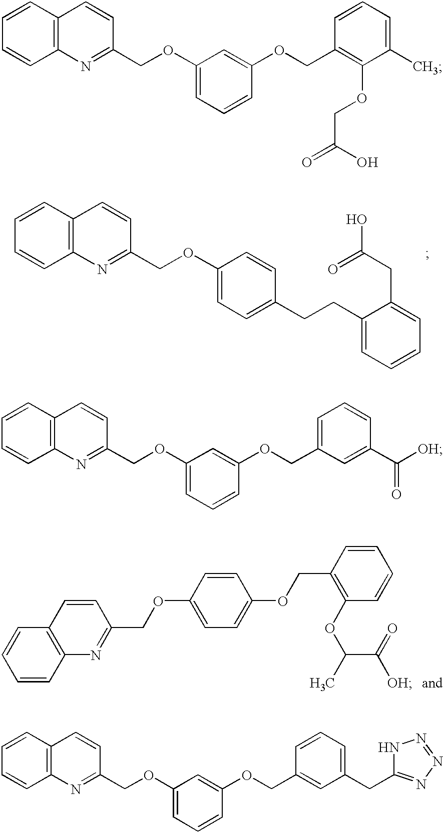

A preferred compound according to the invention having PPARα and PPARγ activity is selected from the group consisting of

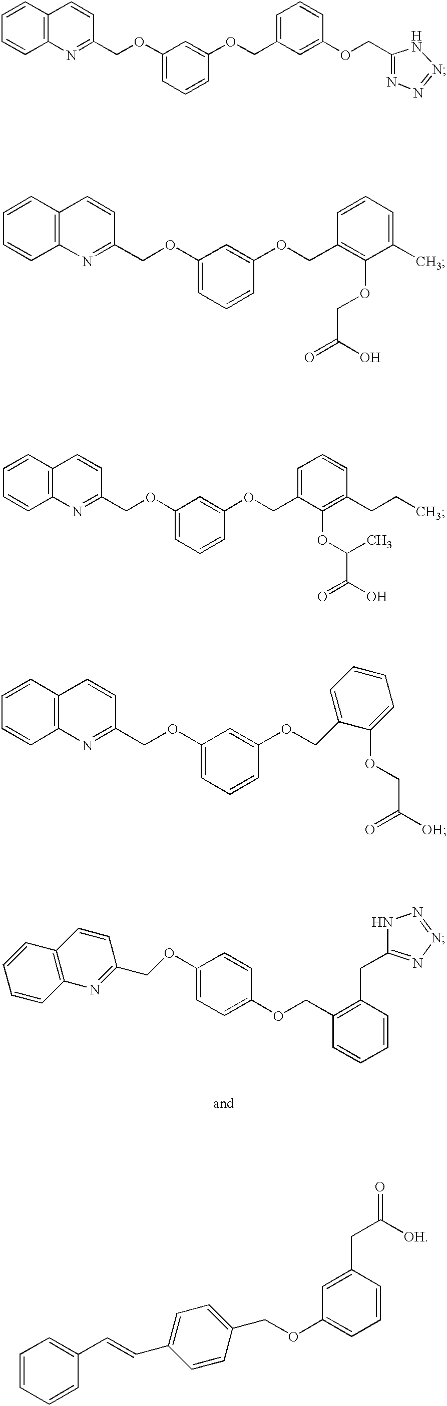

A preferred compound according to the invention that is selective for PPARα is selected from the group consisting of

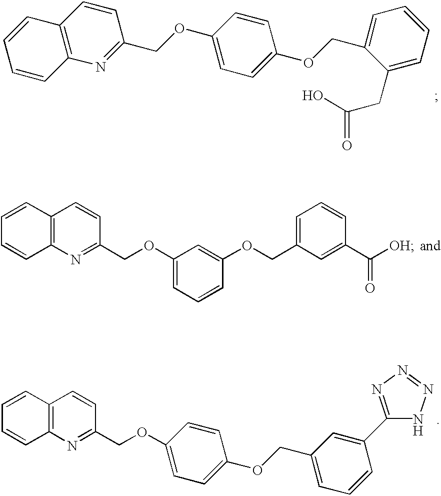

A preferred compound according to the invention that is selective for PPARδ is selected from the group consisting of:

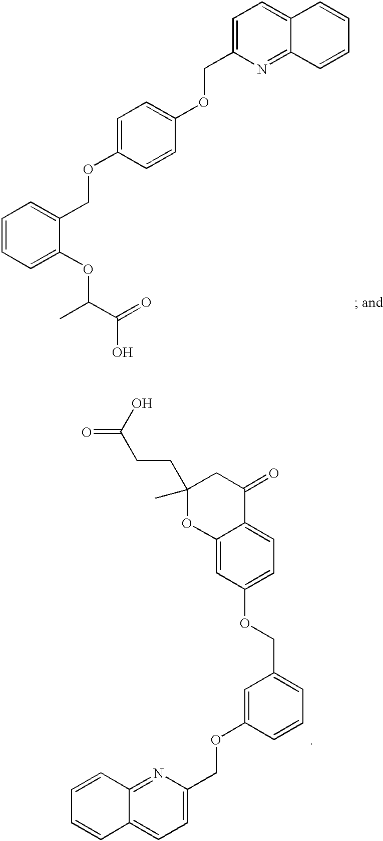

A preferred compound according to the invention that is selective for PPARδ and PPARγ is selected from the group consisting of:

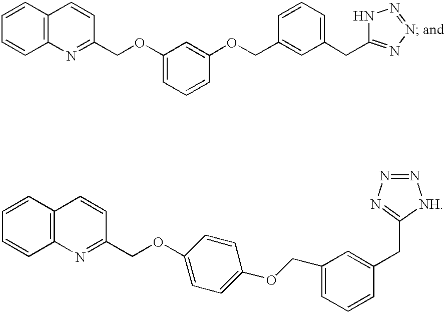

A preferred compound according to the invention that is selective for PPARα and PPARδ is selected from the group consisting of:

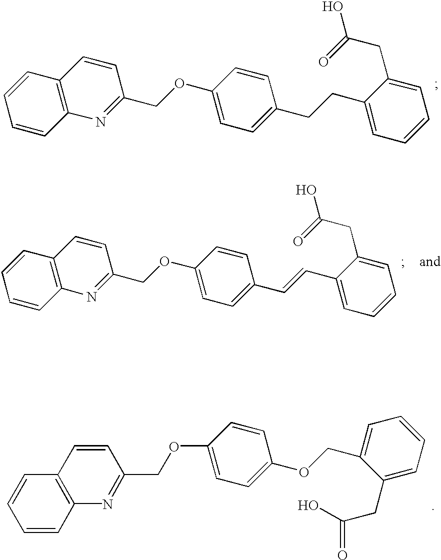

A more preferred compound of the invention having PPARγ activity has the formula VI:

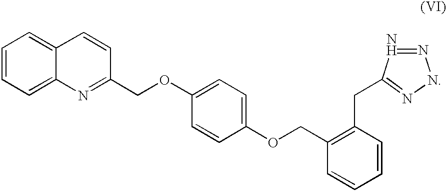

This invention also encompasses all combinations of preferred aspects of the invention noted herein.

Compounds useful according to this invention can be prepared in segments as is common to a long chain molecule. Thus it is convenient to synthesize these molecules by employing condensation reactions at the A, B and D sites of the molecule. Compounds of Formula I can be prepared by the application or adaptation of known methods, by which is meant methods used heretofore or described in the literature. Thus, compounds of Formula I are preparable by art recognized procedures from known compounds or readily preparable intermediates. Exemplary general procedures are as follows. These are illustrative for the synthesis of compounds of formula II wherein ArI is quinolinyl, ArII is aryl, ArIII is aryl, R, R′, R_{1 and R}₂ are all hydrogen; b, d and e are 0; a, c, and f are 1; or b, c, e and f are 0 and a and d are 1. B is O, S or NR₄ and Z is —CN, COOR₃ or tetrazolyl. Thus, in order to prepare a compound of the below formula

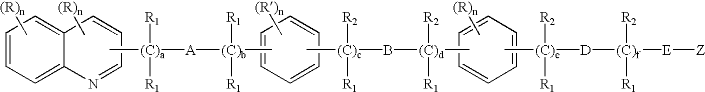

the following reactions or combinations of reactions are employable:

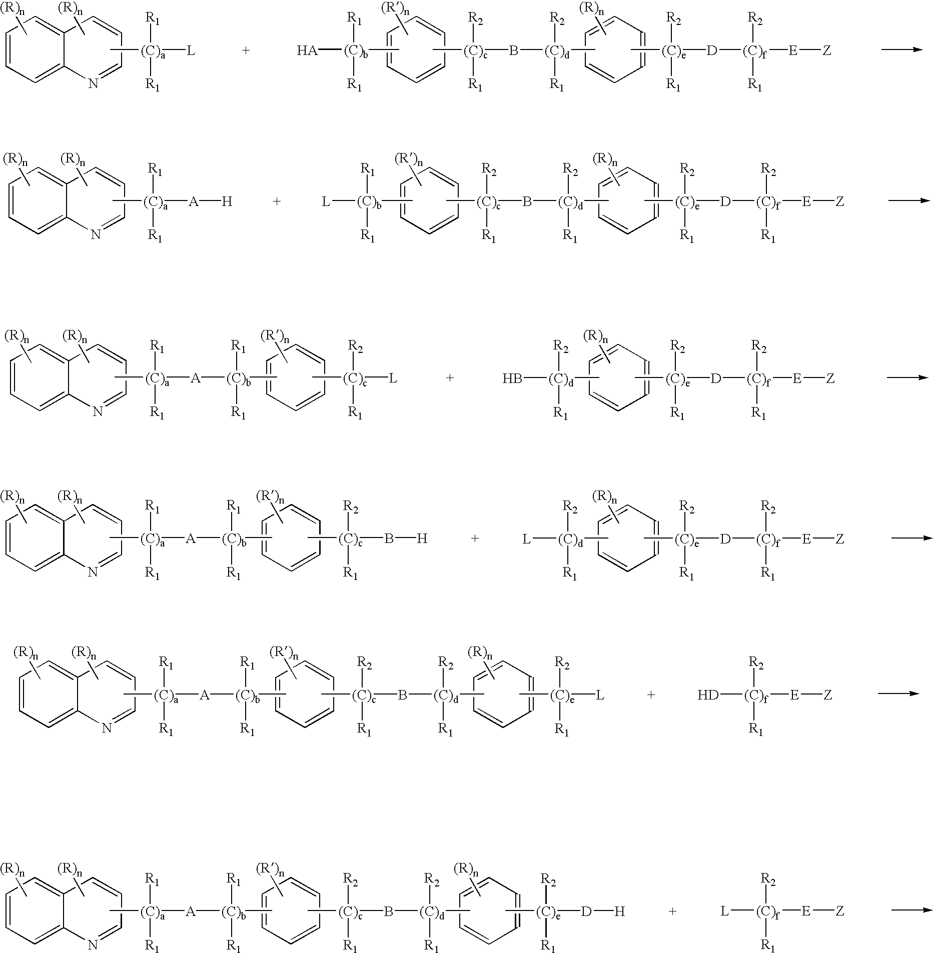

wherein:

R, R′, R₁, R₂, a, b, c, d, e, f, n, A, and Dare as defined above; B is O, NR₄ or S; E is a chemical bond; Z is —CN, —COOR₃ or tetrazol, and L is a leaving group, such as halo, tosylate, or mesylate. Where B is O or S, any base normally employed to deprotonate an alcohol or thiol may be used, such as sodium hydride, sodium hydroxide, triethylamine, sodium bicarbonate or diisopropyl/ethylamine.

Reaction temperatures are in the range of about room temperature to reflux and reaction times vary from about 2 to about 96 hours. The reactions are usually carried out in a solvent that will dissolve both reactants and is inert to both as well. Solvents include, but are not limited to, diethyl ether, tetrahydrofuran, N,N-dimethylformamide, dimethylsulfoxide, dioxane and the like.

In the case where B is SO or SO₂ then treatment of the thio compound with m-chlorobenzoic acid or sodium periodate results in the sulfinyl compound. Preparation of the sulfonyl compound may be accomplished by known procedures such as dissolving the sulfinyl compound in acetic acid and treating with 30% H₂O₂.

Those compounds where B is

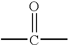

may be prepared by the following reaction sequence:

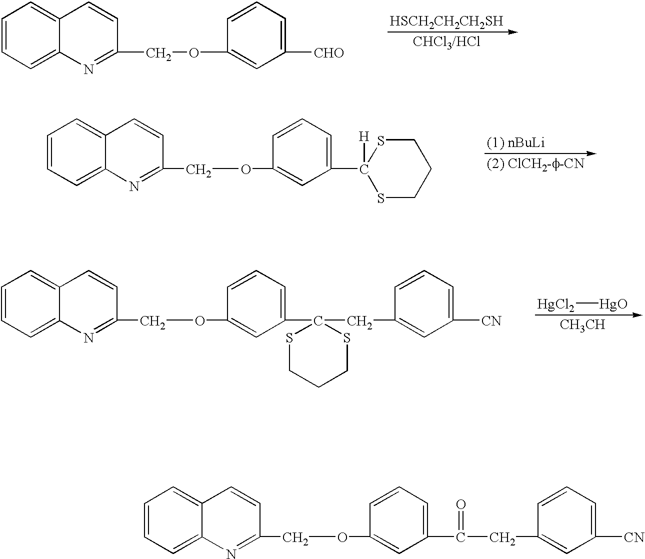

Condensation of the aldehyde with 1,3-propenedithiol results in the dithiane compound. This may be carried out in chloroform at reduced temperatures of about −20° C., while bubbling HCl gas into the reaction mixture. The dithiane compound is then treated with N-butyl lithium in nonpolar solvent at about −78° C. and then reacted with the substituted benzyl chloride. This results in addition of the Ring III to the molecule. The dithiane moiety is then treated with a mercuric chloride-mercuric oxide mixture to form the complex which is then split off leaving the desired compound.

Those compounds where A is

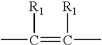

are prepared by reacting the appropriate aldehyde or ketone with a substituted Wittig reagent of the formula

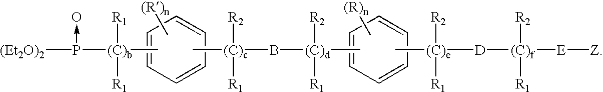

Subsequent condensation results in formation of the double bond. The Wittig reagent is prepared by known art recognized procedure such as reaction of triphenyl phosphine or diethylphosphone, with a suitable substituted alkyl/aryl bromide followed by treatment with a strong organometallic base such as n-BuLi or NaOH, which results in the desired ylide. Conventional Wittig reaction conditions may be used in accordance with standard practice. For examples, see Bestmann and Vostrowsky, Top. Curr. Chem. 109, 85-164 (1983), and Pommer and Thieme, Top. Curr. Chem. 109, 165-188 (1983).

There is no particular restriction on the nature of the solvent to be employed, provided that it has no adverse effect on the reaction or on the reagents involved.

Of course, this Wittig condensation may also take place when the Wittig reagent is formed on Ring I portion of the molecule, which is then condensed with the aldehyde from the Ring II portion.

Those compounds where A is a chemical bond may be prepared by known coupling methods, for example, the reaction of an appropriate alkyl halide with an appropriate organometallic reagent such as a lithium organocopper reagent (See Posner, Org. React. 22, 235-400 (1975), Normant, Synthesis 63-80 (1972), Posner, “An introduction to Synthesis Using Organocopper Reagents” p. 68-81, Wiley, New York, 1980); coupling of an appropriate lithium organocopper reagent, or Grignard reagent, with a suitable ester of sulfuric or sulfonic acid (see “An introduction to Synthesis Using Organocopper Reagents” p. 68-81, Wiley, New York, 1980, Kharasch and Reinmuth “Grignard Reactions of Non Metallic Substances”, pp1277-1286, Prentice-Hall, Englewood Cliffs, N.J., 1954); or other known reactions for forming alkyl bonds (See March “Advanced Organic Chemistry” p. 1149, Third Edition, Wiley, NY, 1985).

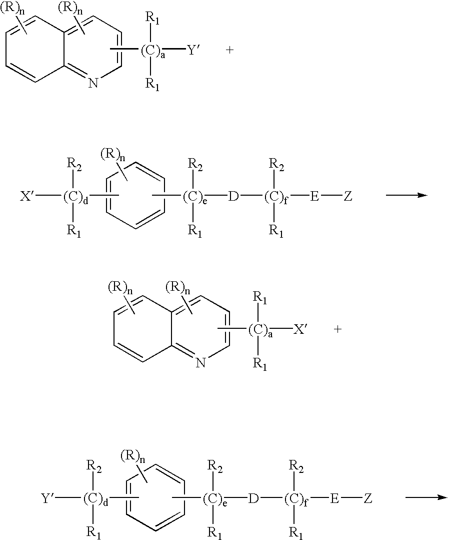

where X′ is halide, an ester of a sulfuric acid, or a sulfonic ester, and Y′ is a lithium organocopper reagent or Grignard reagent.

There is no particular restriction on the nature of the reagent or solvent to be employed, provided that it has no adverse effect on the reaction or on the reagents involved.

Alternatively, compounds where A is a chemical bond may be prepared by reduction of appropriate compounds where A is

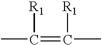

with a suitable reducing agent, for example H₂/Pd/C.

There is no particular restriction on the solvent or nature of the reducing agent to be used in this reaction, and any solvent and reducing agent conventionally used in reactions of this type may equally be used here, provided that it has no adverse effect on other parts of the molecule. An example of a suitable reducing agent is H₂/Pd/C. Other reducing reagents are known in the art. For example, see: Mitsui and Kasahara, in Zabicky, “The Chemistry of Alkenes”, vol. 2, pp. 175-214, Interscience, NY, 1970; and Rylander “Catalytic Hydrogenation over Platinum Metals”, pp. 59-120, Academic Press, NY 1967.

Those compounds where B is

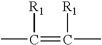

are prepared by reacting the appropriate aldehyde or ketone with a substituted Wittig reagent of the formula

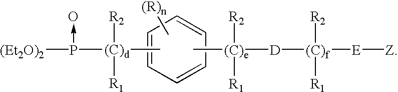

Condensation results in formation of the double bond. The Wittig reagent is prepared by known art recognized procedure, such as reaction of triphenyl phosphine or diethylphosphone, with a suitable substituted alkyl/aryl bromide followed by treatment with a strong organometallic base such as n-BuLi or NaOH results in the desired ylide. Conventional Wittig reaction conditions may be used in accordance with standard practice, for examples see Bestmann and Vostrowsky, Top. Curr. Chem. 109, 85-164 (1983), and Pommer and Thieme, Top. Curr. Chem. 109, 165-188 (1983).

There is no particular restriction on the nature of the solvent to be employed, provided that it has no adverse effect on the reaction or on the reagents involved.

Of course this Wittig condensation may also take place when the Wittig reagent is formed on Ring II portion of the molecule which is then condensed with the aldehyde from the Ring III portion.

Those compounds where B or A is a chemical bond may be prepared by known coupling methods, for example, the reaction of an appropriate alkyl halide with an appropriate organometallic reagent such as a lithium organocopper reagent (See Posner, Org. React. 22, 235-400 (1975), Normant, Synthesis 63-80 (1972), Posner, “An introduction to Synthesis Using Organocopper Reagents” p. 68-81, Wiley, New York, 1980); coupling of an appropriate lithium organocopper reagent, or Grignard reagent, with a suitable ester of sulfuric or sulfonic acid (see “An introduction to Synthesis Using Organocopper Reagents” p. 68-81, Wiley, New York, 1980, Kharasch and Reinmuth “Grignard Reactions of Non Metallic Substances”, p.1277-1286, Prentice-Hall, Englewood Cliffs, N.J., 1954); or other known reactions for forming alkyl bonds (see March “Advanced Organic Chemistry” p. 1149, Third Edition, Wiley, NY, 1985).

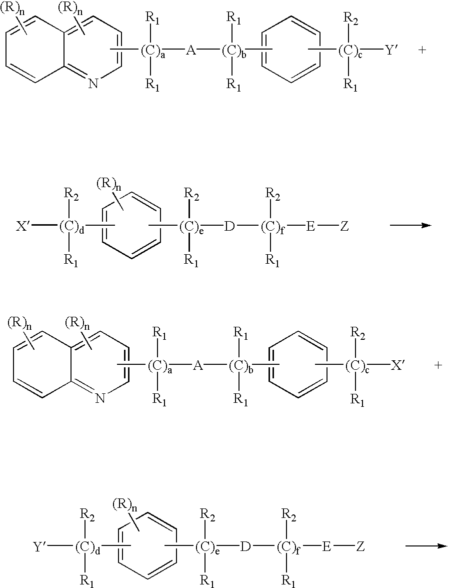

where X′ is halide, an ester of a sulfuric acid, or a sulfonic ester, Y′ is a lithium organocopper reagent or Grignard reagent.

There is no particular restriction on the nature of the reagent or solvent to be employed, provided that it has no adverse effect on the reaction or on the reagents involved.

Alternatively, compounds where B is a chemical bond may be prepared by reduction of appropriate compounds where B is

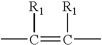

with a suitable reducing agent, for example H₂/Pd/C.

There is no particular restriction on the nature of the solvent to be employed, provided that it has no adverse effect on the reaction or on the reagents involved.

There is no particular restriction on the solvent or nature of the reducing agent to be used in this reaction, and any solvent and reducing agent conventionally used in reactions of this type may equally be used here, provided that it has no adverse effect on other parts of the molecule. An Example of a suitable reducing agent is H₂/Pd/C. Other reducing reagents are known in the art. For example, see: Mitsui and Kasahara, in Zabicky, “The Chemistry of Alkenes”, vol. 2, p. 175-214, Interscience, NY, 1970; and Rylander “Catalytic Hydrogenation over Platinum Metals”, p. 59-120, Academic Press, NY, 1967.

The tetrazole may be formed from the nitrite at various stages of the synthesis by treatment with hydrazoic acid formed in situ from sodium azide and an acid.

When B is

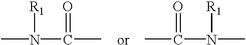

then condensation of the acid halide with the appropriate aniline will give the desired compound as shown below in the following scheme.

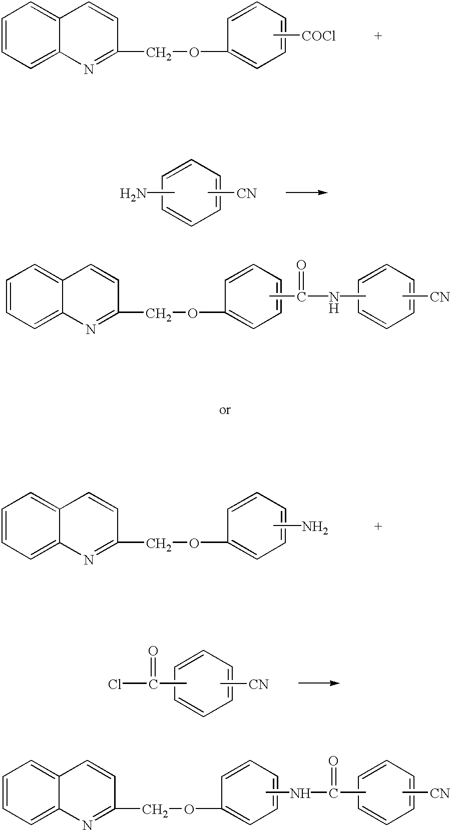

Those compounds where D and/or E are

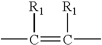

are prepared by reacting the appropriate aldehyde or ketone with a substituted Wittig reagent of the formula

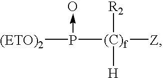

where Z is cyano or carbalkoxy. Reaction conditions would be similar to those for A and B above.

Those compounds where D and/or E are a chemical bond may also be synthesized by coupling methods analogous to those for compounds where A and B are a chemical bond as described above.

In one particular embodiment of this invention, ArI, ArII, or ArIII is defined as a heterocycle such as pyridine, pyrimidine and pyridazine. In principle, appropriately functionalized ring systems of this kind can be prepared by functionalization of specific precursors followed by ring synthesis or by derivatization of a preformed ring system. There are numerous approaches to the synthesis and functionalization of the aforementioned heterocyclic frameworks in the chemical literature (for examples, see (a) Katritzky, A. R.; Rees, C. W.; Scriven, E. F. V. Eds. Comprehensive Heterocyclic Chemstry II, Vol 5 and Vol 6. Elsevier Science 1996 and references therein). A particularly useful protocol with regard to the current invention involves Mitsunobu etherification of hydroxyl substituted heterocycles such as outlined in Scheme A. Treatment of 5-bromo-pyridin-2-one (1, G, J=CH), 5-bromo-pyrimidin-2-one (2, G=N, J=CH) or 6-bromo-pyrazin-3-one (3, G=CH, J=N) with an alcohol under Mitsunobu's conditions provides the corresponding bromo-substituted heterocyclic ethers (4) (for typical procedures see Mitsunobu. O., Synthesis, 1981, 1).

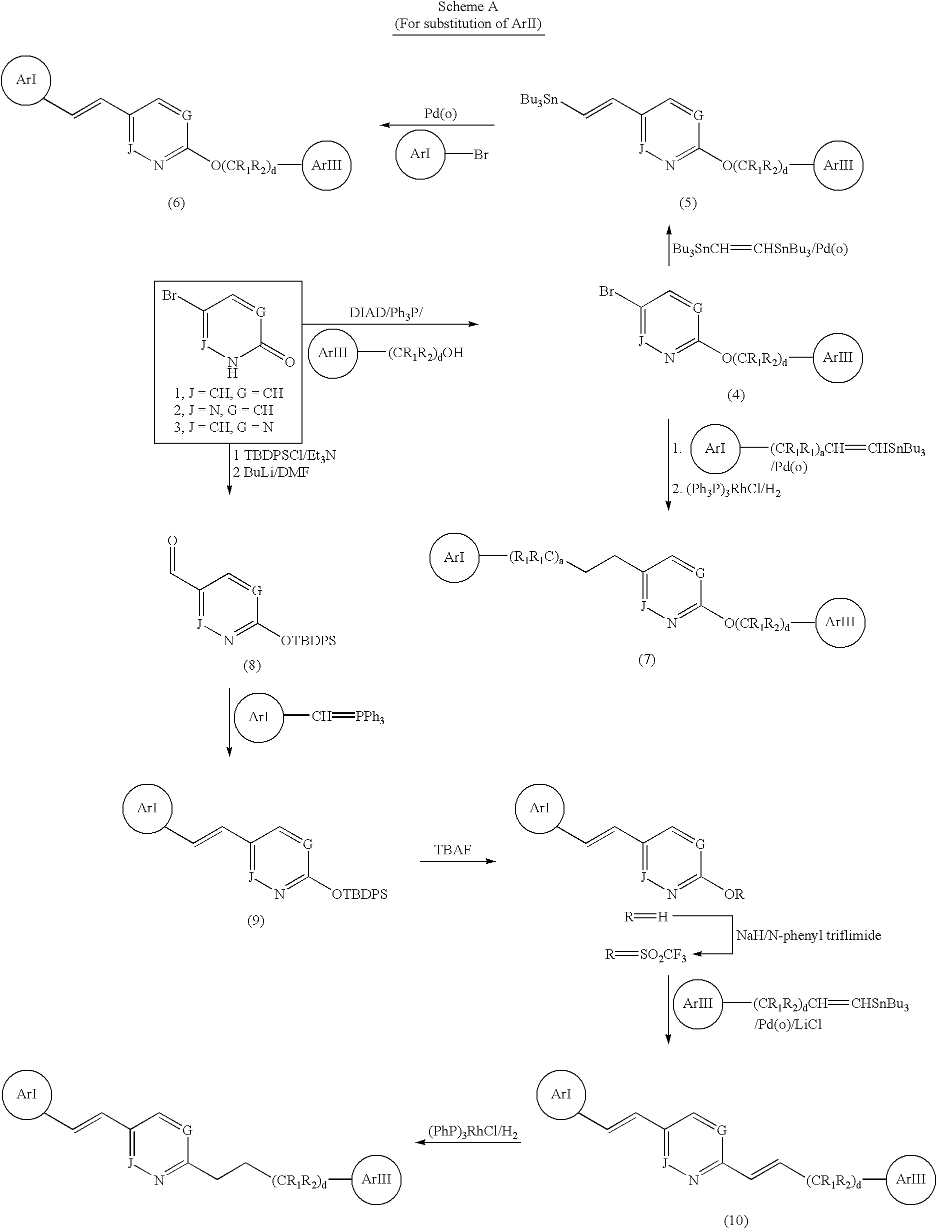

These heterocyclic bromides can be further functionalized in a number of ways. For example, coupling with a vinyl stannane can be effected under palladium (o) catalysis to provide systems with an alkenyl side chain (5 and 6). The choice of catalyst and reaction temperature depends on the substrate employed but is most commonly tetrakistriphenylphosphine palladium,bis(triphenylphosphine)palladium chloride, 1,1′-bis(diphenylphosphino)ferrocene/bis-dibenzylideneacetone palladium or 1,2 bis-(diphenylphosphino)ethane/bis(acetonitrile)dichloropalladium at a temperature between 50 and 150° C. Suitable solvents include DMF, DMPU, HMPA, DMSO, toluene, and DME. (for examples see Farina, V. Krishnamurthy, V.; Scott, W. J. Organic Reactions, 1997, 50, 1). Reduction of the olefin using, for example Wilkinson's catalyst in a solvent such as toluene, THF or an alcohol at a temperature between about 20 and 80° C. provides the corresponding alkane (7). Heterocyclic bromides such as (1) can also be metalated (after protection of the carbonyl functionality as a O-silyl ether by reaction with an appropriate silyl chloride or triflate in the presence of a base such as triethylamine or imidazole in a solvent such as dichloromethane or DMF) with an alkyl lithium reagent generally at low temperature (below −50° C.) Suitable solvents for this process include THF or diethyl ether, either alone or as mixtures with additives such as HMPA, TMEDA or DABCO. The resulting aryl lithium species can then be reacted with a variety of electrophiles such as aldehydes, alkyl halides, oxiranes, aziridines or ab-unaturated carbonyls to provide heterocycles substituted with a variety of functionalized side chains. In particular, by using DMF as the electrophile, this procedure can be used to install an aldehyde functional group on the heterocycle (8). The aldehyde can then be further functionalized by Wittig or Horner Emons reaction to produce olefin substituted heterocyclic silyl ethers (9). (For examples see Cadogan, J. I. G. Organophosphorus Reagents in Organic Synthesis, Academic Press, 1979 and references therein). The silyl ether can be cleaved using tetrabutyl ammonium fluoride in THF at room temperature or above (For examples see Protective Groups in Organic Synthesis, T. W. Greene and P. G. M. Wuts; John Wiley Publications 1998 and references therein). The resulting hydroxyl functionality can be converted to the corresponding triflate using N-phenyl triflimide and a base such as sodium hydride or sodium hexamethyldisilazide in a solvent such as THF or DME at or below room temperature. Coupling of the resulting triflate with a vinyl (or alkynyl ) stannane in the presence of lithium chloride and a Pd (o) catalyst as described above produces the corresponding bisalkenyl substituted heterocycles (10). Similarly, the substitution of Ar III can be accomplished according to Scheme A-I

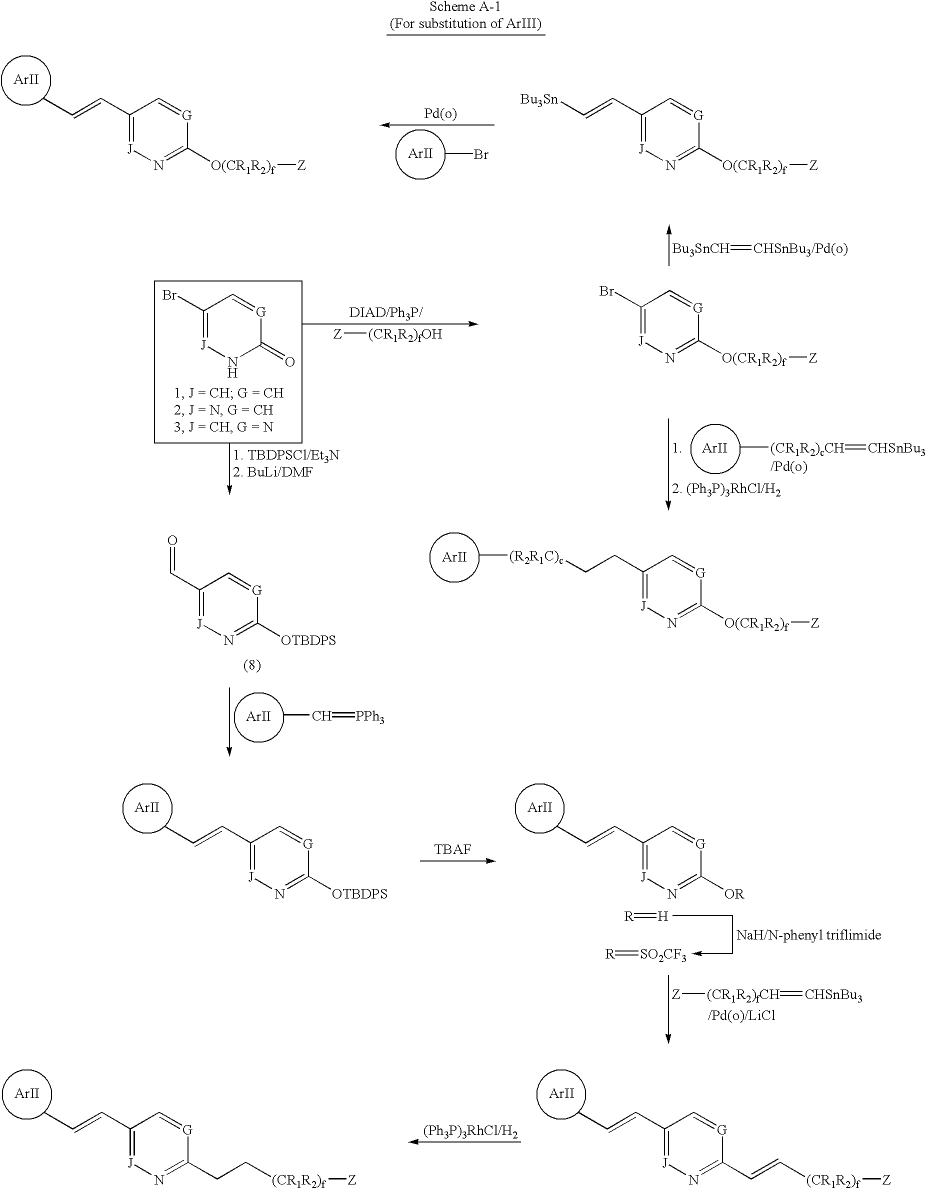

Bromo substituted heterocycles such as (11 and 12 scheme B) can be converted into the analogous hydroxyl substituted system by first, conversion to the borate ester (13) then oxidative cleavage of the carbon boron bond with an oxidant such as aqueous hydrogen peroxide in the presence of acid or base (such as acetic acid, sodium carbonate or sodium hydroxide) or oxone in the presence of a base (such as sodium carbonate) at or above 0° C. (For examples see Webb, K. S.; Levy, D. Tetrahedron Letts., 1995, 36, 5117, and Koster, R.; Morita, Y. Angew. Chem., 1966, 78, 589).

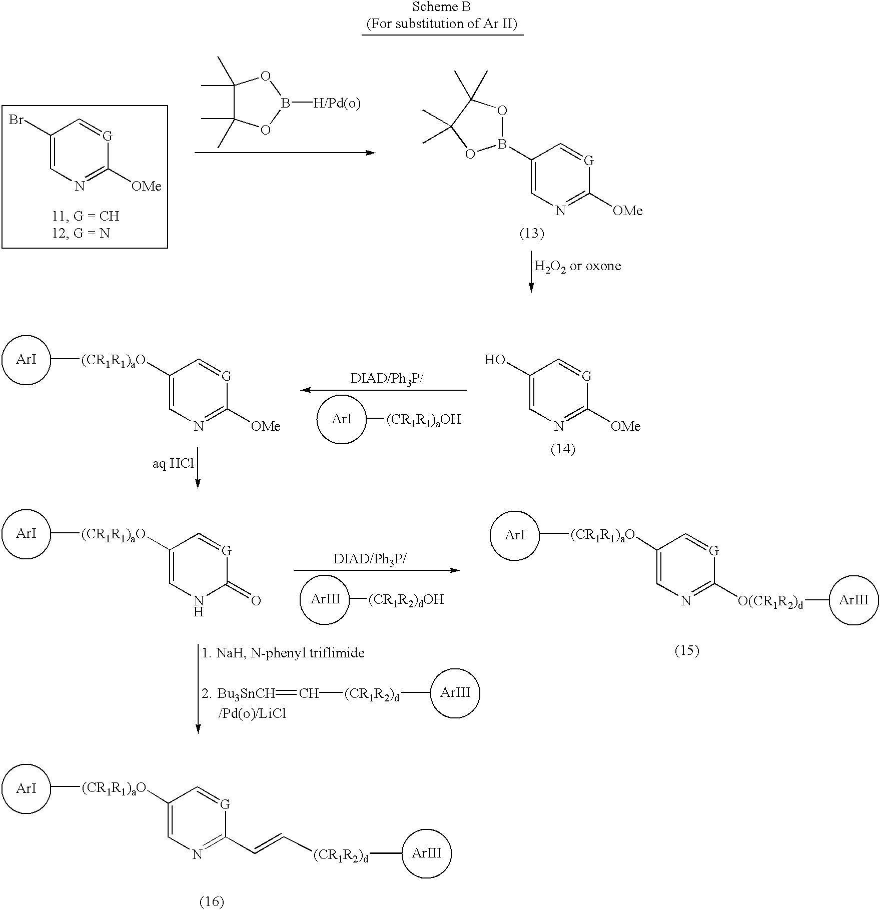

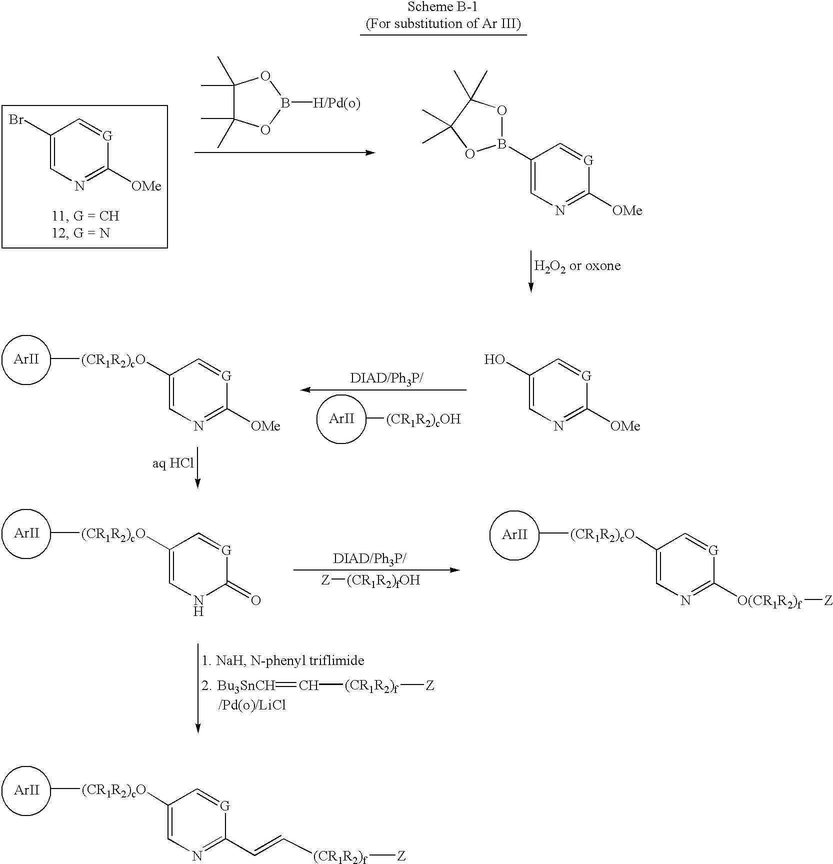

The resulting hydroxy substituted heterocycles (14) can be further derivatized as already described above to give ether (15) or alkenyl (16) substituted side chains. Certain heterocyclic bromides or chlorides situated ortho or para to a ring nitrogen can be readily displaced with an alcohol in the presence of base such as sodium hydride in a solvent such as Toluene, DMSO, THF, DMPU or HMPA at or above room temperature (For examples see Kelly, T. R. et al. J. Amer. Chem. Soc., 1994, 116, 3657 and Newkome, G. R. et al. J Org. Chem., 1977, 42, 1500). In particular, alcoholysis of a 2,6-dibromo-pyridine using a controlled stoichiometric amount of alcohol reagent provides the alkoxy substituted-bromo-pyridine. Subsequent reaction of this product with a further equivalent of another alcohol provides the unsymmetrically dialkoxy-substituted heterocycle.

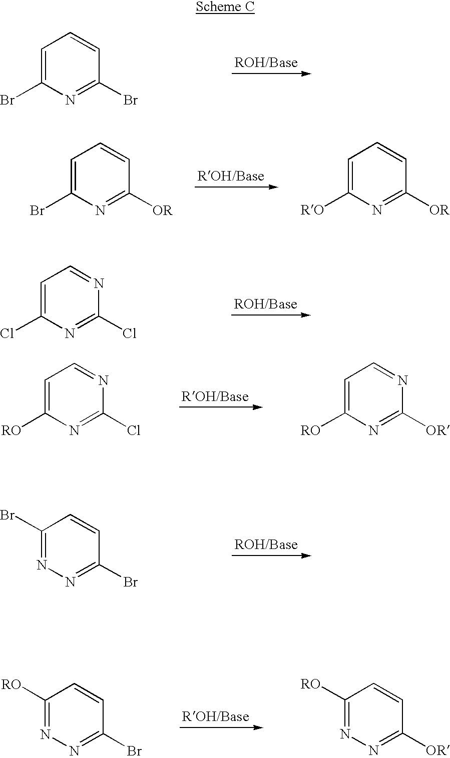

Similar procedures using 2,4-dichloro-pyrimidine or 2,6-dibromo-pyridazine provides the corresponding dialkoxy-substituted pyrimidines and pyridazines. A simple alkoxy group positioned ortho to a nitrogen in these heterocyclic systems can be hydrolysed to the corresponding hydroxy substituent using aqueous hydrochloric acid normally at or above room temperature (Scheme D).

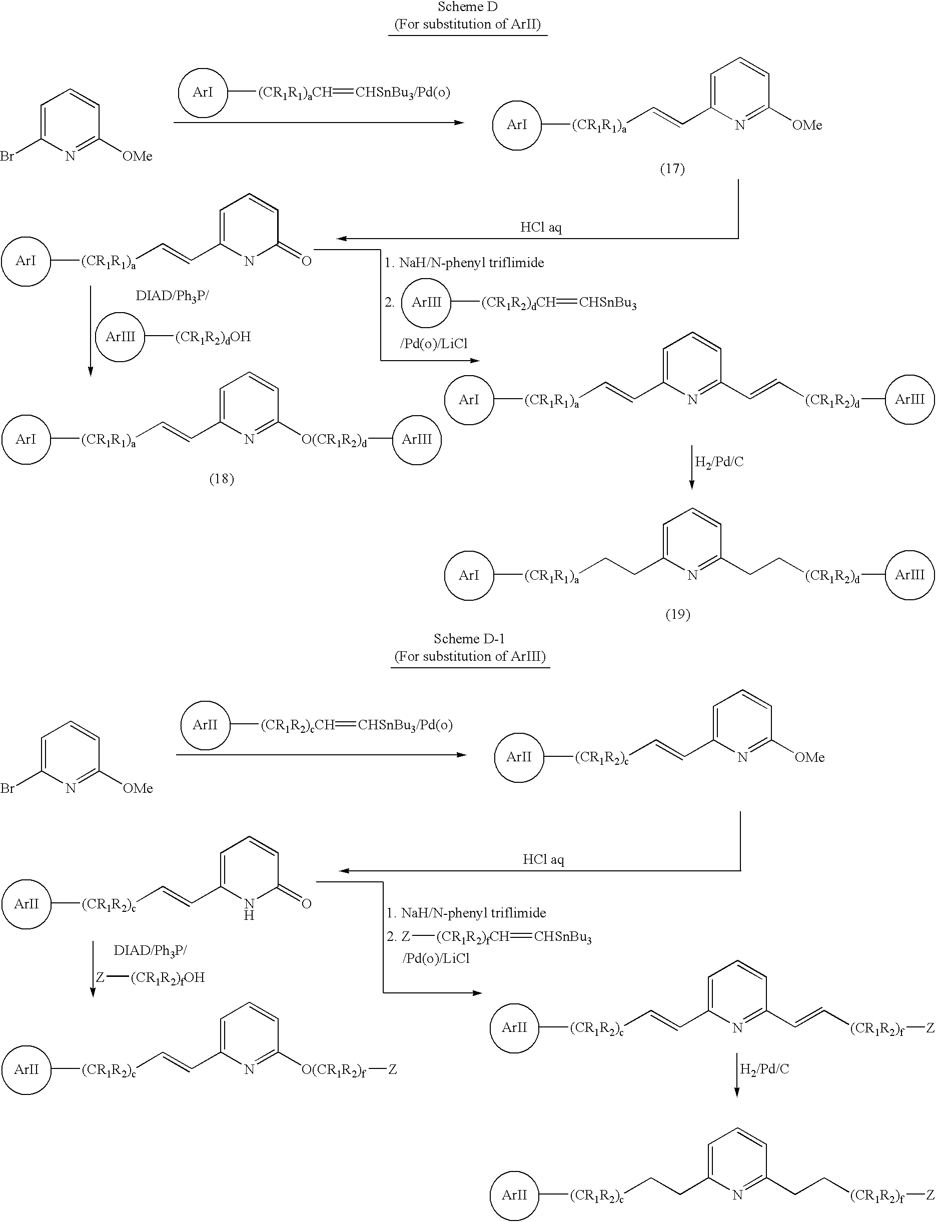

For example, treatment of the 2-methoxy-6-alkenyl-substituted pyridine (17) with hydrochloric acid provides the 6-alkenyl substituted pyridin-2-one. This intermediate, in turn, can be further derivatized to the corresponding 2-alkoxy (18) or 2-alkyl (19) substituted systems as previously described. A methyl, methylene or methine group positioned ortho to a ring nitrogen in these heterocyclic systems can be deprotonated with a base such as an alkyl lithium or LDA in a solvent such as THF ether or HMPA, generally at low temperature (below 0° C.) and the resulting anion reacted with electrophiles such as aldehydes epoxides alkyl halides or a,b-unsaturated carbonyl compounds to provide a variety of functionalized side chain substituents.

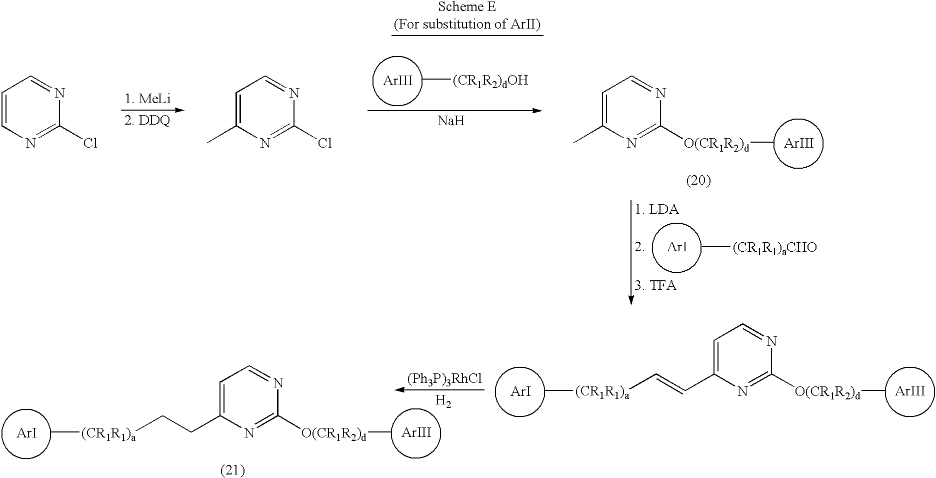

For example (Scheme E), 2-alkoxy4-methyl-pyrimidine (20) is treated with LDA at −78° C. followed by an aldehyde to give the corresponding hydroxy adduct. Subsequent dehydration with trifluoroacetic acid in a solvent such as dichloromethane followed by hydrogenation of the resulting olefin provides the 4-alkyl-2-alkoxy-pyrimidine (21).

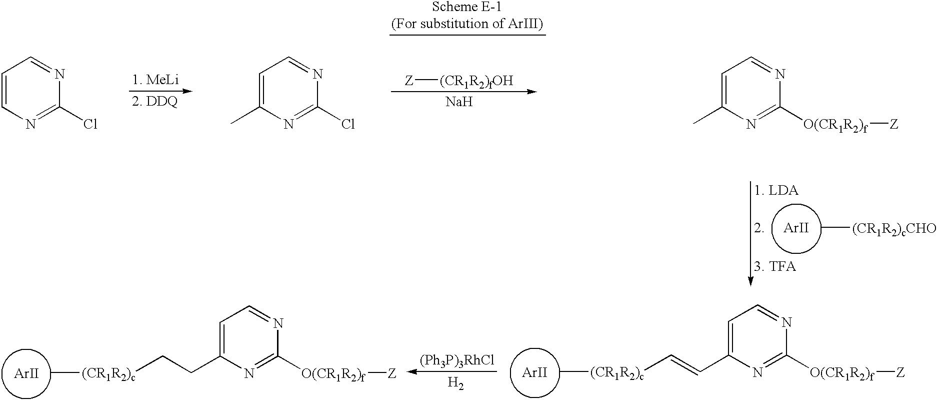

Furthermore, compounds of the invention may be easily synthesized by solid phase methods, as outlined below, using imputs (XII)-(XVII) as listed in the schemes F and G and Table 3 below:

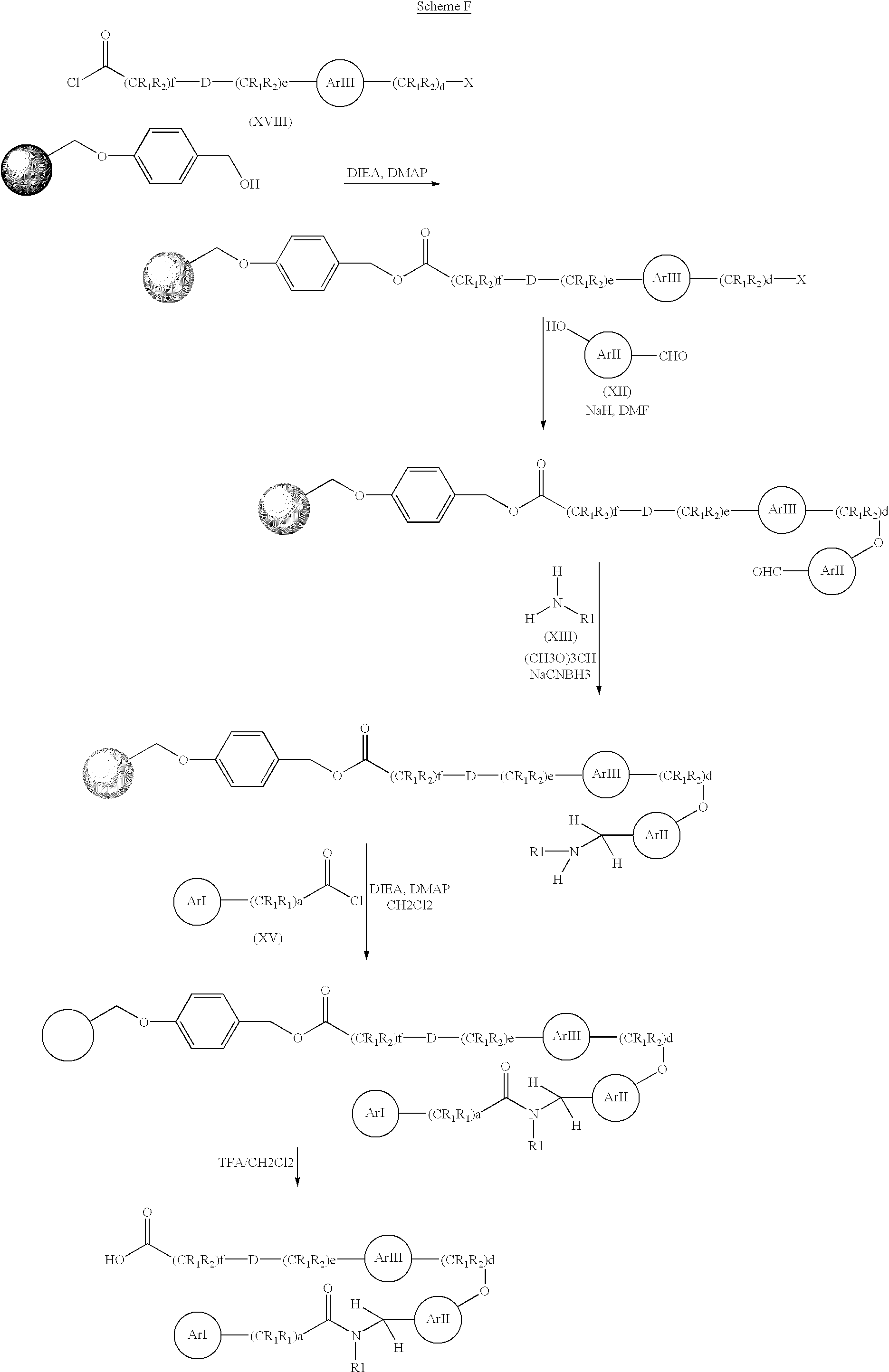

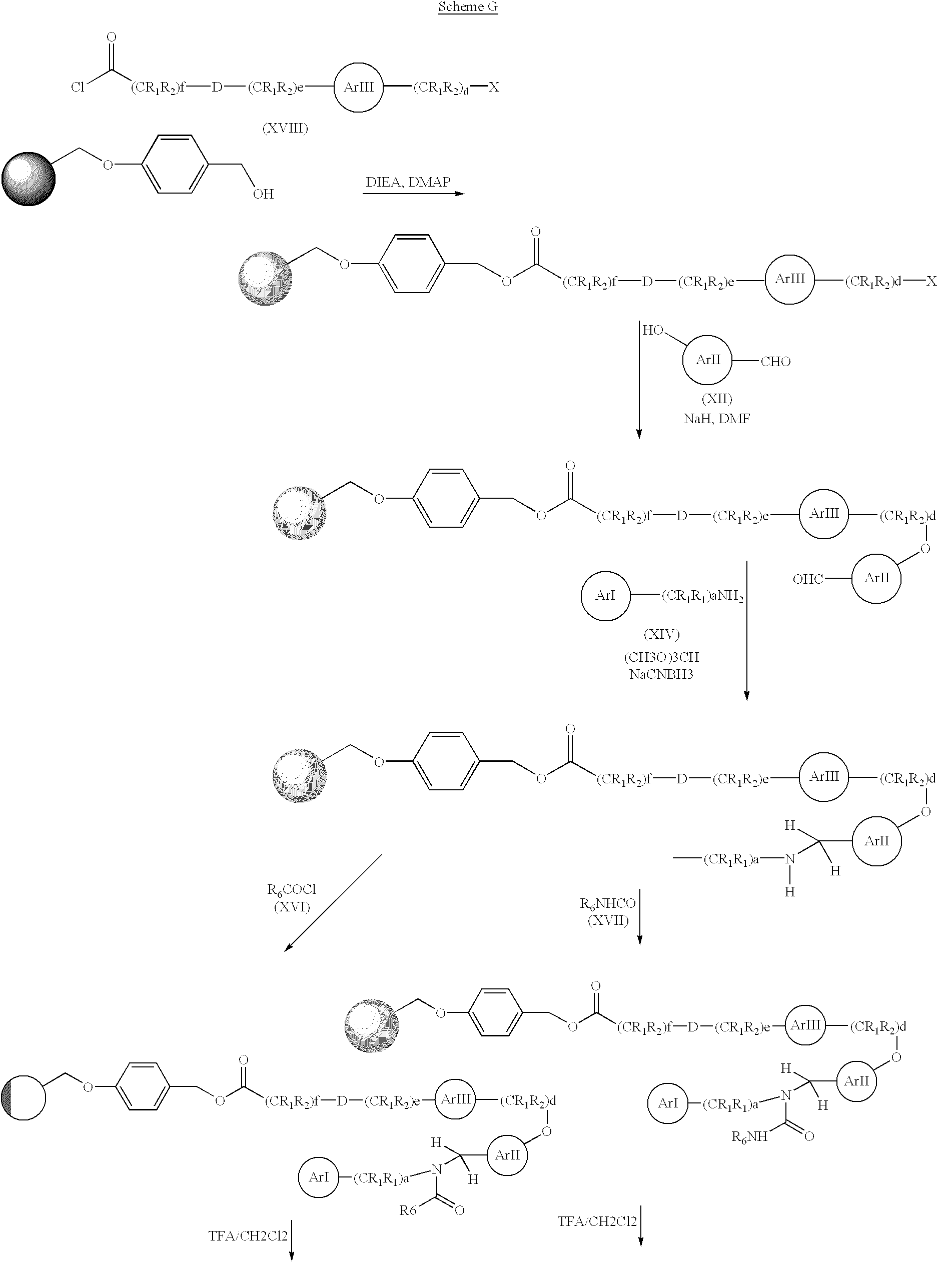

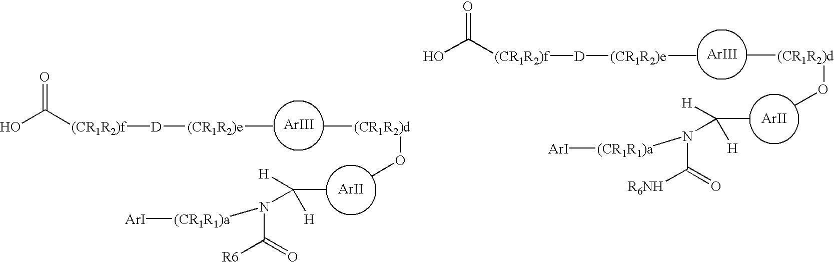

TABLE 3

| 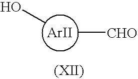 | 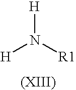 | 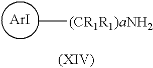 | 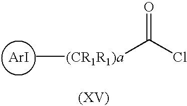 |
|---|---|---|---|
| 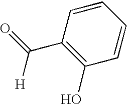 | 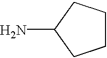 | 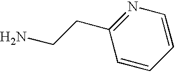 | 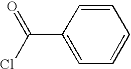 |
| 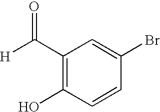 | 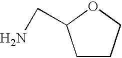 | 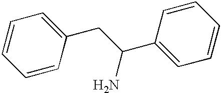 | 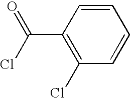 |
| 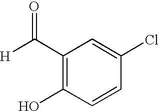 | 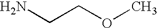 | 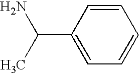 | 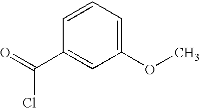 |
| 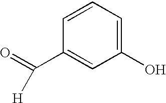 | 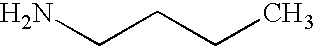 | 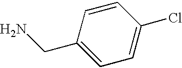 | 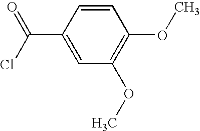 |
| 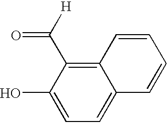 | | 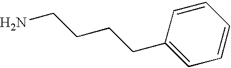 | 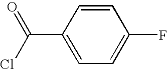 |
| 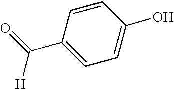 | | 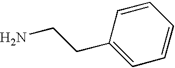 | 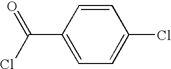 |
| 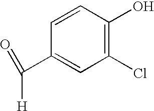 | | 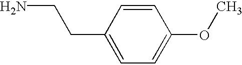 | 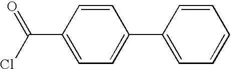 |
| 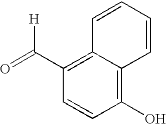 | | 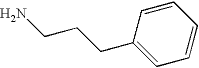 | 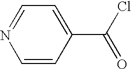 |
| | | 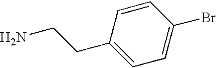 | |

| R₆COCl (XVI) | R₆NCO (XVII) | 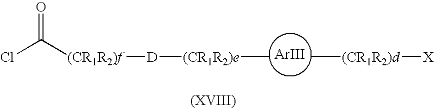 |
|---|---|---|
| 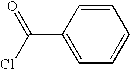 | 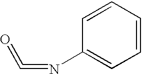 | 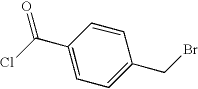 |
| 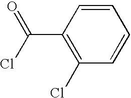 | | 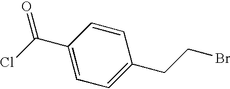 |
| 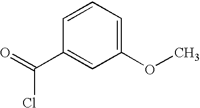 | | |
| 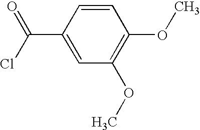 | | |
| 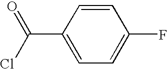 | | |
| 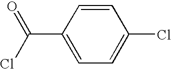 | | |
| 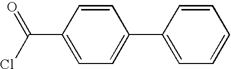 | | |
| 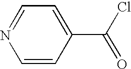 | | |
| 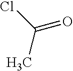 | | |
| 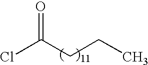 | | |
| 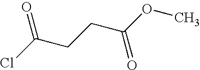 | | |
| 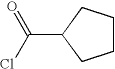 | | |

Compounds useful according to the invention may also be prepared by the application or adaptation of known methods, by which is meant methods used heretofore or described in the literature, for example those described by R. C. Larock in Comprehensive Organic Transformations, VCH publishers, 1989.

In the reactions described hereinafter, it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see T. W. Green and P. G. M. Wuts in “Protective Groups in Organic Chemistry” John Wiley and Sons, 1991; J. F. W. McOmie in “Protective Groups in Organic Chemistry” Plenum Press, 1973.

According to a further feature of the present invention, compounds useful according to the invention may be prepared by interconversion of other compounds of the invention.

A compound of the invention including a group containing one or more nitrogen ring atoms, preferably imine (═N—), may be converted to the corresponding compound wherein one or more nitrogen ring atom of the group is oxidized to an N-oxide, preferably by reacting with a peracid, for example peracetic acid in acetic acid or m-chloroperoxybenzoic acid in an inert solvent such as dichloromethane, at a temperature from about room temperature to reflux, preferably at elevated temperature.

The products of this invention may be obtained as racemic mixtures of their dextro and levorotatory isomers since at least one asymmetric carbon atom may be present. When two asymmetric carbon atoms are present, the product may exist as a mixtures of diastereomers based on syn and anti configurations. These diastereomers may be separated by fractional crystallization. Each diastereomer may then be resolved into dextro and levorotatory optical isomers by conventional methods.

It will also be apparent to those skilled in the art that certain compounds of Formula I may exhibit geometrical isomerism. Geometrical isomers include the cis and trans forms of compounds of the invention having an alkenyl moiety. The present invention comprises the individual geometrical isomers and stereoisomers and mixtures thereof.

Such isomers can be separated from their mixtures, by the application or adaptation of known methods, for example chromatographic techniques and recrystallization techniques, or they are separately prepared from the appropriate isomers of their intermediates, for example by the application or adaptation of methods described herein.

Resolution may best be carried out in the intermediate stage where it is convenient to combine the racemic compound with an optically active compound by salt formation, ester formation, or amide formation to form two diasteromeric products. If an acid is added to an optically active base, then two diastereomeric salts are produced which possesses different properties and different solubilities and can be separated by fractional crystallization. When the salts have been completely separated by repeated crystallization, the base is split off by acid hydrolysis and enantiomerically purified acids are obtained.

Compounds useful according to the invention are useful in the form of the free base or acid or in the form of a pharmaceutically acceptable salt thereof. All forms are within the scope of the invention.

Where a compound useful according to the invention is substituted with a basic moiety, acid addition salts are formed and are simply a more convenient form for use; in practice, use of the salt form inherently amounts to use of the free base form. The acids which can be used to prepare the acid addition salts include preferably those which produce, when combined with the free base, pharmaceutically acceptable salts, that is, salts whose anions are non-toxic to the patient in pharmaceutical doses of the salts, so that the beneficial pharmaceutical effects of these compounds in the free base are not vitiated by side effects ascribable to the anions. Although pharmaceutically acceptable salts of said basic compounds are preferred, all acid addition salts are useful as sources of the free base form even if the particular salt, per se, is desired only as an intermediate product as, for example, when the salt is formed only for purposes of purification, and identification, or when it is used as an intermediate in preparing a pharmaceutically acceptable salt by ion exchange procedures. Pharmaceutically acceptable salts useful within the scope of the invention are those derived from the following acids: mineral acids such as hydrochloric acid, trifluoroacetic acid, sulfuric acid, phosphoric acid and sulfamic acid; and organic acids such as acetic acid, citric acid, lactic acid, tartaric acid, malonic acid, methanesufonic acid, ethanesulfonic acid, benzenesulfonic acid, p-toluenesulfonic acid, cyclohexylsulfamic acid, quinic acid, and the like. The corresponding acid addition salts comprise the following: hydrohalides, e.g. hydrochloride and hydrobromide, trifluoroacetate, sulfate, phosphate, nitrate, sulfamate, acetate, citrate, lactate, tartarate, malonate, oxalate, salicylate, propionate, succinate, fumarate, maleate, methylene-bis-β-hydroxynaphthoates, gentisates, mesylates, isothionates, di-p-toluoyltartrates, methanesulfonates, ethanesulfonates, benzenesulfonates, p-toluenesulfonates, cyclohexylsulfamate and quinate, respectively.

The acid addition salts of the compounds useful according to the invention are prepared by reaction of the free base with the appropriate acid, by the application or adaptation of known methods. For example, the acid addition salts of the compounds of this invention are prepared either by dissolving the free base in aqueous or aqueous-alcohol solution or other suitable solvents containing the appropriate acid and isolating the salt by evaporating the solution, or by reacting the free base and acid in an organic solvent, in which case the salt separates directly or can be obtained by concentration of the solution.

The compounds useful according to the invention may be regenerated from the acid addition salts by the application or adaptation of known methods. For example, parent compounds useful according to the invention can be regenerated from their acid addition salts by treatment with an alkali, e.g., aqueous sodium bicarbonate solution or aqueous ammonia solution.

Where the compound useful according to the invention is substituted with an acidic moiety, base addition salts may be formed and are simply a more convenient form for use; in practice, use of the salt form inherently amounts to use of the free acid form. The bases which can be used to prepare the base addition salts include preferably those which produce, when combined with the free acid, pharmaceutically acceptable salts, that is, salts whose cations are non-toxic to the animal organism in pharmaceutical doses of the salts, so that the beneficial pharmaceutical effects on the activity of the compounds of the present invention in the free acid are not vitiated by side effects ascribable to the cations. Pharmaceutically acceptable salts useful according to the invention, include for example alkali and alkaline earth metal salts, including those derived from the following bases: sodium hydride, sodium hydroxide, potassium hydroxide, calcium hydroxide, aluminum hydroxide, lithium hydroxide, magnesium hydroxide, zinc hydroxide, ammonia, ethylenediamine, N-methyl-glucamine, lysine, arginine, ornithine, choline, N,N′-dibenzylethylenediamine, chloroprocaine, diethanolamine, procaine, diethylamine, N-benzylphenethylamine, piperazine, tris(hydroxymethyl)aminomethane, tetramethylammonium hydroxide, and the like.

Metal salts of compounds useful according to the present invention may be obtained by contacting a hydride, hydroxide, carbonate or similar reactive compound of the chosen metal in an aqueous or organic solvent with the free acid form of the compound. The aqueous solvent employed may be water or it may be a mixture of water with an organic solvent, preferably an alcohol such as methanol or ethanol, a ketone such as acetone, an aliphatic ether such as tetrahydrofuran, or an ester such as ethyl acetate. Such reactions are normally conducted at ambient temperature but they may, if desired, be conducted with heating.

Amine salts of compounds useful according to the present invention may be obtained by contacting an amine in an aqueous or organic solvent with the free acid form of the compound. Suitable aqueous solvents include water and mixtures of water with alcohols such as methanol or ethanol, ethers such as tetrahydrofuran, nitriles such as acetonitrile, or ketones such as acetone. Amino acid salts may be similarly prepared.

The base addition salts of the compounds useful according to the invention can be regenerated from the salts by the application or adaptation of known methods. For example, parent compounds useful according to the invention can be regenerated from their base addition salts by treatment with an acid, e.g. hydrochloric acid.

Salt forms useful according to the invention also include compounds having a quarternarized nitrogen. The quarternarized salts are formed by methods such as by alkylation of sp³ or sp² hybridized nitrogen in the compounds.

As will be self-evident to those skilled in the art, some of the compounds useful according to the invention do not form stable salts. However, acid addition salts are most likely to be formed by compounds useful according to the invention having a nitrogen-containing heteroaryl group and/or wherein the compounds contain an amino group as a substituent. Preferable acid addition salts of the compounds useful according to the invention are those wherein there is not an acid labile group.

As well as being useful in themselves as active compounds, the salts of the compounds useful according to the invention are useful for the purposes of purification of the compounds, for example by exploitation of the solubility differences between the salts and the parent compounds, side products and/or starting materials by techniques well known to those skilled in the art.

Various substituents on the compounds useful according to the invention, e.g., as defined in R, R₁ and R₂ can be present in the starting compounds, added to any one of the intermediates or added after formation of the final products by known methods of substitution or conversion reactions. If the substituents themselves are reactive, then the substituents can themselves be protected according to the techniques known in the art. A variety of protecting groups known in the art may be employed. Examples of many of these possible groups may be found in “Protective Groups in Organic Synthesis” by T. W. Green, John Wiley and Sons, 1981. For example, nitro groups can be added to the aromatic ring by nitration, and the nitro group then converted to other groups, such as amino, by reduction, and halo, by diazotization of the amino group and replacement of the diazo group. Acyl groups can be substituted onto the aryl groups by Friedel-Crafts acylation. The acyl groups then can be transformed to the corresponding alkyl groups by various methods, including the Wolff-Kishner reduction and Clemmenson reduction. Amino groups can be alkylated to form mono and dialkylamino groups; and mercapto and hydroxy groups can be alkylated to form corresponding ethers. Primary alcohols can be oxidized by oxidizing agents known in the art to form carboxylic acids or aldehydes, and secondary alcohols can be oxidized to form ketones. Thus, substitution or alteration reactions can be employed to provide a variety of substituents throughout the molecule of the starting material, intermediates, or the final product.

The starting materials and intermediates are prepared by the application or adaptation of known methods, for example methods as described in the Reference Examples or their obvious chemical equivalents.

The present invention is further exemplified but not limited by the following examples, which illustrate the preparation of the compounds according to the invention.

### EXAMPLE 1

### 3-(2-QUINOLINYLMETHYLOXY)BENZYL ALCOHOL

A mixture of 12.8 g (0.06 mol) of 2-quinolinylmethyl chloride HCl, 7.5 g (0.06 mol) of 3-hydroxybenzyl alcohol, and 18 g of potassium carbonate in 50 ml of DMF is heated at 70° C. overnight. The reaction mixture is poured into water, and the precipitated product is collected, filtered and dried to give 3-(2-quinolinylmethyloxy)benzyl alcohol.

### EXAMPLE 2

When 2-quinolinylmethyl chloride of Example 1 above is replaced by the quinoline compounds of Table I below then the corresponding product is obtained.

TABLE I

2-chloromethylquinoline
2-bromomethylquinoline
2-(1-chloroethyl)quinoline
2-(2-chloroethyl)quinoline
2-bromoethylquinoline
3-chloromethylquinoline
4-chloromethylquinoline
2-(β-chloroethyl)quinoline
2-(β-chloropropyl)quinoline
2-(β-chloro-β-phenethyl)quinoline
2-chloromethyl-4-methylquinoline
2-chloromethyl-6-methylquinoline
2-chloromethyl-8-methylquinoline
2-chloromethyl-6-methoxyquinoline
2-chloromethyl-6-nitroquinoline
2-chloromethyl-6,8-dimethylquinoline

### EXAMPLE 3

When 3-hydroxybenzyl alcohol of Example 1 above is replaced by the compounds of Table II below then the corresponding product is obtained.

TABLE II

1,2-benzenediol
1,3-benzenediol
1,4-benzenediol
2-mercaptophenol
3-mercaptophenol
4-mercaptophenol
1,3-dimercaptobenzene
1,4-dimercaptobenzene
3-hydroxybenzyl alcohol
3-hydroxyethylphenol
4-hydroxybenzyl alcohol
4-hydroxyethylphenol
2-methylresorsinol
5-methylresorsinol
5-methoxyresorsinol
5-methyl-1,4-dihydroxybenzene
3-(N-acetylamino)phenol
3-(N-acetylamino)benzyl alcohol
2-hydroxy-α-methylbenzyl alcohol
2-hydroxy-α-ethylbenzyl alcohol
2-hydroxy-α-propylbenzyl alcohol
3-hydroxy-α-methylbenzyl alcohol
3-hydroxy-α-ethylbenzyl alcohol
3-hydroxy-α-propylbenzyl alcohol
4-hydroxy-α-methylbenzyl alcohol
4-hydroxy-α-ethylbenzyl alcohol
4-hydroxy-α-propylbenzyl alcohol

### EXAMPLE 4

When the compounds of Table I, Example 2 are reacted with the compounds of Table II, Example 3 under the conditions of Example 1 then the corresponding products are obtained.

### EXAMPLE 5

### 3-(2-QUINOLINYLMETHYLOXY)BENZYL CHLORIDE

To a stirred solution of 14.5 g of 3-(2-quinolinylmethyloxy)benzyl alcohol in 150 ml of CHCl₃ is added dropwise 7.5 ml of thionyl chloride during 10 min. The reaction mixture is stirred for 4 hours at room temperature, and then washed with NaHCO₃ solution. The organic solution is separated, dried, and evaporated to give 3-(2-quinolinylmethyloxy)benzyl chloride which is used without further purification in the next step.

### EXAMPLE 6

When the compounds prepared by Examples 2-4 are used in place of 3-(2-quinolinylmethyloxy)benzyl alcohol in Example 5, then the corresponding chloride is prepared.

### EXAMPLE 7

### 3-[3-(2-QUINOLINYLMETHYLOXY)BENZYLOXY]BENZONITRILE

A solution of 0.65 g (5.4 mmol) 3-hydroxybenzonitrile, 1.5 g (5.3 mmol) of 3-(2-quinolinylmethyloxy)benzyl chloride, and 0.75 g (5.4 mmol) of potassium carbonate in 15 ml of DMF is heated at 60° C. overnight. The reaction mixture is poured into water. The precipitated product is collected on a filter and purified by dry column chromatography to give 3-[3-(2-quinolinylmethyloxy)benzyloxy]benzonitrile. (MP 86-87° C.)

### EXAMPLE 8

When 3-hydroxybenzonitrile of Example 7 above is replaced by the compounds of Table III below then the corresponding product is obtained.

TABLE III

2-hydroxybenzonitrile
4-hydroxybenzonitrile
2-cyanomethylphenol
3-cyanomethylphenol
4-cyanomethylphenol
2-cyanoethylphenol
3-cyanoethylphenol
4-cyanoethylphenol
2-cyanopropylphenol
3-cyanopropylphenol
4-cyanopropylphenol
3-cyanobutylphenol
4-cyanobutylphenol
2-methyl-3-hydroxybenzonitrile
4-methyl-3-hydroxybenzonitrile
5-methyl-3-hydroxybenzonitrile
2-methyl-4-hydroxybenzonitrile
3-methyl-4-hydroxybenzonitrile
5-methyl-4-hydroxybenzonitrile
4-methoxy-3-hydroxybenzonitrile
3-methoxy-4-hydroxybenzonitrile
2-methoxy-4-hydroxybenzonitrile
2-methoxy-4-hydroxybenzonitrile
4-carbomethoxy-3-hydroxybenzonitrile
5-carbomethoxy-3-hydroxybenzonitrile
3-carbomethoxy-4-hydroxybenzonitrile
2,5-dimethyl-4-hydroxybenzonitrile
3-methyl-4-cyanomethylphenol.
2-methyl-4-cyanomethylphenol
2-methyl-3-cyanomethylphenol
4-methyl-3-cyanomethylphenol
5-methyl-3-cyanomethylphenol
2-mercaptobenzonitrile
3-mercaptobenzonitrile
4-mercaptobenzonitrile
3-mercaptobenzylnitrile
4-mercaptobenzylnitrile
4-methyl-3-mercaptobenzonitrile
2-cyanomethyl-1-hydroxymethylbenzene
3-cyanomethyl-1-hydroxymethylbenzene
4-cyanomethyl-1-hydroxymethylbenzene
2-hydroxymethylbenzonitrile
3-hydroxymethylbenzonitrile
4-hydroxymethylbenzonitrile
3-(N-acetylamino)benzonitrile
4-(N-acetylamino)benzonitrile

### EXAMPLE 9

When the compounds of Example 6 are used in place of 3-(2quinolinylmethyloxy)benzyl chloride in Examples 7 and 8 then the corresponding nitrites are obtained.

### EXAMPLE 10

### 5-[3-(3-(2-QUINOLINYLMETHYLOXY)BENZYLOXY)PHENYL]TETRAZOLE

A mixture of 1.2 g (3.28 mmol) of 3-[3-(2-quinolinylmethyloxy)benzyloxy]benzonitrile, 1.89 g (16.4 mmol) of pyridine hydrochloride, and 1.06 g (16.4 mmol) of sodium azide in 10 ml of DMF is heated at 100° C. for 4 days. The reaction mixture is poured into water. The crude product collected on a filter and recrystallized from ethyl acetate to give 5-[3-(3-(2-quinolinylmethyloxy)benzyloxy)-phenyl]tetrazole. (M.P. 169-172° C.)

### EXAMPLE 11

When 4-hydroxybenzyl alcohol is used in place of 3-hydroxybenzyl alcohol in Example 1 and 4-hydroxybenzonitrile is used in place of 3-hydroxybenzonitrile in Example 7 then the product obtained is 5-[4-(4-(2-quinolinylmethyloxy)benzyloxy)phenyl]tetrazole. (M.P. 210-213° C.)

### EXAMPLE 12

When 4-cyanomethylphenol is used in place of 4-hydroxybenzonitrile in Example 11 then the product obtained is 5-[4(4-(2-quinolinylmethyloxy)benzyloxy)benzyl]tetrazole. (M.P. 179-181° C.)

### EXAMPLE 13

When the nitrile compounds of Example 9 are used in place of 3-[3-(2-quinolinylmethyloxy)benzyloxy]benzonitrile in Example 10 the corresponding tetrazole product is obtained. Representative examples of compounds obtained by this invention are shown in Table IV below.

TABLE IV
5-[3-(4-(2-quinolinylmethyloxy)benzyloxy)phenyl]tetrazole
5-[2-(4-(2-quinolinylmethyloxy)benzyloxy)phenyl]tetrazole
5-[4-(3-(2-quinolinylmethyloxy)benzyloxy)phenyl]tetrazole
5-[4-(2-(2-quinolinylmethyloxy)benzyloxy)phenyl]tetrazole
5-[2-(3-(2-quinolinylmethyloxy)benzyloxy)phenyl]tetrazole
5-[3-(3-(2-quinolinylmethyloxy)benzyloxy)benzyl]tetrazole
5-[4-(3-(2-quinolinylmethyloxy)benzyloxy)benzyl]tetrazole
5-[3-(4-(2-quinolinylmethyloxy)benzyloxy)benzyl]tetrazole
5-[2-(3-(2-quinolinylmethyloxy)benzyloxy)benzyl]tetrazole
5-[4-(2-(2-quinolinylmethyloxy)benzyloxy)benzyl]tetrazole
5-[2-(4-(2-quinolinylmethyloxy)benzyloxy)benzyl]tetrazole
5-[2-(3-(4-(2-quinolinylmethyloxy)benzyloxy)phenyl)propyl]tetrazole
5-[2-(3-(4-(2-quinolinylmethyloxy)benzyloxy)phenyl)butyl]tetrazole
5-[3-(3-(4-(2-quinolinylmethyloxy)benzyloxy)phenyl)butyl]tetrazole
5-[3-(3-(2-quinolinylmethylthio)benzyloxy)phenyl]tetrazole
5-[3-(3-(2-quinolinylmethylthio)benzylthio)phenyl]tetrazole
5-[3-(3-(2-quinolinylmethyloxy)benzylthio)phenyl]tetrazole
5-[4-(3-(2-quinolinylmethyloxy)benzyloxy)-3-methoxyphenyl]tetrazole
5-[3-(3-(2-quinolinylmethyloxy)benzyloxy)-4-methoxyphenyl]tetrazole
5-[4-(4-(2-quinolinylmethyloxy)benzyloxy)-3-methoxyphenyl]tetrazole
5-[3-(4-(2-quinolinylmethyloxy)benzyloxy)-4-methoxyphenyl]tetrazole
5-[4-(3-(2-quinolinylmethyloxy)benzyloxy)-2-methoxyphenyl]tetrazole
5-[4-(3-(2-quinolinylmethyloxy)benzyloxy)-3-
carbomethoxyphenyl]tetrazole
5-[4-(3-(2-quinolinylmethyloxyjbenzyloxy)-3-methoxybenzyl]tetrazole
5-[4-(4-(2-quinolinylmethyloxy)benzyloxy)-3-methoxybenzyl]tetrazole
5-[4-(4-(2-quinolinylmethyloxy)benzyloxy)-3-
carbomethoxybenzyl]tetrazole
5-[4-(3-(2-quinolinylmethyloxy)benzyloxy)-3-
carbomethoxybenzyl]tetrazole
5-[4-(3-(2-quinolinylmethyloxy)benzylthio)phenyl]tetrazole
5-[3-(4-(2-quinolinylmethyloxy)benzylthio)phenyl]tetrazole
5-[4-(3-(2-quinolinylmethyloxy)-N-acetyl-benzylamino)phenyl]tetrazole
5-[4-(4-(2-quinolinylmethyloxy)-N-acetyl-benzylamino)phenyl]tetrazole

### EXAMPLE 14

### METHYL 3-METHOXY-4-[3-(2-QUINOLINYLMETHYLOXY)BENZYLOXY]-BENZOATE

A mixture of 3 g of 3-(2-quinolinylmethyloxy) benzyl chloride, 1.93 g of methyl 4-hydroxy-3-methoxy benzoate, and 1.5 g of potassium carbonate in 30 ml of DMF is heated at 50° C. overnight. The reaction mixture is poured into water, the solid product collected on a filter and purified by dry column chromatography to give methyl 3-methoxy-4-(3-(2-quinolinylmethyloxy)benzyloxy)-benzoate. (M.P. 100-101° C.)

### EXAMPLE 15

### 3-METHOXY4-[3-(2-QUINOLINYLMETHYLOXY)BENZYLOXY]-BENZOIC ACID

A mixture of 2.6 g of methyl 3-methoxy-4-[3-(2-quinolinylmethyloxy)benzyloxy]benzoate and 0.6 g of NaOH in 15 ml of THF and 2 ml of H₂0 are heated at 60° C. overnight. The reaction mixture is diluted with 20 ml of H₂O and acidified to pH 4. The product is collected on a filter and dried to give 3-methoxy-4-(3-(2-quinolinylmethyloxy)benzyloxy)benzoic acid. (M.P. 188-190° C.)

### EXAMPLE 16

When methyl 4-hydroxy-3-methoxybenzoate is replaced in the procedure of Example 14 with the compounds of Table V, below, then the corresponding products are obtained. Representative examples of compounds prepared by this invention are shown in Table VI.

TABLE V

methyl 2-hydroxybenzoate
methyl 3-hydroxybenzoate
methyl 4-hydroxybenzoate
methyl 3-hydroxy-4-methoxybenzoate
methyl 4-hydroxy-2-methoxybenzoate
methyl 3-hydroxy-4-methoxybenzoate
ethyl 4-hydroxy-3-ethoxybenzoate
methyl 4-hydroxy-3-methylbenzoate
methyl 3-hydroxy-4-methylbenzoate
methyl 4-hydroxy-2-methylbenzoate
methyl 3-hydroxy-4-methylbenzoate
methyl 4-hydroxy-2,6-dimethylbenzoate
methyl 4-hydroxy-2,5-dimethylbenzoate
methyl 2-hydroxyphenylacetate
methyl 3-hydroxyphenylacetate
methyl 4-hydroxyphenylacetate
methyl 4-hydroxyphenylpropionate
methyl 4-hydroxyphenylbutyrate
methyl 4-hydroxyphenyl-3-methylbutyrate
methyl 4-hydroxy-3-methylphenylacetate
methyl 3-hydroxy-4-methylphenylacetate
methyl 4-hydroxy-3-methoxyphenylacetate
methyl 3-hydroxy-4-methoxyphenylacetate
methyl 2-hydroxymethylbenzoate
methyl 3-hydroxymethylbenzoate
methyl 4-hydroxymethylbenzoate
methyl 2-hydroxymethylphenylacetate
methyl 3-hydroxymethylphenylacetate
methyl 4-hydroxymethylphenylacetate
3-mercaptobenzoate
4-mercaptobenzoate
3-mercaptomethylbenzoate
3-(N-acetylamino)benzoate
4-(N-acetylamino)benzoate
4-(N-benzylamino)benzoate

TABLE VI

4-(3-(2-quinolinylmethyloxy)benzyloxy)benzoic acid
4-(4-(2-quinolinylmethyloxy)benzyloxy)benzoic acid
3-(4-(2-quinolinylmethyloxy)benzyloxy)benzoic acid
3-(3-(2-quinolinylmethyloxy)benzyloxy)benzoic acid
2-(4-(2-quinolinylmethyloxy)benzyloxy)benzoic acid
4-(3-(2-quinolinylmethyloxy)benzyloxy)phenylacetic acid
4-(3-(2-quinolinylmethyloxy)phenoxy)benzoic acid
4-(3-(2-quinolinylmethyloxy)benzyloxymethyl)benzoic acid
3-methyl-4-(3-(2-quinolinylmethyloxy)benzyloxy)benzoic acid
4-methyl-3-(3-(2-quinolinylmethyloxy)benzyloxy)benzoic acid
2-methyl-4-(3-(2-quinolinylmethyloxy)benzyloxy)benzoic acid
3-methoxy-4-(3-(2-quinolinylmethyloxy)benzyloxy)benzoic acid
4-methoxy-3-(3-(2-quinolinylmethyloxy)benzyloxy)benzoic acid
2,6-dimethyl-4-(3-(2-quinolinylmethyloxy)benzyloxybenzoic acid
4-(3-(2-quinolinylmethyloxy)benzylthio)benzoic acid
4-(3-(2-quinolinylmethyloxy)benzylamino)benzoic acid

### EXAMPLE 17

### 3-METHOXY-4-(3-(2-QUINOLINYLMETHYLOXY)PHENOXYMETHYL)BENZOYL-N-BENZESULFONAMIDE

A reaction mixture of 0.73 g of 3-methoxy-4-(3-(2quinolinyl-methyloxy)phenoxy)benzoic acid, 0.28 g of benzenesulfonamide, 0.28 g of 4-dimethylpyridine, and 0.44 g of 1-(3-dimethylamino-propyl)-3-ethylcarbodimide hydrochloride in 50 ml of CH₂Cl₂ is stirred at room temperature overnight. The solvent is removed and the residue is extracted into ethyl acetate. The organic solution is washed with water, and evaporated. The product is purified by dry column chromatography to give 3-methoxy-4-(3-(2quinolinylmethyloxy) phenoxymethyl)benzoyl-N-benzenesulfonamide. (M.P. 156-158° C.)

### EXAMPLE 18

When 3-methoxy-4-(3-(2-quinolinylmethyloxy)phenoxymethyl)benzoic acid of Example 17 is replaced by the acids of this invention such as those of Example 16, Table VI and Example 25, Table IX then the corresponding benzenesulfonamide compound is prepared.

When benzenesulfonamide is replaced in the above Examples by a sulfonamide of formula NH₂SO₂R₃ or an amine of formula HN(R₃)₂, then the corresponding product is obtained.

### EXAMPLE 19

### METHYL 3-(3-(2-QUINOLINYLMETHYLOXY)PHENOXYMETHYL)BENZOATE

A mixture of 3-(2-quinolinylmethyloxy)phenol (2.51 g, 0.01 mol), 1.85 g (0.01 mol) of methyl 3-chloromethyl benzoate, and 1.5 g of potassium carbonate in 30 ml of DMS is heated at 50° C. overnight. The reaction mixture is poured into water, extracted with ethyl acetate and the organic solution separated, dried and evaporated to dryness. Recrystallization from ethyl acetate gives methyl 3-(3-(2-quinolinylmethyloxy)phenoxymethyl)benzoate. (M.P. 93-94 C.)

### EXAMPLE 20

A mixture of 1.6 g of methyl 3-(3-(2-quinolinylmethyloxy)phenoxymethyl)benzoate and 0.5 g of NaOH in 20 ml of THF and 5 ml of H₂0 is heated at 50° C. overnight. The reaction mixture is acidified to pH 4 by 1N HCl solution, filtered and dried to give 3-(3-(2-quinolinylmethyloxy)phenoxymethyl)-benzoic acid. (M.P. 149-151° C.)

### EXAMPLE 21

When the procedures of Examples 19 and 20 are followed and methyl 3-chloromethylbenzoate is replaced by methyl 4-chloromethylbenzoate, then the product prepared is 4-(3-(2-quinol-inylmethyloxy)phenoxymethyl)benzoic acid. (M.P. 190-191° C.)

### EXAMPLE 22

When the procedures of Examples 19 and 20 are followed and methyl 3-chloromethylbenzoate is replaced by methyl 3-methoxy4-chloromethylbenzoate then the product prepared is 3-methoxy-4-(3-(2-quinolinylmethyloxy)phenoxymethyl)benzoic acid. (M.P. 208-210° C.)

### EXAMPLE 23

When the procedure of Example 19 is followed and the compounds of Table VII below are used in place of methyl-3-chloromethyl-benzoate then the corresponding product is obtained.

TABLE VII

ethyl 2-chloromethylbenzoate
ethyl 3-chloromethylbenzoate
ethyl 4-chloromethylbenzoate
ethyl 3-chloromethylbenzoate
methyl 4-chloromethylbenzoate
methyl 2-methyl-5-chloromethylbenzoate
methyl 2-methyl-3-chloromethylbenzoate
methyl 3-methyl-5-chloromethylbenzoate
methyl 4-methyl-5-chloromethylbenzoate
methyl 2-methyl-4-chloromethylbenzoate
methyl 3-methyl-4-chloromethylbenzoate
methyl 2-methoxy-5-chloromethylbenzoate
methyl 2-methoxy-3-chloromethylbenzoate
methyl 2-methoxy-4-chloromethylbenzoate
methyl 3-methoxy-4-chloromethylbenzoate
methyl 3-chloromethylphenylacetate
methyl 4-chloromethylphenylacetate
methyl 3-chloromethylphenylpropionate
methyl 4-chloromethylphenylpropionate
methyl 3-chloromethylphenylbutyrate
methyl 4-chloromethylphenylbutyrate
methyl 3-chloromethylphenylisopropionate
methyl 4-chloromethylphenylisopropionate
methyl 3-chloromethylphenylisopropionate
methyl 4-chloromethylphenylisobutyrate

### EXAMPLE 24

When the procedure of Example 19 is followed and the compound of Table VIII below are used in place of 3-(2quinolinyl-methyloxy)phenol then the corresponding product is obtained.

TABLE VIII

3-(2-quinolinylmethyloxy)phenol
4-(2-quinolinylmethyloxy)phenol
3-(2-quinolinylmethylthio)phenol
4-(2-quinolinylmethylthio)phenol
5-methyl-3-(2-quinolinylmethyloxy) phenol
2-methyl-3-(2-quinolinylmethyloxy)phenol
5-methoxy-3-(2-quinolinylmethyloxy)phenol
2-methyl-4-(2-quinolinylmethyloxy)phenol
2-methoxy-4-(2-quinolinylmethyloxy)phenol
3-methoxy-4-(2-quinolinylmethyloxy)phenol
3-methyl-4-(2-quinolinylmethyloxy)phenol
3-(2-quinolinylmethyloxy)phenyl mercaptan
4-(quinolinylmethyloxy)phenyl mercaptan
3-(2-quinolinylmethylthio)phenyl mercaptan
4-(2-quinolinylmethylthio)phenyl mercaptan
N-benzyl-3-(2-quinolinylmethyloxy)phenylamine
N-methyl-3-(2-quinolinylmethyloxy)phenylamine
N-acetyl-3-(2-quinolinylmethyloxy)phenylamine
N-acetyl-4-(2-quinolinylmethyloxy)phenylamine

### EXAMPLE 25

When the procedures of Examples 19 and 20 are followed using the compounds of Table VII, Example 23 and Table VIII, Example 24, then the corresponding product is obtained. Representative examples of compounds prepared by this invention are shown in Table IX.

TABLE IX
3-(4-(2-quinolinylmethyloxy)phenoxymethyl)benzoic acid
4-(4-(2-quinolinylmethyloxy)phenoxymethyl)benzoic acid
2-(3-(2-quinolinylmethyloxy)phenoxymethyl)benzoic acid
2-(4-(2-quinolinylmethyloxy)phenoxymethyl)benzoic acid
2-methyl-3-(3-(2-quinolinylmethyloxy)phenoxymethyl)benzoic acid
2-ethyl-3-(3-(2-quinolinylmethyloxy)phenoxymethyl)benzoic acid
2-methoxy-3-(3-(2-quinolinylmethyloxy)phenoxymethyl)benzoic acid
3-methyl-4-(3-(2-quinolinylmethyloxy)phenoxymethyl)benzoic acid
2-methyl-4-(3-(2-quinolinylmethyloxy)phenoxymethyl)benzoic acid
2-methoxy-4-(3-(2-quinolinylmethyloxy)phenoxymethyl)benzoic acid
3-(3-(2-quinolinylmethyloxy)-5-methylphenoxymethyl)benzoic acid
3-(3-(2-quinolinylmethyloxy)-5-methoxyphenoxymethyl)benzoic acid
3-(4-(2-quinolinylmethyloxy)-3-methylphenoxymethyl)benzoic acid
3-(4-(2-quinolinylmethyloxy)-2-methylphenoxymethyl)benzoic acid
2-methyl-3-(3-(2-quinolinylmethyloxy)-2-methylphenoxymethyl)benzoic
acid
3-(3-(2-quinolinylmethylthio)phenoxymethyl)benzoic acid
4-(4-(2-quinolinylmethylthio)phenoxymethyl)benzoic acid
3-(3-(2-quinolinylmethyloxy)phenoxymethyl)phenylacetic acid
3-(3-(2-quinolinylmethyloxy)phenoxymethyl)phenylpropionic acid
3-(3-(2-quinolinylmethyloxy)phenylthiomethyl)benzoic acid
4-(3-(2-quinolinylmethyloxy)phenylthiomethyl)benzoic acid
3-(4-(2-quinolinylmethyloxy)phenylthiomethyl)benzoic acid
3-(3-(2-quinolinylmethyloxy)phenyl-N-acetylamino-methyl)benzoic acid
4-(4-(2-quinolinylmethyloxy)phenyl-N-acetylaminomethyl)benzoic acid

### EXAMPLE 26

### 4-(3-(2-QUINOLINYLMETHYLOXY)PHENOXYMETHYL)BENZONITRILE

A solution of 7.24 g (19.92 mmol) of sodium 3-(2quinolinylmethyloxy)phenoxide pentahydrate and 4.68 g (23.90 mmol) of p-cyanobenzyl bromide in 34 ml of dry DMF is stirred at 75° C. under nitrogen for 2 days. The reaction mixture is cooled to room temperature, then poured into 400 ml of 3:1 H₂O/Et₂O, shaken; and the phases separated. The aqueous layer is extracted and washed with 1:1 brine/H₂O and brine. The ether solution is dried over 1:1 Na₂SO₄MgSO₄, filtered and concentrated. The crude product is recrystallized from 70% EtOAc/hexane to obtain 4-(3-(2-quinolinylmethyloxy)phenoxy-methyl)benzonitrile. (M.P. 112.5° C.)

### EXAMPLE 27

### 5-(4-(3-(2-QUINOLINYLMETHYLOXY)PHENOXYMETHYL)PHENYL)TETRAZOLE

A slurry of 2.0 g (5.48 mol) of 4-(3-(2-quinolinylmethyloxy)phenoxymethyl)benzonitrile, 1.78 g (27.4 mmol) of sodium azide, and 3.16 g (27.4 mmol) of pyridinium hydrochloride in 12 ml of dry DMF is stirred under nitrogen at 100° C. for 20 hrs. The reaction mixture is then cooled to room temperature and concentrated. The residue is taken up on 100 ml of 1N aqueous NaOH and the solution extracted with ether. The aqueous layer is acidified to pH 6 with 1N aqueous HCl, and the precipitate collected, triturated with water, filtered and lyophilized to obtain 5-(4-(3-(2-quinolinylmethyloxy)phenoxy-methyl)phenyl)tetrazole. (M.P. 91° C. dec.)

### EXAMPLE 28

When the procedures of Examples 26 and 27 are followed and p-cyanobenzyl bromide is replaced by o-cyanobenzyl bromide, m-cyanobenzyl bromide, o-(cyanomethyl)benzyl bromide, m-(cyanomethyl)benzyl bromide, and p-(cyanomethyl)-benzyl bromide, then the products prepared are:

5-(2-(3-(2-quinolinylmethyloxy)phenoxymethyl)phenyl)tetrazole (M.P. 166-170° C.);

5-(3-(3-(2-quinolinylmethyloxy)phenoxymethyl)phenyl)tetrazole (M.P. 115° C. dec.);

5-(2-(3-(2-quinolinylmethyloxy)phenoxymethyl)benzyl)tetrazole (M.P. 145.5-147° C.);

5-3-(3-(2-quinolinylmethyloxy)phenoxymethyl)benzyl)tetrazole (M.P. 161-164° C.); and

5-(4-(3-(2-quinolinylmethyloxy)phenoxymethyl)benzyl)tetrazole (M.P. 149-1 52° C.).

### EXAMPLE 29

When the procedure of Example 26 is followed and the compounds of Table X below are used in place of p-cyanobenzyl bromide then the corresponding product is obtained.

TABLE X

2-methyl-4-cyanobenzyl bromide
3-methyl-4-cyanobenzyl bromide
3-methoxy-2-cyanobenzyl bromide
2-methyl-3-cyanobenzyl bromide
3-cyano-4-methylbenzyl bromide
4-methoxy-2-cyanobenzyl bromide
3-cyano-5-methylbenzyl bromide
2-methyl-5-cyanobenzyl bromide
2-methoxy-5-cyanobenzyl bromide
2-methoxy-4-cyanobenzyl bromide
2-methoxy-3-cyanobenzyl bromide
2,6-dimethyl-4-cyanobenzyl bromide
3-methoxy-4-cyanobenzyl bromide
2-methyl-6-cyanobenzyl bromide
o-cyanobenzyl bromide
m-cyanobenzyl bromide
p-cyanobenzyl bromide
2-cyanomethylbenzyl bromide
3-cyanomethylbenzyl bromide
4-cyanomethylbenzyl bromide
3-(1′-cyanoethyl)benzyl bromide
3-(2′-cyanoethyl)benzyl bromide
4-(1′-cyanoethyl)benzyl bromide
4-(2′-cyanoethyl)benzyl bromide
3-(1′-cyanopropyl)benzyl bromide
3-(2′-cyanopropyl)benzyl bromide
3-(3′-cyanopropyl)benzyl bromide
4-(1′-cyanopropyl)benzyl bromide
4-(2′-cyanopropyl)benzyl bromide
4-(3′-cyanopropyl)benzyl bromide
3-(1′-cyanobutyl)benzyl bromide
3-(2′-cyanobutyl)benzyl bromide
3-(3′-cyanobutyl)benzyl bromide
3-(4′-cyanobutyl)benzyl bromide
4-(1′-cyanobutyl)benzyl bromide
4-(2′-cyanobutyl)benzyl bromide
4-(3′-cyanobutyl)benzyl bromide
4-(4′-cyanobutyl)benzyl bromide
3-(2′-methyl-1′-cyanobutyl)benzyl bromide
3-(3′-methyl-1′-cyanobutyl)benzyl bromide
4-(2′-methyl-1′-cyanobutyl)benzyl bromide
4-(3′-methyl-1′-cyanobutyl)benzyl bromide

### EXAMPLE 30

When the procedure of Example 26 is followed and the sodium or other appropriate salt of the alcohol or mercaptan of Table VIII, Example 24 is used is place of sodium 3-(2-quinolinylmethyloxy)-phenoxide then the corresponding product is obtained.

### EXAMPLE 31

When the procedures of Examples 26 and 27 are followed using the compounds of Table X, Example 29 and the appropriate alcohol, thio or amino salt formed in Example 30, then the corresponding products are obtained. Representative examples of compounds prepared by this invention are shown in Table XI.

TABLE XI
5-(4-(4-(2-quinolinylmethyloxy)phenoxymethyl)phenyl)tetrazole
5-(3-(4-(2-quinolinylmethyloxy)phenoxymethyl)phenyl)tetrazole
5-(3-(2-(2-quinolinylmethyloxy)phenoxymethyl)phenyl)tetrazole
5-(2-(4-(2-quinolinylmethyloxy)phenoxymethyl)phenyl)tetrazole
5-(4-(2-(2-quinolinylmethyloxy)phenoxymethyl)phenyl)tetrazole
5-(2-(2-(2-quinolinylmethyloxy)phenoxymethyl)phenyl)tetrazole
5-(3-(3-(2-quinolinylmethyloxy)phenoxymethyl)phenyl)tetrazole
5-(4-(3-(2-quinolinylmethyloxy)-5-
methoxyphenoxymethyl)phenyl)tetrazole
5-(4-(3-(2-quinolinylmethyloxy)-5-
methylphenoxymethyl)phenyl)tetrazole
5-(3-(4-(2-quinolinylmethyloxy)-2-
methylphenoxymethyl)phenyl)tetrazole
5-(3-(4-(2-quinolinylmethyloxy)-2-
methoxyphenoxymethyl)phenyl)tetrazole
5-(4-(3-(2-quinolinylmethyloxy)-2-
methylphenoxymethyl)phenyl)tetrazole
5-(4-(4-(2-quinolinylmethyloxy)-2-methylphenoxymethyl)phenyl)tetrazole
5-(4-(4-(2-quinolinylmethyloxy)-3-methylphenoxymethyl)phenyl)tetrazole
5-(4-(3-(2-quinolinylmethylthio)phenoxymethyl)phenyl)tetrazole
5-(3-(3-(2-quinolinylmethylthio)phenoxymethyl)phenyl)tetrazole
5-(2-(3-(2-quinolinylmethylthio)phenoxymethyl)phenyl)tetrazole
5-(2-(4-(2-quinolinylmethyloxy)phenoxymethyl)benzyl)tetrazole
5-(4-(4-(2-quinolinylmethyloxy)phenoxymethyl)benzyl)tetrazole
5-(3-(4-(2-quinolinylmethyloxy)phenoxymethyl)benzyl)tetrazole
5-(4-(3-(2-quinolinylmethyloxy)phenoxymethyl)phenethyl)tetrazole
5-(3-(2-(4-(2-quinolinylmethyloxy)phenoxymethyl)phenyl)propyl)tetrazole
5-(4-(3-(2-(2-quinolinylmethyloxy)phenoxymethyl)phenyl)butyl)tetrazole
5-(2-(4-(3-(2-quinolinylmethyloxy)phenoxymethyl)phenyl)propyl)tetrazole
5-(3-(4-(3-(2-quinolinylmethyloxy)phenoxymethyl)phenyl)butyl)tetrazole
5-(4-(4-(3-(2-quinolinylmethyloxy)phenoxymethyl)phenyl)-3-
methylbutyl)tetrazole
5-(4-(3-(2-quinolinylmethyloxy)phenylthiomethyl)phenyl)tetrazole
5-(4-(3-(2-quinolinylmethylthio)phenylthiomethyl)phenyl)tetrazole
5-(4-(3-(2-quinolinylmethyloxy)phenoxymethyl)-3-methylphenyl)tetrazole
5-(4-(3-(2-quinolinylmethyloxy)phenoxymethyl)-2-methylphenyl)tetrazole
5-(4-(3-(2-quinolinylmethyloxy)phenoxymethyl)-2-
methoxyphenyl)tetrazole
5-(4-(3-(2-quinolinylmethyloxy)phenoxymethyl)-3-
methoxyphenyl)tetrazole
5-(2-(4-(2-quinolinylmethyloxy)phenoxymethyl)-3-
methylphenyl)tetrazole
5-(3-(4-(2-quinolinylmethyloxy)phenoxymethyl)-4-
methoxyphenyl)tetrazole
5-(3-(3-(2-quinolinylmethyloxy)phenoxymethyl)-4-
methoxyphenyl)tetrazole
5-(4-(3-(2-quinolinylmethyloxy)-5-methylphenoxymethyl)-2-
methoxyphenyl)tetrazole
5-(4-(3-(2-quinolinylmethyloxy)-N-
acetylphenylaminomethyl)phenyl)tetrazole
5-(4-(3-(2-quinolinylmethylthio)-N-
acetylphenylaminomethyl)phenyl)tetrazole

### EXAMPLE 32

### 5-(3-(4-(2-QUINOLINYLMETHYLOXY)PHENOXYMETHYL)PHENOXYMETHYL)TETRAZOLE

A. α-(3-hydroxymethylphenoxy)acetonitrile

A mixture of 3-hydroxymethyl phenol (0.081 mol), bromoacetonitrile (0.081 mol) and anhydrous potassium carbonate (0.081 mol) in acetone (160 ml) and dimethylformamide (20 ml) are heated at reflux for 48 hrs. The reaction mixture is filtered and evaporated. The residue is diluted with ethyl acetate (150 ml), washed with 10% aqueous sodium hydroxide solution (3×100 ml) and then with brine (3×100 ml). The ethyl acetate solution is dried (magnesium sulfate) and chromatographed using a silica gel column (ca. 100 g) and eluted with 1:1 petroleum ether: ethylacetate (2 1). The resultant oil is used directly in the next step.

B. α-(3-chloromethylphenoxy)acetonitrile

α-(3-Hydroxymethylphenoxy)acetonitrile (0.055 mol) in diethylether (150 ml) is stirred with thionyl chloride (0.060 mol) and a few drops of dimethylformamide at 40° C. for 1 hr. the solution is washed with water and brine, then evaporated to give α-(3-chloromethylphenoxy)acetonitrile as a yellow oil which is used directly in the next step.

C. α-(3-(4-(2-quinolinylmethyloxy)phenoxymethyl)phenoxy)acetonitrile

A mixture of α-(3-chloromethylphenoxy)acetonitrile (0.025 mol), sodium 4-(2-quinolinylmethyloxy)phenoxide (0.025 mol) and anhydrous potassium carbonate (0.125 mol) in dimethylsulfoxide (50 ml) is stirred at ambient temperature for 18 hrs. The reaction is diluted with water (600 ml) and extracted with ethyl acetate (3×150 ml). The ethyl acetate solution is washed with water (3×100 ml) and brine (100 ml) then dried and evaporated to give α-(3-(4-(2-quinolinyl-methyloxy)phenoxymethyl)phenoxy)acetonitrile. (M.P. 110-114° C.)

D. 5-(3-(4-(2-quinolinylmethyloxy)phenoxymethyl)phenoxymethyl)tetrazole

α-(3-(4-(2-quinolinylmethyloxy)phenoxymethyl)phenoxy)acetonitrile (8.12 mmol), sodium azide (24.4 mmol) and ammonium chloride (24.4 mmol) in dimethylformamide (10 ml) are heated at 115-120° C. for 6 hrs. After cooling, the reaction mixture is diluted with ethyl acetate (150 ml), washed with water (6×100 ml) then dried and evaporated. The residue is chromatographed on a column of silica gel (360 g) and eluted with a gradient of isopropanol in methylene chloride to give 5-(3-(4-(2-quinolinylmethyloxy)phenoxymethyl)phenoxymethyl)tetrazole. (M.P. 131-32° C.)

### EXAMPLE 33

When sodium 4-(2-quinolinylmethyloxy)phenoxide of Example 32, Step C, is replaced with sodium 3-(2-quinolinylmethyloxy)phenoxide, the product prepared is 5-(3-(3-(2-quinolinyl-methyloxy)phenoxymethyl)phenoxymethyl)tetrazole. (M.P. 135-137° C.)

### EXAMPLE 34

When α-(3-hydroxymethylphenoxy)acetonitrile of Example 32, Step B. is replaced with α-(4-hydroxymethylphenoxy)acetonitrile then the product prepared is 5-(4-(3-(2 quinolinylmethyloxy)phenoxymethyl)phenoxymethyl)tetrazole. (M.P. 154-156° C.)

### EXAMPLE 35

When α-(3-hydroxymethylphenoxy)acetonitrile of Example 32, Step B. is replaced with α-(2-hydroxymethylphenoxy)acetonitrile or α-((2-hydroxymethyl-5-carbomethoxy)phenoxy)acetonitrile then the products prepared are 5-(2-(3-(2quinolinylmethyloxy)phenoxymethyl)phenoxymethyl)tetrazole (M.P. 118-120° C.) or 5-(2-(3-(2-quinolinylmethyloxy)-phenoxymethyl)-5-carbomethoxy-phenoxymethyl)tetrazole. (M.P. 159-162° C.)

### EXAMPLE 36

When bromoacetonitrile of Example 32, Step A is replaced by the nitriles of Table XII below then the corresponding product is prepared:

TABLE XII

bromoacetonitrile
α-bromo-α-methylacetonitrile
α-bromo-β-ethylacetonitrile
α-bromopropionitrile
β-bromopropionitrile
β-bromo-β-methylpropionitrile-bromobutyronitrile
β-bromobutyronitrile
α-bromobutyronitrile

### EXAMPLE 37

When 3-hydroxymethylphenol of Example 32, Step A is replaced by the compounds of Table XIIIa below, then the corresponding products are prepared.

TABLE XIIIa

2-hydroxymethylphenol
4-hydroxymethylphenol
3-mercaptobenzylalcohol
4-mercaptobenzylalcohol
3-hydroxymethyl-N-acetylamidine
4-hydroxymethyl-N-acetylamidine
4-hydroxymethylamidine
4-methyl-2-hydroxymethylphenol
2-methyl-5-hydroxymethylphenol
4-methyl-3-hydroxymethylphenol
5-methyl-3-hydroxymethylphenol
3-methyl-4-hydroxymethylphenol
2-methyl-4-hydroxymethylphenol
3-methyl-5-hydroxymethylphenol
4-methoxy-3-hydroxymethylphenol
3-methoxy-4-hydroxymethylphenol
2-methoxy-4-hydroxymethylphenol
5-methoxy-3-hydroxymethylphenol
3-methoxy-5-hydroxymethylphenol
2-methoxy-5-hydroxymethylphenol
2-(1′-hydroxyethyl)phenol
3-(1′-hydroxyethyl)phenol
4-(1′-hydroxyethyl)phenol
2-(2′-hydroxyethyl)phenol
3-(2′-hydroxyethyl)phenol
4-(2′-hydroxyethyl)phenol
2-(3′-hydroxypropyl)phenol
3-(3′-hydroxypropyl)phenol
4-(3′-hydroxypropyl)phenol
2-(2′-hydroxypropyl)phenol
3-(2′-hydroxypropyl)phenol
4-(2′-hydroxypropyl)phenol
2-(1′-hydroxypropyl)phenol
3-(1′-hydroxypropyl)phenol
4-(1′-hydroxypropyl)phenol
3-(4′-hydroxybutyl)phenyl
4-(4′-hydroxybutyl)phenyl

### EXAMPLE 38

Following the procedures of Examples 32 to 34, when sodium 4-(2-quinolinylmethyloxy)phenoxide of Example 32, Step C, is replaced by the metal hydroxy, thio or amino salts of the compounds of Table VIII, Example 24, then the corresponding product is prepared. Representative examples of compounds prepared by this invention are shown in Table XIIIb.

TABLE XIIIb
5-(4-(4-(2-quinolinylmethyloxy)phenoxymethyl)phenoxymethyl)tetrazole
5-(4-(2-(2-quinolinylmethyloxy)phenoxymethyl)phenoxymethyl)tetrazole
5-(3-(2-(2-quinolinylmethyloxy)phenoxymethyl)phenoxymethyl)tetrazole
5-(2-(4-(2-quinolinylmethyloxy)phenoxymethyl)phenoxymethyl)tetrazole
5-(2-(3-(2-quinolinylmethyloxy)phenoxymethyl)phenoxymethyl)tetrazole
5-(2-(2-(2-quinolinylme.thyloxy)phenoxymethyl)phenoxymethyl)tetrazole
5-(3-(4-(2-quinolinylmethyloxy)phenoxymethyl)-2-methoxyphenoxymethyl)tetrazole
5-(3-(4-(2-quinolinylmethyloxy)phenoxymethyl)-3-methoxyphenoxymethyl)tetrazole
5-(4-(3-(2-quinolinylmethyloxy)phenoxymethyl)-2-methoxyphenoxymethyl)tetrazole
5-(4-(3-(2-quinolinylmethyloxy)phenoxymethyl)-3-methoxyphenoxymethyl)tetrazole
5-(4-(3-(2-quinolinylmethyloxy)phenoxymethyl)-3-methylphenoxymethyl)tetrazole
5-(4-(4-(2-quinolinylmethyloxy)phenoxymethyl)-2-methoxyphenoxymethyl)tetrazole
5-(4-(4-(2-quinolinylmethyloxy)phenoxymethyl)-3-methoxyphenoxymethyl)tetrazole
5-(4-(4-(2-quinolinylmethyloxy)phenoxymethyl)-3-methylphenoxymethyl)tetrazole
5-(4-(4-(2-quinolinylmethyloxy)phenoxymethyl)-2-methylphenoxymethyl)tetrazole
5-(4-(4-(2-quinolinylmethyloxy)-2-methylphenoxymethyl) phenoxymethyl)tetrazole
5-(4-(4-(2-quinolinylmethyloxy)-3-methylphenoxymethyl)phenoxymethyl)tetrazole
5-(4-(4-(2-quinolinylmethyloxy)-3-methoxyphenoxymethyl)phenoxymethyl)tetrazole
5-(3-(3-(2-quinolinylmethyloxy)-4-methoxyphenoxymethyl)phenoxymethyl)tetrazole
5-(3-(3-(2-quinolinylmethyloxy)-4-methylphenoxymethyl)phenoxymethyl)tetrazole
5-(4-(4-(2-quinolinylmethyloxy)-2-methylphenoxymethyl)-3-methylphenoxymethyl)tetrazole
5-(4-(4-(2-quinolinylmethyloxy)-3-methylphenoxymethyl)-2-methylphenoxymethyl)tetrazole
5-(2-(3-(4-(2-quinolinylmethyloxy)phenoxymethyl)phenoxy)ethyl)tetrazole
5-(3-(3-(4-(2-quinolinylmethyloxy)phenoxymethyl)phenoxy)propyl)tetrazole
5-(2-(3-(4-(2-quinolinylmethyloxy)phenoxymethyl)phenoxy)propyl)tetrazole
5-(3-(3-(4-(2-quinolinylmethyloxy)phenoxymethyl)phenoxy)butyl)tetrazole
5-(4-(4-(2-quinolinylmethyloxy)phenylthiomethyl)phenoxymethyl)tetrazole
5-(4-(4-(2-quinolinylmethyloxy)phenylthiomethyl)phenylthiomethyl)tetrazole
5-(4-(4-(2-quinolinylmethylthio)phenoxymethyl)phenoxymethyl)tetrazole
5-(4-(4-(2-quinolinylmethyloxy)phenoxymethyl)phenyl-N-acetylaminomethyl)tetrazole
5-(3-(4-(4-(2-quinolinylmethyloxy)phenoxymethyl)phenylthio)butyl)tetrazole
5-(3-(3-(4-(2-quinolinylmethyloxy)phenoxy-1′-ethyl)phenoxymethyl)tetrazole
5-(3-(3-(4-(2-quinolinylmethyloxy)phenoxy-2′-propyl)phenoxymethyl)tetrazole
5-(3-(3-(4-(2-quinolinylmethyloxy)phenoxy-3′-butyl)phenoxymethyl)tetrazole

### EXAMPLE 39

### 3-(3-(2-QUINOLINYLMETHYLOXY)BENZYLOXY)BENZALDEHYDE

When 3-hydroxybenzonitrile in Example 7 is replaced by 3-hydroxybenzaldehyde then the product prepared is 3-[3-(2-quinolinylmethyloxy)benzyloxy)benzaldehyde.

### EXAMPLE 40

When 3-hydroxybenzaldehyde of Example 39 is replaced by the compounds of Table XIV below, then the corresponding product is obtained.

TABLE XIV

2-hydroxybenzaldehyde
4-hydroxybenzaldehyde
2-methyl-3-hydroxybenzaldehyde
5-methyl-3-hydroxybenzaldehyde
2-methyl-4-hydroxybenzaldehyde
3-methyl-4-hydroxybenzaldehyde
5-methoxy-3-hydroxybenzaldehyde
4-methoxy-3-hydroxybenzaldehyde
2-methoxy-3-hydroxybenzaldehyde
5-carbomethoxy-3-hydroxybenzaldehyde
3-hydroxyphenylacetaldehyde
4-hydroxyphenylacetaldehyde
3-hydroxyphenylpropionaldehyde
4-hydroxyphenylpropionaldehyde
3-hydroxyphenylisopropionaldehyde
4-hydroxyphenylisopropionaldehyde
3-hydroxyphenoxyacetaldehyde
4-hydroxyphenylthiopropionaldehyde

### EXAMPLE 41

When 3-(2-quinolinylmethyloxy)benzyl chloride of Example 39 is replaced by the compounds prepared by Examples 2-6 and 3-hydroxybenzaldehyde of Example 39 is replaced by the compounds of Table XIV, Example 40, then the corresponding products are obtained.

### EXAMPLE 42

### 3-(3-(2-QUINOLINYLMETHYLOXY)BENZYLOXY)CINNAMYLNITRILE

Sodium hydride (60% oil dispersion, 1.2 g) and diethyl cyanomethylphosphonate (5 ml) are combined and stirred in THF (50 ml) for 5 minutes. This is then added to a THF solution of 3-(3-(2-quinolinylmethyloxy)benzyloxy)benzaldehyde (9.59 g). The reaction mixture is stirred for an additional 30 minutes and poured into ice water. The crude product is filtered and chromatographed through a silica gel dry column using chloroform as the eluant to give 3-(3-(2-quinolinylmethyloxy)benzyloxy)cinnamylnitrile.

### EXAMPLE 43

When 3-(3-(2-quinolinylmethyloxy)benzyloxy)benzaldehyde of Example 42 is replaced by the compounds of Example 41, the corresponding product is prepared.

When diethylcyanomethylphosphonate in the above Example is replaced by diethylcyanoethylphosphate, diethylcyanopropylphospate or diethylcyanoisopropylphosphate then the corresponding products are obtained.

### EXAMPLE 44

### 5-(3-(3-(2-QUINOLINYLMETHYLOXY)BENZYLOXY)STYRYLTETRAZOLE HYDROCHLORIDE

A mixture of 3-(3-(2-quinolinylmethyloxy)benzyloxy)cinnamylnitrile (0.03 mol), anhydrous aluminum chloride (0.03 mol) and sodium azide (0.09 mol) in THF (30 ml) is stirred and refluxed for 18 hours. Hydrochloric acid (18% HCl 15 ml) is added and thereafter the reaction mixture is poured into ice water. The precipitate is collected and then recrystalized from methanol-ethyl acetate to obtain pure 5-(3-(3-(2quinolinylmethyloxy)benzyloxy)styryl)tetrazole hydrochloride.

The free base is obtained by treatment of the salt with one equivalent of sodium hydroxide solution followed by removal of sodium chloride and water.

### EXAMPLE 45

When 3-(3-(2-quinolinylmethyloxy)benzyloxy)cinnamylnitrile of Example 44 is replaced by the compounds formed in Example 43, then the corresponding product is prepared. Representative compounds prepared by this invention are described in Table XV.

TABLE XV
5-(4-(3-(2-quinolinylmethyloxy)phenoxy)styryl)tetrazole
5-(4-(3-(2-quinolinylmethyloxy)benzyloxy)styryl)tetrazole
5-(3-(4-(2-quinolinylmethyloxy)benzyloxy)styryl)tetrazole
5-(4-(4-(2-quinolinylmethyloxy)benzyloxy)styryl)tetrazole
5-(4-(3-(2-quinolinylmethyloxy)-4-methylbenzyloxy)styryl)tetrazole
5-(4-(3-(2-quinolinylmethyloxy)benzyloxy)3-methylstyryl)tetrazole
5-(3-(3-(2-quinolinylmethylthio)benzyloxy)styryl) tetrazole
5-(3-(4-(2-quinolinylmethylthio)phenoxy)styryl)tetrazole
5-(3-(4-(2-quinolinylmethyloxy)benzylthio)styryl)tetrazole
5-(3-(4-(3-(2-quinolinylmethyloxy)benzyloxy)phenoxy)-2-propen-1-
yl)tetrazole

### EXAMPLE 46

### 3-METHYLCARBOETHOXY-5-(4-(3-(2-QUINOLINYLMETHYLOXY)PHENOXYMETHYL)PHENYL)TETRAZOLE

To a solution of 0.2 g sodium in 30 ml ethanol is first added 1 g of 5-(4-(3-(2-quinolinylmethyloxy)phenoxymethyl)phenyl)tetrazole and then after 30 minutes 0.6 g of ethylbromoacetate and stirring is continued at 80° C. for 16 hours. The solvent is then removed, diluted with water, filtered, washed with ether and dried to give the desired compound, also referred to as ethyl 5-(4-(3-(2-quinolnylmethyloxy)phenoxymethyl)phenyl)tetrazol-3-yl acetate.

When ethylbromoacetate in the above procedure is replaced with N,N-diethyl-α-bromoacetamide, N,N-diethyl-aminoethyl bromide or N-acetylaminoethyl bromide or N-acetyl-α-bromoacetamide, then the corresponding products are obtained.

### EXAMPLE 47

### 5-(4-(3-(2-QUINOLINYLMETHYLOXY)PHENOXYMETHYL)PHENYL)TETRAZOL-3-YL) ACETIC ACID

A mixture of 1 g of ethyl [5-(4-(3-(2-quinolinylmethyl-oxy)phenoxymethyl)phenyl)tetrazol-3-yl]acetate in 5 ml ethanol and 40 ml of 1N NaOH is stirred at 70° C. for 4 hours. This is cooled, diluted with water, acidified with acetic acid, filtered, washed with water, and then ethyl acetate to give 5-(4-(3-(2-quinolinylmethyloxy)phenoxymethyl)phenyl)tetrazol-3-yl acetic acid.

In a similar manner, the substituted tetrazoles of this invention may be prepared.

### EXAMPLE 48

### 4-(4-(2-QUINOLINYLMETHYLSULFONYL)PHENOXYMETHYL)BENZOIC ACID

A. 4-(4-(2-quinolinylmethylthio)phenoxymethyl)benzoic acid (4 mmol) in dichloroethene (50 ml) is stirred with m-chloroperbenzoic acid (4 mmol) and solid potassium hydrogen carbonate (1.0 g). The reaction is assayed by TLC and upon consumption of the starting thio compound, the mixture is filtered, washed with dilute aqueous sodium bisulfite, dried and evaporated to give 4-(4-(2-quinolinylmethylsulfinyl)phenoxymethyl)benzoic acid.

B. To 3 mmol of the sulfinyl compound from Step A in acetic acid (40 mmol) is added 30% hydrogen peroxide (2 ml). The mixture is stirred at ambient temperature and assayed by TLC. Upon disappearance of the sulfinyl starting compound, the reaction mixture is diluted with dichloromethane, washed with dilute aqueous sodium bisulfite and water, dried and evaporated to give 4-(4-(2-quinolinylmethylsulfonyl)phenoxymethyl)benzoic acid.

In a similar manner, the sulfinyl and sulfonyl compounds of this invention may be prepared.

### EXAMPLE 49

### 5-(3-METHYL-4-(4-(4-(2-QUINOLINYLMETHYLOXY)BENZYLOXY)-PHENYL)BUTYL)TETRAZOLE

A. 4-benzyloxy-α-methyl-cinnamic acid ethyl ester.

To a solution of sodium hydride (60% oil dispersion, 3.1 g) and diethyl 2-phosphonopropionate (15.5 g) in tetrahydrofuran (50 ml) is added dropwise a tetrahydrofuran solution of 4-benzyloxy-benzaldehyde (10.6 g). After stirring at room temperature for 2 hours, the reaction mixture is poured into ice water. The insoluble solid is collected, and used directly in the next step.

B. 4-benzyloxy-α-methyl-cinnamic alcohol.

Under argon and with stirring, a tetrahydrofuran solution of 4-benzyloxy-α-methyl-cinnamic acid ethyl ester ( 11.9 g) is added dropwise to a cooled tetrahydrofuran solution of lithium aluminum hydride (2.5 g). The reaction mixture is allowed to stir for 18 hours and afterward, the excess reagent is destroyed in a conventional manner. The residue which results from the evaporation of the solvent is partitioned in a water/ethyl acetate mixture and from the organic layer, the desired product is obtained. This is used directly in the next step.

C. 4-benzyloxy-α-methyl-cinnamyl aldehyde.

Manganese dioxide (15 g total) is added portionwise to a dichloromethane solution (100 ml) of 4-benzyloxymethylcinnamic alcohol with stirring over a period of one week. After two filtrations, the filtrate is evaporated to yield a gum. Upon treatment with cold hexane, the crude product results which is used directly in the next step.

D. 5-(p-benzyloxyphenyl)-4-methyl-2,4-pentadienenitrile.

To a solution of sodium hydride (60 % oil dispersion, 1.5 g) and diethyl cyanomethylphosphonate (5.4 g) in tetrahydrofuran (50 ml) is added dropwise a tetrahydrofuran solution of 4-benzyloxy-α-methyl-cinnamyl aldehyde (4.8 g). After stirring at room temperature for 2 hours, the reaction mixture is poured into ice water. The insoluble material is collected and used directly in the next step.

E. 5-(p-hydroxyphenyl-4-methylvaleronitrile.

5-(p-Benzyloxyphenyl)-4-methyl-2,4-pentadienenitrile (4.3 g) dissolved in ethanol is hydrogenated (0.8 g of 5% palladium over charcoal as catalyst) around 30 psi overnight. After filtering off the catalyst, the solvent is evaporated to give an oil which is used directly in the next step.

F. 4-methyl-5-(4-(4-(2-quinolinyloxymethyl)benzyloxy)phenyl)valeronitrile.

A reaction mixture of 5-hydroxyphenyl-4-methyl-valeronitrile (2.9 g), 4-(2-quinolinylmethyloxy)benzyl chloride hydrochloride (6.3 g) and anhydrous potassium carbonate (30 g) in dimethylformamide (60 ml) is stirred and heated (110° C.) for 5 hours. Afterward, the solvent is removed under vacuum and the residue is partitioned in a mixture of chloroform/water. The organic layer is evaporated and the resultant oil is purified on a silica gel dry column (chloroform as eluant) to give product which may used directly in the next step.

G. 5-(3-methyl4-(4-(4-(2-quinolinylmethyloxy)- benzyloxy)phenyl)butyl)tetrazole.

A mixture of 4-methyl- 5(4-(4-(2-quinolinylmethyloxy)benzyloxy)phenyl)valeronitrile (1.5 g.), sodium azide (3 g), ammonium chloride (1.9 g) in dimethylformamide (20 ml) is stirred and heated at 135° C. for 18 hours. After cooling, the reaction mixture is poured into ice water and the insoluble material is taken up by chloroform. The residue from the evaporation of chloroform is purified by silica gel dry column (5% methanol chloroform as eluant) to yield 5-(3-methyl-4-(4-(4-(2-quinolinylmethyloxy)benzyloxy)-phenyl)butyl)tetrazole.

### EXAMPLE 50

When 2-chloromethylquinoline of Example 49, Part F is replaced by the quinoline compounds of Examples 5 and 6, then the corresponding product is obtained. When the products are treated according to the procedures of Steps F and G. then the corresponding tetrazole products are obtained.

### EXAMPLE 51

When diethyl 2-phosponopropionate of Example 49, Step A is replaced by the Wittig reagents of Table XVI below then the corresponding products are obtained.

TABLE XVI

diethyl 2-phosphonoacetate
diethyl 2-phosphonopropionate
diethyl 3-phosphonopropionate
diethyl 4-phosphonobutyrate
diethyl 3-phosphonobutyrate
diethyl 2-phosphonobutyrate
diethyl 5-phosphonopentanoate
diethyl 4-phosphonopentanoate
diethyl 3-phosphonopentanoate
diethyl 4-phosphono-3-methylbutyrate
diethyl 4-phosphono-2,3-dimethylbutyrate
diethyl 5-phosphono-4-methylpentanoate
diethyl 5-phosphono-3,4-dimethylpentanoate
diethyl 4-phosphono-3,3-dimethylbutyrate
diethyl 4-phosphono-3-phenylbutyrate
diethyl 4-phosphono-3-benzylbutyrate
diethyl 3-phosphono-2,2-dimethylpropionate
diethyl 4-phosphono-2-propylbutyrate
diethyl 4-phosphono-3-propylbutyrate
diethyl 3-phosphonomethylhexanoate
diethyl 4-phosphonoheptanoate

### EXAMPLE 52

When diethylcyanomethylphosphonate of Example 49, Step D is replaced by the Wittig reagents of Table XVII below then the corresponding products are obtained.

TABLE XVII

diethyl 2-phosphonoacetonitrile
diethyl 3-phosphonopropionitrile
diethyl 2-phosphonopropionitrile
diethyl 4-phosphonobutyronitrile
diethyl 3-phosphonobutyronitrile
diethyl 2-phosphonobutyronitrile
diethyl 5-phosphonopentanonitrile
diethyl 4-phosphonopentanonitrile
diethyl 3-phosphonopentanonitrile
diethyl 2-phosphonopentanonitrile
diethyl 4-phosphono-5-phenylpentanonitrile
diethyl 4-phosphono-3-phenylbutyronitrile
diethyl 4-phosphono-5-cyclopropylpentanonitrile
diethyl 4-phosphonohexanonitrile
diethyl 4-phosphonoheptanonitrile
diethyl 4-phosphono-5-carbethoxypentanonitrile
diethyl 4-phosphono-3-methylenebutyronitrile
diethyl 4-phosphono-3-ethylidenebutyronitrile
diethyl 1-phosphonomethyl-1-cyanoethylcyclopropane
diethyl 1-phosphonomethyl-1-cyanomethylcyclobutane
diethyl 1-phosphonomethyl-2-cyanomethylcyclobutane
diethyl 1-phosphonomethyl-2-cyanomethylcyclopentane

### EXAMPLE 53

When diethyl 2-phosphonopropionate of Example 49, Step A is replaced by the Wittig reagents of Table XVII, Example 52, then the corresponding products are obtained. When these products are treated according to the procedure of Example 50, then the corresponding product is obtained.

### EXAMPLE 54

When 4-hydroxy-3-methoxybenzoate of Example 14 is replaced with 3-hydroxymethylphenol, then the product prepared is 3(3-(2-quinolinylmethyloxy)benzyloxy)benzyl alcohol.

### EXAMPLE 55

When 4-hydroxy-3-methoxybenzoate of Example 14 is replaced with the compounds of Table XVIII below and 3-(2-quinolinylmethyloxy)benzyl chloride is replaced by the compounds of Example 6, then the corresponding products are prepared.

TABLE XVIII

1,2-dihydroxybenzene
1,3-dihydroxybenzene
1,4-dihydroxybenzene
2-mercaptophenol
3-mercaptophenol
4-mtercaptophenol
1,3-dimercaptobenzene
3-hydroxymethylphenol
3-hydroxyethylphenol
3-mercaptomethylphenol
4-hydroxymethylphenol
4-hydroxyethylphenol
2-methylresorsinol
5-methylresorsinol
5-methyl-1,4-dihydroxybenzene

### EXAMPLE 56

### 5-(3-CHLOROPROPYL)TETRAZOLE

A mixture of 3.5 g of 4-chlorobutyronitrile, 2.3 g of sodium azide and 1.9 g of ammonium chloride in 50 ml of dimethyl-formamide is stirred at 140° C. for 20 hours. The reaction mixture is poured onto ice, basified with 1N sodium hydroxide and extracted twice with ethyl acetate. The aqueous fraction is acidified with acetic acid and extracted with ethylacetate. Evaporation of the ethyl acetate gives 5-(3-chloropropyl)-tetrazole which is used directly in the next step.

### EXAMPLE 57

When 4-chlorobutyronitrile of Example 56 above is replaced by the nitrides of Table XIX below then the corresponding tetrazole product is obtained.

TABLE XIX

chloroacetonitrile
bromoacetonitrile
3-chloropropionitrile
4-chlorobutyronitrile
5-chloropentanonitrile
6-chlorohexanonitrile
2-chloropropionitrile
2-methyl-3-chloropropionitrile
2-chlorobutyronitrile
3-chlorobutyronitrile
4-methyl-5-chloropentanonitrile
2-methyl-3-chloropropionitrile
3-benzyl-4-chlorobutyronitrile
3-carbethoxymethyl-4-chlorobutyronitrile
3-methoxymethyl-4-chlorobutyronitrile
2,3-dimethyl-4-chloropentanonitrile
3,3-dimethyl-4-chloropentanonitrile
spiro-(3,3-cyclopropane)-4-chlorobutyronitrile
1-chloromethyl-2-cyanomethylcyclobutane
1-chloromethyl-2-cyanomethylcyclohexane
3-cyclopropylmethyl-4-chlorobutyronitrile
3-dimethylaminomethyl-4-chlorobutyronitrile
3-methylene-4-chlorobutyronitrile
3-propylidene-4-chlorobutyronitrile

### EXAMPLE 58

### 5-(4-(3-(3-(2-QUINOLINYLMETHYLOXY)BENZYLOXY)PHENYL)BUTYL)-TETRAZOLE

A mixture of (0.014 mol) 3-(3-(2-quinolinylmethyloxy)benzyloxy)benzyl alcohol (0.14 mol) 5-(3-chloropropyl)tetrazole and 2 g (0.036 mol) KOH in 5 ml water and 50 ml ethanol is heated over a steam bath for a period of 3 hours. Reaction mixture is concentrated to dryness and slurried into water and extracted with methylene chloride. The methylene chloride extract is washed with water, dried over MgSO₄ and concentrated under reduced pressure to obtain solid which is passed through a silica gel column using hexane/ethyl acetate as eluent. Evaporation of eluent gives 5-(4-(3-(3-(2-quinolinylmethyloxy)benzyloxy)phenyl)butyl)tetrazole.

### EXAMPLE 59

When 3-(3-(2-quinolinylmethyloxy)benzyloxy)benzyl alcohol of Example 58 is replaced by the compounds prepared by Examples 54 and 55 and 5-(3-chloropropyl)tetrazole is replaced by the compounds prepared by Example 57, then the corresponding product is obtained.

TABLE XX
5-(4-(4-(3-(2-quinolinylmethyloxy)benzyloxy)phenyl)butyl)tetrazole
5-(3-(4-(3-(2-quinolinylmethyloxy)benzyloxy)phenyl)butyl)tetrazole
5-(3-(4-(4-(2-quinolinylmethyloxy)benzyloxy)phenyl)butyl)tetrazole
5-(2-(3-(3-(2-quinolinylmethyloxy)benzyloxy)phenyl)propyl)tetrazole
5-(3-(3-(3-(2-quinolinylmethylthio)benzyloxy)phenyl)butyl)tetrazole
5-(3-(3-(3-(2-quinolinylmethyloxy)benzyloxy)phenyl)butyl)tetrazole
5-(3-(3-(3-(2-quinolinylmethyloxy)benzylthio)phenyl)butyl)tetrazole
5-(4-(3-(3-(2-quinolinylmethyloxy)benzyloxy)phenyl)butyl)tetrazole
5-(3-(3-(3-(2-quinolinylmethyloxy)phenoxy)phenyl)butyl)tetrazole

### EXAMPLE 60

When 3-hydroxybenzonitrile in Example 7 is replaced by 3-hydroxybenzaldehyde then the product prepared is 3-(2quinolinylmethyloxy)benzaldehyde.

### EXAMPLE 61

When 3-hydroxybenzaldehyde in Example 60 is replaced by the compounds of Table XIV, Example 40 and 3-(2-quinolinylmethyloxy)benzyl chloride is replaced by the chlorides prepared in Examples 5 and 6, then the corresponding product is prepared.

### EXAMPLE 62

### 5-(4-(3-(2-QUINOLINYLMETHYLOXY)BENZOYLMETHYL)PHENYL)TETRAZOLE

A. 2-(3-(2-quinolinylmethyloxy(phenyl)-1,3-dithiane.

A 1M solution of 3-(2-quinolinylmethyloxy)benzaldehyde (0.01 mol) in chloroform is combined with an equimolar amount of 1,3 propane-dithiol at −20° C. Dry HCl gas is slowly passed through the solution for 5-10 minutes. The reaction mixture is then allowed to come to room temperature. After 3 hours, the reaction mixture is worked up by successively washing with water, 10% aqueous KOH and water and drying over K2CO3. Evaporation of the solvent furnishes the desired product which is purified by column chromatography to give product which is used directly in the next step.

B. 2-(3-(2-quinolinylmethyloxy)phenyl-2-(p-cyanobenzyl)- 1,3-dithiane.

To a 0.2M THF solution of the 2-(3-(2quinolinyl-methyloxy)phenyl)-1,3-dithiane (0.01 mol) under is added a 5% excess of N-butyl lithium in N-hexane (2.5M) at a rate if 3-5 ml/min at −78° C. After 3 hours, 4-cyanobenzylchloride (0.01 mol in 20 ml of THF) is added dropwise over a period of 10 minutes. Let stir 3 hours at −78° C. and then allow the reaction mixture to come to 0° C. slowly. The mixture is poured into 3 volumes of water, extracted with chloroform furnishing an organic solution which is washed twice with water, 7% aqueous KOH and again with water. The organic layer is dried over K2CO3 and is concentrated. The crude product is purified by column chromatography to give the desired product which is used directly in the next step.

C. 4-(3-(2-quinolinylmethyloxy)benzoylmethyl)benzonitrile.

To a solution of 2-(3-(2-quinolinylmethyloxy)-1,3- dithiane (1.0 mmol) in 80% aqueous acetonitrile (10 ml) is added mercuric chloride (2.2 mmol) as a solution in the same solvent mixture. Mercuric oxide (1.1 mmol) is then added to buffer the reaction mixture near pH=7. The dithiane-mercuric chloride complex separates as a white precipitate. The reaction mixture is refluxed under nitrogen for 5 hours, then cooled and filtered through Super Gel. The filter cake is washed thoroughly with 1:1 hexane-dichloromethane. The organic phase is washed with 5 M aqueous ammonium acetate, water and brine. The organic phase is then dried with MgSO₄, and is concentrated to give the crude product which is purified by column chromatography to give 4-(3-(2-quinolinylmethyloxy)benzoylmethyl)benzonitrile.

D. 5-(4-(3-(2-quinolinylmethyloxy)benzoylmethyl)- phenyl)tetrazole.

A heterogenous mixture of 4-(3-(2-quinolinylmethyloxy)benzoylmethyl)benzonitrile (1.35 mmol). NaN₃ (6.77 mmol), pyridinium hydrochloride (6.77 mmol) in DMF (3 ml) is heated at 100° C. for 3 hours under nitrogen. The reaction mixture is poured into water and the product is collected on a filter. Recrystallization from EtOAc-DMF gives 5-(4-(3-(2-quinolinylmethyloxy)benzoylmethyl)phenyl)tetrazole.

### EXAMPLE 63

When 3-(2-quinolinylmethyloxy)benzaldehyde in Example 62, Step A is replaced by the aldehydes of Example 61, and 4-cyanobenzyl chloride of Example 62, Step B is replaced by the compounds of Table X, Example 29 or Table VII, Example 23, then the corresponding products are obtained. Representative compounds prepared by this invention are shown in Table XXI.

TABLE XXI
5-(4-(4-(2-quinolinylmethyloxy)benzoylmethyl)phenyl)tetrazole
5-(4-(3-(2-quinolinylmethyloxy)benzoylmethyl)benzyl)tetrazole
5-(3-(4-(3-(2-quinolinylmethyloxy)benzoylmethyl)phenyl)propyl)tetrazole
5-(3-(3-(2-quinolinylmethylthio)benzoylmethyl)phenyl)tetrazole
5-(4-(3-(2-quinolinylmethyloxy)benzoylethyl)benzyl)tetrazole

### EXAMPLE 64

### 5-(3-(3-(2-QUINOLINYLMETHYLOXY)BENZOYLAMINO)PHENYL)TETRAZOLE

A. 3-(2-quinolinylmethyloxy)benzoic acid.

A mixture of 28.16 g (0.132 mol) of 2-quinolinylmethyl chloride HCl, 18 g (0.132 mol) of 3-hydroxybenzoic acid and 39.6 g of potassium carbonate in 110 ml of DMF is heated at 70° C. overnight. The reaction mixture is poured into water, and the precipitated product is collected, filtered and dried to give 3-(2quinolinylmethyloxy)benzoic acid.

B. 3-(2-quinolinylmethyloxy)benzoic acid chloride.

A mixture of 15.6 g (0.1 mol) of 3-(2-quinolinylmethyloxy)benzoic acid and 11.9 g (0.1 mol) of thionyl chloride is refluxed for 4 hours. The reaction mixture is then evaporated to dryness at room temperature and used directly in the next step.

C. 3-(3 -(2-quinolinylmethyloxy)benzoylamino)benzonitrile.

A solution of 3-aminobenzonitrile (10 mmol) in 50 ml of chloroform and triethylamine (11 mmol) is added to a solution of 10 mmol of 3-(2-quinolinylmethyloxy)benzoic acid chloride in 20 ml of chloroform over a period of 10 minutes. The reaction is stirred at room temperature for 2 hours and is poured into water and then extracted into chloroform. The organic solution is dried and evaporated to give 3-(3-(2-quinolinylmethyloxy)benzoylamino)benzonitrile.

D. 5-(3-(3-(2-quinolinylmethyloxy)benzoylamino)phenyl)tetrazole.

A mixture of 10 mmol of 3-(3-(2-quinolinylmethyloxy)benzoylamino)benzonitrile, 50 mmol of sodium azide, and 50 mmol of pyridine HCl in 30 ml of DMF is heated at 100° C. for 2 days. The reaction mixture is poured into water, and the product is collected on a filter. Recrystallization from ethyl acetate and DMF gives 5-(3-(3-(2-quinolinylmethyloxy)benzoylamino)phenyl)tetrazole.

In a similar manner, the compounds of this invention where B is

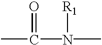

may be made.

### EXAMPLE 65

### 5-(3-(3-(2-QUINOLINYLMETHYLOXY)-ANILINOCARBONYL)PHENYL)TETRAZOLE

When the procedure of Example 64 is followed and 3-(2-quinolinylmethyloxy)aniline is used in place of 3-aminobenzonitrile and 3-cyanobenzoic acid is used in place of 3-(2-quinolinylmethyloxy) benzoic acid, then the product prepared is 5-(3-(3-(2-quinolinylmethyloxy)aminocarbonyl)phenyl)tetrazole.

In a similar manner, the compounds of this invention where B is

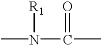

may be made.

### Synthesis of a Compound of Formula (VI)

A compound of Formula (VI) is prepared in a multi-step synthesis illustrated in the below scheme. The key starting material is quinaldine. In the first stage it is chlorinated to form 2-chloromethylquinoline which, without isolation, is reacted with hydroquinone to form the intermediate 4-(quinolin-2-yl-methoxy)phenol (VIII). This intermediate is then treated with α,α′-dichloro-o-xylene to form 2-[4-quinolin-2-yl-methoxy)phenoxymethyl]benzyl chloride, which is converted in situ to 2-[4-quinolin-2-yl-methoxy)phenoxymethyl]phenylacetonitrile (IX), the penultimate precursor to (VI).

(IX) is converted to (VI) crude, in a reaction with sodium azide and ammonium chloride which transforms the nitrile group into the tetrazole ring. The purification of the final product is accomplished by recrystallization of the crude material from methanol to afford pure (VI).

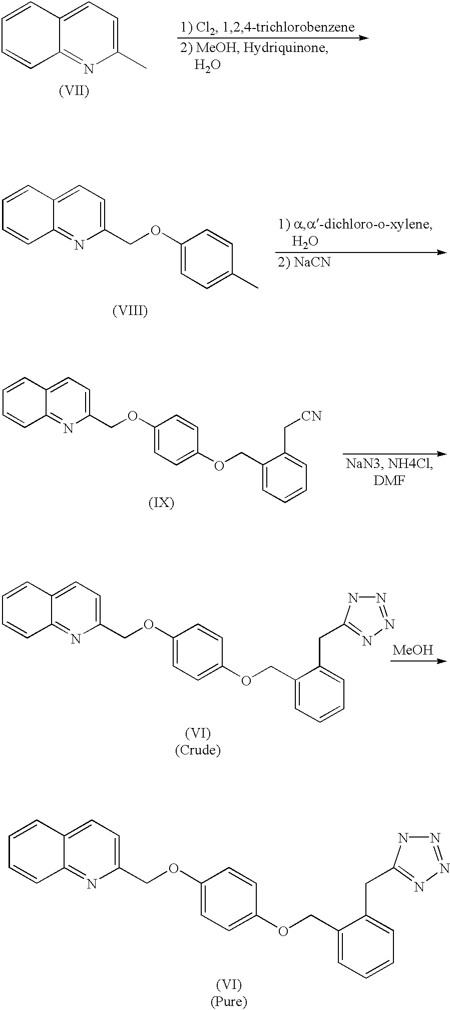

Solid Phase Synthesis of a Compound of Formula:

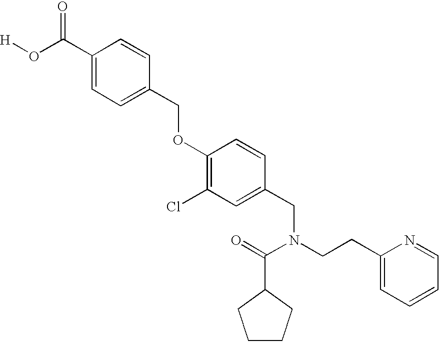

1. Acid Loading

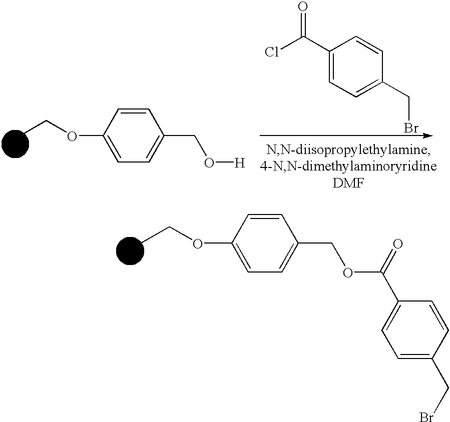

A 1L round bottom flask is charged with 4-(bromomethyl)benzoic acid (32.26 g, 150.0 mmole) and dichloromethane (650 mL). A stir bar is carefully added and the reaction flask is immersed in an ice-water bath. After approximately 15 minutes, oxallyl chloride (15.7 mL, 180 moles) is added. After approximately 15 minutes, N,N-dimethylformaide (500 mL, cat.) is added. The reaction began to bubble. After stirring for 1.5 hours, the ice-water bath is removed. After stirring for 3 hours at ambient temperature, the effervescence has ceased. At the end of this period, the stirbar is removed from the reaction mixture and the reaction solvent is removed in vacuo. After the solvent has been removed, more dichloromethane is added to the reaction flask and this too is removed in vacuo.

A three neck 3L round bottom flask is charged with dry N,N-dimethylformamide (1.3 L), N,N-diisopropylethylamine (39.19 mL, 225 mmoles), 4-N,N-dimethylaminopyridine (3.67 g, 30 mmole) and MicroKANS [1456, 15 mg of Wang resin (1.7 mmole/g loading) per MicroKANs, 25.5 micromoles/microKAN, 37.1 mmoles]. The flask is fitted with an overhead stirring apparatus. After stirring for approximately 15 minutes, a solution of the acid chloride as prepared above in dry N,N-dimethylformamide (200 mL) is transferred into the reaction flask. After 14 hours, the reaction solvent is removed. DMF (1.5 L) is added to the reaction flask. The flask was allowed to stir for approximately 15 minutes and the solvent is drained. The MicroKANs are washed, stirred for 20 minutes and drained in the following sequence repeatedly: DMF (2×6 L), THF (3×6 L), dichloromethane (3×6 L) and ether (2×6 L). After the final washing the MicroKANs are dried by blowing a stream of nitrogen through the flask with intermittent agitation. After sufficient drying, the MicroKANs are sorted for the next reaction.

2. Phenol Displacement

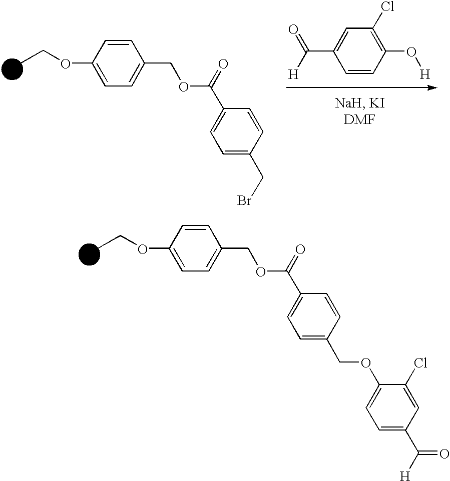

A three neck 3 L round bottom flask is charged with 3-chloro-4-hydroxybenzaldehyde (21.9 g, 140 mmoles) and DMF (1.5 L). The reaction flask is fitted with an overhead stirrer and immersed in an ice-water bath. After approximately 15 minutes sodium hydride (60% dispersion in oil, 6.48 g, 180 mmoles) is carefully added. After approximately 30 minutes, the ice-water bath is removed and the reaction allowed to stir at ambient temperature for 1 hour. At the end of this time, the MicroKANs [1274, 25.5 micromoles/microKAN, 32.5 mmoles] and potassium iodide (1.0 g) are added to the reaction mixture. The reaction flask is immersed into an oil bath which is heated to 60° C. After 14 hours, the reaction flask is removed from the oil bath and allowed to cool to ambient temperature. The reaction solvent is removed. DMF (1.2 L) is added to the reaction flask. The flask is allowed to stir for approximately 15 minutes and the solvent is drained. DMF:water (1:1, 1.2 L) is added to the reaction flask. The flask is allowed to stir for approximately 15 minutes and the solvent is drained. This sequence is repeated at least three times or until the effluent from the washing is clear, the reaction flasks are washed repeatedly in the following sequence: THF (2×4 L), dichloromethane (1×4 L) then methanol (1×4 L), dichloromethane (1×4 L) then methanol (1×4 L), dichloromethane (1×4 L) then methanol (1×4 L), dichloromethane (1×4 L) and ether ( 1×4 L). After the final washing the MicroKANs are dried by blowing a stream of nitrogen through the flask with intermittent agitation. After sufficient drying, the MicroKANs are sorted for the next reaction.

3. Reductive Amination

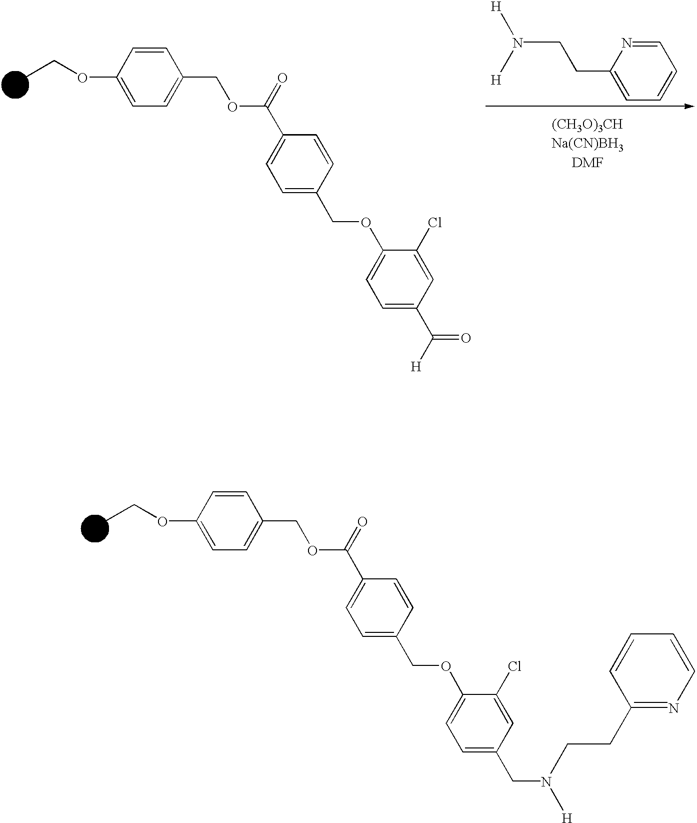

A three neck 2 L round bottom flask is charged with the MicroKANs [784, 25.5 micromoles/microKAN, 20.0 mmoles], trimethylorthoformate (850 mL) and 2-(2-aminoethyl)pyridine 20.79 g, 170 mmoles). The reaction flask is fitted with an overhead stirrer. After 2 hours, sodium cyanoborohydride (21.37 g, 340 mmoles) is added. After approximately 10 minutes, acetic acid (17.0 mL, 297 mmoles) is added. After stirring for an additional hour, the reaction flask is drained. Methanol (800 mL) is added to the flask. After stirring for approximately 10 minutes, the flask is drained. the reaction flask is washed repeatedly in the following sequence: DMF (3×4 L), dichloromethane (1×4 L) then methanol (1×4 L), dichloromethane (1×4 L) then methanol (1×4 L), dichloromethane (1×4 L) then methanol (1×4 L), dichloromethane (1×4 L) and ether ( 1×4 L). After the final washing the microKANS are dried by blowing a stream of nitrogen through the flask with intermittent agitation. After sufficient drying, the MicroKANs are sorted for the next reaction.

4. Acylation

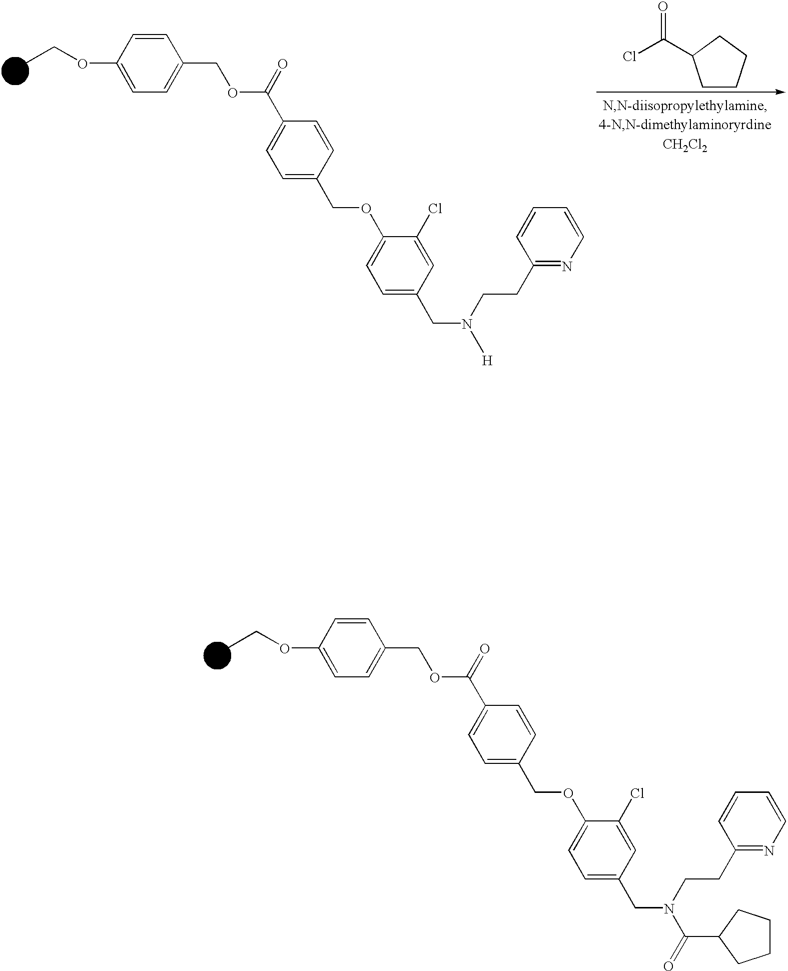

A three neck 2 L round bottom flask is charged with the MicroKANs [784, 15 mg of resin (1.7 mmole/g loading) per MicroKAN, 25.5 micromoles/microKAN, 20.0 mmoles], and dichloromethane (800 mL). The reaction flask is fitted with an overhead stirrer. N,N-diisopropylethylamine (20.9 mL, 120 mmoles) and 4-N,N-dimethylaminopyridine (195 mg, 1.6 mmoles) are added. After approximately 15 minutes, the cyclopentanecarbonyl chloride (10.6 g, 80.0 mmoles) is added. The reaction was allowed to stir for 61 hours, the reaction flask is drained. Dichloromethane (800 mL) is added to the reaction flask. After stirring for approximately 10 minutes, the flask is drained. This is repeated. The MicroKANs from all of the acylation reactions are randomly combined into two separate large flasks and washed repeatedly in the following sequence: dichloromethane (1×4 L), THF (2×4 L), dichloromethane (1×4 L) then methanol (1×4 L), dichloromethane (1×4 L) then methanol (1×4 L), dichloromethane (1×4 L) then methanol (1×4 L), dichloromethane (1×4 L) and ether (1×4 L).

5. Cleavage

The MicroKAN is sorted into individual wells of IRORI AccuCleave 96 cleavage station. The well is charged with dichloromethane (600 mL) and then with a TFA: dichloromethane mixture (1:1, 600 mL). After agitating for approximately forty minutes, the reaction well is drained into 2 mL microtube in an 96-well format. The reaction well is again charged with dichloromethane (600 mL). After manual agitation, this too is drained into the 2 mL microtube in an 96-well format. The cleavage cocktail is removed in vacuo using a Savant Speedvac. The concentrated products from the cleavage mother plates are reconstituted with THF and transferred into two daughter plates utilizing a Packard MultiProbe liquid handler. The daughter plates are concentrated in vacuo utilizing a Genie Vac. Analytical: MS: m/z 493 (M⁺).

The methods described above are used to prepare the following compounds of this invention.

5-[2-(4-(2-quinolinylmethoxy)phenoxymethyl)benzyl]tetrazole (M.P. 108-111° C.)

| | |
|---|---|
| CALC: | C, 59.87; H, 5.96; N, 13.96 |
| FOUND: | C, 59.67, 60.01; H, 5.62, 5.63; N,. 13.73, 13.77 |

5-[4-Methoxy-3-(3-(2-quinolinylmethoxy)phenoxymethyl)phenyl]tetrazole (M.P. 184-87° C.)

| | |
|---|---|
| CALC: | C, 67.63; H, 4.88; N, 15.78 |
| FOUND: | C, 67.18; H, 5.13; N, 15.40 |

5-[3-(4-(2-quinolinylmethyloxy)phenoxymethyl)phenyl]tetrazole (M.P. 176-177° C.)

| | |
|---|---|
| CALC: | C, 69.63; H, 4.75; N, 16.92 |
| FOUND: | C, 69.58, 69.64; H, 5.00, 4.98; N, 16.66, 16.63 |

5-[3-Methoxy-4-(4-(2-quinolinylmethyloxy)benzyloxy)phenyl]tetrazole (M.P. 195-97° C.)

| | |
|---|---|
| CALC: | C, 67.63; H, 4.88; N, 15.77 |
| FOUND: | C, 67.27; H, 4.89; N, 15.41 |

5-[4-(3-(2-quinolinylmethyloxy)phenoxymethyl)-3methoxyphenyl]tetrazole (M.P. 189-91° C.)

| | |
|---|---|
| CALC: | C, 66.95; H, 4.95; N, 15.61 |
| FOUND: | C, 66.48; H, 5.14; N, 14.93 |

5-[3-(4-(2-quinolinylmethyloxy)phenoxymethyl)benzyl]tetrazole (M.P. 139-44° C.)

| | |
|---|---|
| CALC: | C, 70.53; H, 5.03; N, 16.45 |
| FOUND: | C, 70.33, 70.54; H, 5.25, 5.36; N, 16.38, 16.41 |

5-[4-(4-(2-quinolinylmethyloxy)phenoxymethyl)benzyl]tetrazole (M.P. 167-71° C.)

| | |
|---|---|
| CALC: | C, 67.33; H, 5.31; N, 15.70 |
| FOUND: | C, 67.54, 67.67,; H, 5.33, 5.33; N, 15.48, 15.52 |

5-[4-Methoxy-3-(4-(2-quinolinylmethyloxy)phenylmethyloxy)phenyl]tetrazole (M.P. 210-13° C.)

| | |
|---|---|
| CALC: | C, 68.33; H, 4.82; N, 4.90 |
| FOUND: | C, 68.32; H, 4.90; N, 14.79 |

4-[3-(2-Quinolinylmethyloxy)phenoxymethyl]phenoxyacetic acid (M.P. 164 (dec))

| | |
|---|---|
| CALC: | C, 69.27; H, 5.35; N, 3.23 |
| FOUND: | C, 69.53, 69.65; H, 5.11, 5.05; N, 3.21, 3.12 |

5-[2-(4-(2-Quinolinylmethyloxy)phenoxymethyl)phenoxymethyl]tetrazole (M.P. 183-85° C.)

| | |
|---|---|
| CALC: | C, 65.63; H, 5.08; N, 15.31 |
| FOUND: | C, 65.77, 65.52; H, 4.99, 5.03; N, 14.92, 15.03 |

4-[4-(2-Quinolinylmethyloxy)phenoxymethyl]phenoxyacetic acid (176° C. (dec))

| | |
|---|---|
| CALC: | C, 71.50; H, 5.16; N, 3.34 |
| FOUND: | C, 71.10, 71.17; H, 5.27, 5.33; N, 3.37, 3.34 |

4-[3-(2-Quinolinylmethyloxy)phenoxymethyl]phenylacetic acid (M.P. 158-60° C.)

| | |
|---|---|
| CALC: | C, 75.17; H, 5.30; N, 3.51 |
| FOUND: | C, 74.89; H, 5.36; N, 3.37 |

2-[3-(3-(2-Quinolinylmethyloxy)phenoxymethyl)phenoxy]pentanoic acid (M.P. 133-35° C.)

| | |
|---|---|
| CALC: | C, 73.51; H, 5.95; N, 3.06 |
| FOUND: | C, 73.35, 73.60; H, 5.95, 5.98; N, 3.08, 3.05 |

2-[3-(2-Quinolinylmethyloxy)phenoxymethyl]phenoxyacetic acid (M.P. 169-172° C.)

| | |
|---|---|
| CALC: | C, 72.28; H, 5.10; N, 3.37 |
| FOUND: | C, 69.34, 69.69; H, 5.10, 5.13; N, 3.00, 3.08 |
| CALC: | C, 69.27; H. 5.35; N. 3.23 (as Hydrate) |

2-[4-(2-Quinolinylmethyloxy)phenoxymethyl]cinnamic acid (M.P. 175-178° C.)

| | |
|---|---|
| CALC: | C, 75.90; H. 5.14; N. 3.40 |
| FOUND: | C, 73.92; H. 5.20; N. 3.01 |
| CALC: | C, 74.27; H. 5.27; N, 3.33 (as Hydrate) |

6-Acetyl-2-propyl-3-[3-(2-quinolinylmethyloxy)-benzyloxy]phenoxyacetic acid (M.P. 153-58° C.)

| | |
|---|---|
| CALC: | C, 72.13; H, 5.85; N, 2.90 |
| FOUND: | C, 71.68, 72.08; H, 5.88, 5.83; N, 2.65, 2.70 |

2-[2-(4-(7-Chloroquinolin-2-ylmethyloxy)-phenoxymethyl)phenoxy]propionic acid (M.P. 169-173° C.)

| | |
|---|---|
| CALC: | C, 67.32; H, 4.78; N, 3.02; CI, 7.64 |
| FOUND: | C, 65.18; H, 4.90; N, 2.84; CI, 8.33 |
| CALC: | C, 65.41; H, 4.96; N, 2.93; CI, 7.42 (as HYDRATE) |

2-[4-(2-Quinolinylmethyloxy)phenoxymethyl]phenylacetic acid (M.P. 181-83 ° C.)

| | |
|---|---|
| CALC: | C, 75.17; H, 5.30; N, 3.51 |
| FOUND: | C, 75.12, 74.96; H, 5.50, 5.49; N, 3.16, 3.16 |

3-[3-(2-Quinolinylmethyloxy)phcnoxymethyl]phenoxyacetic acid (M.P. 146-51° C.)

| | |
|---|---|
| CALC: | C, 72.28; H. 5.10; N. 3.37 |
| FOUND: | C, 71.82, 71.80; H. 5.24, 5.23; N, 2.98, 3.00 |
| CALC: | C, 71.50; H, 5.16; N, 3.34 (as HYDRATE) |

2-[4-(2-Quinolinylmethyloxy)phenoxymethyl]phenoxyacetic acid (M.P. 153-57° C.)

| | |
|---|---|
| CALC: | C, 72.28; H, 5.10; N, 3.37 |
| FOUND: | C, 72.30, 71.72; H, 5.39, 5.30; N, 2.94, 2.89 |

5-[2-(4-(7-Chloroquinolin-2-ylmethyloxy)-phenoxymethyl)benzyl]tetrazole (M.P. 159-63° C.)

| | |
|---|---|
| CALC: | C, 65.57; H, 4.40; N, 15.29 |
| FOUND: | C, 64.16; H, 4.72; N, 14.98 |
| CALC: | C, 64.30; H, 4.53; N, 14.99 (as HYDRATE) |

2-Carbomethoxy-5-[3-(2-quinolinylmethyloxy)-phenoxymethyl]phenoxyacetic acid (M.P. 187-89° C.)

| | |
|---|---|
| CALC: | C, 68.49; H, 4.90; N, 2.95 |
| FOUND: | C, 66.71; H, 4.96; N, 2.70 |
| CALC: | C, 66.59; H, 5.07; N, 2.87(as HYDRATE) |

2-[3-(2-Quinolinylmethyloxy)phenoxymethyl]-6-methylphdnoxyacetic acid (M.P. 149-53° C.)

| | |
|---|---|
| CALC; | C, 72.71; H, 5.40; N, 3.26 |
| FOUND: | C, 71.23; H, 5.46; N, 3.08 |
| CALC: | C, 71.22; H, 5.51; N, 3.19 (as HYDRATE) |

2-[3-(3-(2-Quinolinylmethyloxy)phenoxymethyl)phenoxy]glutaric acid (M.P. 129-30° C.)

| | |
|---|---|
| CALC: | C, 69.00; H, 5.17; N, 2.87 |
| FOUND: | C, 58.19; H, 4.93; N, 2.23 |
| CALC: | C, 58.23; H, 5.17; N, 2.43 (as HYDRATE) |

2-[3-(2-Quinolinylmethyloxy)phenoxymethyl]benzylmalonic acid (M.P. 164-65° C.)

| | |
|---|---|
| CALC: | C, 70.89; H, 4.08; N, 3.06 |
| FOUND: | C, 70.51, 70.61; H, 5.03, 5.24; N, 3.03, 2.90 |

2-[2-(3-(2-Quinolinylmethyloxy)phenoxymethyl)phenoxy]pentanoic acid (M.P. 118-20° C.)

| | |
|---|---|
| CALC: | C, 73.51; H, 5.95; N, 3.06 |
| FOUND: | C, 73.26; H, 6.07; N, 2.79 |

2-[4-(2-Quinolinylmethyloxy)phenoxymethyl]-6-methylphenoxy acetic acid (M.P.- 151-53° C.)

| | |
|---|---|
| CALC: | C, 72.71; H, 5.40; N, 3.26 |
| FOUND: | C, 71.41; H, 5.58; N, 3.03 |
| CALC: | C, 71.22; H, 5.51; N, 3.19 (as HYDRATE) |

2-[2-(4-(2-Quinolinylmethyloxy)phenoxymethyl)phenoxy]pentanoic acid (M.P. 85-92° C.)

| | |
|---|---|
| CALC: | C, 73.51; H, 5.95; N, 3.06 |
| FOUND: | C, 71.73, 71.79; H, 5.96, 5.91; N, 3.06, 2.83 |
| CALC: | C, 72.09; H, 6.05; N, 3.00 (as HYDRATE) |

2-Carbomethoxy-5-[4-(2-quinolinylmcthyloxy)-phenoxymethyl]phenoxyacetic acid (M.P. 149-51° C.)

| | |
|---|---|
| CALC: | C, 68.49; H, 4.90; N, 2.95 |
| FOUND: | C, 68.00, 68.08; H, 4.98, 5.04; N, 2.90, 2.90 |

2-[2-(4-(2-Quinolinylmethyloxy)phenoxymethylphenoxy]propionic acid (M.P. 161-64° C.)

| | |
|---|---|
| CALC: | C, 72.71; H, 5.40; N, 3.26 |
| FOUND: | C, 70.96, 71.10; H, 5.51, 5.58; N, 3.08, 3.10 |
| CALC: | C, 71.22; H, 5.52; N, 3.19 (as HYDRATE) |

2-[2-(3-(2-Quinolinylmethyloxy)phenoxymethyl)phenoxy]glutaric acid (M.P. 83° C. dec)

| | |
|---|---|
| CALC: | C, 68.98; H, 5.17; N, 2.87 |
| FOUND: | C, 64.10, 63.75; H, 4.89, 4.92; N, 2.64, 2.69 |
| CALC: | C, 63.74; H, 5.63; N, 2.65(as HYDRATE) |

2-(3-[2-Quinolinylmethyloxy]benzyloxy)phenoxyacetic acid (M.P. 153-55° C.)

| | |
|---|---|
| CALC: | C, 72.28; H. 5.10; N. 3.37 |
| FOUND: | C, 71.75; H. 5.14; N. 3.38 |
| CALC: | C, 71.50; H. 5.16; N. 3.34 (as HYDRATE) |

2-(2-[4-(2-Quinolinylmethyloxy)phenoxymethyl]-4chlorophenoxy)propionic acid (M.P. 196-99° C.)

| | |
|---|---|
| CALC: | C, 67.32; H, 4.78; N, 3.02 |
| FOUND: | C, 67.40, 67.43; H, 4.89, 4.94; N, 3.01, 3.13 |

2-(2-[3-(2-Quinolinylmethyloxy)phenoxymethyl]-4chlorophenoxy)propionic acid (M.P. 169-71° C.)

| | |
|---|---|
| CALC: | C, 67.32; H, 4.78; N, 3.02 |
| FOUND: | C, 65.47; H, 5.31; N, 2.78 |
| CALC: | C, 65.41; H, 4.96; N, 2.93 (as HYDRATE) |

2-(2-[3-(2-Quinolinylmethyloxyphenoxymethyl]-4chlorophenoxy)pentanoic acid (M.P. 144-45° C.)

| | |
|---|---|
| CALC: | C, 68.36; H, 5,33; N, 2.85 |
| FOUND: | C, 67.74, 67.86; H, 5.39, 5.47; N, 2.91, 2.84 |
| CALC: | C, 67.74; H, 5.38; N, 2.82 (as HYDRATE) |

2-(2-[4-(2-Quinolinylmethyloxy)phenoxymethyl]-4-chlorophenoxy)pentanoic acid (M.P. 155-56° C.)

| | |
|---|---|
| CALC: | C, 68.36; H, 5.33; N, 2.85 |
| FOUND: | C, 65.96; H, 5.59; N, 2.66 |
| CALC: | C, 65.95; H, 5.53; N, 2.75 (as HYDRATE) |

2-(2-[4-(2-Quinolinylmethyloxy)phenoxymethyl]-4-chlorophenoxy)pentanoic acid (M.P. 155-56° C.)

| | |
|---|---|
| CALC: | C, 68.36; H, 5.33; N, 2.85 |
| FOUND: | C, 66.15; H, 5.58; N, 2.68 |
| CALC: | C, 65.95; H, 5.53; N, 2.75 (as HYDRATE) |

2-(2-[4-(2-Quinolinylmethyloxy)phenoxymethyl]-6-chlorophenoxy)pentanoic acid (M.P. 161-62° C.)

| | |
|---|---|
| CALC: | C, 68.36; H, 5.33; N, 2.85 |
| FOUND: | C, 68.15; H, 5.36; N, 2.72 |

2-(2-[3-(2-Quinolinylmethyloxy)phenoxymethyl]-6-chlorophenoxy)pentanoic acid (M.P. 169-70° C.)

| | |
|---|---|
| CALC: | C, 68.36; H, 5.33; N, 2.85 |
| FOUND: | C, 68.10; H, 5.39; N, 2.72 |

2-(2-[3-(2-Quinolinylmethyloxy)phenoxymethyl]-6-chlorophenoxy)-4-methylpentanoic acid (M.P. 164-66° C.)

| | |
|---|---|
| CALC: | C, 68.84; H, 5.58; N, 2.77 |
| FOUND: | C, 68.84; H, 5.70; N, 2.69 |

2-(2-[4-(2-Quinolinylmethyloxy)phenoxymethyl]-6-chlorophenoxy)-4-methylpentanoic acid (M.P. 167-69° C.)

| | |
|---|---|
| CALC: | C, 68.84; H, 5.58; N, 2.77 |
| FOUND: | C, 68.78; H, 5.67; N, 2.68 |

5-[3-(3-(2-quinolinylmethyloxy)benzyloxy)-4-methoxyphenyl]tetrazole (M.P. 204-07° C.)

| | |
|---|---|
| CALC: | C, 67.63; H, 4.88; N, 15.78 |
| FOUND: | C, 67.11; H, 5.15; N, 15.86 |

N-[3-Methoxy-4-(3-(2-quinolinylmethyloxy)benzyloxy)benzoyl)benzene sulfonamide hydrochloride (M.P. dec.88)

| | |
|---|---|
| CALC: | C, 62.99; H, 4.60; N, 4.74 |
| FOUND: | C, 63.88; H, 5.13; N, 4.80 |

5-Carboxy-2-(3-(2-quinolinylmethyloxy)phenoxymethyl)phenoxy acetic acid (M.P. 226-28° C.)

| | |
|---|---|
| CALC: | C, 61.90; H, 5.18; N, 2.77 |
| FOUND: | C, 61.62; H, 5.11; N, 2.67 |

5-[3-Methoxy-4-(3-(2-quinolinylmethyloxy)benzyloxy)phenyl]tetrazole (M.P. 204-05° C.)

| | |
|---|---|
| CALC: | C, 67.67; H, 5.14; N, 15.87 |
| FOUND: | C, 67.63; H, 4.88; N, 15.78 |

5-(4-(3-(2-Quinolinylmethyloxy)benzyloxy)phenyl)tetrazole (M.P. 233-36° C.)

| | |
|---|---|
| CALC: | C, 69.58; H, 4.73; N, 16.91 |
| FOUND: | C, 69.59; H, 4.89; N, 16.91 |

| | |
|---|---|
| 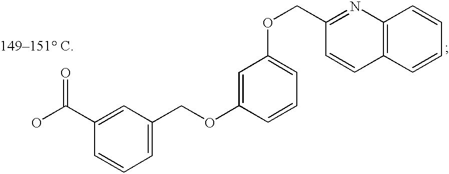 | 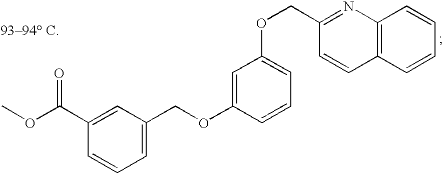 |

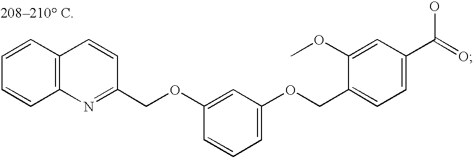

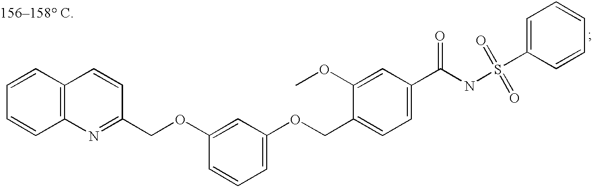

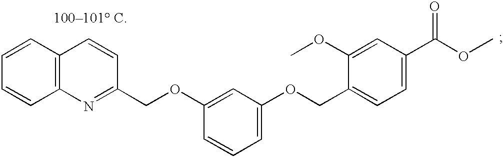

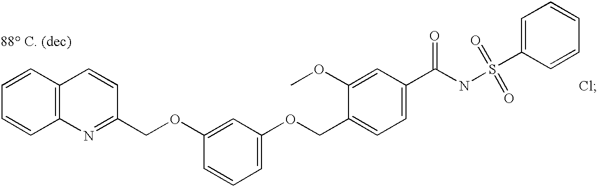

| | |
|---|---|
| 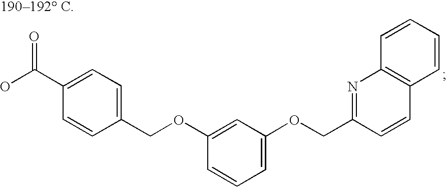 | 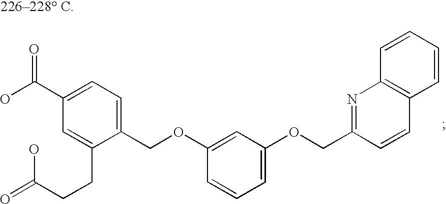 |
| 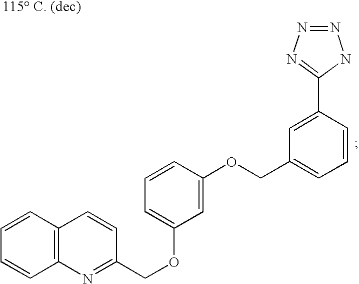 | 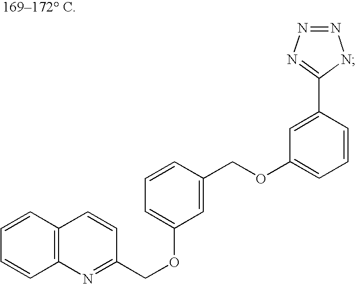 |

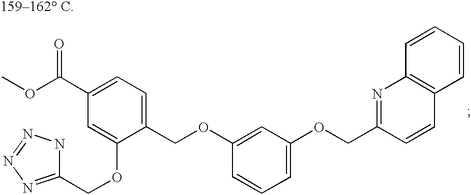

| | |
|---|---|
| 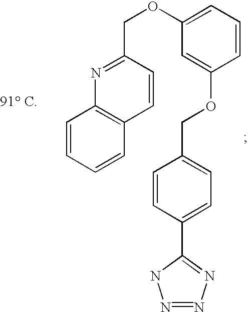 | 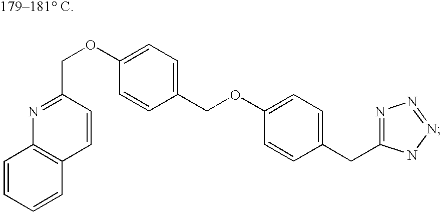 |
| 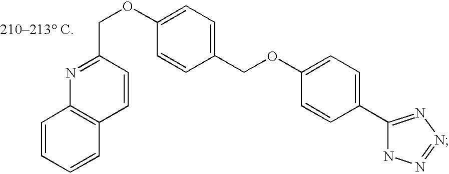 | 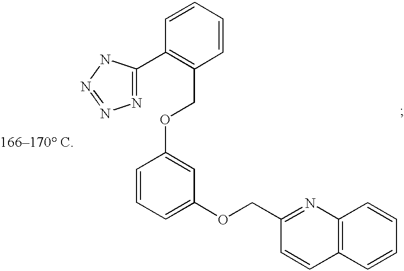 |
| 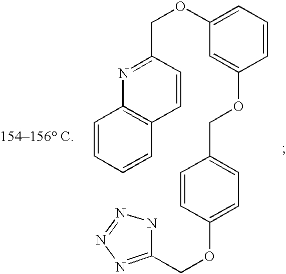 | 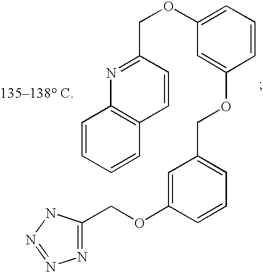 |
| 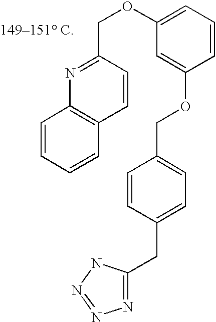 | 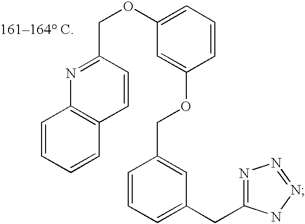 |

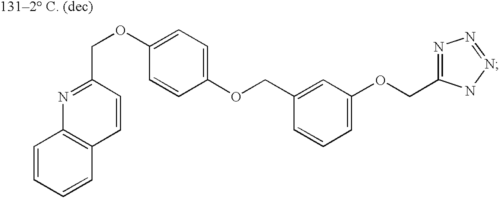

| | |
|---|---|
| 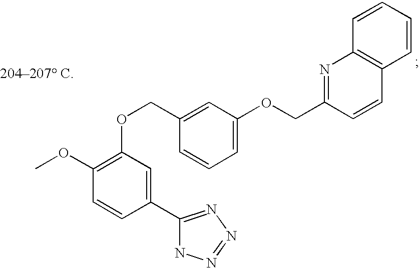 | 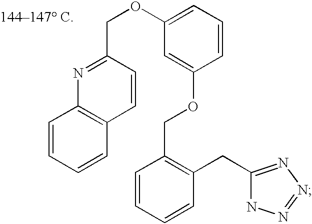 |
| 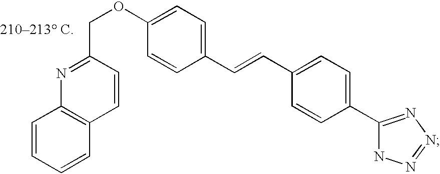 | 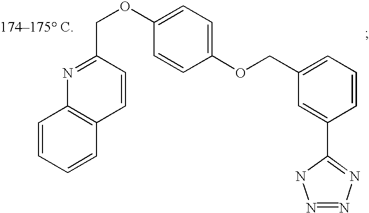 |
| 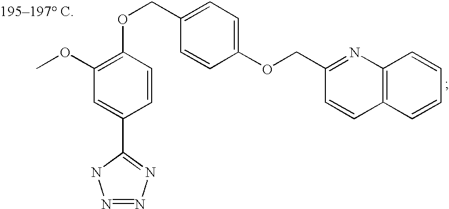 | 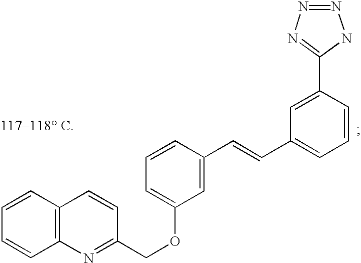 |
| 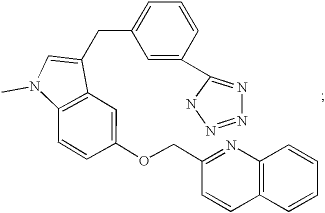 | 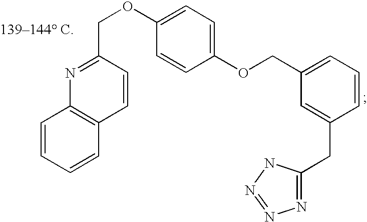 |
| 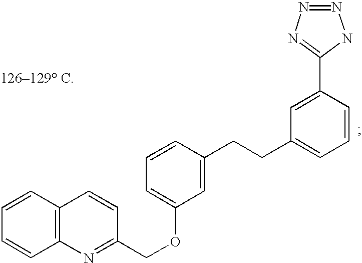 | 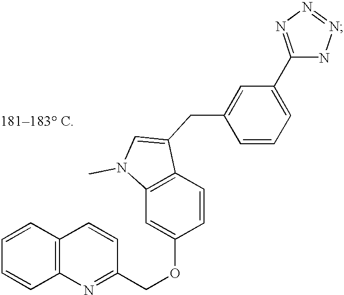 |
| 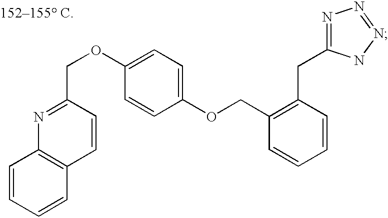 | 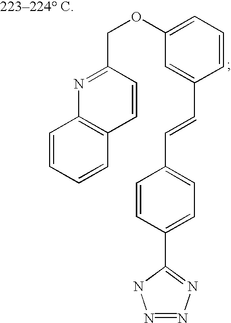 |

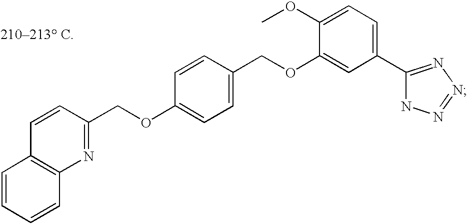

| | |
|---|---|
| 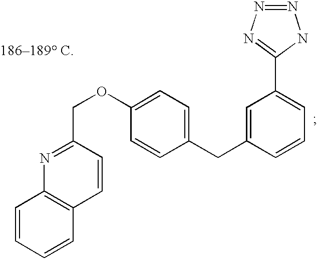 | 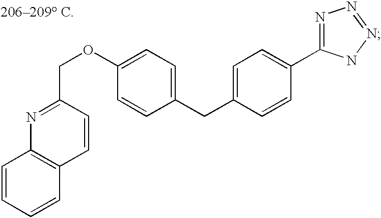 |

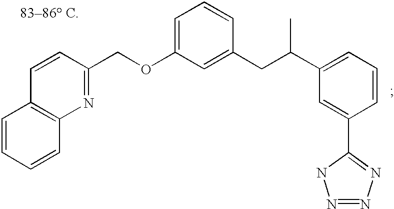

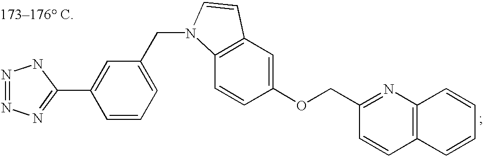

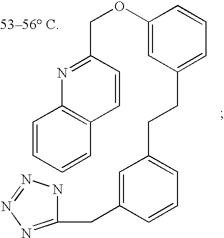

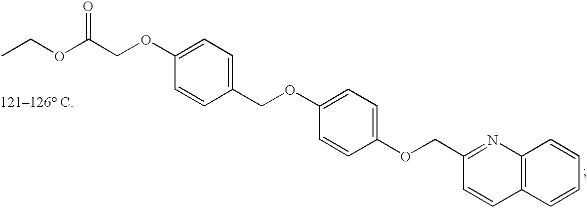

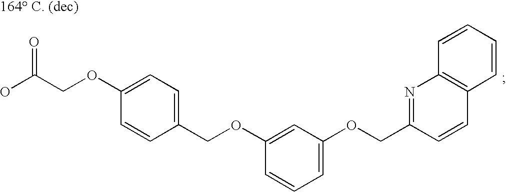

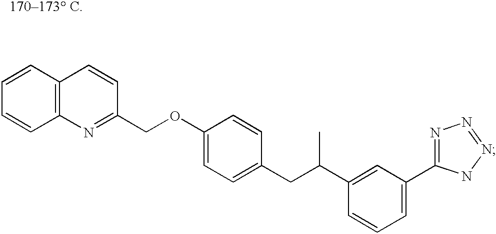

| | |
|---|---|
| 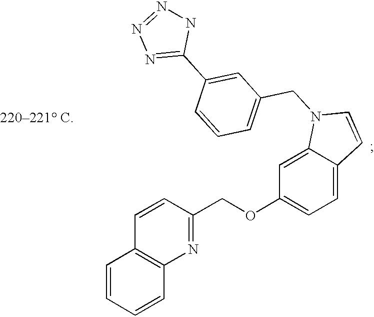 | 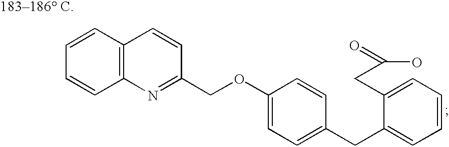 |

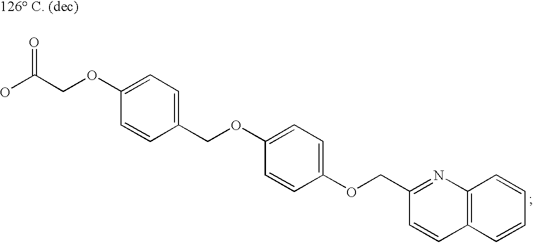

| | |
|---|---|
| 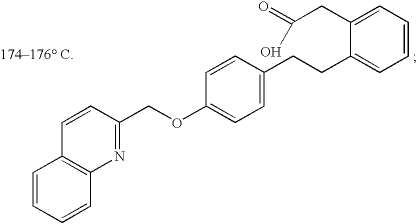 | 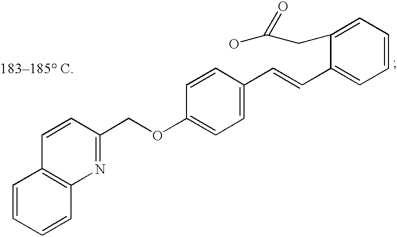 |

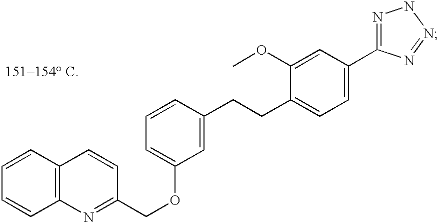

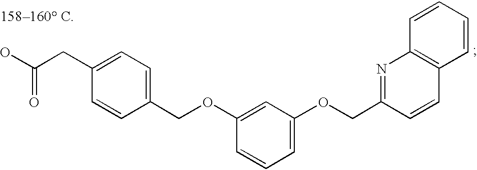

| | |
|---|---|
| 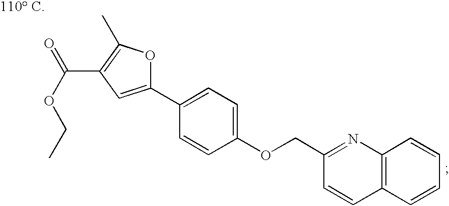 | 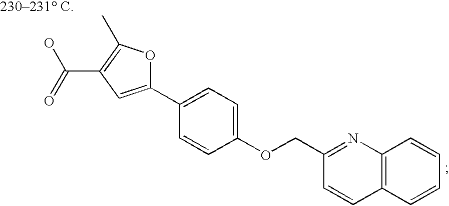 |
| 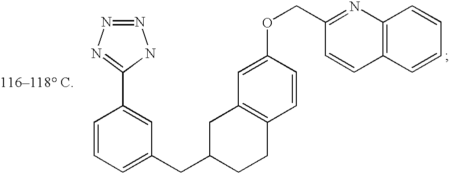 | 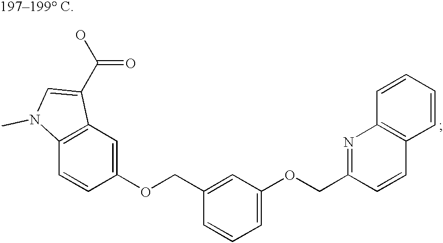 |

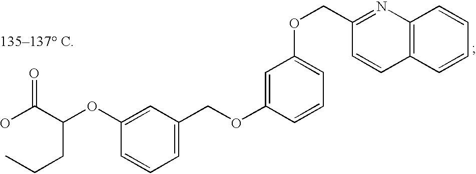

| | |
|---|---|
| 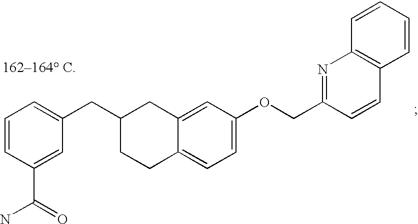 | 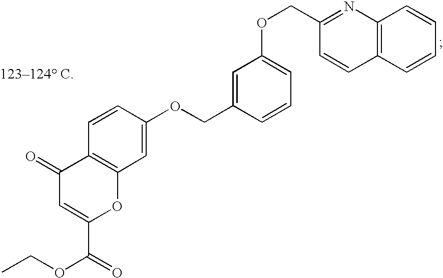 |

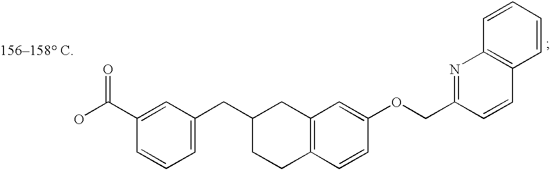

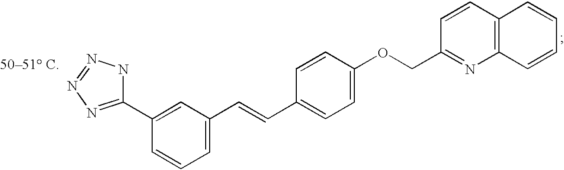

| | |
|---|---|
| 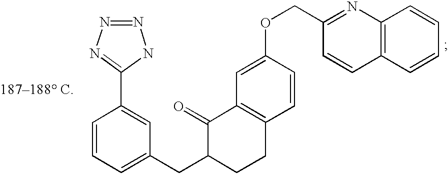 | 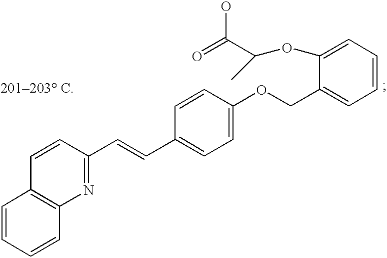 |
| 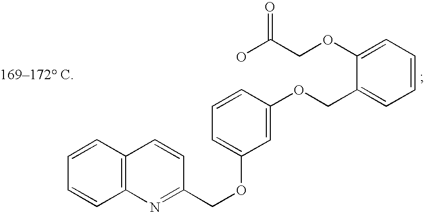 | 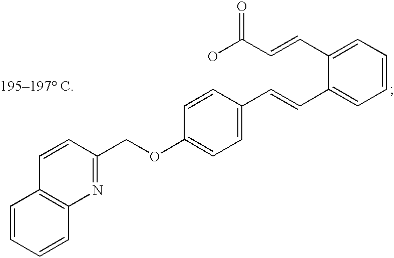 |
| 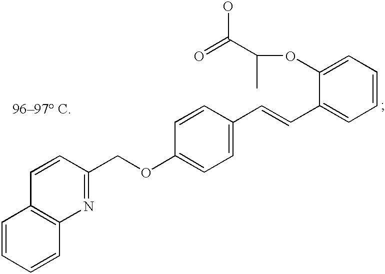 | 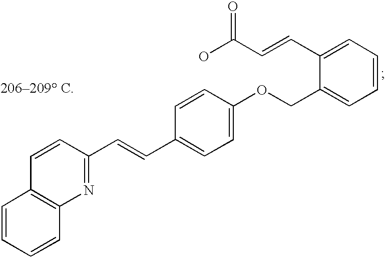 |
| 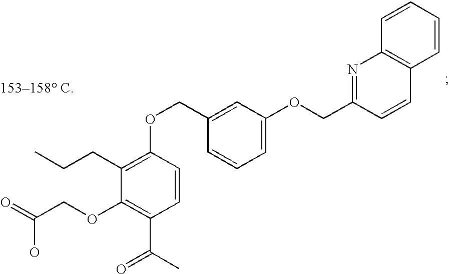 | 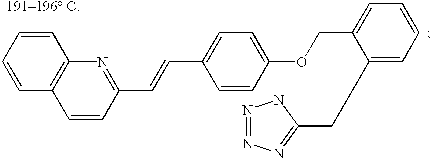 |
| 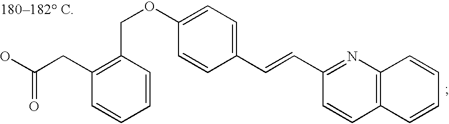 | 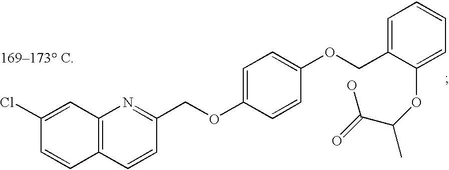 |
| 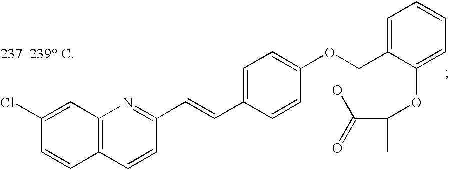 | 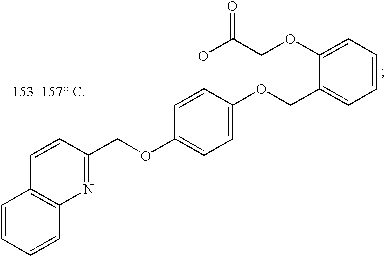 |
| 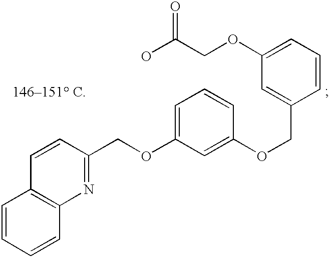 | 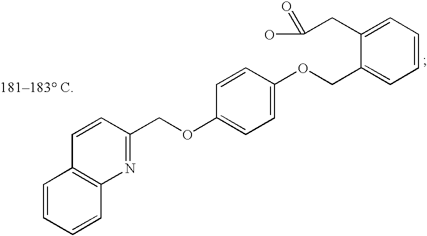 |

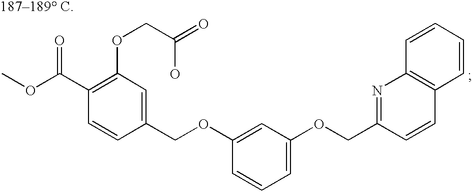

| | |
|---|---|
| 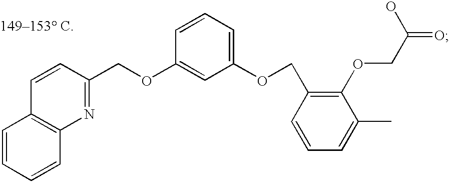 | 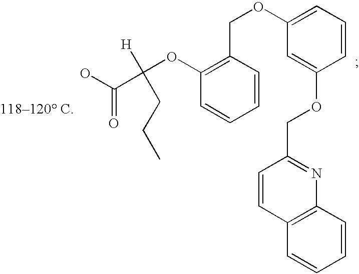 |

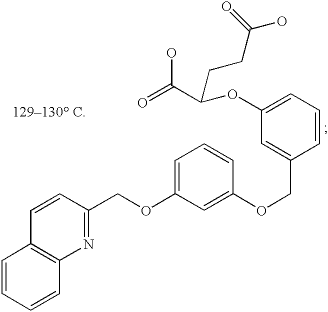

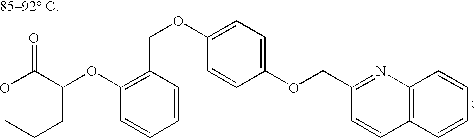

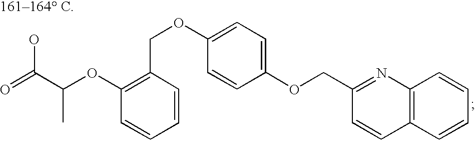

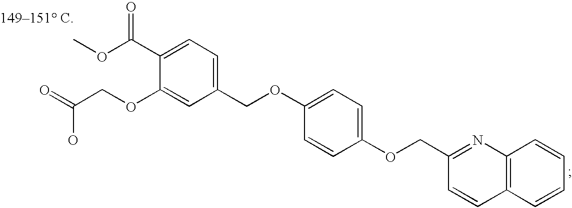

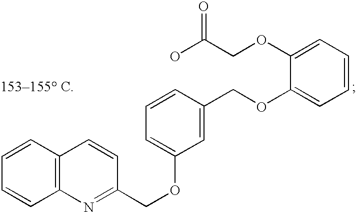

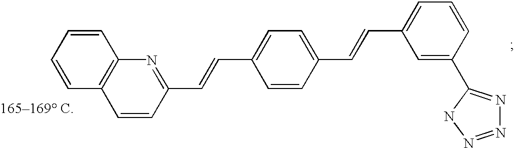

| | |
|---|---|
| 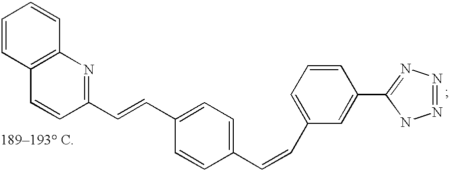 | 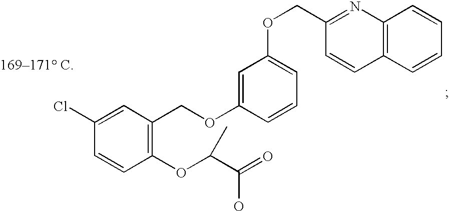 |

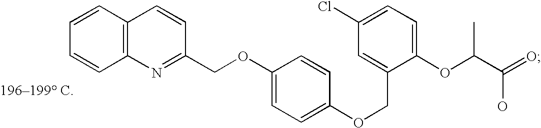

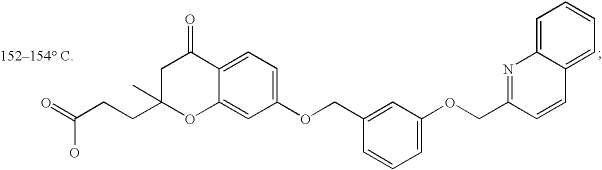

| | |
|---|---|
| 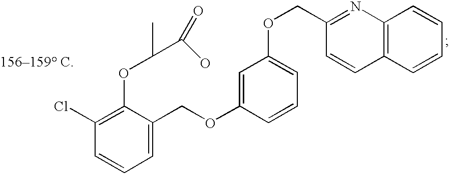 | 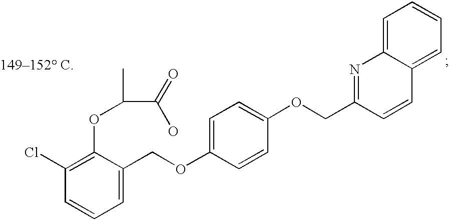 |

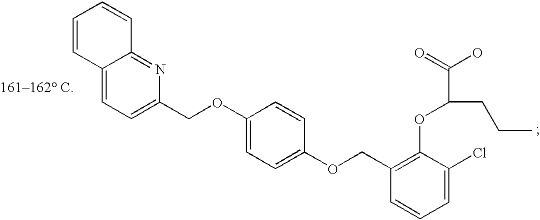

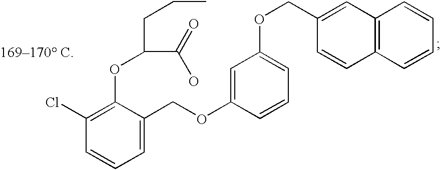

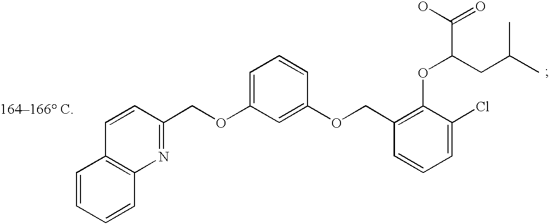

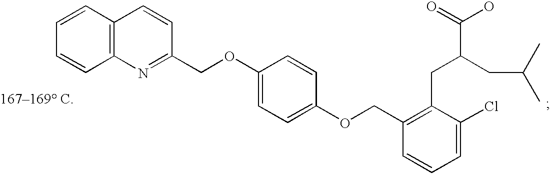

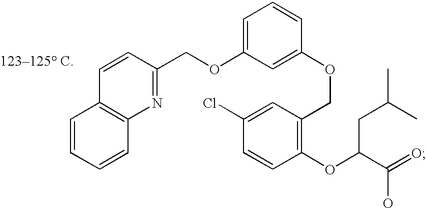

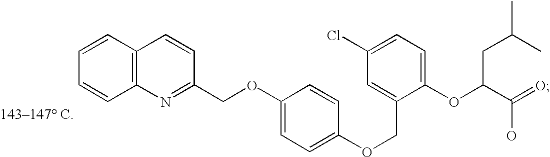

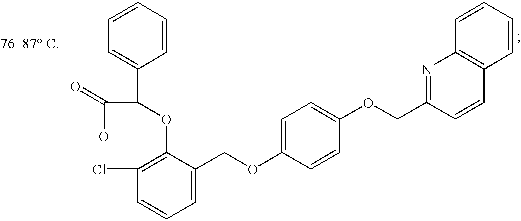

| | |
|---|---|
| 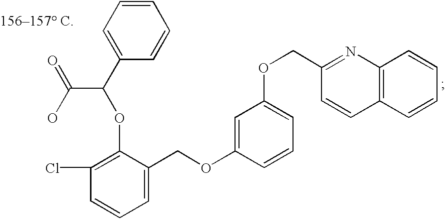 | 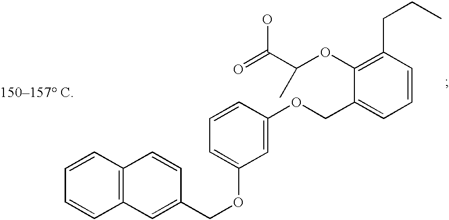 |

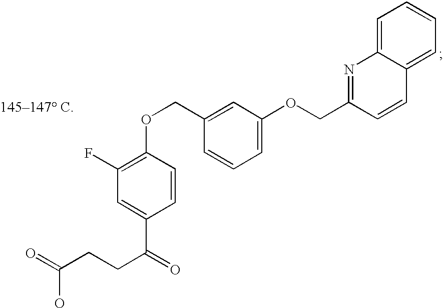

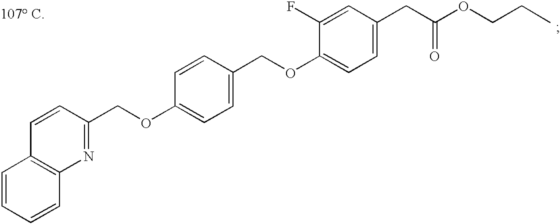

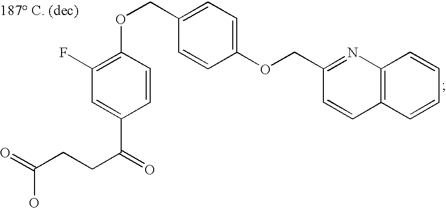

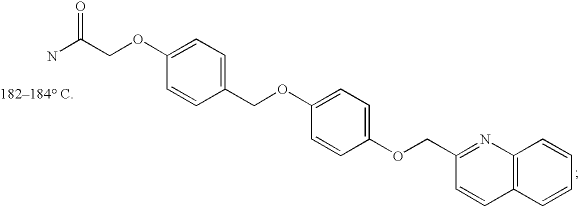

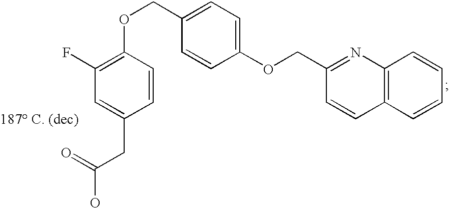

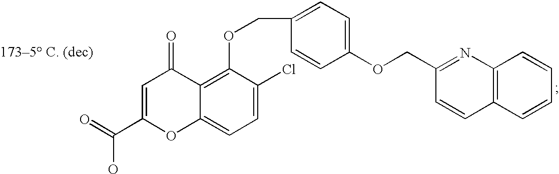

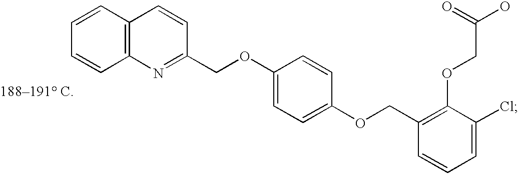

| | |
|---|---|
| 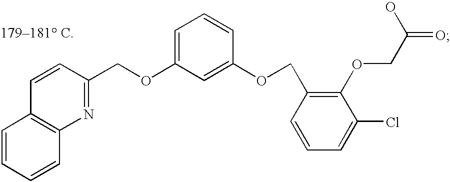 | 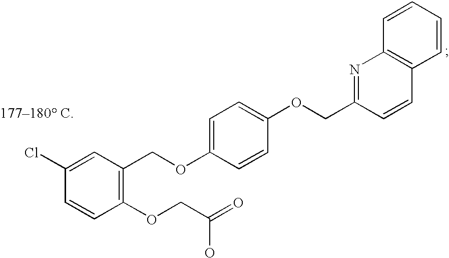 |

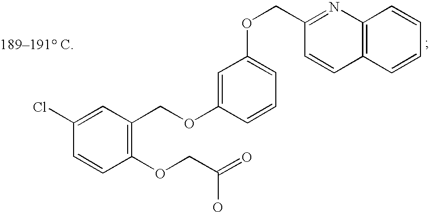

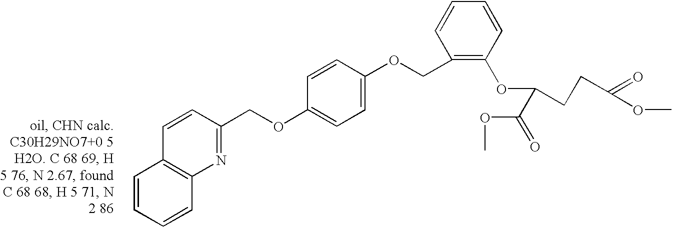

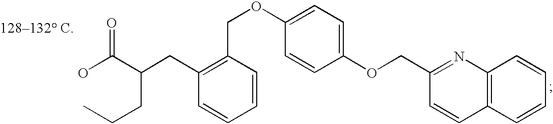

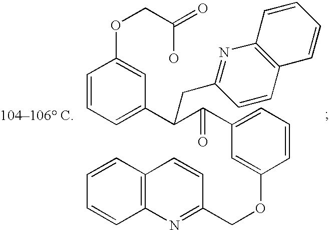

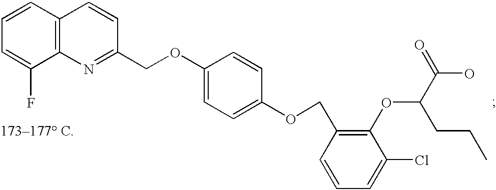

| | |
|---|---|
| 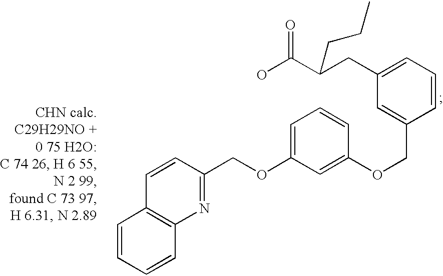 | 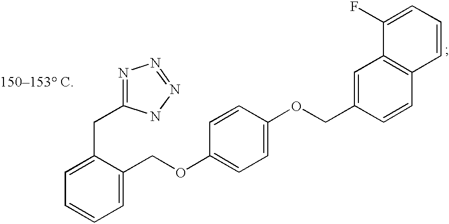 |
| 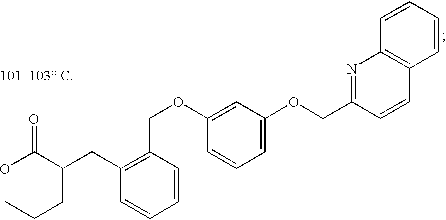 | 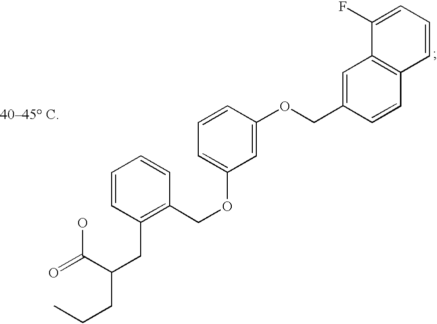 |
| 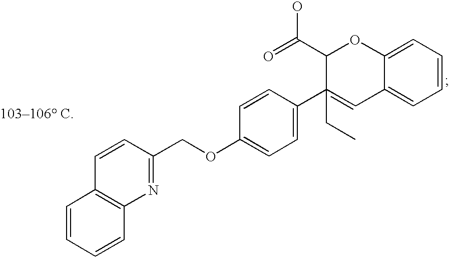 | 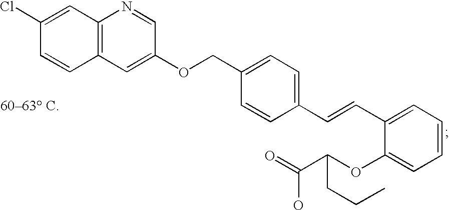 |

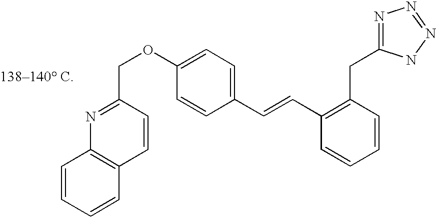

Using a combination of the above Examples, various compounds may be made within the scope of this invention.

Compounds according to the invention exhibit marked pharmacological activities according to tests described in the literature which tests results are believed to correlate to pharmacological activity in humans and other mammals. The following pharmacological test results are typical characteristics of compounds of the present invention.

The compounds of the present invention have potent activity as PPAR ligand receptor binders and possess anti-diabetic, anti-lipidemic, anti-hypertensive, and anti-arteriosclerotic activity and are also anticipated to be effective in the treatment of diabetes, obesity and other related diseases.

hPPARα Binding Assay

The activity of the compounds of the invention as PPARα modulators may be examined in several relevant in vitro and in vivo preclinical assays, for example benchmarking with a known PPARα modulator, for example, [³H]-GW2331(2-(4-[2-(3 -[2,4-Difluorophenyl]-1-heptylureido)-ethyl]phenoxy-2-methylbutyric acid). (S. Kliewer, et al. Proc. Natl. Acad. Sci. USA 94 (1997).

Human Peroxime Proliferator-activated Receptor a Ligand Binding Domain(hPPARα-LBD)

A binding assay for PPARα could be carried out by the following procedure: cDNAs encoding the putative ligand binding domain of human PPARα (amino acids 167-468) ( Sher, T., Yi, H.-F., McBride, O. W. & Gonzalez, F. J. (1993) Biochemistry 32, 5598-5604) are amplified by PCR (Polymerase Chain Reaction) and inserted in frame into the BamHI site of pGEX-2T plasmid (Pharmacia). The soluble fraction of GST-hPPARα fusion proteins or glutathione S-transferase (GST) alone are overexpressed in E. coli BL21(DE3)pLysS cells and purified from bacteria extracts as described in (S. Kliewer, et al. Proc. Natl. Acad. Sci. USA 94 (1997), 4318-4323).

Gel-Filtration Assays: 30 ml of 90 nM GST-hPPARα-LBD is mixed with 20 ml of 50 nM ³H-GW2331 with or without 5 ml of 10 mM test compounds in the binding buffer containing 10 mM Tris, 50 mM KCl, 0.05% Tween 20 and 10 mM DTT. The reaction mixtures are incubated in 96-well plates for 2 h at room temperature. 50 ml of the reaction mixtures are then loaded on a 96-well gel filtration block (following manufacture instructions)(EdgeBioSystems). The block placed on top of a clean 96-well plate is centrifuged at 1,500 rpm for 2 min. The block is discarded. 100 ml of Scintillation fluid is added to each well of the 96-well plate. After overnight equilibration, the plate is counted in the Microbeta counter (Wallac.).

Homogenous Scintillation Proximity Binding Assay

For the Scatchard analysis, glutathione coated SPA beads (1.5 mg/ml )(Amersham) are mixed with GST-hPPARα-LBD (10 mg/ml) in the binding buffer. The resulting slurry is incubated at room temperature with agitation for 15 min. 20 ml of the slurry is then added in 30 ml of binding buffer containing various amount ³H-GW2331(10˜500 nM). Nonspecific binding is determined in the present of 100 mM of GW2331. For the competition binding assay, 20 ml of the slurry is then added in 30 ml of the binding buffer containing 75 nM of ³H-GW2331 and 0.03˜20 mM of the test compounds. For the control experiments, the glutathione coated SPA beads (1.5 mg/ml) are coated with GST proteins (10 mg/ml). 20 ml of the slurry are mixed with 30 ml of 75 nM of ³H-GW2331 with or without 10 mM of GW2331. The above experiments are all performed in a 96-well plates. The sealed plates with the reaction mixtures are allowed to equilibrate for 2 h and counted in the Microbeta counter (Wallac.).

hPPARγ Binding Assay

The activity of the compounds of the invention as PPARγ modulators may be examined in several relevant in vitro and in vivo preclinical assays, for example benchmarking with a known PPARγ modulator, for example, [³H]-BRL 49853 (Lehman L. J. et al, J. Biol. Chem. 270, 12953-12956; Lehman L. J. et al, J. Biol. Chem. 272, 3406-3410 (1997), and Nichols, J. S.; et al Analytical Biochemistry 257, 112-119(1998)).

Human Peroxime Proliferator-activated Receptor a Ligand Binding Domain(hPPARγ-LBD)

A binding assay for PPARγ could be carried out by the following procedure: cDNAs encoding the putative ligand binding domain of human PPARγ (amino acids 176-477) (Green, M. E. et al. Gene expression 281-299(1995)) are amplified by PCR (polymerase chain reaction) and inserted in frame into the BamHI site of pGEX-2T plasmid (Pharmacia). The soluble fraction of GST-hPPARγ fusion proteins or glutathione S-transferase (GST) alone are overexpressed in E. coli BL21(DE3)pLysS cells and purified from bacteria extracts.

Binding Assay

The fusion proteins, GST-PPARγ-LBD in PBS (5 mg/100 ml/well) are incubated in the glutathione coated 96 well plates for 4 hours. Unbound proteins are then discarded and the plates are washed two times with the wash buffer (10 mM Tris, 50 mM KCl and 0.05% Tween-20). 100 ml of reaction mixtures containing 60 nM of ³H-BRL-49853 and 10 mM of the testing compounds (10 ml of 0.1 mM compounds from each well of the child plates) in the binding buffer (10 mM Tris, 50 mM KCl and 10 mM DTT) are then added and incubated at room temperature for 2.5 h. The reaction mixtures are discarded and the plates are washed two times with the wash buffer. 100 ml of scintillation fluid is added to each well and plates are counted on β-counter.

hPPARδ Binding Assay

The activity of the compounds of the invention as PPARδ modulators may be examined in several relevant in vitro and in vivo preclinical assays (See references WO 97/28149; Brown P. et al Chemistry & Biology, 4, 909-18, (1997)), for example benchmarking with a known PPARδ modulator, for example [³H₂] GW2433 or [³H₂] Compound X

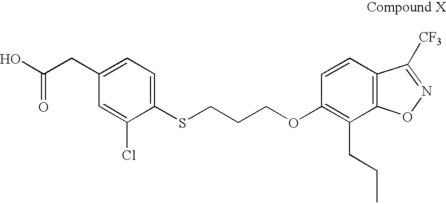

The hPPARδ binding assay comprises the steps of:

(a) preparing multiple test samples by incubating separate aliquots of the receptor hPPARδ with a test compound in TEGM containing 5-10% COS-1 cell cytoplasmic lysate and 2.5 nM labeled ([³H]Compound X, 17 Ci/mmol) for a minimum of 12 hours, and preferably for about 16 hours, at 4° C., wherein the concentration of the test compound in each test sample is different, and preparing a control sample by incubating a further separate aliquot of the receptor hPPARδ under the same conditions but without the test compound; then

(b) removing unbound ligand by adding dextran/gelatin-coated charcoal to each sample while maintaining the samples at 4° C. and allowing at least 10 minutes to pass, then

(c) subjecting each of the test samples and control sample from step (b) to centrifugation at 4° C. until the charcoal is pelleted; then

(d) counting a portion of the supernatant fraction of each of the test samples and the control sample from step (c) in a liquid scinitillation counter and analyzing the results to determine the IC₅₀ of the test compound.

In the hPPARδ binding assay, preferably at least four test samples of varying concentrations of a single test compound are prepared in order to determine the IC₅₀.

ABC-1 Assays

### Assay Example 1: ABC1 Up-regulation in Human THP-1 Cell by PPAR Mediators

THP-1 cells, a human monocytic cell line, are maintained in RPMI with 10% FCS (fetal calf serum)/20 mg/ml gentamycin/25 mM Hepes. Cells are plated at approximately 1×105 per cm² in RPMI/10% charcoal-stripped FCS (Hyclone) the presence or absence of 100 ng/ml PMA (phorbol myritic acid)(Gibco BRL) and the indicated concentrations of test compound or DMSO (dimethyl sulfoxide). Test compounds are refreshed daily. Alternatively, cells are incubated with 100 mg/ml AcLDL (acetylated LDL) as positive control. After 48 or 72 hours, cellular RNA is isolated with Trizol® (Gibco) according to the manufacturer's instructions. Total RNA (10-15 mg) is subjected to Northern blotting. The fragment used as a probe is a 431bp PCR product of ABC1 corresponding to nucleotides (nt's) 3306-3737 of Genbank Ace # AJ012376 (T. Langmann et al,1999, BBRC 257, 29-33). The sequences of the primers usd to generate the fragment are: gggaacaggctactacctgac nt. pos 3306-3326 (forward); aaggtaccatctgaggtctcagcatcc nt. pos 3737-3711 (reverse). Blots are hybridized with this probe labelled with [a32P]dCTP (Amersham) with ExpressHyb® (Clontech, Palo Alto Calif.) according to manufacturer's protocol, washed, and exposed to X-ray film. Resulting signals are quantitated by densitometry.

By way of Example, treatment of THP-1 cells with RPR64 and RPR52 at 1 and 10 μM resulted in an up-regulation of ABC1 expression.

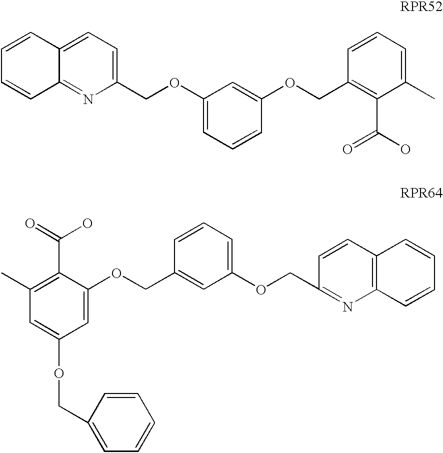

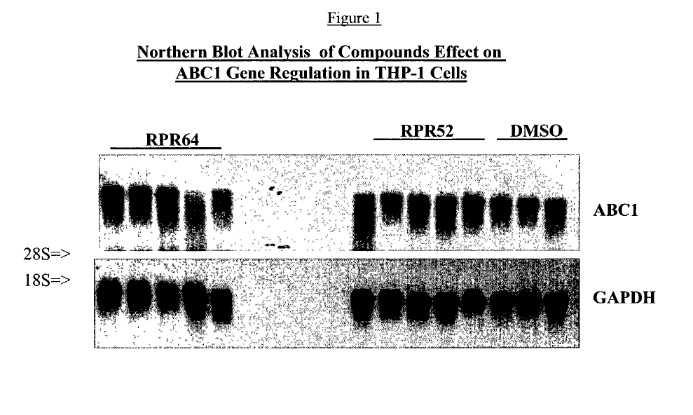

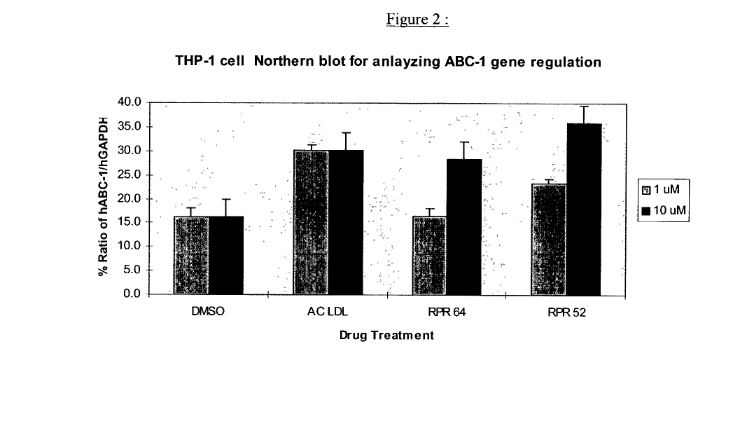

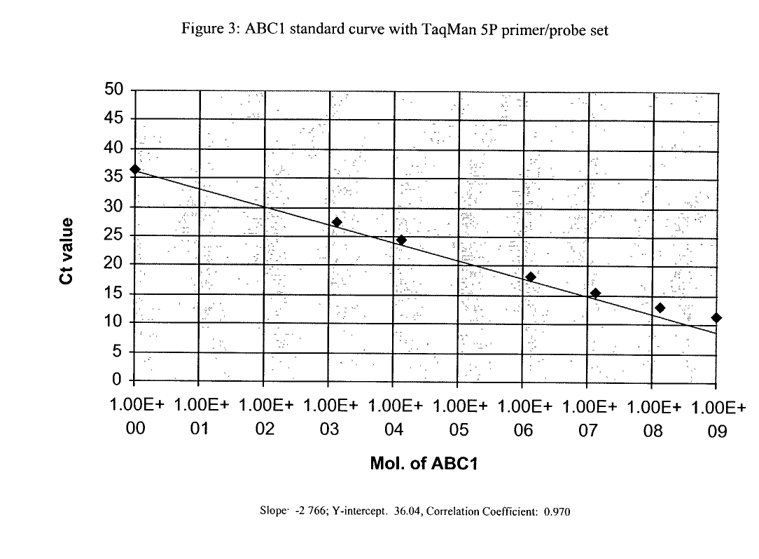

A representative example of a Northern blotting analysis is represented in FIG. 1 and corresponding graph bar in FIG. 2. Analysis of ABC1 up-regulation is also analyzed by quantitative PCR using Taqman apparatus. Standard curve is shown in FIG. 3. Similarly, treatment of THP-1 cells with the compound of formula VI, shows a fourteen fold increase in up-regulation of ABC 1 expression relative to treatment with DMSO.

### Assay Example 2: ABC 1 Up-regulation in Human Hepatocytes and Human Macrophages Derived Monocytes by Fenofibric Acid, and for Wy 14,643 and Related Cholesterol Efflux in Macrophages

Cell Culture

Mononuclear cells are isolated from blood of healthy normolipidemic donors (thrombopheresis residues). Monocytes isolated by Ficoll gradient centrifugation are suspended in RPMI 1640 medium containing gentamycin (40 mg/ml), glutamine (0.05%) (Sigma) and 10% of pooled human serum. Cells are cultured at a density of 3×10⁶ cells/well in 6-well plastic culture dishes (Primaria, Polylabo, France). Differentiation of monocytes into macrophages occured spontaneously by adhesion of cells to the culture dishes. Mature monocyte-derived macrophages as characterized by immunocytochemistry with anti CD-68 antibody, are used for experiments after 9 days of culture. For treatment with the different activators, medium is changed to RPMI 1640 medium without serum but supplemented with 1% Nutridoma HU (Boehringer Mannheim).

Human liver specimens are collected from healthy multiorgan donors for transplantation who died after severe traumatic brain injury. Hepatocytes are obtained by a two-step collagenase perfusion (REF). Cells are resuspended in minimal essential medium with Earl's salts with 10% FCS, 2 mM glutamine, 50 mg/ml gentamycin, seeded at density of 1.5×10⁵ cells/cm² in plastic culture dishes coated with 20 mg rat tail collagene type I (Sigma).Medium is renewed after 4 hours of adhesion. After 20 hours the medium is discarded and differents compounds added at the indicated concentrations in serum-free medium.

RNA Extraction and Analysis

Total cellular RNA is extracted from differentated macrophages treated for 6 hours with different compounds using the RNA plus kit (Bioprobe System, Montreuil, france). RNA from human hepatocytes are prepared as described by Chomczynski and Sacchi. For RT-PCR analysis, total RNA is reverse transcribed using random hexamer primers and Superscript reverse transcriptase (Life Technologies) as sebsequently amplified by PCR. The resulting products are separated on a 1% agrose gel and stained with ethidium bromide.

Cholesterol Loading and Efflux:

9 days-old human macrophages are pretreated for 24 hours with different PPAR activators and cholesterol loaded by incubation with acetylated LDL (50 μg of proteins in 2 ml/well of RPMI1640 supplemented with 1% of Nutridoma) for 48 hours. After this period cells are washed twice in PBS and 1 ml of fresh RPMI medium without Nutridoma containing 100 μg of Apo AI is added in each well for 24 hours. At the end of this incubation, intracellular lipids are extracted by isopropanol and cellular proteins are collected by digestion in NaOH. Where indicated, PPAR activators are added to culture medium each day at concentrations of 20 μM for Wy 14,643.

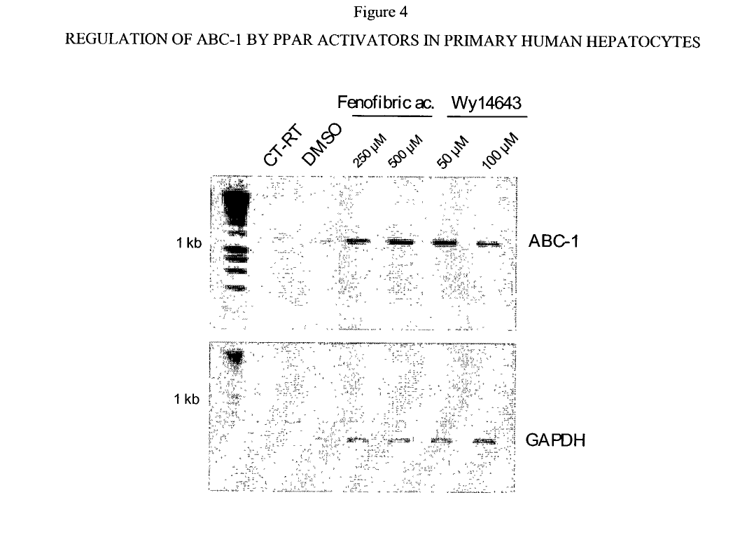

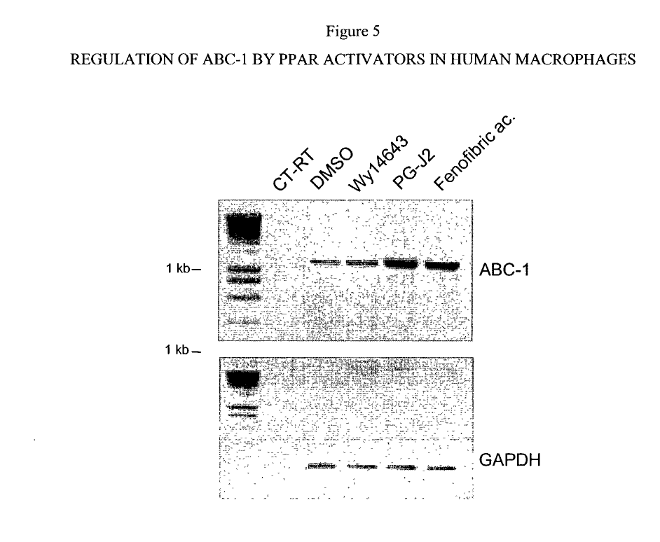

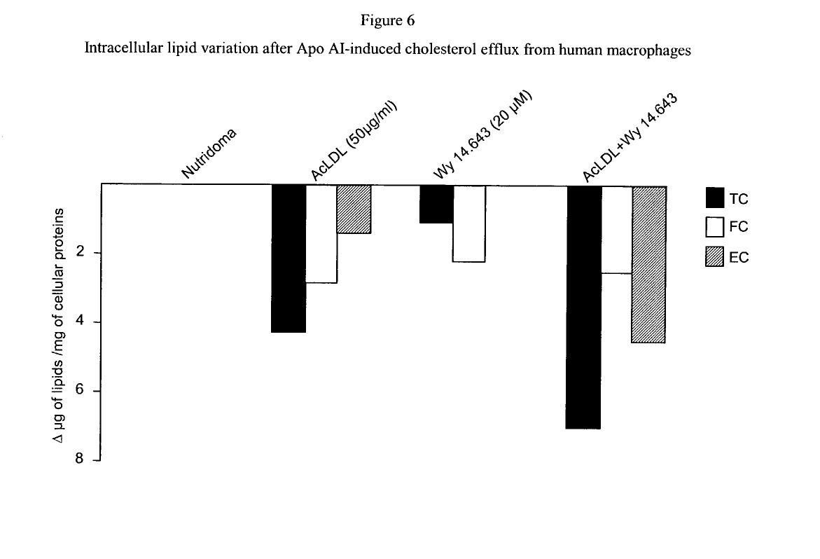

By way of example, treatment of human primary hepatocytes with the Fenofibric acid and Wy 14,643 resulted in ABC1 up-regulation. Representative data are shown in FIG. 4. Similar results were observed with treatment of human monocytes derived macrophages using Fenofibric acid, PG-J2 and the Wy 14,643 compounds as shown in FIG. 5. Apolipoprotein A-I-mediated cholesterol efflux was studied in human monocytes derived macrophages treated with AcLDL, Wy 14,643 and AcLDL+Wy 14,643 (FIG. 6).

Summary of ABC-1 Assay

Present results indicated that human ABC1 gene is regulated by PPAR activators. Up-regulation of human ABC1 is demonstrated in human THP-1 cells by RPR64 and RPR52 compounds already described as PPAR-alpha agonists. This up-regulation is assessed by Northern blotting analysis as well as by quantitative RT-PCR TaqMan analysis. In addition, up-regulation of human ABC1 is demonstrated in human primary hepatocytes and human macrophages derived monocytes by Fenofibric acid, Wy 14,643 already described as PPAR-alpha agonists as well as by PG-J2 already described as a PPAR-gamma agonist. In addition, treatment of cells by PPAR-alpha or -gamma agonists increase cellular cholesterol efflux mediated by apolipoprotein which is the critical step for reverse cholesterol transport, thus, peripheral cellular cholesterol excess removal from the body. In summary, PPAR-alpha and gamma agonists treatment are clearly of interest for patients with ABC1 defects.

The compounds useful according to the invention can be administered to a patient in a variety of forms adapted to the chosen route of administration, i.e., orally, or parenterally. Parenteral administration in this respect includes administration by the following routes: intravenous, intramuscular, subcutaneous, intraocular, intrasynovial, transepthelially including transdermal, opthalmic, sublingual and buccal; topically including opthalmic, dermal, ocular, rectal and nasal inhalation via insufflation and aerosol and rectal systemic.

The active compound may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the active compound may be incorporated with excipient and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be from about 2% to about 6% of the weight of the unit. The amount of active compound in such therapeutically useful compositions is such that a suitable dosage will be obtained. Preferred compositions or preparations according to the present invention are prepared so that an oral dosage unit form contains between about 50 and 300 mg of active compound.

The tablets, troches, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavoring agent such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens a preservatives, a dye and flavoring such as cherry or orange flavor. Of course, any material used in preparing any dosage unit form should be pharmaceutically pure and substantially non-toxic in the amounts employed. In addition, the active compound may be incorporated into sustained-release preparations and formulations.

The active compound may also be administered parenterally or intraperitoneally. Solutions of the active compound as a free base or pharmacologically acceptable salt can be prepared in water suitably mixed with a surfactant such as hydroxypropyl-cellulose. Dispersion can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It may be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained , for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from previously sterile-filtered solution thereof.

The therapeutic compounds useful according to this invention may be administered to a patient alone or in combination with pharmaceutically acceptable carriers, as noted above, the proportion of which is determined by the solubility and chemical nature of the compound, chosen route of administration and standard pharmaceutical practice.

The physician will determine the dosage of the present therapeutic agents which will be most suitable for prophylaxis or treatment and it will vary with the form of administration and the particular compound chosen, and also, it will vary with the particular patient under treatment. He will generally wish to initiate treatment with small dosages by small increments until the optimum effect under the circumstances is reached. The therapeutic dosage will generally be from 0.1 to 100 mM/day or from about 0.1 mg to about 50 mg/kg of body weight per day, or 10 mg to about 50 mg/kg of body weight per day, or more preferably 30 mg to about 50 mg/kg of body weight per day, and higher, although it may be administered in several different dosage units. Higher dosages are required for oral administration.

The compounds useful according to the invention may be administered as frequently as necessary in order to obtain the desired therapeutic effect. Some patients may respond rapidly to a higher or lower dose and may find much weaker maintenance doses adequate. For other patients, it may be necessary to have long-term treatments at the rate of 1 to 4 doses per day, in accordance with the physiological requirements of each particular patient. Generally, the active product may be administered orally 1 to 4 times per day. It goes without saying that, for other patients, it will be necessary to prescribe not more than one or two doses per day.

One skilled in the art will readily appreciate that the present invention is well adapted to carry out the objects of the invention and obtain the ends and advantages mentioned, as well as those inherent therein. The compounds, compositions and methods described herein are presented as representative of the preferred embodiments, or intended to be exemplary and not intended as limitations on the scope of the present invention.

1. A method for modulating ABC-1 gene expression comprising contacting a PPAR receptor with a PPAR mediator.

2. A method according to claim 1 wherein the PPAR receptor is a PPAR-γ receptor.

3. A method according to claim 1 wherein the PPAR receptor is a PPAR-α receptor.

4. A method according to claim 1 wherein the PPAR receptor is a PPAR-δ receptor.

5. A method according to claim 1 wherein the PPAR mediator is a PPAR agonist.

6. A method according to claim 1 wherein the PPAR mediator is a PPAR antagonist.

7. A method according to claim 1 wherein ABC-1 gene expression is induced by a PPAR agonists.

8. A method according to claim 1 wherein ABC-1 gene expression is repressed by a PPAR antagonist.

9. 9. A method of treating a physiological condition in a patient associated with ABC-1 gene expression comprising administering to a patient in need of such treatment, a pharmaceutically effective amount of a PPAR mediator.

10. A method according to claim 9 wherein the physiological condition is associated with ABC-1 deficiency.

11. A method according to claim 10 wherein the physiological condition is low levels of HDL.

12. A method according to claim 10 wherein the physiological condition is atherosclerosis, fish-eye disease, familial HDL deficiencies (FHD), Tangier disease, LCAT deficiency, cholesterol efflux, malaria or diabetes.

13. A method according to claim 9 wherein the physiological condition is associated with elevated levels of ABC-1.

14. A method according to claim 12 wherein the physiological condition is inflammation.

15. A method according to claim 1 or 9 wherein the PPAR mediator is selected from the group consisting of Nafenopn, UF-5, ETYA, GW2331, 15-deoxy-Δ^{12,14}-prostaglandin J₂, clofibric, linoleic acid, BRL-49653, fenofibrate, WR-1339, Pioglitazone, Ciglitazone, Englitazone, Troglitazone, LY-171883, AD 5075, 5-[[4-[2-(methyl-2-pyridinylamino)ethoxy]phenyl]methyl]-2,4-thiazolidinedione, WAY-120,744, and Darglitazone and their pharmaceutically acceptable salts.

16. A method according to claim 1 or 9 wherein the PPAR mediator is a compound of formula (I) 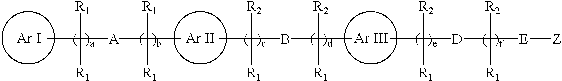
wherein: 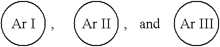
are independently aryl, fused arylcycloalkenyl, fused arylcycloalkyl, fused arylheterocyclenyl, fused arylheterocyclyl, heteroaryl, fused heteroarylcycloalkenyl, fused heteroarylcycloalkyl, fused heteroarylheterocyclenyl, or fused heteroarylheterocyclyl;
A is O, S, SO, SO₂, NR₅, a chemical bond, 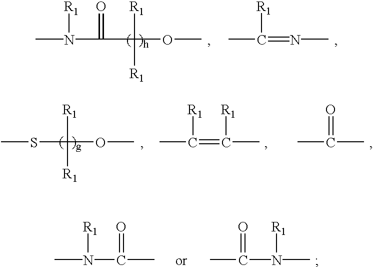
B is O, S, SO, SO₂, NR₄, a chemical bond, 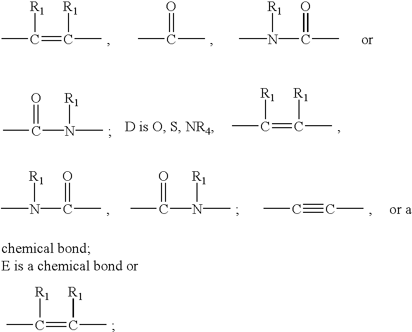
a is 0-4;
b is 0-4;
c is 0-4;
d is 0-5;
e is 0-4;
f is 0-6;
g is 2-4;
h is 0-4;
R₁ is independently hydrogen, halogen, alkyl, carboxyl, alkoxycarbonyl or aralkyl, or geminal R₁ radicals, taken together with the carbon atom to which the geminal R₁ radicals are attached, form ═CHR₁ or carbonyl, or two R₁ radicals taken together with the carbon atoms to which the R₁ are linked, form cycloalkylene, or two vicinal R₁ radicals, taken together with the carbon atoms to which the vicinal R₁ radicals are linked form 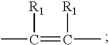
R₂ is independently —(CH₂)_{q}-X, or two R₂ radicals taken together with the carbon atoms through which the two R₂ radicals are linked form cycloalkylene, or geminal R₁ and R₂ radicals, taken together with the carbon atom to which the geminal R₁ and R₂ radicals are attached, form cycloalkylene, ═CHR₁, or carbonyl, or two vicinal R₂ radicals, taken together with the carbon atoms to which the vicinal R₂ radicals are linked, form 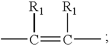
q is 0-3;
X is hydrogen, halogen, alkyl, alkenyl, cycloalkyl, heterocyclyl, aryl, heteroaryl, aralkyl, heteroaralkyl, hydroxy, alkoxy, aralkoxy, heteroaralkoxy, carboxy, alkoxycarbonyl, tetrazolyl, acyl, acylHNSO₂—, —SR₃, Y¹Y²N— or Y³Y⁴NCO—;
Y¹ and Y² are independently hydrogen, alkyl, aryl, aralkyl or heteroaralkyl, or one of Y¹ and Y² is hydrogen or alkyl and the other of Y¹ and Y² is acyl or aroyl;
Y³ and Y⁴ are independently hydrogen, alkyl, aryl, aralkyl or heteroaralkyl;
Z is R₃O₂C—, R₃OC—, cyclo-imide, —CN, R₃O₂SHNCO—, R₃O₂SHN—, (R₃)₂NCO—,R₃O— or tetrazolyl; and
R₃ and R₄ are independently hydrogen, alkyl, aryl, cycloalkyl, or aralkyl;
R₅ is R₆OC—, R₆NHOC—, hydrogen, alkyl, aryl, heteroaryl, cycloalkyl, heterocyclyl, heteroaralkyl, or aralkyl; and
R₆ is hydrogen, alkyl, aryl, heteroaryl, cycloalkyl, heterocyclyl, heteroaralkyl, or aralkyl; or a pharmaceutically acceptable salt thereof.

17. A method according to claim 1 or 9 wherein the PPAR mediator is selected from the group consisting of

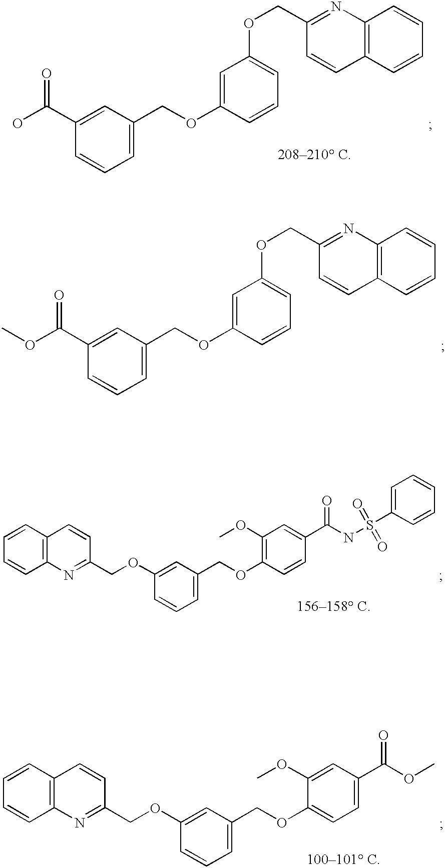

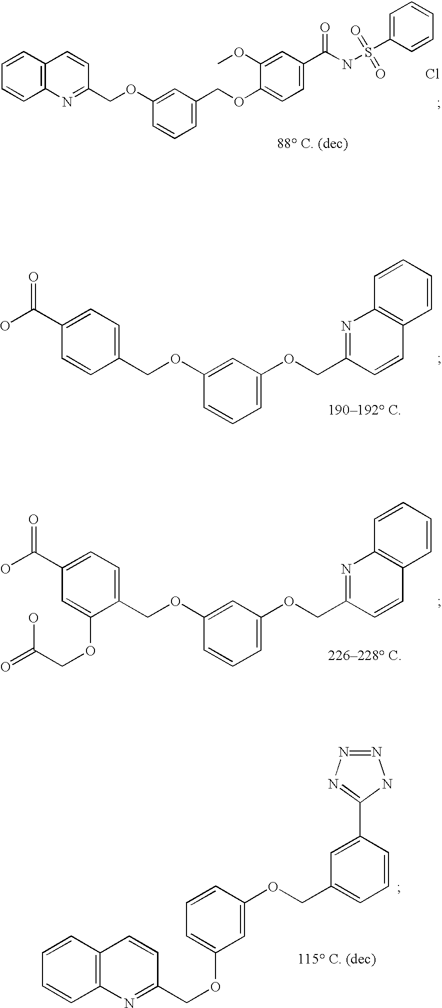

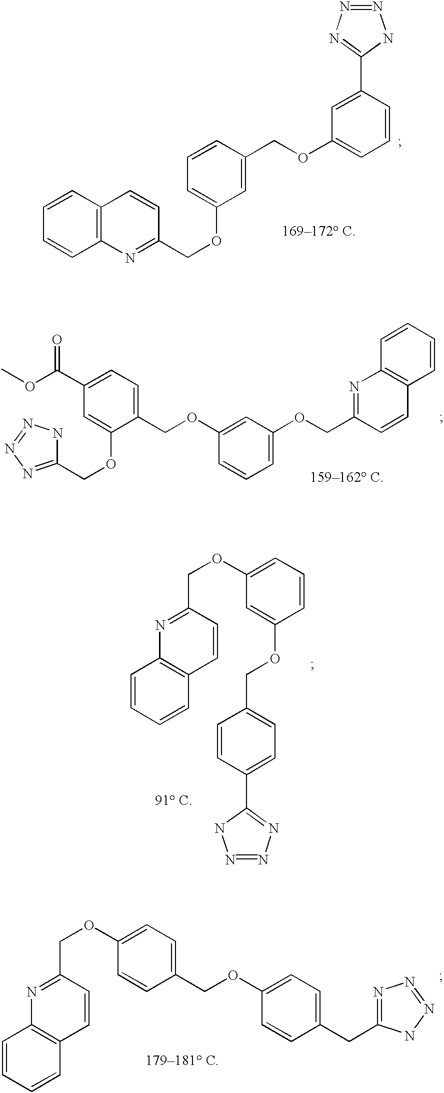

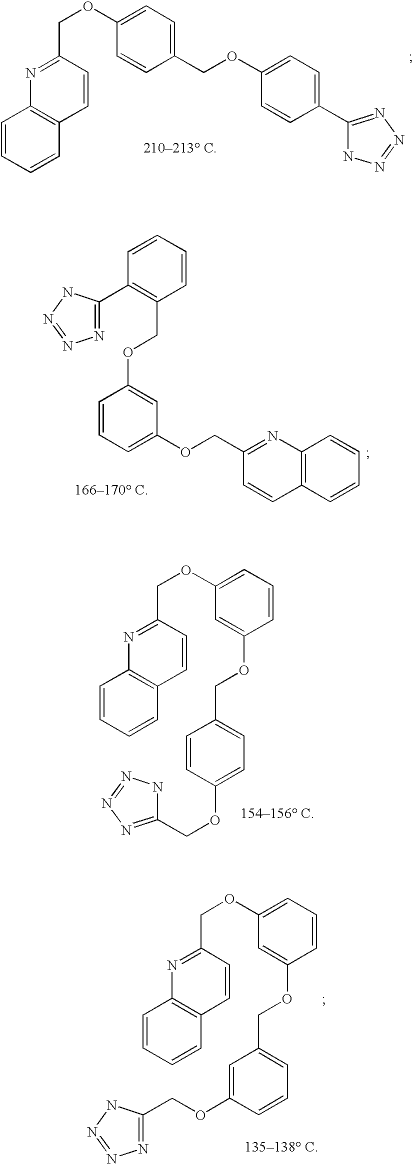

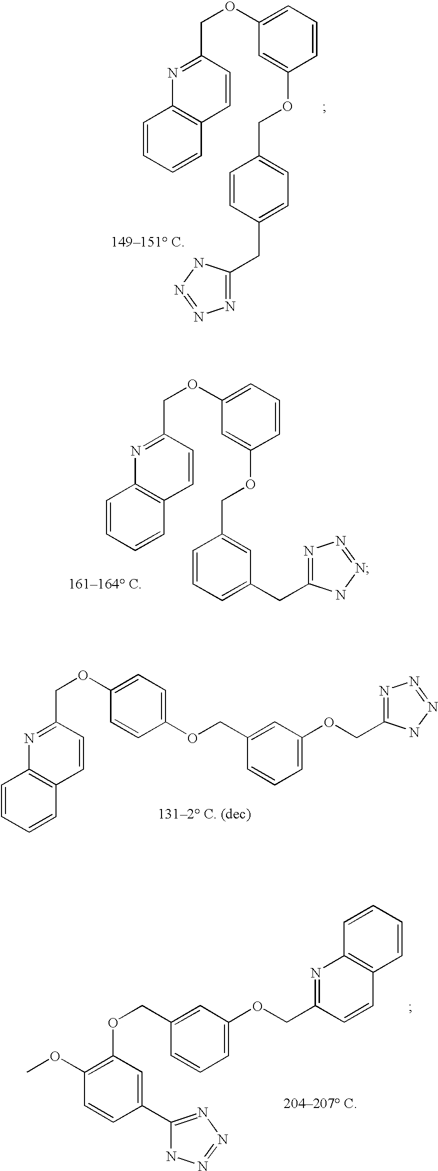

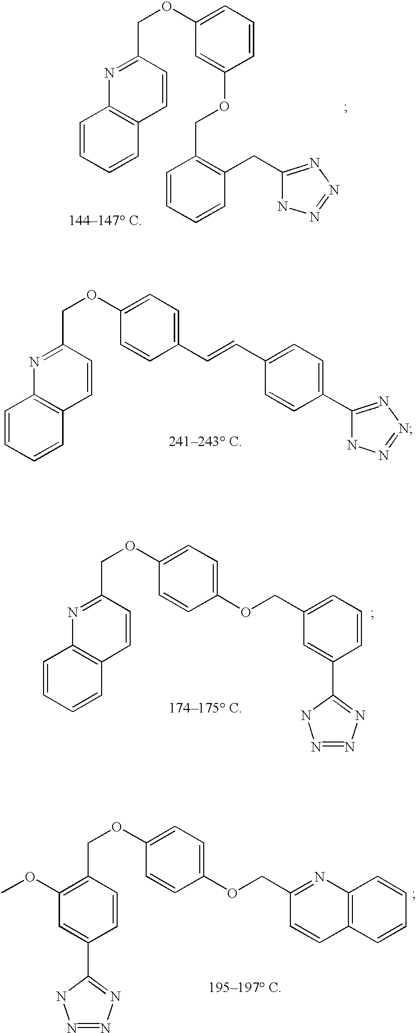

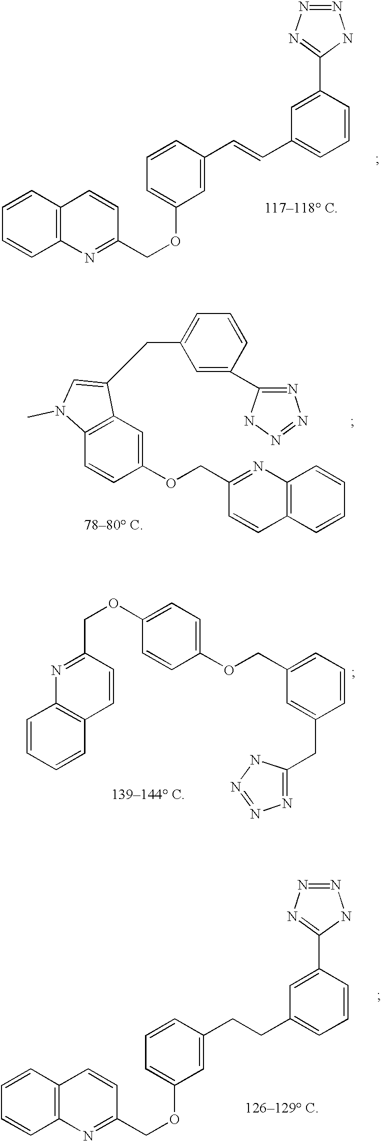

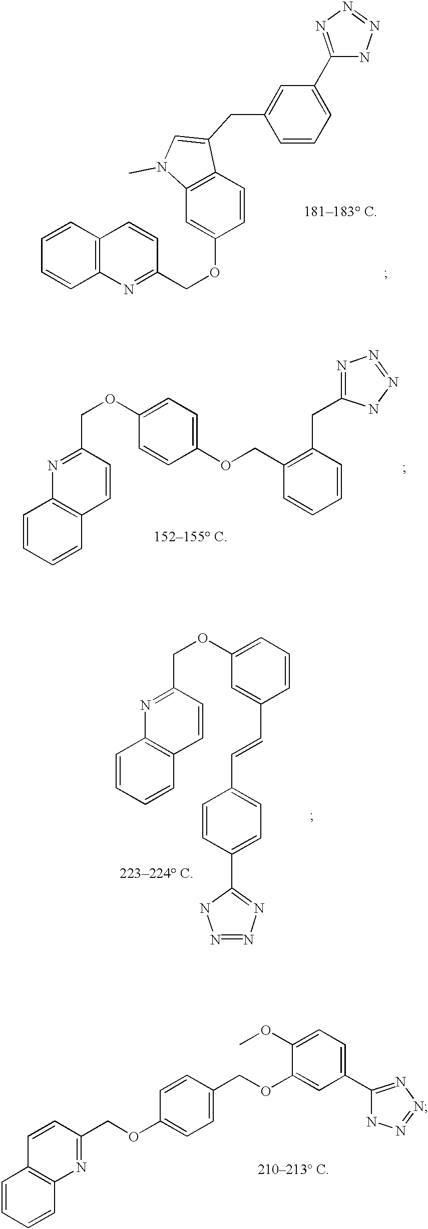

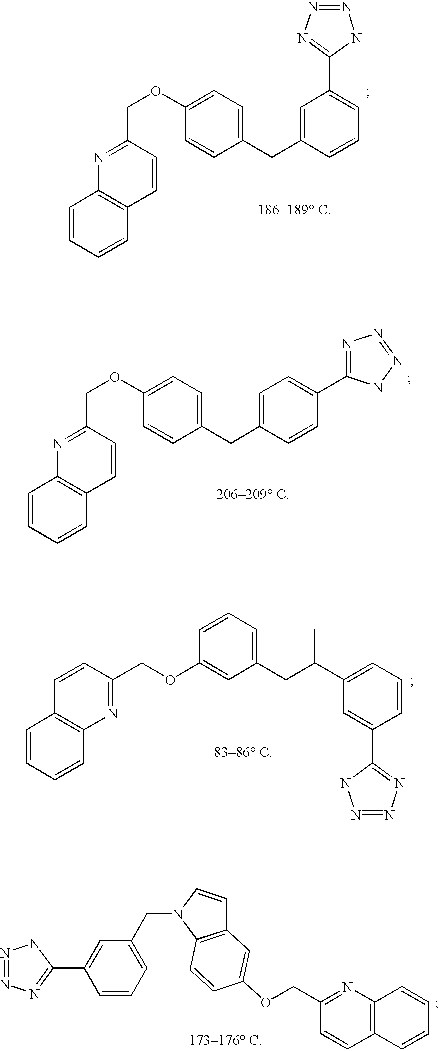

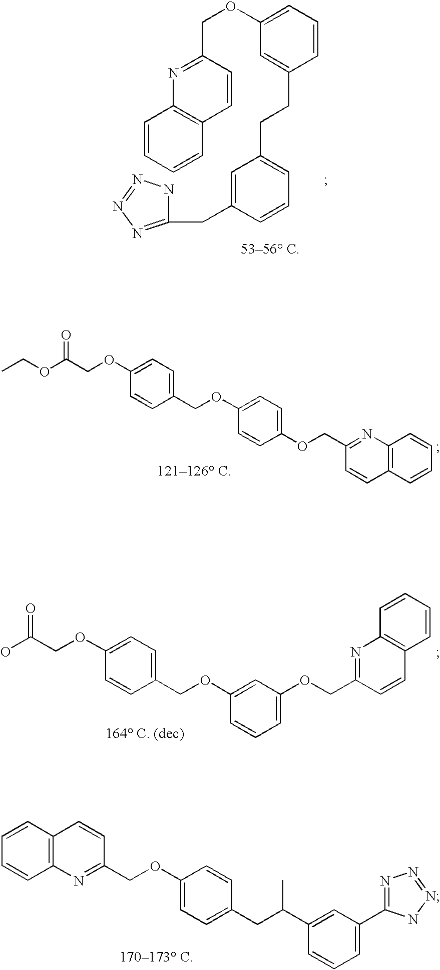

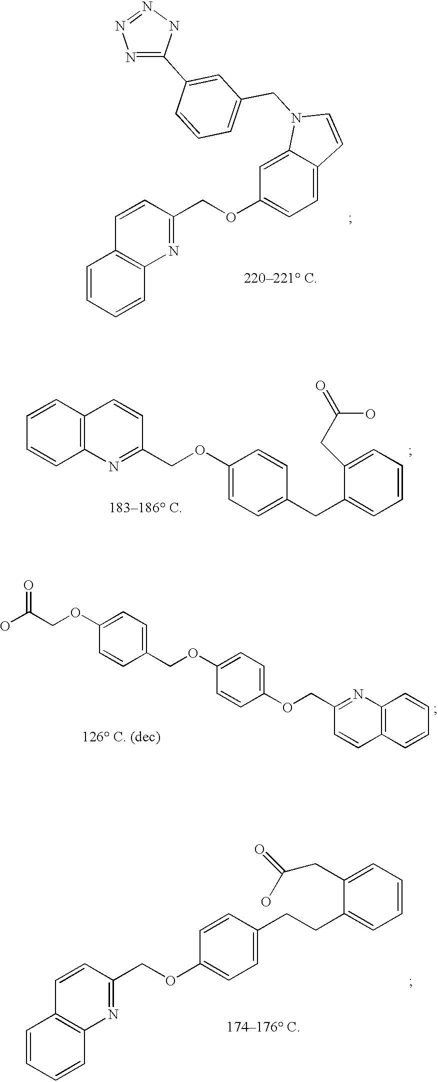

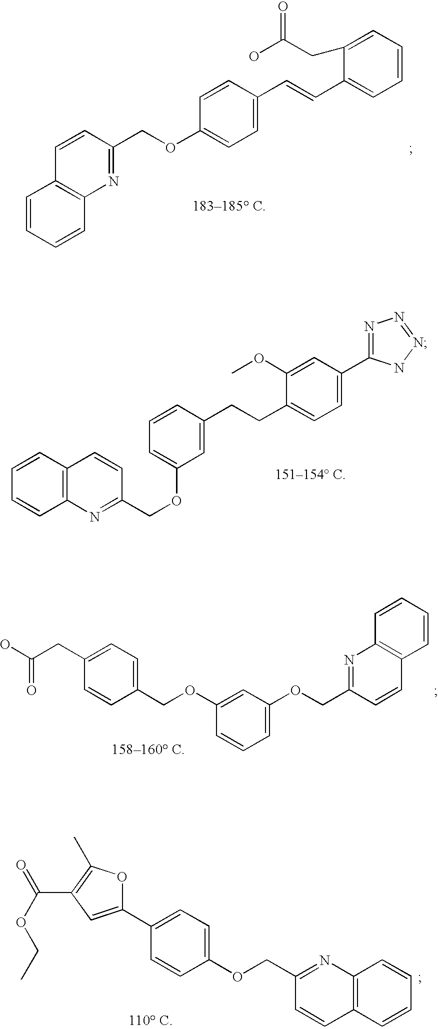

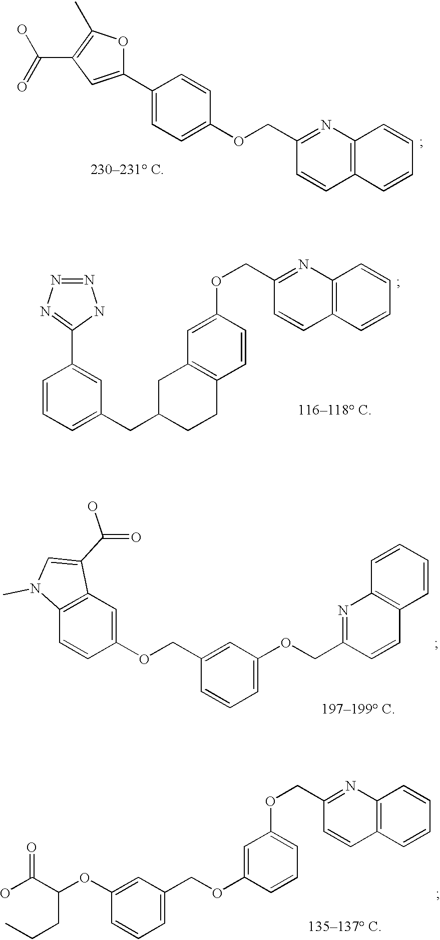

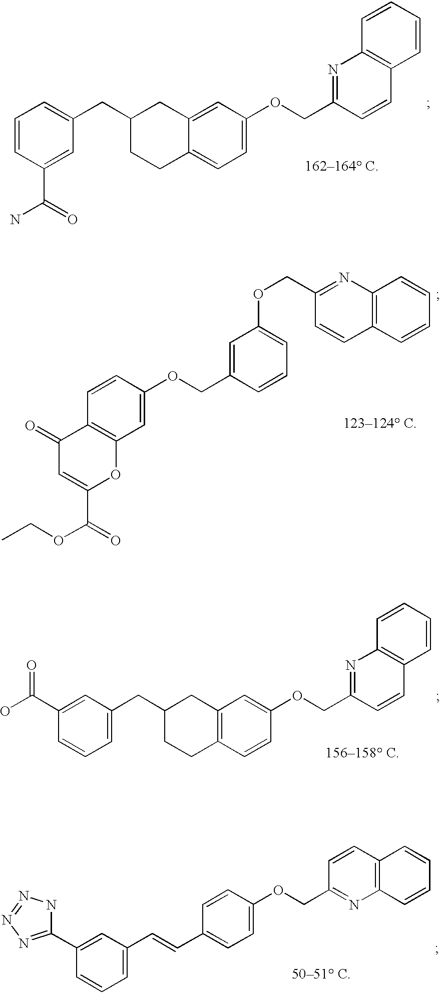

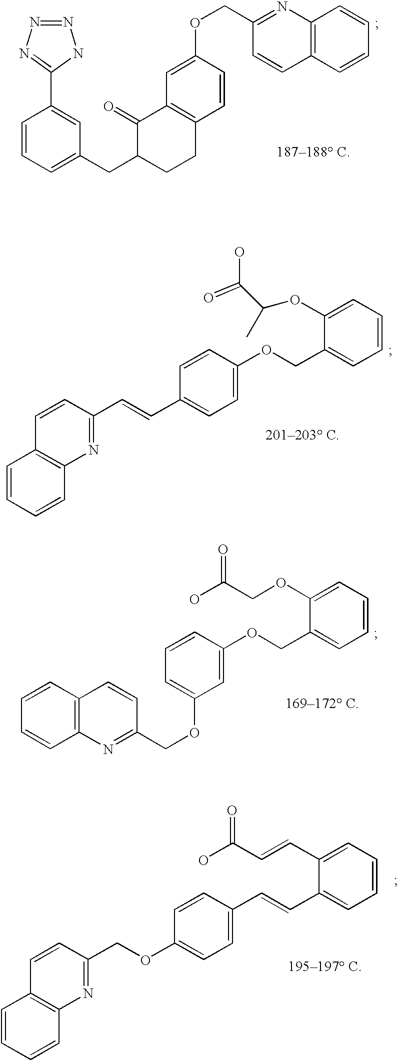

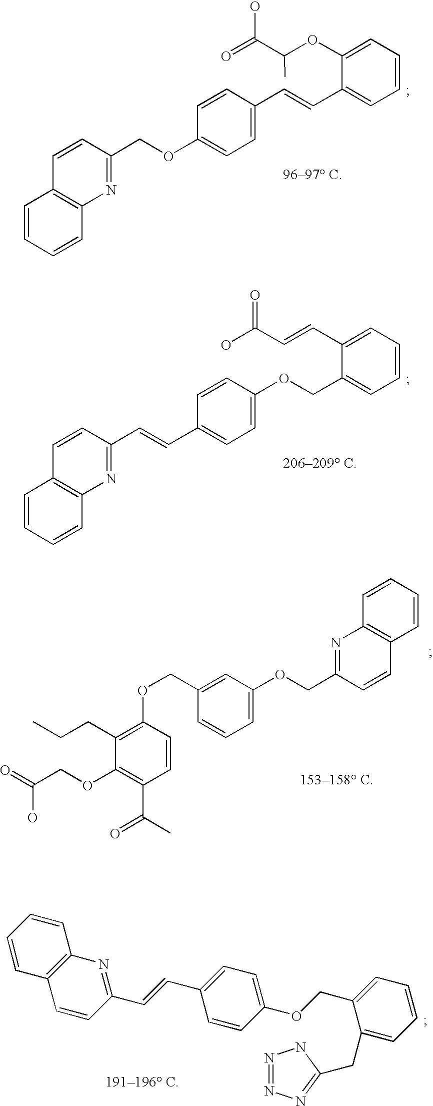

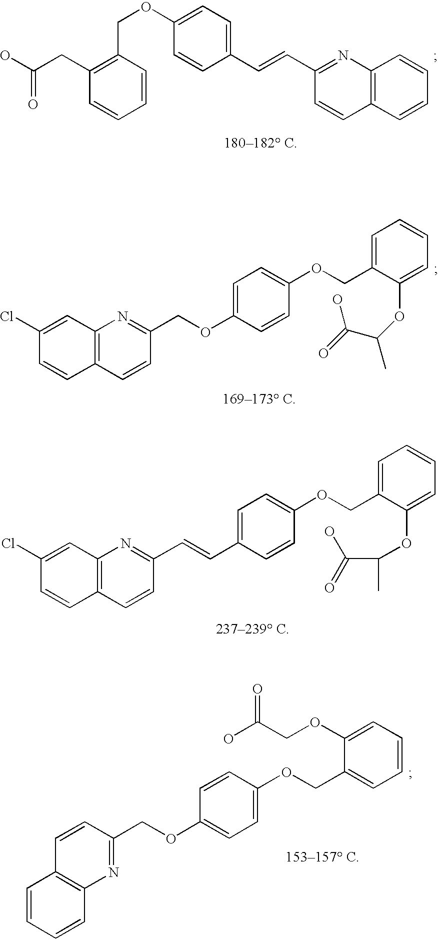

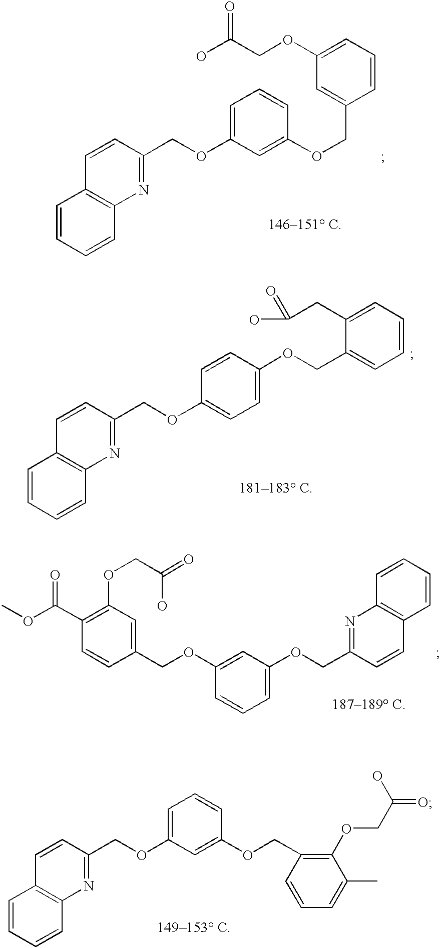

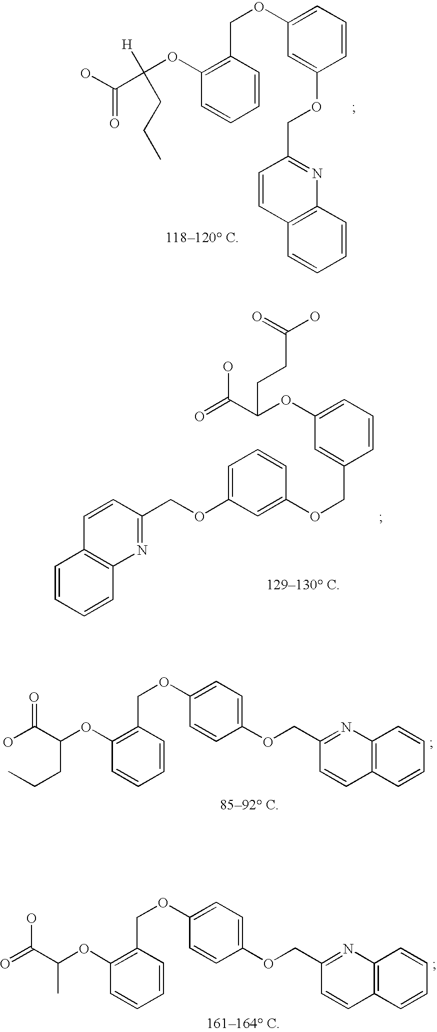

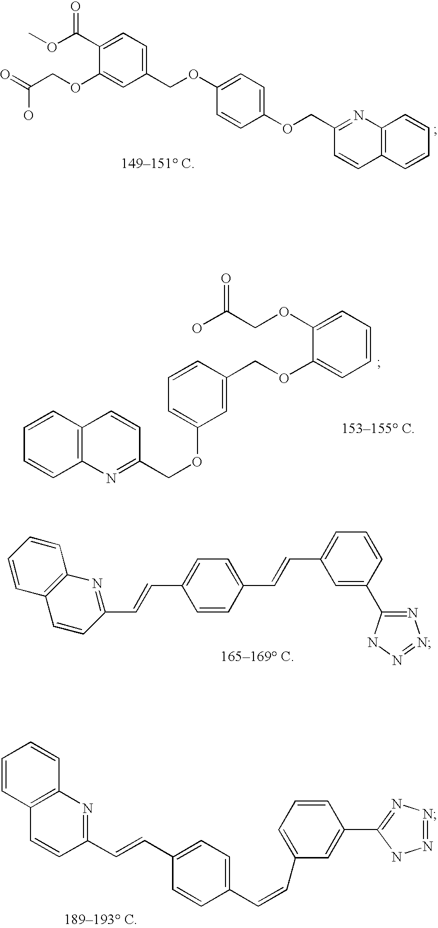

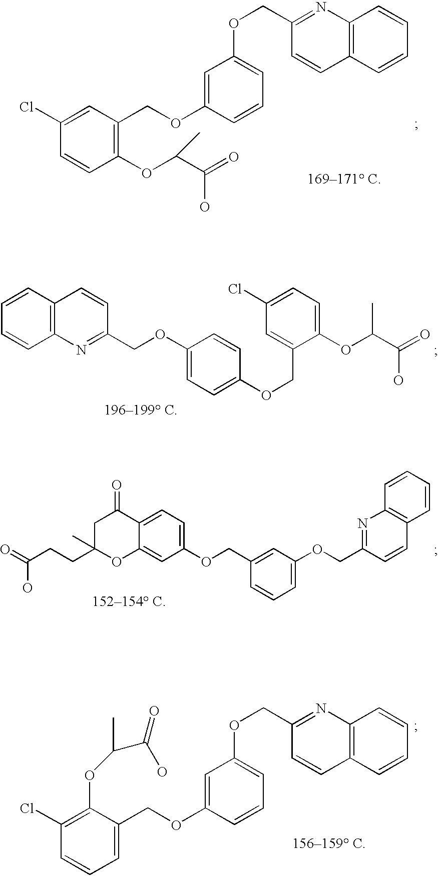

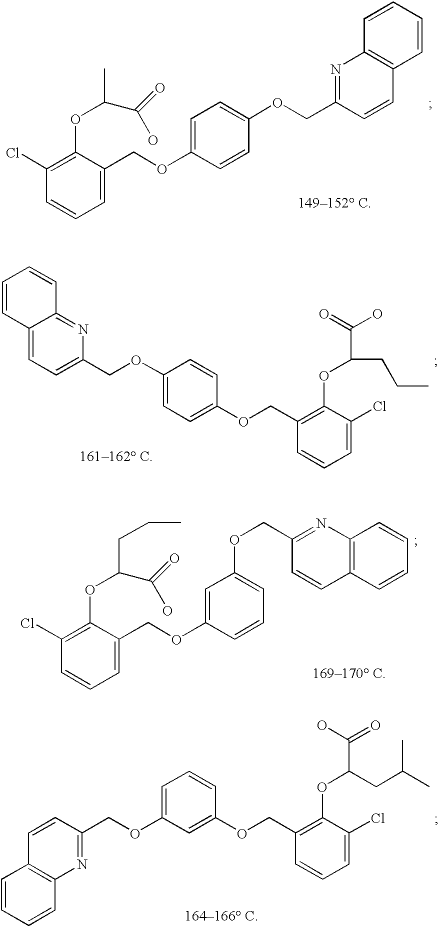

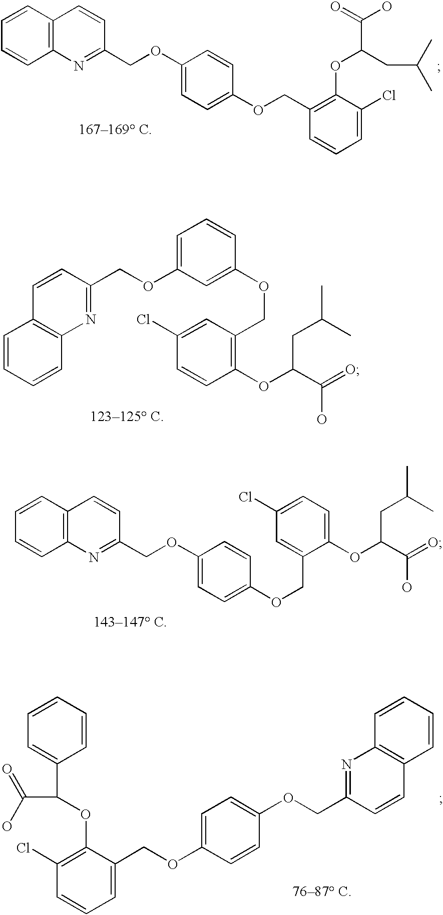

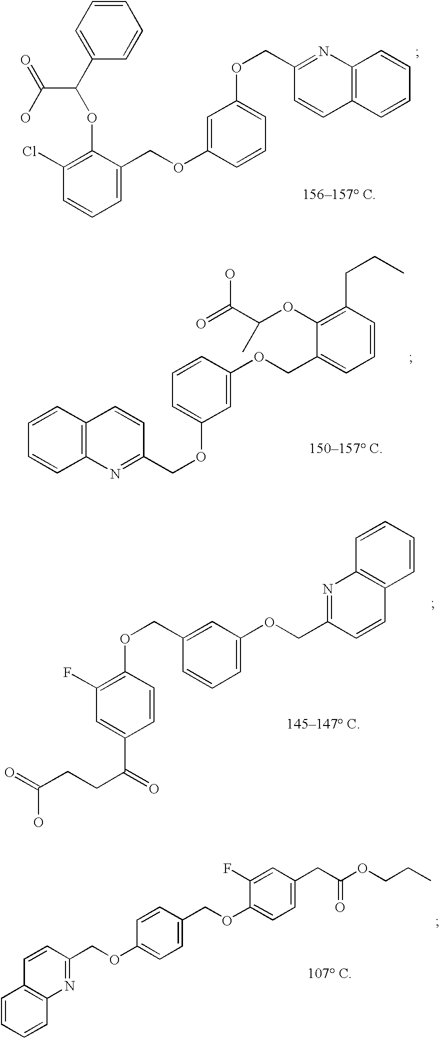

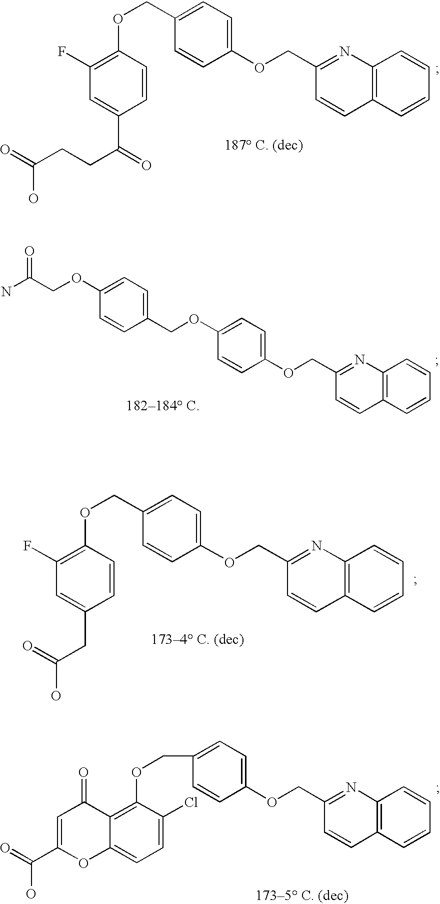

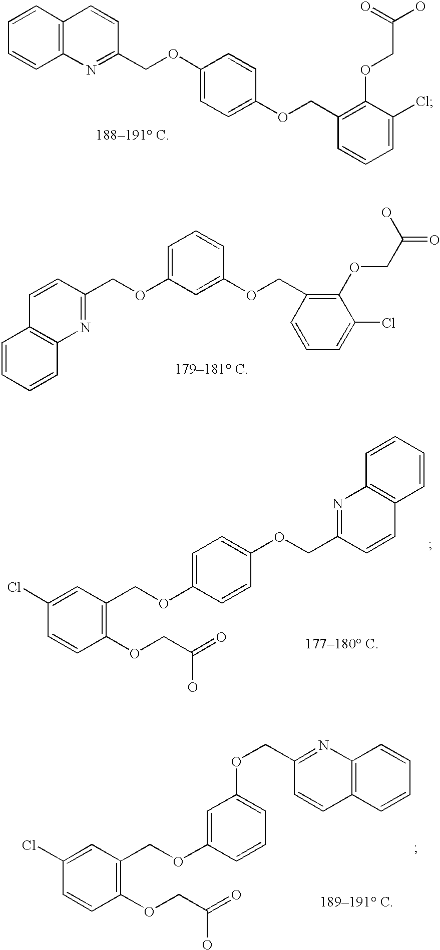

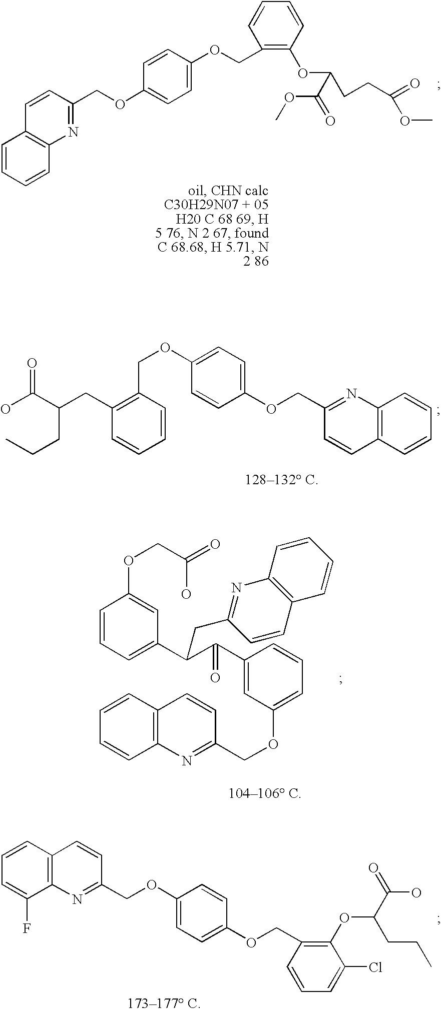

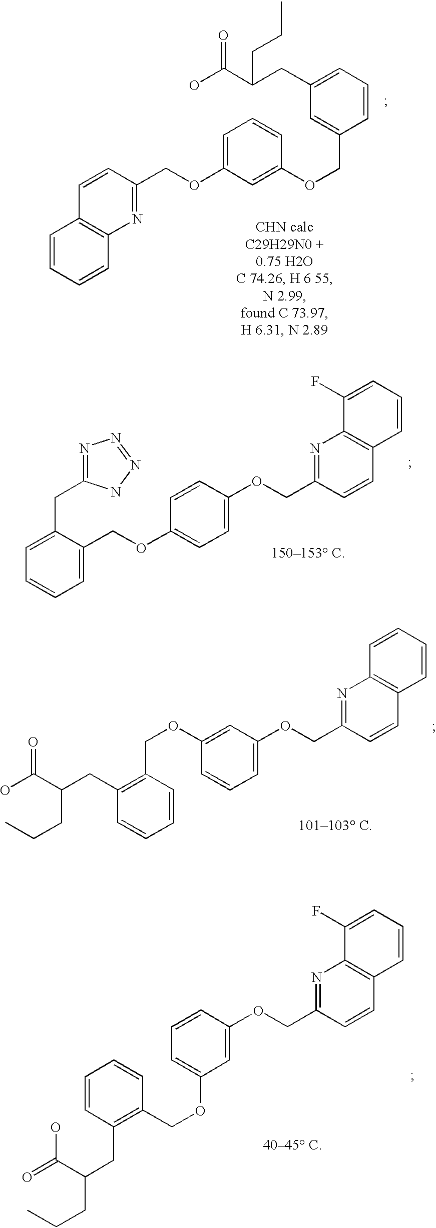

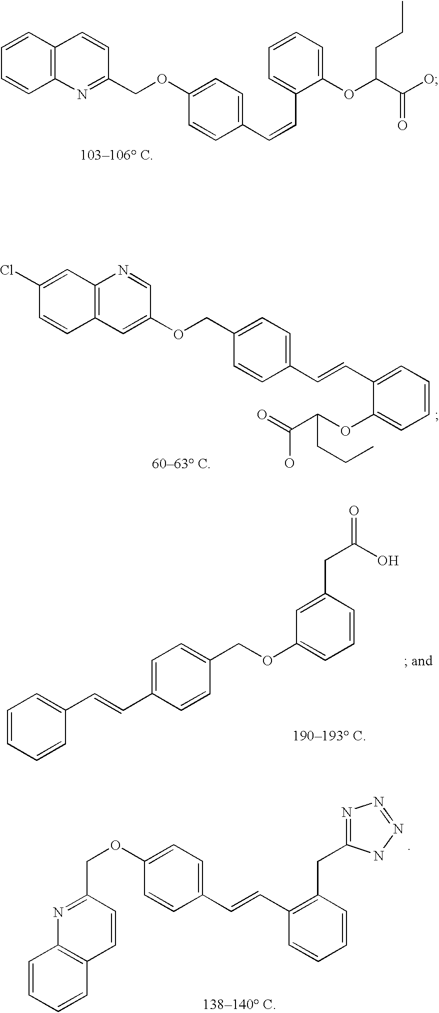

18. A method according to claim 1 or 9 wherein the PPAR mediator is selected from the group consisting of

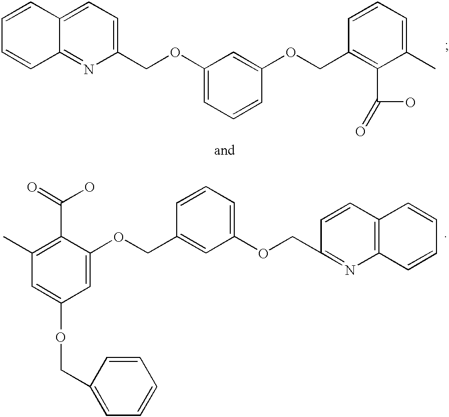

19. A method according to claim 1 or 9 wherein the PPAR mediator is [chemical structure]
